SEQUENCE LISTING (10 sequences)

SEQ ID NO: 1 - DNA, 41 bp, Artificial Sequence
  primer
gaagatctcaccatggaagcaaaactgtttgtattattctg

SEQ ID NO: 2 - DNA, 33 bp, Artificial Sequence
  primer
tccccgcggaatctggtaaactcccattgattc

SEQ ID NO: 3 - DNA, 1590 bp, Influenza virus
atggaagcaaaactgtttgtattattctgtgtattcaatgcgctgaaagctgacaccatttgtgtaggctaccatgctaacaattccacagacactgtcgacacaatactggagaaaaatgtgactgttacccattcagttaatttactagaaaacagccataatggaaaactctgcagcctgaatggaaaagcccccctacaactggggaactgcaacgtagcaggatggatccttggcaacccagaatgtgacttgttgctcacagcgaattcatggtcttacataatagagacttcaaattcaaaaaatggaaaatgctaccccggagaattcgctgattatgaggaattaagggagcagctgagtacagtttcttcatttgaaagatttgaaattttcccaaaagcaacctcatggccagatcatgagacaaccaaaggtaccacaactgcatgctcccactctggaaccagcagtttttaccggaacttgctatggatagtaaagaagggaaactcctatcctaagctcagcaagtcatacacaaacaacaaaggaaaagaagtgcttgtaatctggggagtgcaccaccctccgactaacagtgaccaacaaaccctctaccagaatgcttatacatatgtttcagttgaatcatcaaaatactaccgaaggttcacaccagaaatagcagctagacctaaagtcagaggacaagcaggcagaatgaattattattggacactgttagatcaaggagacaccataacatttgaagccactgggaacttaatagcaccatggtacgcatttgctttgaataagggctctaattctggaattatgatgtcggatgctcatgttcacaattgcactacaaagtgccaaactcctcatggggccttgaaaagtaatcttccttttcagaacgtacatcccatcactattggagaatgccctaaatatgttaaaagcacccaactaagaatggcaacaggattaagaaacgtcccctctatccaatccagaggactttttggagcaattgctgggttcattgaaggaggatggacaggaatgatagatggatggtatggatatcaccatcaaaatgagcagggatctggttacgcagcagatcagaaaagcacacaaattgcaattgatgggatcagcaacaaagtgaactcagtaattgaaaaaatgaacattcaatttacttcagtgggcaaggagttcaataatctggagaaaaggattgagaatttgaataagaaggtcgatgatgggtttttggatatatggacatataatgctgagttgctcattttgctcgagaatgaaaggactctagatttccatgactttaacgtaaaaaatttatatgaaaaggtcaaatcacaattgagaaacaatgccaaggaagtcggtaatggttgttttgagttctatcacaaatgtgataatgaatgcatggagagcgtaaagaatggcacatacaattatcccaaatattcagaagaatccaaattgaatagagaggaaatagacggtgtgaaattagaatcaatgggagtttaccagatt

SEQ ID NO: 4 - protein, 530 aa, Influenza virus
MEAKLFVLFCVFNALKADTICVGYHANNSTDTVDTILEKNVTVTHSVNLLENSHNGKLCSLNGKAPLQLGNCNVAGWILGNPECDLLLTANSWSYIIETSNSKNGKCYPGEFADYEELREQLSTVSSFERFEIFPKATSWPDHETTKGTTTACSHSGTSSFYRNLLWIVKKGNSYPKLSKSYTNNKGKEVLVIWGVHHPPTNSDQQTLYQNAYTYVSVESSKYYRRFTPEIAARPKVRGQAGRMNYYWTLLDQGDTITFEATGNLIAPWYAFALNKGSNSGIMMSDAHVHNCTTKCQTPHGALKSNLPFQNVHPITIGECPKYVKSTQLRMATGLRNVPSIQSRGLFGAIAGFIEGGWTGMIDGWYGYHHQNEQGSGYAADQKSTQIAIDGISNKVNSVIEKMNIQFTSVGKEFNNLEKRIENLNKKVDDGFLDIWTYNAELLILLENERTLDFHDFNVKNLYEKVKSQLRNNAKEVGNGCFEFYHKCDNECMESVKNGTYNYPKYSEESKLNREEIDGVKLESMGVYQI

SEQ ID NO: 5 - protein, 1730 aa, Sus scrofa
MDFLLLLLLLASSALAGLASWTVSSPETVQGIKGSCLIIPCTFGFPANVEVPHGITAIWYYDYSGKRLVVSHSRNPKVVENHFQGRALLLGQVEQRTCSLLLKDLQPQDSGSYNFRFEISEGNRWSDVKGTVVTVTEVPSVPTIALPAKLHEGMEVDFNCSTPYVCPTEPVNLQWQGQDPTRSVTSHLQKLEPSGTSHMETLHMALSWQDHGRILSCQVSAAERRMQKEIHLQVQYAPKGVEILFSHSGRNVLPGDLVTLSCQVNSSNPQVSSVQWVKDGTKLKDQKRVLQLRRAAWADAGVYTCQAGNAVGSSVSPPVSLHVFMAEVQVSPVGSILENQTVTLACNTPKEAPSELRYSWYKNHALLEGSHSRTLRLHSVTRADSGFYFCEVQNARGRERSPPVSVVVSHPPLTPDLTAFLETQAGLVGILQCSVVSEPPATLVLSHGGLILASTSGEGDHSPRFSVASAPNSLRLEIQDLGPTDSGEYMCSASSSLGNASSTLDFHANAARLLISPAAEVVEGQAVTLSCRSSLSLMPDTRFSWYLNGALILEGPSSSLLLPAASSTDAGSYHCRAQNSHSTSGPSSPAVLTVLYAPRQPVFTAQLDPDTAGAGAGRQGLLLCRVDSDPPAQLQLLHRGRVVASSLSWGGGCCTCGGCFHRMKVTKAPNLLRVEIRDPVLEDEGVYLCEASSALGNASASATLDAQATVLVITPSHTLQEGIEANLTCNVSREASGPANFSWFRDGALWAQGPLDTVTLLPVARTDAALYACRIVTEAGAGLSTPVALNVLYPPDPPKLSALLDVDQGHTAVFVCTVDSRPLAQLALFRGEHLLAASSALRLPPRGRLQAKASANSLQLEVRDLSLGDSGSYHCEATNILGSANTSLTFQVRGAWVRVSPSPELQEGQAVVLSCQVPIGVLEGTSYRWYRDGQPLQESTSATLRFAAITLSQAGAYHCQAQAPGSATTDLAAPVSLHVTYAPRQATLTTLMDSGLGRLGLLLCRVNSDPPAQLRLLHGSRLVASTLQGVEELAGSSPRLQVATAPNTLRLEIHNAVLEDEGVYTCEATNTLGQTLASAAFDAQAMRVQVWPNATVQEGQLVNLTCLVWTTHLAQLTYTWYRDQQQLPGAAHSILLPNVTVTDAASYRCGILIPGQALRLSRPVALDVLYAPRRLRLTHLLESRGGQLAVVLCTVDSRPAAQLTLSHAGRLLASSTAASVPNTLRLELWEPRPSDEGLYSCSARSPLGQANTSLELRLEGVQVALAPSATVPEGAPVTVTCEDPAARPPTLYVWYHNSRWLQEGSAASLSFPAATRAHAGAYTCQVQDAQGTRISQPAALHILYAPRDAVLSSFWDSRASPMAVVQCTVDSEPPAEMTLSHDGKVLATSHGVHGLAVGTGHVQVARNALQLRVQNVPSRDKDTYVCMDRNSLGSVSTMGQLQPEGVHVVAEPGLDVPEGTALNLSCRLPSGPGHIGNSTFAWFRNGRQLHTESVPTLTFTHVARAQAGLYHCQAELPAGAATSAPVLLRVLYPPKTPTMTVFVEPEGGIQGILDCRVDSEPLASLTLHLGSRLVASSQPQAAPAKPHIRVSASPNALRVDMEELKPSDQGEYVCSASNALGSASAATYFGTRALHRLHLFQHLLWFLGLLASLLFLLLGLGVWYAWRRGNFYKLRMGEYSVEMVSRKETTQMSTDQEEVTGIGDDAGSVNQAAFDPAHLCENTQSVKSTV

SEQ ID NO: 6 - DNA, 5193 bp, Sus scrofa
atggacttcctgctcctgctcctcctcctggcttcatctgctctagcaggcctggcctcgtggacggtttccagccccgagaccgtgcagggcatcaagggctcctgcctcatcatcccctgcaccttcggcttcccggccaacgtggaggtgccccatggcatcacagccatctggtactatgactactcaggcaagcgcctggtagtgagccactccaggaacccaaaggtggtggagaaccacttccaaggccgggccctgctgttggggcaggttgaacagaggacgtgcagcctgctgctgaaggacctgcagccccaggactcgggctcctataacttccgctttgagatcagcgagggcaaccgctggtcagatgtcaaaggcacagttgtcaccgtgacagaggtgcccagcgtgcccaccattgccttgccagccaagctgcatgagggcatggaggtggacttcaactgctccactccctatgtgtgcccgacggagccggtcaacctacagtggcaaggccaggatcccacccgctccgtcacctcccacctccagaagcttgagccctcgggcaccagccacatggagaccctgcacatggccctgtcctggcaggaccatggccggatcctgagctgccaggtctcagcagccgaacgcaggatgcagaaggagattcacctccaagtgcagtatgcccccaagggtgtggagatccttttcagccactccggacggaacgtccttccaggtgatctggtcaccctcagctgccaggtgaatagcagcaaccctcaggtcagttccgtgcagtgggtcaaggatgggacgaagctcaaagaccagaaacgtgtactgcagttgcgccgggcagcctgggctgatgctggcgtctacacctgccaagccgggaatgccgtgggctcttcagtctcacccccggtcagcctccacgtcttcatggctgaggtccaggtaagccctgtgggctccatcctggagaaccagacggtgacgctggcctgcaatacacctaaggaagcgcccagcgagctgcgctacagctggtacaagaaccacgccctgctggagggctctcacagccgcaccctccggctgcactcagttaccagggcggattcgggcttctacttctgcgaggtgcagaacgcccggggcagagagcgctctccccctgtcagcgtggtggtcagccacccacccctcaccccggacctaactgccttcctggagacacaggcggggctggtgggcatcctccaatgctctgtggtcagcgagcccccagctactctggtgttgtcacacgggggcctcatcttggcctctacctccggggagggtgaccacagcccacgcttcagtgtcgcctctgcccccaactccctgcgcctggagattcaagacctggggccaacagacagtggggaatacatgtgctcagccagcagttctcttgggaatgcgtcctccaccctggacttccatgccaatgcagcccgcctcctcatcagcccagcagcagaggtggtggaagggcaggcggtgacactgagctgcaggagcagcctgagcctgatgcctgacacccgtttttcctggtacctgaacggggccctgattctcgaggggcccagcagcagcctcctgctcccagcagcctccagcacagatgccggctcataccactgccgggcccagaacagccacagcaccagcgggccctcctcacctgctgttctcaccgtgctctacgccccacgccagcccgtgttcactgcccagctggaccctgatactgcaggagctggggccggacgccaaggcctcctcttgtgccgtgtggacagcgaccccccagcccagctgcagctgctccacaggggccgtgttgtggcctcttctctgtcatgggggggcggctgctgcacctgcggaggctgtttccaccgcatgaaggtcaccaaagcacccaacctactgcgtgtagagatccgagacccggtgctggaggatgagggtgtgtacctgtgcgaggccagcagcgccctgggcaacgcctccgcctctgcaaccttggatgcccaggccactgtcctggtcatcacaccgtcacacacgctgcaggaaggcattgaagccaacctgacttgcaacgtgagccgtgaagccagcggccctgccaacttctcctggttccgagatggggcgctatgggcccagggccctctggacaccgtgacgctgctacctgtggccagaactgatgctgccctctatgcttgccgcatcgtcaccgaggctggtgctggcctctccacccctgtggccctgaatgtgctctatccccccgatcctccaaagttgtcagccctcctggacgtggaccagggccacacggctgtgttcgtctgtactgtggacagtcgccctcttgcccagttggccctgttccgtggggaacacctcctggccgccagctcggcactccggctcccccctcgtggccgcctccaggccaaagcctcggccaactccttgcagctagaggtccgagacttgagccttggggactctggcagctaccactgtgaggccaccaacatccttggatcagccaacacttctcttaccttccaggtccgaggagcctgggtccgggtgtcaccgtcgcctgagctccaggagggccaggctgtggtcctgagctgccaggtacccataggggtcctggaggggacctcatatcgttggtatcgggatggccagcccctccaggagtccacttcggccacgctccgttttgcagccataactctgagccaggctggagcctaccattgccaagcccaagctccaggctcagccaccacggacctggctgcccctgtcagcctccacgtgacctacgcacctcgccaggccacactcaccaccctgatggactcaggcctcgggcgactgggcctccttctgtgccgtgtgaacagtgaccctcctgcccagctccgactgctccatgggagccgcctcgtggcctctactctacaaggtgtggaggagcttgcaggcagctctccccgcctacaggtggccacagcccccaacacgctgcgcctggagatccacaacgcagtgctggaggatgaaggcgtctacacctgcgaggccaccaacaccctgggtcagaccttggcctccgccgccttcgatgcccaggctatgagagtgcaggtgtggcccaatgccaccgtgcaagaggggcagctggtgaacctgacctgccttgtatggaccacgcacctggcccagctcacctacacgtggtaccgagaccagcagcagctcccaggtgctgcccactccatcctcctgcccaatgtcactgtcacagatgccgcctcctaccgctgtggcatattgatccctggccaggcactccgcctctccagacctgtcgccctggatgtcctctacgcaccccgcagactgcgcctgacccatctcttggagagccgtggtgggcagctggccgtggtgctgtgcactgtggacagtcgcccagctgcccagctgaccctcagccatgctggccgcctcctggcctcctcaaccgcagcctctgtccccaacaccctgcgcctggagctgtgggagccccggcccagtgatgagggtctctacagctgctcggcccgcagtcctctgggccaggccaacacatccctggagctgcggctagagggcgtgcaggtggcactggctccatcggccactgtgccggagggggcccctgtcacagtgacctgtgaagaccctgctgcccgcccacccactctctatgtctggtaccacaacagccgttggctgcaggaggggtcggctgcctccctctcgtttccagcggctacacgggctcacgcgggcgcctatacctgccaggtccaggatgcccagggcacacgcatctcccagcccgcagcactgcacatcctctatgcccctcgggatgctgtcctttcctccttctgggactcaagggccagccctatggccgtggtacagtgcactgtggacagcgagccacctgccgagatgaccctgtcccatgatggcaaggtgctggccaccagccatggggtccacggcttagcagtggggacaggccatgtccaggtggcccgcaacgccctgcagctgcgggtgcagaatgtgccctcacgtgacaaggacacctacgtctgcatggaccgcaactccttgggctcagtcagcaccatggggcagctgcagccagaaggtgtgcacgtggtagctgagccagggctggatgtgcctgaaggcacagcgctgaacctgagctgtcgcctccctagtggccctgggcacataggcaactccacctttgcttggttccggaacggtcggcagctacacacagagtctgtgcccacccttaccttcacccatgtggcccgcgcccaagctggcttgtaccactgccaggctgagctccccgccggggctgccacctctgctccagtcttgctccgggtgctctaccctcccaagacgcccaccatgactgtttttgtggagcccgagggtggcatccagggcattctggactgccgagtggacagtgagcccctagccagcctgaccctccacctgggcagtcggctggtggcctccagccagcctcaggctgcccctgccaagccgcacatccgcgtctcagccagtcccaatgccttgcgagtggacatggaggagctgaagcccagtgaccagggggagtatgtgtgctcggcctccaatgccctgggctctgcctctgctgccacctacttcggaaccagagccctgcatcgcctgcatctgttccagcaccttctctggttcctggggctgctggcgagcctcctcttcctactgttgggcctgggggtctggtacgcctggagacggggaaatttttacaagctgagaatgggcgaatattcagtagagatggtatctcggaaggaaaccacgcagatgtccactgaccaggaagaagttactggaatcggtgatgatgcgggctctgtgaaccaggcggcatttgatcctgcccacctctgtgaaaacacacagtctgtgaaaagcacagtctga

SEQ ID NO: 7 - protein, 1695 aa, Mus musculus
MCVLFSLLLLASVFSLGQTTWGVSSPKNVQGLSGSCLLIPCIFSYPADVPVSNGITAIWYYDYSGKRQVVIHSGDPKLVDKRFRGRAELMGNMDHKVCNLLLKDLKPEDSGTYNFRFEISDSNRWLDVKGTTVTVTTDPSPPTITIPEELREGMERNFNCSTPYLCLQEKQVSLQWRGQDPTHSVTSSFQSLEPTGVYHQTTLHMALSWQDHGRTLLCQFSLGAHSSRKEVYLQVPHAPKGVEILLSSSGRNILPGDPVTLTCRVNSSYPAVSAVQWARDGVNLGVTGHVLRLFSAAWNDSGAYTCQATNDMGSLVSSPLSLHVFMAEVKMNPAGPVLENETVTLLCSTPKEAPQELRYSWYKNHILLEDAHASTLHLPAVTRADTGFYFCEVQNAQGSERSSPLSVVVRYPPLTPDLTTFLETQAGLVGILHCSVVSEPLATVVLSHGGLTLASNSGENDFNPRFRISSAPNSLRLEIRDLQPADSGEYTCLAVNSLGNSTSSLDFYANVARLLINPSAEVVEGQAVTLSCRSGLSPAPDTRFSWYLNGALLLEGSSSSLLLPAASSTDAGSYYCRTQAGPNTSGPSLPTVLTVFYPPRKPTFTARLDLDTSGVGDGRRGILLCHVDSDPPAQLRLLHKGHVVATSLPSRCGSCSQRTKVSRTSNSLHVEIQKPVLEDEGVYLCEASNTLGNSSAAASFNAKATVLVITPSNTLREGTEANLTCNVNQEVAVSPANFSWFRNGVLWTQGSLETVRLQPVARTDAAVYACRLLTEDGAQLSAPVVLSVLYAPDPPKLSALLDVGQGHMAVFICTVDSYPLAHLSLFRGDHLLATNLEPQRPSHGRIQAKATANSLQLEVRELGLVDSGNYHCEATNILGSANSSLFFQVRGAWVQVSPSPELREGQAVVLSCQVPTGVSEGTSYSWYQDGRPLQESTSSTLRIAAISLRQAGAYHCQAQAPDTAIASLAAPVSLHVSYTPRHVTLSALLSTDPERLGHLVCSVQSDPPAQLQLFHRNRLVASTLQGADELAGSNPRLHVTVLPNELRLQIHFPELEDDGTYTCEASNTLGQASAAADFDAQAVRVTVWPNATVQEGQQVNLTCLVWSTHQDSLSYTWYKGGQQLLGARSITLPSVKVLDATSYRCGVGLPGHAPHLSRPVTLDVLHAPRNLRLTYLLETQGRQLALVLCTVDSRPPAQLTLSHGDQLVASSTEASVPNTLRLELQDPRPSNEGLYSCSAHSPLGKANTSLELLLEGVRVKMNPSGSVPEGEPVTVTCEDPAALSSALYAWFHNGHWLQEGPASSLQFLVTTRAHAGAYFCQVHDTQGTRSSRPASLQILYAPRDAVLSSFRDSRTRLMVVIQCTVDSEPPAEMVLSHNGKVLAASHERHSSASGIGHIQVARNALRLQVQDVTLGDGNTYVCTAQNTLGSISTTQRLLTETDIRVTAEPGLDVPEGTALNLSCLLPGGSGPTGNSSFTWFWNRHRLHSAPVPTLSFTPVVRAQAGLYHCRADLPTGATTSAPVMLRVLYPPKTPTLIVFVEPQGGHQGILDCRVDSEPLAILTLHRGSQLVASNQLHDAPTKPHIRVTAPPNALRVDIEELGPSNQGEYVCTASNTLGSASASAYFGTRALHQLQLFQRLLWVLGFLAGFLCLLLGLVAYHTWRKKSSTKLNEDENSAEMATKKNTIQEEVVAAL

SEQ ID NO: 8 - DNA, 6387 bp, Mus musculus
agacaagattaggcctagagtaagtctatgaaacacagagaaaggggacagcataggggttaagaaatgaggtctttcaaaatctcagggggcaatgaggagttttttgagagaggaaggactctttaaaggaagttgaaggaggattctgtgaacttgagaccaccctgagctgccaagttgagaactttgtctacaaacaagccaggcagcctcagcgtgtgctcagtccgacttgtagctggagaggcaggagaccaatttccggtgcttacggtgcttgctggatgccctggagtaagtgacagggtctcactggactccaggttctgttggtttgagtaataggaggcggcaggggagaagtgaagagagacatgcactgctgatctgccttgaggctgtgtccttaaggggtggagccaaggggcacagaagactctctgggacatgccaccaagtgagagcatttccaatcactccctgagccaggaacaggggcttctggttccctgctggtggctgccacagcagtccttcctgttgggttgaccaacacagcaggtgagataaaccctatagacttgggccctggagtgctccaggcagtctctgtgtgcctacccacccggcttccctaggcacctgaatgcacctgggcactgggatgtgtgtcctgttctccctgctcctgctggcctctgtcttctcactaggccagaccacatggggtgtctccagtcccaagaatgtgcagggcttgtcgggatcctgcctgctcattccctgcatcttcagctaccctgccgatgtcccagtgtccaatggcatcacagccatctggtactatgactactcgggcaagcggcaggtggtaatccactcaggggaccccaagctggtggacaagcgtttcaggggtcgagctgaactgatggggaacatggaccacaaggtgtgcaacctgttgctcaaagacttgaagcctgaagactctggcacctacaacttccgctttgagatcagtgatagcaaccgctggttagatgtcaaaggcaccacggtcactgtgacaacggatcccagcccccccactattaccattcctgaggagctgcgtgaaggcatggagaggaacttcaactgttccacaccctacctgtgcctgcaggagaagcaagtcagcctgcagtggcgaggccaggaccccacccactctgtcacctccagcttccagagcctcgagcccactggcgtctatcaccagacgaccctacatatggccctatcctggcaggaccacggtcggaccctgctctgccagttctcattgggcgcacacagtagtcggaaagaggtttacctgcaagtgccacatgcccccaaaggtgtggagatcctcctcagctcctcagggaggaacatccttcccggggatccagtcacactcacctgcagagtgaacagcagctatcctgctgtcagtgccgtgcagtgggccagggacggagtgaacctcggagtcacgggacatgtgcttcggctgttctcagcagcctggaatgattctggggcctacacctgccaagcaacaaatgatatgggctctctggtgtcatccccgctcagcctccatgtttttatggctgaagtcaaaatgaaccccgcagggcccgtcttggaaaatgagacagtgactctgctctgtagcacgccgaaggaggctccccaggagctccgctatagctggtacaagaaccacattctcctggaagatgcccatgcctcaaccttgcacctgcctgcagtcaccagggctgatactggcttctacttctgtgaagtgcagaatgcccagggcagtgagcgctccagtccattgagtgtggtggtcagatatccaccccttactccagacctgaccaccttcctggagacacaggccggacttgtgggcatcttgcattgctccgtggtcagtgagcccctggctactgtggtgctgtcacacggaggcctcacgttggcctccaactctggagaaaatgacttcaacccccgattcaggatctcctctgcccccaactccctgcgcctagaaatccgagacttgcagccagcagacagcggagagtacacatgcttagctgtcaactcccttggaaactcaacgtccagcctagacttctatgctaatgtggcccgactcctcatcaacccttcagcagaggttgtggaagggcaggcggtgaccctgagctgcaggagtggcctgagcccagctcctgacactcgcttctcctggtacctgaacggagctctacttctggaaggatccagcagcagcctcctgcttcctgcggcttccagcactgatgcgggctcatactactgtaggacgcaggctggccccaacaccagcggcccctccctgcctactgtcctcactgtgttctatcccccaagaaagcccacattcactgccaggctggatttggatacctctggagtcggggatggacgacggggcatcctcttgtgccacgtagacagcgatcccccagcccagctacggcttctccacaaaggccatgttgtggccacttctctgccatcaaggtgtgggagctgttcccagcgcacaaaagtcagcagaacctccaactcactgcacgtggagatccagaagcctgtattagaggatgagggcgtgtacctgtgtgaggctagcaacacattgggcaactcctcagccgcagcctctttcaatgctaaggccactgtactggtcatcacaccgtcaaatacactgcgtgaaggcacagaggccaacctaacttgcaacgtgaaccaggaggttgctgtcagccctgccaacttctcctggttccggaatggagtgctgtggacccagggatcactggagactgtgaggctgcagcctgtggccagaactgatgctgctgtctatgcctgccgcctcctcaccgaggatggggctcagctctcggctcctgtggtcctaagtgtgctgtatgccccagaccctccaaagctgtcagccctcctagatgtgggtcagggccacatggccgtgttcatctgcactgtggacagctatcccctggctcacctgtctctgttccgtggggaccatctcctggccaccaacttggaaccccagcgtccctcccatggcaggatccaggccaaggccacagccaactccctgcagctagaggtccgagaactaggtcttgtggactctggaaactaccactgtgaagccaccaatattcttgggtcagccaacagttcactcttcttccaggtcagaggagcctgggtccaggtttcaccatcacctgagctccgggagggccaggctgtggtcctgagctgccaggtgcccacaggagtctctgaggggacctcatacagctggtatcaggatggccgccccctccaggagtcaacctcatctacactccgcattgcagccataagtctgaggcaagctggtgcctaccattgccaagctcaggccccagacacagctattgccagcctggctgcccctgtcagcctccatgtgtcctataccccacgtcatgttacactcagtgccctgctgagcacggaccctgagcgactaggccacctggtgtgcagtgtacaaagtgaccctccagcgcagctgcaactgtttcaccggaatcgcctcgtggcctctaccctacaaggcgcggacgaattggcaggcagtaatccccggctgcatgtgactgtgctccccaatgagctgcgcctgcagatccactttccagagctggaggatgacgggacctatacatgcgaagccagcaacacactgggccaggcctcggctgcagctgacttcgatgcccaggctgtgcgagtgactgtgtggcccaatgccactgtgcaagaggggcagcaggtgaacctgacctgcttggtgtggagcacccaccaggactcactcagctacacatggtacaagggcgggcaacaactccttggtgccagatccatcaccctgcccagtgttaaggttttggatgctacctcctaccgctgtggtgtggggctccccggccacgcaccccatctctccagacccgtgaccctggatgtcctccatgctccccgaaacctgcggctgacctacctcctagagacccagggcaggcagctggccctggtactgtgtacggtggatagtcgtccacctgcccagctaactctcagccatggtgaccagcttgtagcctcctcaactgaagcctctgtccccaacaccctgcgcctagagcttcaggatccaaggcctagtaatgaggggctctatagctgctctgcccacagcccattgggcaaggccaacacgtccctggaacttctgctggaaggtgtccgagtgaaaatgaatccctctggtagtgtacccgagggagagcctgtcacagtgacctgcgaggaccctgctgccctctcatccgccctctatgcctggtttcacaatggccattggcttcaggagggaccggcttcctcactccagttcctggtgactacacgggctcacgctggtgcttacttttgccaggtgcatgatacacaaggcacacggagctccagacctgccagcctgcaaattctctatgccccccgggatgctgtcctgtcttcctttcgagactcaaggaccaggctcatggtcgtgattcagtgcaccgtggacagtgagccacctgctgagatggtcctatcccacaatggcaaggtgctagctgccagccacgagcgtcacagctcagcatcagggataggccacatccaggtagcccgaaatgctcttcgactacaagtgcaagatgtgactctgggtgatggcaacacctatgtttgcacagcccagaatacactgggctccatcagtaccacccagaggcttctgacggagactgatatacgtgtgacagctgagccaggcttggatgtgccagagggcacagctctgaacttaagctgcctcctccctggtggctctgggcccacgggcaactcttccttcacgtggttctggaatcgccaccgactacattcagctcctgtgcccacactctccttcacccctgtggtccgggctcaggctgggctgtaccactgcagggctgatctccccaccggggccactacctctgctccagttatgctccgtgtcctctatccccccaagacgcccactctcatagtgtttgtggagcctcagggtggccaccagggcatcctcgactgtcgagtggacagtgagcccctggccatcctcactcttcaccggggcagtcaactagtagcctccaaccaacttcacgatgctcccaccaagccccacatccgagtcactgctcctcccaatgccttgagagtggacatagaggagctcggccctagcaatcaaggggagtatgtgtgcactgcctccaacactctgggctctgcctcagcctctgcctactttgggaccagagctctgcaccaactgcagctgttccagaggctgctctgggtcctgggatttctggcaggcttcctgtgcctgctgctgggtctggtggcctatcacacctggagaaagaagagttctaccaagctgaatgaggatgagaattcagcagagatggccactaagaaaaatactatccaggaggaagtggttgctgctctctgacaactcaggtgctgtgaacaagatcctgcctacctctgtataagcagtacagagacatctggctttcctgacctgcccgacttgccttccaagcctcttgatcctaagaaaaatggacgaagggaggtttggggttggaggtcaacctgccgcctccagggctctgagacggactcagccatgttgcccacgtctctctgtgtggttttcctctgtatccctttgcctttctcttcaaagctcaccttggactttcttggtgggttagagcaacatccagtttctcacagactttctaagacggtctgtaccagccaggatatcagtcaggttgctctaacagagactcaatacagtgaccacagcatgacagggtcttagttttccctcctggcctggttatgttgttgtggtatcagaatccttcttgcttgattttctccattccccaagtgttgcctttgattatgaagctcaggtaactgcagtgcccatggaccctacagggagaaggaagagtgaagggaagacatacccatccccatggtccatggactgtgtgtgcaattgcaccccacccaacttctcatccgctagaaactggtcacataaacataccatgctgaaaggga

SEQ ID NO: 9 - protein, 1709 aa, Homo sapiens
MGFLPKLLLLASFFPAGQASWGVSSPQDVQGVKGSCLLIPCIFSFPADVEVPDGITAIWYYDYSGQRQVVSHSADPKLVEARFRGRTEFMGNPEHRVCNLLLKDLQPEDSGSYNFRFEISEVNRWSDVKGTLVTVTEEPRVPTIASPVELLEGTEVDFNCSTPYVCLQEQVRLQWQGQDPARSVTFNSQKFEPTGVGHLETLHMAMSWQDHGRILRCQLSVANHRAQSEIHLQVKYAPKGVKILLSPSGRNILPGELVTLTCQVNSSYPAVSSIKWLKDGVRLQTKTGVLHLPQAAWSDAGVYTCQAENGVGSLVSPPISLHIFMAEVQVSPAGPILENQTVTLVCNTPNEAPSDLRYSWYKNHVLLEDAHSHTLRLHLATRADTGFYFCEVQNVHGSERSGPVSVVVNHPPLTPVLTAFLETQAGLVGILHCSVVSEPLATLVLSHGGHILASTSGDSDHSPRFSGTSGPNSLRLEIRDLEETDSGEYKCSATNSLGNATSTLDFHANAARLLISPAAEVVEGQAVTLSCRSGLSPTPDARFSWYLNGALLHEGPGSSLLLPAASSTDAGSYHCRARDGHSASGPSSPAVLTVLYPPRQPTFTTRLDLDAAGAGAGRRGLLLCRVDSDPPARLQLLHKDRVVATSLPSGGGCSTCGGCSPRMKVTKAPNLLRVEIHNPLLEEEGLYLCEASNALGNASTSATFNGQATVLAIAPSHTLQEGTEANLTCNVSREAAGSPANFSWFRNGVLWAQGPLETVTLLPVARTDAALYACRILTEAGAQLSTPVLLSVLYPPDRPKLSALLDMGQGHMALFICTVDSRPLALLALFHGEHLLATSLGPQVPSHGRFQAKAEANSLKLEVRELGLGDSGSYRCEATNVLGSSNTSLFFQVRGAWVQVSPSPELQEGQAVVLSCQVHTGVPEGTSYRWYRDGQPLQESTSATLRFAAITLTQAGAYHCQAQAPGSATTSLAAPISLHVSYAPRHVTLTTLMDTGPGRLGLLLCRVDSDPPAQLRLLHGDRLVASTLQGVGGPEGSSPRLHVAVAPNTLRLEIHGAMLEDEGVYICEASNTLGQASASADFDAQAVNVQVWPGATVREGQLVNLTCLVWTTHPAQLTYTWYQDGQQRLDAHSIPLPNVTVRDATSYRCGVGPPGRAPRLSRPITLDVLYAPRNLRLTYLLESHGGQLALVLCTVDSRPPAQLALSHAGRLLASSTAASVPNTLRLELRGPQPRDEGFYSCSARSPLGQANTSLELRLEGVRVILAPEAAVPEGAPITVTCADPAAHAPTLYTWYHNGRWLQEGPAASLSFLVATRAHAGAYSCQAQDAQGTRSSRPAALQVLYAPQDAVLSSFRDSRARSMAVIQCTVDSEPPAELALSHDGKVLATSSGVHSLASGTGHVQVARNALRLQVQDVPAGDDTYVCTAQNLLGSISTIGRLQVEGARVVAEPGLDVPEGAALNLSCRLLGGPGPVGNSTFAWFWNDRRLHAEPVPTLAFTHVARAQAGMYHCLAELPTGAAASAPVMLRVLYPPKTPTMMVFVEPEGGLRGILDCRVDSEPLASLTLHLGSRLVASSQPQGAPAEPHIHVLASPNALRVDIEALRPSDQGEYICSASNVLGSASTSTYFGVRALHRLHQFQQLLWVLGLLVGLLLLLLGLGACYTWRRRRVCKQSMGENSVEMAFQKETTQLIDPDAATCETSTCAPPLG

SEQ ID NO: 10 - DNA, 6736 bp, Homo sapiens
atgggcttcttgcccaagcttctcctcctggcctcattcttcccagcaggccaggcctcatggggcgtctccagtccccaggacgtgcagggtgtgaaggggtcttgcctgcttatcccctgcatcttcagcttccctgccgacgtggaggtgcccgacggcatcacggccatctggtactacgactactcgggccagcggcaggtggtgagccactcggcggaccccaagctggtggaggcccgcttccgcggccgcaccgagttcatggggaaccccgagcacagggtgtgcaacctgctgctgaaggacctgcagcccgaggactctggttcctacaacttccgcttcgagatcagtgaggtcaaccgctggtcagatgtgaaaggcaccttggtcacagtaacagaggagcccagggtgcccaccattgcctccccggtggagcttctcgagggcacagaggtggacttcaactgctccactccctacgtatgcctgcaggagcaggtcagactgcagtggcaaggccaggaccctgctcgctctgtcaccttcaacagccagaagtttgagcccaccggcgtcggccacctggagaccctccacatggccatgtcctggcaggaccacggccggatcctgcgctgccagctctccgtggccaatcacagggctcagagcgagattcacctccaagtgaagtatgcccccaagggtgtgaagatcctcctcagcccctcggggaggaacatccttccaggtgagctggtcacactcacctgccaggtgaacagcagctaccctgcagtcagttccattaagtggctcaaggatggggtacgcctccaaaccaagactggtgtgctgcacctgccccaggcagcctggagcgatgctggcgtctacacctgccaagctgagaacggcgtgggctctttggtctcaccccccatcagcctccacatcttcatggctgaggtccaggtgagcccagcaggtcccatcctggagaaccagacagtgacactagtctgcaacacacccaatgaggcacccagtgatctccgctacagctggtacaagaaccatgtcctgctggaggatgcccactcccataccctccggctgcacttggccactagggctgatactggcttctacttctgtgaggtgcagaacgtccatggcagcgagcgctcgggccctgtcagcgtggtagtcaaccacccgcctctcactccagtcctgacagccttcctggagacccaggcgggacttgtgggcatccttcactgctctgtggtcagtgagcccctggccacactggtgctgtcacatgggggtcatatcctggcctccacctccggggacagtgatcacagcccacgcttcagtggtacctctggtcccaactccctgcgcctggagatccgagacctggaggaaactgacagtggggagtacaagtgctcagccaccaactcccttggaaatgcaacctccaccctggacttccatgccaatgccgcccgtctcctcatcagcccggcagccgaggtggtggaaggacaggcagtgacactgagctgcagaagcggcctaagccccacacctgatgcccgcttctcctggtacctgaatggagccctgcttcacgagggtcccggcagcagcctcctgctccccgcggcctccagcactgacgccggctcataccactgccgggcccgggacggccacagtgccagtggcccctcttcgccagctgttctcactgtgctctacccccctcgacaaccaacattcaccaccaggctggaccttgatgccgctggggccggggctggacggcgaggcctccttttgtgccgtgtggacagcgacccccccgccaggctgcagctgctccacaaggaccgtgttgtggccacttccctgccatcagggggtggctgcagcacctgtgggggctgttccccacgcatgaaggtcaccaaagcccccaacttgctgcgtgtggagattcacaaccctttgctggaagaggagggcttgtacctctgtgaggccagcaatgccctgggcaacgcctccacctcagccaccttcaatggccaggccactgtcctggccattgcaccatcacacacacttcaggagggcacagaagccaacttgacttgcaacgtgagccgggaagctgctggcagccctgctaacttctcctggttccgaaatggggtgctgtgggcccagggtcccctggagaccgtgacactgctgcccgtggccagaactgatgctgccctttacgcctgccgcatcctgactgaggctggtgcccagctctccactcccgtgctcctgagtgtactctatcccccggaccgtccaaagctgtcagccctcctagacatgggccagggccacatggctctgttcatctgcactgtggacagccgccccctggccttgctggccttgttccatggggagcacctcctggccaccagcctgggtccccaggtcccatcccatggtcggttccaggctaaagctgaggccaactccctgaagttagaggtccgagaactgggccttggggactctggcagctaccgctgtgaggccacaaatgttcttggatcatccaacacctcactcttcttccaggtccgaggagcctgggtccaggtgtcaccatcacctgagctccaagagggccaggctgtggtcctgagctgccaggtacacacaggagtcccagaggggacctcatatcgttggtatcgggatggccagcccctccaggagtcgacctcggccacgctccgctttgcagccataactttgacacaagctggggcctatcattgccaagcccaggccccaggctcagccaccacgagcctagctgcacccatcagcctccacgtgtcctatgccccacgccacgtcacactcactaccctgatggacacaggccctggacgactgggcctcctcctgtgccgtgtggacagtgaccctccggcccagctgcggctgctccacggggatcgccttgtggcctccaccctacaaggtgtggggggacccgaaggcagctctcccaggctgcatgtggctgtggcccccaacacactgcgtctggagatccacggggctatgctggaggatgagggtgtctatatctgtgaggcctccaacaccctgggccaggcctcggcctcagctgacttcgacgctcaagctgtgaatgtgcaggtgtggcccggggctaccgtgcgggaggggcagctggtgaacctgacctgccttgtgtggaccactcacccggcccagctcacctacacatggtaccaggatgggcagcagcgcctggatgcccactccatccccctgcccaacgtcacagtcagggatgccacctcctaccgctgcggtgtgggcccccctggtcgggcaccccgcctctccagacctatcaccttggacgtcctctacgcgccccgcaacctgcgcctgacctacctcctggagagccatggcgggcagctggccctggtactgtgcactgtggacagccgcccgcccgcccagctggccctcagccacgccggtcgcctcttggcctcctcgacagcagcctctgtccccaacaccctgcgcctggagctgcgagggccacagcccagggatgagggtttctacagctgctctgcccgcagccctctgggccaggccaacacgtccctggagctgcggctggagggtgtgcgggtgatcctggctccggaggctgccgtgcctgaaggtgcccccatcacagtgacctgtgcggaccctgctgcccacgcacccacactctatacttggtaccacaacggtcgttggctgcaggagggtccagctgcctcactctcattcctggtggccacgcgggctcatgcaggcgcctactcttgccaggcccaggatgcccagggcacccgcagctcccgtcctgctgccctgcaagtcctctatgcccctcaggacgctgtcctgtcctccttccgggactccagggccagatccatggctgtgatacagtgcactgtggacagtgagccacctgctgagctggccctatctcatgatggcaaggtgctggccacgagcagcggggtccacagcttggcatcagggacaggccatgtccaggtggcccgaaacgccctacggctgcaggtgcaagatgtgcctgcaggtgatgacacctatgtttgcacagcccaaaacttgctgggctcaatcagcaccatcgggcggttgcaggtagaaggtgcacgcgtggtggcagagcctggcctggacgtgcctgagggcgctgccctgaacctcagctgccgcctcctgggtggccctgggcctgtgggcaactccacctttgcatggttctggaatgaccggcggctgcacgcggagcctgtgcccactctcgccttcacccacgtggctcgtgctcaagctgggatgtaccactgcctggctgagctccccactggggctgctgcctctgctccagtcatgctccgtgtgctctaccctcccaagacgcccaccatgatggtcttcgtggagcctgagggtggcctccggggcatcctggattgccgagtggacagcgagccgctcgccagcctgactctccaccttggcagtcgactggtggcctccagtcagccccagggtgctcctgcagagccacacatccatgtcctggcttcccccaatgccctgagggtggacatcgaggcgctgaggcccagcgaccaaggggaatacatctgttctgcctcaaatgtcctgggctctgcctctacctccacctactttggggtcagagccctgcaccgcctgcatcagttccagcagctgctctgggtcctgggactgctggtgggcctcctgctcctgctgttgggcctgggggcctgctacacctggagaaggaggcgtgtttgtaagcagagcatgggcgagaattcggtggagatggcttttcagaaagagaccacgcagctcattgatcctgatgcagccacatgtgagacctcaacctgtgccccacccctgggctgaccagtggtgttgcctgccctccggaggagaaagtggccagaatctgtgatgactccagcctatgaatgtgaatgaggcagtgttgagtcctgcccgcctctacgaaaacagctctgtgacatctgactttttatgacctggccccaagcctcttgcccccccaaaaatgggtggtgagaggtctgcccaggagggtgttgaccctggaggacactgaagagcactgagctgatctcgctctctcttctctggatctcctcccttctctccatttctccctcaaaggaagccctgccctttcacatccttctcctcgaaagtcaccctggactttggttggattgcagcatcctgcatcctcagaggctcaccaaggcattctgtattcaacagagtatcagtcagcctgctctaacaagagaccaaatacagtgacttcaacatgatagaattttatttttctctcccacgctagtctggctgttacgatggtttatgatgttggggctcaggatccttctatcttccttttctctatccctaaaatgatgcctttgattgtgaggctcaccatggccccgctttgtccacatgccctccagccagaagaaggaagagtggaggtagaagcacacccatgcccatggtggacgcaactcagaagctgcacaggacttttccactcacttcccattggctggagtattgtcacatggctactgcaagctacaagggagactgggaaatgtagtttttattttgagtccagaggacatttggaattggacttccaaaggactcccaactgtgagctcatccctgagacttttgacattgttgggaatgccaccagcaggccatgttttgtctcagtgcccatctactgagggccagggtgtgcccctggccattctggttgtgggcttcctggaagaggtgatcactctcacactaagactgaggaaataaaaaaggtttggtgttttcctagggagagagcatgccaggcagtggagttgcctaagcagacatccttgtgccagatttggcccctgaaagaagagatgccctcattcccaccaccaccccccctacccccagggactgggtactaccttactggcccttacaagagtggagggcagacacagatgttgtcagcatccttattcctgctccagatgcatctctgttcatgactgtgtgagctcctgtccttttcctggagaccctgtgtcgggctgttaaagagaatgagttaccaagaaggaatgacgtgcccctgcgaatcagggaccaacaggagagagctcttgagtgggctagtgactccccctgcagcctggtggagatggtgtgaggagcgaagagccctctgctctaggatttgggttgaaaaacagagagagaagtggggagttgccacaggagctaacacgctgggaggcagttgggggcgggtgaactttgtgtagccgaggccgcaccctccctcattccaggctcattcattttcatgctccattgccagactcttgctgggagcccgtccagaatgtcctcccaataaaactccatcctatgacgcaaaaaaaaaaaaaaaaaa

(19) United States
(12) Patent Application Publication
(10) Pub. No.: US 2013/0273080 A1
(43) Pub. Date: Oct. 17, 2013

(54) SIALOADHESIN-RELATED COMPOSITIONS AND METHODS

(71) Applicant: Universiteit Gent, Gent (BE)

(72) Inventors: Dirk Elewaut, Gent (BE); Els Louagie, Torhout (BE); Peter Delputte, Kortrijk (BE)

(21) Appl. No.: 13/833,516

(22) Filed: Mar. 15, 2013

Related U.S. Application Data
(63) Continuation-in-part of application No. 13/066,341, filed on Apr. 11, 2011.
Application No. 13/066,341 is a division of application No. 12/227,106, filed on Nov. 7, 2008, now Pat. No. 7,998,485.
(60) Provisional application No. 60/799,566, filed on May 11, 2006.

(51) Int. Cl. A61K 47/48

(52) U.S. Cl. CPC A61K 47/48384
USPC 424/178.1; 435/375; 530/391.7

(57) ABSTRACT

Methods of delivering a cargo moiety to a cell is provided that include contacting a cell expressing sialoadhesin with a conjugate including a sialoadhesin binding moiety and a cargo moiety. The sialoadhesin binding moiety binds to the sialoadhesin expressed by the cell and is internalized along with the cargo, delivering the cargo moiety to the cell. Particular methods include induction or enhancement of sialoadhesin expression in a cell which naturally produces little or no sialoadhesin. Induction or enhancement of sialoadhesin expression includes transfection of a sialoadhesin expression construct and/or administration of an agent effective to induce or enhance sialoadhesin expression. Methods and compositions for stimulating an immune response in a subject are detailed. Particular methods and compositions for stimulating an immune response to a virus are provided herein.

### REFERENCE TO RELATED APPLICATIONS

This application is a continuation in part of co-pending U.S. Ser. No. 13/066,341, filed on Apr. 11, 2011, which is a divisional of co-pending U.S. patent application Ser. No. 12/227,106, filed Nov. 7, 2008, now U.S. Pat. No. 7,998,485 (issued Aug. 16, 2011), which is a national phase entry under 35 U.S.C. §371 of International Patent Application PCT/IB2007/004499, filed May 11, 2007, published in English as International Patent Publication WO 2008/093166 A2 on Aug. 7, 2008, which application claims the benefit under 35 U.S.C. §119(e) and under Article 8 of the PCT to U.S. Provisional Patent Application Ser. No. 60/799,566, filed May 11, 2006, the entire contents of each of which are incorporated herein by reference.

### STATEMENT ACCORDING TO 37 C.F.R. §1.821(c) or (e)—SEQUENCE LISTING SUBMITTED AS A TXT AND PDF FILES

Pursuant to 37 C.F.R. §1.821(c) or (e), files containing a TXT version and a PDF version of the Sequence Listing have been submitted concomitant with this application, the contents of which are hereby incorporated by reference.

### TECHNICAL FIELD

The disclosure relates generally to biotechnology, and more particularly to compositions and methods for targeted cargo delivery to a cell. In particular, the disclosure relates to compositions and methods for targeted cargo delivery to a sialoadhesin-expressing cell.

### BACKGROUND

Specific delivery of a substance to a targeted cell is desirable for various purposes, including pharmacological intervention as well as clinical and research bioassays.

Targeted delivery is particularly desirable where exposure of non-targeted cells to a substance to be delivered is preferably avoided, such as where exposure of non-targeted cells can result in undesirable side effects. For example, a therapeutic intervention may require elimination, inhibition, stimulation and/or activation of a particular cell or cell type. In such a situation, it is advantageous to deliver a substance effective to achieve a desired result preferentially to a targeted cell in order to avoid an undesirable side effect such as inhibition or stimulation of non-targeted cells and cell types. Targeted delivery also allows use of less of the substance to be delivered to achieve a desired effect.

### SUMMARY OF THE DISCLOSURE

Provided are methods of delivering a cargo moiety to a cell that includes contacting a cell expressing sialoadhesin with a conjugate including a sialoadhesin binding moiety and a cargo moiety. The sialoadhesin binding moiety binds to the sialoadhesin expressed by the cell and is internalized along with the cargo moiety, thereby delivering the cargo moiety to the cell. Cells naturally expressing sialoadhesin are known, particularly including macrophages. Particular methods provided herein include induction or enhancement of sialoadhesin expression in a cell which naturally produces little or no sialoadhesin. Induction or enhancement of sialoadhesin expression includes transfection of a sialoadhesin expression construct and/or administration of an agent effective to induce or enhance sialoadhesin expression. Expression of sialoadhesin is determined by any of various methods including binding of sialoadhesin-specific antibodies, detection of sialoadhesin encoding mRNA and the like.

In certain embodiments, the sialoadhesin binding moiety is an antibody that binds substantially specifically to sialoadhesin. Such antibodies include, but are not limited to, mouse anti-porcine sialoadhesin mAb 41D3, mouse anti-human sialoadhesin mAb 7D2, and mouse anti-porcine sialoadhesin mAb MCA2316.

In further embodiments, a sialoadhesin binding moiety is a sialoadhesin ligand.

A cargo moiety included in a conjugate is a stimulator of a response in the cell in particular embodiments. For example, in certain embodiments, a conjugate is a stimulator of an immune response in the cell. In further embodiments, a conjugate which stimulates an immune response in the cell stimulates an immune response in a subject. Thus, a cargo moiety may be an antigen.

Also provided are embodiments hereof in which the cargo moiety is an inhibitor of the cell. For example, a cargo moiety included in a conjugate is a cytotoxic agent in particular embodiments. A cytotoxic agent is exemplified by, but not limited to, a ribosome inactivating protein. A specific cytotoxic agent which is a ribosome inactivating protein is saporin.

In further embodiments, a cargo moiety is an antimicrobial agent. An antimicrobial agent included in a conjugate is effective to inhibit a microbe such as, but not limited to, a bacterium, a virus, a fungus or a protozoan.

A cytokine is a cargo moiety in certain embodiments of the method. Optionally, the cargo moiety is a nucleic acid. A delivered nucleic acid may be an expression construct. Further optionally an expression construct is included in a vector, such as, but not limited to, a bacterial plasmid or a viral vector. A nucleic acid cargo may be an antisense construct such as, but not limited to, an antisense oligonucleotide, an siRNA, an shRNA or an expression vector for expressing an antisense nucleic acid.

Where a cargo moiety is an antigen, the antigen may be a protein, a peptide, a glycoprotein or a glycopeptide. Such antigens may be synthetic, such as, but not limited to, recombinantly produced or chemically synthesized proteins or peptides; or natural, such as, but not limited to, an antigen isolated from a cell, virus or organism. In particular embodiments, a cargo which is an antigen is a viral protein, a viral peptide, a viral glycoprotein or a viral glycopeptide.

An antigen conjugated to a sialoadhesin binding moiety may be an influenza virus hemagglutinin or an antigenic portion thereof. A specific antigenic portion of an influenza virus hemagglutinin is encoded by SEQ ID NO:3 (of the accompanying and incorporated herein SEQUENCE LISTING) or a homologue thereof. In particular embodiments, a virus hemagglutinin included in a conjugate hereof is an influenza virus hemagglutinin of SEQ ID NO:4 or a homologue thereof.

A cell contacted by a conjugate for delivery of a cargo to the cell is in vitro, or in vivo.

In further embodiments, a cell is treated with a cytokine effective to induce or enhance expression of sialoadhesin in the cell. For example, a cell treated with a cytokine effective to induce or enhance expression of sialoadhesin is a monocyte, a monocyte cell line, a macrophage and a macrophage cell line. In particular embodiments, a human cell and/or a human-derived cell line is treated with a cytokine effective to induce or enhance expression of sialoadhesin. An example of a human-derived cell line is human monocyte cell line THP-1. In further embodiments, a porcine cell and/or a porcine-derived cell line is treated with a cytokine effective to induce or enhance expression of sialoadhesin. Suitable cytokines effective to induce or enhance expression of sialoadhesin include interferon alpha (IFN-alpha), and a combination of tumor necrosis factor-alpha (TNF-alpha) and interferon-gamma (IFN-gamma).

Compositions are provided herein that include a sialoadhesin binding moiety conjugated to a cargo moiety. The sialoadhesin binding moiety is an antibody or a sialoadhesin ligand in particular embodiments of a composition hereof.

A method of stimulating an immune response in a subject to a viral antigen is provided herein which includes administering a composition including a sialoadhesin binding moiety conjugated to a viral antigen to a subject. In particular embodiments, a cargo which is a viral antigen is a viral protein, a viral peptide, a viral glycoprotein or a viral glycopeptide.

In particular embodiments, a viral antigen conjugated to a sialoadhesin binding moiety is an influenza virus hemagglutinin or an antigenic portion thereof. A specific antigenic portion of an influenza virus hemagglutinin is encoded by SEQ ID NO:3.

Also provided are methods and compositions in which a viral antigen conjugated to a sialoadhesin binding moiety is a Porcine Reproductive and Respiratory Syndrome virus (PRRSV), a PRRSV protein or an antigenic portion of a PRRSV protein.

Optionally, a sialoadhesin binding moiety is an antibody or a sialoadhesin ligand. Specific antibodies included in a conjugate herein include monoclonal antibody 41D3, monoclonal antibody 7D2 and monoclonal antibody MCA2316.

A conjugate including a sialoadhesin binding moiety and a cargo is produced by chemical bonding between the sialoadhesin binding moiety and cargo in particular embodiments. In further embodiments, a conjugate including a sialoadhesin binding moiety and a cargo is produced by recombinant techniques, including expression of a fusion protein.

In particular embodiments, a method of stimulating an immune response herein includes administering an amount of a cytokine effective to induce or enhance expression of sialoadhesin in a cytokine responsive cell in the subject. A specific cytokine effective to induce or enhance expression of sialoadhesin in an INF-alpha responsive cell is INF-alpha. A, INF-alpha responsive cell is identified by methods known in the art including, but not limited to, detection of an INF-alpha receptor. Particular INF-alpha responsive cells include monocytes, such as, but not limited to, human monocytes, and monocyte-derived cell lines, such as, but not limited to, human monocyte cell line THP-1. A macrophage is a further example of an INF-alpha responsive cell.

A method of stimulating an immune response to an antigen in a subject is provided according to embodiments hereof which includes administering a composition including a sialoadhesin binding moiety conjugated to an antigen to a subject.

In further embodiments hereof, a method of screening a compound for sialoadhesin binding activity and/or sialoadhesin binding stimulated cell internalization activity is provided which includes administering a cytokine effective to induce or enhance sialoadhesin expression; administering the compound; and performing an assay for specific binding of the compound to sialoadhesin and/or performing an assay for sialoadhesin binding stimulated cell internalization activity. In particular embodiments, the cytokine effective to induce or enhance sialoadhesin expression is INF-alpha. A compound is illustratively an anti-sialoadhesin antibody or a sialoadhesin ligand. Examples of assays to determine specific binding of the compound include incubation of the compound with the cell under typical sialoadhesin binding moiety binding conditions, such as under substantially physiological conditions, and detection of binding. Detection of binding may include, for instance, detection of a reporter bound to the compound. Detection of internalization of the compound is illustratively accomplished by permeabilization of a cell and incubation with a reagent that binds to the compound, such as, but not limited to, an antibody, followed by detection of the reagent.

A method of transfecting a cell is provided which includes administering a sialoadhesin binding moiety conjugated to an expression construct to a cell expressing sialoadhesin. The cell expressing sialoadhesin is a cell transfected with a sialoadhesin expression construct in particular embodiments. In further embodiments, the cell is a cell line stably expressing sialoadhesin. In yet further embodiments, the cell is treated with a cytokine to induce or enhance sialoadhesin expression.

A kit for delivering a cargo to a cell is provided herein which includes a cell expressing sialoadhesin and a sialoadhesin binding moiety. Optionally, such a kit includes a reagent for use in conjugation of a cargo to the sialoadhesin binding moiety. A cell included in such a kit may be a cell line. In a further option, the sialoadhesin binding moiety included in the kit is conjugated to a cargo. The cargo may be conjugated to the sialoadhesin binding moiety is an expression construct.

A method of treating a pathological condition in a subject is provided including administering a therapeutically effective amount of a sialoadhesin binding moiety conjugated to a therapeutic cargo moiety to the subject, wherein the therapeutic cargo moiety is delivered to a sialoadhesin expressing cell in the subject, thereby treating the pathological condition. In particular embodiments, the therapeutic cargo moiety is an inhibitor of the cell, such as, but not limited to, a cytotoxic agent. An example of an inhibitor is saporin. A pathological condition treated according to the method is characterized by presence of a pathogen in the cell in particular embodiments. In further embodiments, the pathological condition is an autoimmune disease or cancer. An included cargo moiety is an inhibitor of macrophage activation and/or inflammation in embodiments for treating autoimmune disease. Such inhibitors include, but are not limited to, an inhibitor of macrophage activation and/or inflammation such as IL-10, TGF-beta, 6-(methylsulfinyl)hexyl isothiocyanate, a sesquiterpene chromone, and a combination thereof.

### BRIEF DESCRIPTION OF THE DRAWINGS

FIG. 1 is a graph illustrating specific binding and internalization of a sialoadhesin binding moiety at different times after incubation of macrophages at 37° C. with mAb 41D3;

FIG. 2A is a graph showing the mean±SEM of antigen-specific IgM serum titers after primary immunization with a sialoadhesin binding moiety/antigen conjugate herein in which square symbols indicate pigs immunized with HSA coupled to Sn-specific mAb 41D3; triangle symbols indicate pigs immunized with HSA coupled to irrelevant control mAb; and circle symbols indicate pigs immunized with free HSA;

FIG. 2B is a graph showing the mean±SEM of antigen-specific IgG serum titers after primary immunization with a sialoadhesin binding moiety/antigen conjugate herein in which square symbols indicate pigs immunized with HSA coupled to Sn-specific mAb 41D3; triangle symbols indicate pigs immunized with HSA coupled to irrelevant control mAb; and circle symbols indicate pigs immunized with free HSA;

FIG. 2C is a graph illustrating means±SEM of antigen-specific IgG serum titers after booster immunization in which square symbols indicate pigs immunized with HSA coupled to Sn-specific mAb 41D3; triangle symbols indicate pigs immunized with HSA coupled to irrelevant control mAb; and circle symbols indicate pigs immunized with free HSA;

FIG. 3 is a graph illustrating the percentage of living cells in a population of sialoadhesin expressing cells treated with sialoadhesin binding moiety/cytotoxic agent conjugate compared to cells treated with a non-sialoadhesin binding moiety/cytotoxic agent conjugate;

FIG. 4 is a graph illustrating the percentage of living cells in a population of sialoadhesin expressing cells treated with sialoadhesin binding moiety/cytotoxic agent conjugate compared to non-sialoadhesin expressing cells treated with a sialoadhesin binding moiety/cytotoxic agent conjugate;

FIG. 5 is a set of histograms generated from flow cytometric analysis showing binding and internalization of a particular sialoadhesin binding moiety;

FIG. 6A is a xerographic reproduction of a digital image showing SDS-PAGE analysis of the presence and purity of native influenzavirus hemagglutinin in different fractions obtained during purification includes detection of HA via western blotting using a monoclonal antibody directed against HA of the HIN1 virus;

FIG. 6B is a xerographic reproduction of a digital image showing detection of total protein in the samples shown in FIG. 6A using Coomassie blue staining;

FIG. 7A is a xerographic reproduction of a digital image showing SDS-PAGE analysis of the production of recombinant HA with a V5-His tag where the protein is produced in the absence of fetal bovine serum;

FIG. 7B is a xerographic reproduction of a digital image showing SDS-PAGE analysis of the production of recombinant HA with a V5-His tag where the protein is produced in the presence of fetal bovine serum;

FIG. 8A is a xerographic reproduction of a digital image showing SDS-PAGE Western blot analysis of different fractions taken during the purification process of HA;

FIG. 8B is a xerographic reproduction of a digital image showing SDS-PAGE Coomassie blue analysis of the same fractions taken during the purification process of HA shown in FIG. 8A;

FIG. 9A is a xerographic reproduction of a digital image showing SDS-PAGE Western blot analysis showing visualization of coupling of antibody 13D12 with isolated native HA;

FIG. 9B is a xerographic reproduction of a digital image showing SDS-PAGE Western blot analysis showing visualization of coupling of antibody 41D3 with isolated native HA;

FIG. 10 is a set of histograms generated from flow cytometric analysis showing binding and internalization of a particular sialoadhesin binding moiety;

FIG. 11 is a xerographic reproduction of a digital image showing SDS-PAGE and Coomassie blue staining of different samples taken during the antibody-saporin conjugation protocol; and

FIG. 12 is a graph showing mean immuno-peroxidase monolayer assay titers of pigs immunized with 13D12-HA or 41D3-HA.

FIG. 13. Increase of sialoadhesin expressing cells early in disease and throughout rheumatoid arthritis disease progression in synovial tissue. Semi-quantitative scores on a four-point-scale of Sn expression in the lining layer (a) and sublining layer (b) of synovial tissue biopts. Groups include control persons, patients with UA (undifferentiated arthritis), ERA (early RA) and Mtx res RA (Methotrexate resistant RA). Black lines represent the group medians. Statistical significant differences were calculated by Kruskal Wallis tests, succeeded by multiple Mann Whitney U tests and corrected for multiple testing by HOLM procedure with * indicating p<0.05; ** p<0.01 and *** p<0.001.

FIG. 14. Monoclonal anti-Sn antibody conjugated with Methotrexate kinetics in mouse blood. Detection by ELISA of the anti-Sn antibody at several time-points after intraperitoneal injection of 200 μg Ab-MTX, indicating 11% of the initial amount of conjugate is still present in the blood after 1 week.

FIG. 15. Monoclonal anti-Sn antibody conjugated with a low dose of Methotrexate prevents symptoms of arthritis during collagen induced arthritis in mice. The average clinical score (a) and the incidence of arthritis (b) of all mice in the four groups are represented at the indicated days after induction of arthritis with collagen. Groups include MTX high dose (methotrexate 35 mg/kg); ab-MTX (200 g anti-Sn antibody conjugated with methotrexate with an equivalent dose of 0.2 mg/kg methotrexate); iso-MTX (same but irrelevant isotype as anti-Sn antibody conjugated to the same amount of methotrexate); PBS. Longitudinal clinical scores, determined by mixed model analysis, were significantly (p<0.0001) different between PBS and MTX; PBS and ab-MTX; iso-MTX and ab-MTX; iso-MTX and MTX indicating a significant effect of the treatment over time.

### DETAILED DESCRIPTION

Few effective targeted delivery compositions and methods are capable of delivering a desired cargo to a targeted cell. A particular receptor, sialoadhesin (Sn), is identified as a target for targeted delivery compositions and methods herein.

Sialoadhesin, also called sheep erythrocyte receptor (SER) or sialic acid binding immunoglobulin-like lectin 1 (Siglec-1) belongs to the Siglec family of 1-type lectins and is expressed exclusively on subsets of macrophages that are found mostly in spleen, lymph nodes, bone marrow, liver, colon and lungs but not on blood monocytes as described in P. R. Crocker et al., 1991, Embo. J. 10:1661-9; P. R. Crocker et al., 1994, Embo. J. 13:4490-503; X. Duan et al., 1998, J. Virol. 72:4520-3; A. Hartnell et al., 2001, Blood 97:288-96; and N. Vanderheijden et al., 2003, J. Virol. 77:8207-15. High Sn expression has also been detected on inflammatory macrophages in tissues from patients with rheumatoid arthritis, and on infiltrating macrophages that make close contact with breast carcinoma cells as described in A. Hartnell et al., 2001, Blood 97:288-96; and D. Nath et al., 1999, Immunology 98:213-9. Sialoadhesin (Sn) was initially identified as a sialic acid dependent-sheep erythrocyte receptor (SER) on resident bone marrow cells of mice, and is now also characterized in a number of mammals including human, rat and swine, described in P. R. Crocker and S. Gordon, 1989, J. Exp. Med. 169:1333-46; P. R. Crocker and S. Gordon, 1986, J. Exp. Med. 164:1862-75; and N. Vanderheijden et al., 2003, J. Virol. 77:8207-15.

A conjugate composition is provided herein which includes a sialoadhesin binding moiety conjugated to a cargo moiety. Conjugate compositions including a sialoadhesin binding moiety conjugated to a cargo moiety may be used to deliver a cargo moiety to a sialoadhesin expressing cell.

The term “nucleic acid” as used herein refers to RNA or DNA molecules having more than one nucleotide in any form including single-stranded, double-stranded, oligonucleotide or polynucleotide. The term “nucleotide sequence” is used to refer to the ordering of nucleotides in an oligonucleotide or polynucleotide in a single-stranded form of nucleic acid. It is appreciated that, due to the degeneracy of the genetic code, multiple nucleic acids encode an identical polypeptide.

The terms “protein,” “polypeptide” and “peptide” are used interchangeably herein to refer to two or more amino acids linked by peptide bonds. The term protein includes modified proteins and peptides exemplified by, but not limited to, glycosylated, phosphorylated, ubiquitinated, myristoylated, palmitoylated, and acetylated proteins and peptides.

The term “expression construct” refers to a recombinant or synthetic nucleic acid including a nucleic acid encoding a protein, and one or more regulatory polynucleotides operably linked to the nucleic acid encoding the protein that direct transcription of at least the nucleic acid encoding the protein in a cell.

The term “transfection” refers to introduction of an exogenous nucleic acid into a cell.

The term “operably linked” refers to a nucleic acid in functional relationship with a second nucleic acid. In general, operably linked nucleic acids are contiguous. An exception is operable linkage of an enhancer, which may be non-contiguous and in functional relationship with another nucleic acid.

The term “vaccine” refers to a pharmaceutical composition including at least one antigen that stimulates an immune response in a subject.

The term “vaccination” refers to administration of a vaccine to stimulate an immune response in a subject. Vaccination of a subject may be performed to prevent or treat a disease in the subject.

The term “antigen” refers to a molecule that includes one or more epitopes that stimulate an antigen-specific response by a component of a host immune system, such as an immune cell. An antigen can include peptide, proteins, glycoproteins, polysaccharides, lipids, gangliosides, portions thereof, and combinations thereof.

The term “stimulation of an immune response” means eliciting or enhancing an immune response.

The term “homologue” refers to a protein characterized by an amino acid sequence and/or structural homology to a reference protein. In general, a homologue of the reference protein is at least 50%, preferably at least 75%, more preferably at least 80%, 85%, 90%, 91%, 92%, 93%, 94%, 95%, 96%, 97%, 98%, 99%, or greater, identical to the reference protein. A homologue is illustratively an orthologue of the reference protein isolated from another species. A homologue includes a protein having one or more amino acid substitutions, deletions or insertions compared with the reference protein.

The term “biologically active homologue” of a reference protein refers to a protein characterized by an amino acid sequence and/or structural homology to the reference protein which has substantially similar functional, structural, and/or biochemical properties compared to the reference protein, particularly the naturally occurring reference protein.

One type of homologue is a conservatively modified protein and/or fragment thereof. A conservatively modified protein or fragment thereof is a protein or peptide which includes substitution of an amino acid with a chemically similar amino acid. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate and glutamate; basic amino acids include histidine, lysine and arginine; aliphatic amino acids include isoleucine, glycine, leucine and valine; aromatic amino acids include phenylalanine, tyrosine and tryptophan; polar amino acids include aspartate, glutamate, histidine, lysine, asparagine, glutamine, arginine, serine, threonine and tyrosine; hydrophobic amino acids include alanine, cysteine, phenylalanine, glycine, isoleucine, leucine, methionine, proline, valine, tyrosine and tryptophan; and hydrophilic amino acids include asparagine, aspartate, glutamine, glutamate, histidine, serine and threonine. Amino acids may also be described in terms of relative size, alanine, cysteine, aspartate, glycine, asparagine, proline, threonine, serine, valine, all typically considered to be small.

Percent identity is determined by comparison of amino acid or polynucleotides, including a reference sequence and a putative homologue sequence. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S. Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP, BESTFIT, FASTA, TFASTA; and BLAST, publicly available from the National Center for Biotechnology Information, for instance, on the World Wide Web at ncbi.nlm.nih.gov.

### Sialoadhesin Binding Moiety

A sialoadhesin binding moiety binds specifically to sialoadhesin. The term “binds specifically” as used herein is intended to indicate that a sialoadhesin binding moiety included in a conjugate interacts preferentially with sialoadhesin and does not significantly interact with other proteins or other molecules. A sialoadhesin binding moiety conjugated to a cargo moiety has sialoadhesin-specific binding activity and thus confers sialoadhesin-specific binding activity on a conjugate. In particular, a sialoadhesin binding moiety conjugated to a cargo moiety binds to an extracellular portion of sialoadhesin expressed by a cell. Further, a sialoadhesin binding moiety binds specifically with sialoadhesin present in the cell membrane of a target cell and stimulates uptake of a conjugate into the cell

In certain embodiments, a sialoadhesin binding moiety is an antibody. The term “antibody” refers to polyclonal antibodies, monoclonal antibodies (mAbs), chimeric antibodies, humanized antibodies, as well as antigen binding antibody fragments and molecules having antigen binding functionality.

The term “antibody” includes an intact immunoglobulin having four polypeptide chains, two heavy (H) chains and two light (L) chains linked by disulfide bonds. The term “antibody” also includes sialoadhesin binding antibody fragments illustratively including, but not limited to, such fragments as an Fab fragment, an Fab′ fragment, an F(ab′)2 fragment, an Fd fragment, an Fv fragment, an scFv fragment, and a domain antibody (dAb).

An anti-sialoadhesin antibody and/or sialoadhesin binding antibody fragment included in a conjugate hereof is capable of binding sialoadhesin and stimulating uptake of the conjugate into the cell.

An antibody or antibody fragment included in a conjugate hereof specifically binds to sialoadhesin. A preferred sialoadhesin binding moiety binds sialoadhesin with greater affinity than it binds another member of the Siglec family.

A preferred sialoadhesin binding moiety included in a conjugate is characterized by specific binding activity for sialoadhesin of at least about 1×10⁵ M⁻¹. In further embodiments, a preferred sialoadhesin binding moiety has a specific binding affinity for sialoadhesin of at least about 1×10⁶ M⁻¹. In still further embodiments, a preferred sialoadhesin binding moiety has a specific binding affinity for sialoadhesin of at least about 1×10⁷ M⁻¹.

Anti-sialoadhesin antibodies and sialoadhesin binding antibody fragments may be provided by any method, illustratively including, but not limited to, immunization, isolation and purification, enzymatic cleavage of an intact immunoglobulin, chemical synthesis of a desired sialoadhesin binding peptide or protein, production by recombinant nucleic acid technology. Combinations of such methods may also be used.

An anti-sialoadhesin antibody can be made by immunization using as an antigen a full length sialoadhesin or a peptide fragment of sialoadhesin. Such proteins and peptides may be, illustratively a human, pig, sheep, rat, mouse, or other sialoadhesin protein or peptide. Exemplary human, mouse and porcine sialoadhesin protein sequences and polynucleotides encoding human, mouse and porcine sialoadhesins included herein as SEQ ID NO:5, SEQ ID NO:6, SEQ ID NO:7, SEQ ID NO:8, SEQ ID NO:9, and SEQ ID NO:10.

Extracellular portions of sialoadhesin from various species have been characterized, as have sialic acid binding sites, as exemplified in D. Nath et al., J. Biol. Chem., 270:26184-26191, 1995; M. Vinson et al., J. Biol. Chem., 271:9267-9272, 1996; A. Hartnell et al., Blood, 97:288-296; and N. Vanderheijden et al., 2003, J. Virol. 77:8207-15. For example, an extracellular portion of human sialoadhesin extends from amino acid 1-1642, an extracellular portion of porcine sialoadhesin extends from amino acid 1-1643 and an extracellular portion of mouse sialoadhesin extends from amino acid 1-1638, each with reference to the sequences described herein. A sialoadhesin fragment used as an antigen in preparation of a sialoadhesin binding antibody preferably includes one or more Ig-like domains.

Antigens may be prepared by any of various methods, including isolation from natural sources, recombinant production or by chemical synthetic techniques. Sialoadhesin proteins and peptides for use as antigens in preparation of a sialoadhesin binding antibody are similarly prepared by any of various techniques.

A peptide portion of a sialoadhesin or other antigen may be made more immunogenic if desired by linkage to a carrier molecule such bovine serum albumin or keyhole limpet hemocyanin. Such a linkage may be accomplished by any of various techniques, illustratively including, but not limited to, conjugation and expression of a fusion protein.

Recombinantly expressed proteins and peptides, such as, but not limited to, sialoadhesin and sialoadhesin fragments, may be tagged to allow for easier isolation. For instance, such proteins and peptides may be Fc-tagged.

Antibodies, antigen binding fragments and methods for their generation are known in the art and such antibodies, antigen binding fragments and methods are described in further detail, for instance, in Antibody Engineering, R. Kontermann and S. Dübel (Eds.), Springer, 2001; E. Harlow and D. Lane, Antibodies: A Laboratory Manual, Cold Spring Harbor Laboratory Press, 1988; F. Ausubel et al. (Eds.), Short Protocols in Molecular Biology, Wiley, 2002, particularly chapter 11; J. D. Pound (Ed.) Immunochemical Protocols. Methods in Molecular Biology, Humana Press; 2nd ed., 1998; B. K. C. Lo (Ed.), Antibody Engineering Methods and Protocols. Methods in Molecular Biology, Humana Press, 2003; and G. Kohler and C. Milstein, Nature, 256:495-497 (1975).

The term “antigen” in the context of making a sialoadhesin binding moiety refers to sialoadhesin or an antigenic peptide portion thereof. In a particular embodiment, an antigenic portion of sialoadhesin includes a portion of sialoadhesin present external to a cell expressing sialoadhesin. Such a portion preferably includes a sialic acid binding domain.

An antibody which is a sialoadhesin binding moiety may be made using a native sialoadhesin, such as exemplified by amino acid sequences appended hereto, and/or peptide fragments thereof, as an antigen. An antibody which is a sialoadhesin binding moiety may be also be made using a sialoadhesin homologue, modified sialoadhesin and/or fragment thereof as an antigen.

In a specific embodiment, a sialoadhesin binding moiety is a monoclonal antibody 41D3. Monoclonal antibody 41D3 (mAb 41D3) is a mouse monoclonal anti-porcine sialoadhesin antibody. Monoclonal antibody 41D3 is described in N. Vanderheijden et al., 2003, J. Virol. 77:8207-15; and in X. Duan et al., 1998, J. Virol. 72:4520-3. A hybridoma producing monoclonal antibody 41D3 was deposited with the CNCM (Collection Nationale de Cultures de Microorganisms) at the Institute Pasteur, 28, Rue du Docteur Roux, F-75724 Paris Cedex 15 and given Accession number I-2719.

In a further specific embodiment, a sialoadhesin binding moiety is mouse monoclonal antibody 7D2 (mAb 7D2) which binds human sialoadhesin. MAb 7D2 was raised against an Fc fusion protein containing the N-terminal four domains of human sialoadhesin. MAb 7D2 is further described in A. Hartnell et al., Blood, 97:288-96, 2001 and is commercially available.

Another specific example of a sialoadhesin binding moiety is mouse anti-porcine sialoadhesin monoclonal antibody MCA2316 described, e.g., in R. Bullido, Tissue Antigens, 1997, 49(4):403-13 and commercially available.

A sialoadhesin binding moiety is a sialoadhesin ligand in a further embodiment of a conjugate composition herein. As noted above, sialoadhesin is a sialic acid-binding immunoglobulin-like lectin. Sialoadhesin binds sialic acid, and in particular, α2-3 sialic acid residues and some α2-6 and α2-8 sialic acid residues. Such sialic acid residues illustratively include Siaα2-3Galβ1-3GalNAc; Siaα2-3Galβ1-3GlcNAc; and Siaα2-3Galβ1-4GlcNAc, Siaα2-6Galβ1-3GalNAc and Siaα2-8Neu5Acα2-3Galβ1-3GalNAc. Thus, in an embodiment in which a sialoadhesin binding moiety is a sialoadhesin ligand, a sialoadhesin binding moiety preferably includes a sialylated organic structure such as, but not limited to, a sialylated protein or peptide, lipid, and/or carbohydrate.

In a further embodiment, a sialoadhesin binding moiety includes a natural sialylated ligand for sialoadhesin. A natural sialylated ligand for sialoadhesin is a sialylated structure which occurs naturally and binds sialoadhesin in vivo. Natural sialylated ligands illustratively include CD43, galactose-type C-type lectin 1, and MUC1 antigen. A natural sialylated ligand of sialoadhesin may be isolated from a natural source or recombinantly produced for inclusion in a conjugate composition herein.

A further natural sialoadhesin ligand is a porcine arterivirus protein.

### Cargo Moiety

As noted above, a conjugate composition herein includes a sialoadhesin binding moiety and a cargo moiety. A cargo moiety is a substance to be delivered to a target cell.

In certain embodiments, a cargo moiety is a stimulator of a response in a target cell. For instance, a cargo moiety may be a stimulator of an immune response in a macrophage. A cargo moiety may also be a stimulator of nitric oxide production in a target cell.

Examples of cargo moieties which are macrophage stimulators illustratively include interleukin-4, interleukin-10, interleukin-13, macrophage stimulating protein, CD40 ligand, and interferon-gamma. Additional stimulators include lipoteichoic acid, muramyl tripeptide TNF-alpha, GM-CSF, a lipopolysaccharide and an extracellular matrix protein.

In a particular example, a cargo moiety which is a stimulator of an immune response is an antigen. An antigen included in a conjugate may be any type of antigen, illustratively including, but not limited to, a peptide, a protein, a lipid, a carbohydrate, and combinations of these or other antigenic substances. An antigen may be derived from any source and thus may be an isolated natural substance, a recombinantly produced substance, a chemically synthesized substance, or otherwise provided. The identity of the antigen will depend on the desired result. In general, an antigen is included as a cargo moiety to be delivered to an antigen presenting cell in order to stimulate the immune system of a subject to produce an immune response to the antigen.

In certain embodiments, an antigen included as a cargo moiety is a porcine arterivirus peptide or protein.

Also provided is a conjugate including a cargo moiety which is an inhibitor of a target cell. Exemplary inhibitors include inhibitors of macrophage activation, inhibitors of inflammation and general cell inhibitors.

Inhibitors of macrophage activation and inflammation are useful as cargo moieties to decrease macrophage activation and inflammation where problematic, such as in autoimmune diseases illustratively including, but not limited to, endotoxemia, multiple sclerosis, rheumatoid arthritis, and lupus erythematosus. Inhibitors of macrophage activation and inflammation include anti-inflammatory cytokines and anti-inflammatory compounds such as, but not limited to, IL-10, TGF-beta, 6-(methylsulfinyl)hexyl isothiocyanate, and sesquiterpene chromones including those isolated from Ferula fukanensis.

In a further embodiment, a cargo moiety which is an inhibitor of a target cell is a cytotoxic agent. A cytotoxic agent may be included in a conjugate for delivery to a cell in order to inhibit or destroy the cell. For example, a macrophage may be targeted for inhibition of destruction by a cytotoxic agent in order to inhibit a macrophage activity, such as an inflammatory activity. In a further example, a cytotoxic agent is delivered to a sialoadhesin expressing cell in order to inhibit a microbial infection. A cytotoxic agent may be any cytotoxic agent which can be conjugated with a sialoadhesin binding moiety to produce a conjugate hereof.

Exemplary cytotoxic cargo moieties are drugs used as anti-tumoral agents. Anti-tumoral agents are described, e.g., in Goodman et al., Goodman and Gilman's The Pharmacological Basis of Therapeutics, 8th Ed., Macmillan Publishing Co., 1990.

Such drugs illustratively include acivicin, aclarubicin, acodazole, acronine, adozelesin, aldesleukin, alitretinoin, allopurinol, altretamine, ambomycin, ametantrone, amifostine, aminoglutethimide, amsacrine, anastrozole, anthramycin, arsenic trioxide, asparaginase, asperlin, azacitidine, azetepa, azotomycin, batimastat, benzodepa, bicalutamide, bisantrene, bisnafide dimesylate, bizelesin, bleomycin, brequinar, bropirimine, busulfan, cactinomycin, calusterone, capecitabine, caracemide, carbetimer, carboplatin, carmustine, carubicin, carzelesin, cedefingol, celecoxib, chlorambucil, cirolemycin, cisplatin, cladribine, crisnatol mesylate, cyclophosphamide, cytarabine, dacarbazine, dactinomycin, daunorubicin, decitabine, dexormaplatin, dezaguanine, dezaguanine mesylate, diaziquone, docetaxel, doxorubicin, droloxifene, droloxifene, dromostanolone, duazomycin, edatrexate, eflomithine, elsamitrucin, enloplatin, enpromate, epipropidine, epirubicin, erbulozole, esorubicin, estramustine, estramustine, etanidazole, etoposide, etoposide, etoprine, fadrozole, fazarabine, fenretinide, floxuridine, fludarabine, fluorouracil, fluorocitabine, fosquidone, fostriecin, fulvestrant, gemcitabine, gemcitabine, hydroxyurea, idarubicin, ifosfamide, ilmofosine, interleukin II (IL-2, including recombinant interleukin II or rIL2), interferon alfa-2a, interferon alfa-2b, interferon alfa-n1, interferon alfa-n3, interferon beta-I a, interferon gamma-I b, iproplatin, irinotecan, lanreotide, letrozole, leuprolide, liarozole, lometrexol, lomustine, losoxantrone, masoprocol, maytansine, mechlorethamine hydrochloride, megestrol, melengestrol acetate, melphalan, menogaril, mercaptopurine, methotrexate, methotrexate, metoprine, meturedepa, mitindomide, mitocarcin, mitocromin, mitogillin, mitomalcin, mitomycin, mitosper, mitotane, mitoxantrone, mycophenolic acid, nelarabine, nocodazole, nogalamycin, ormnaplatin, oxisuran, paclitaxel, pegaspargase, peliomycin, pentamustine, peplomycin, perfosfamide, pipobroman, piposulfan, piroxantrone hydrochloride, plicamycin, plomestane, porfimer, porfiromycin, prednimustine, procarbazine, puromycin, puromycin, pyrazofurin, riboprine, rogletimide, safingol, safingol, semustine, simtrazene, sparfosate, sparsomycin, spirogermanium, spiromustine, spiroplatin, streptonigrin, streptozocin, sulofenur, talisomycin, tamoxifen, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine sulfate, vindesine, vindesine, vinepidine, vinglycinate, vinleurosine, vinorelbine, vinrosidine, vinzolidine, vorozole, zeniplatin, zinostatin, zoledronate, and zorubicin. A cytotoxic cargo moiety may also be pharmaceutically acceptable salts, esters, amides, hydrates, and/or prodrugs of any of these or other cytotoxins.

In a further specific example, a cytotoxic cargo moiety is the cytotoxic ribosome-inactivating protein saporin.

In some embodiments, a toxic agent may be included to inhibit or destroy a pathological microbial organism associated with the cell. For example, bacteria, viruses and protozoa are known to be sequestered within certain cells. Pathogens, illustratively including, but not limited to, Trypanosoma cruzi, Mycobacterium tuberculosis, Salmonella sp., Neisseria meningitidis, HIV, and Ross River virus, can hide in macrophages from the host's immune system and thereby cause persistent infections as described in S. Aquaro et al., 2002, Antiviral Res. 55:209-25; I. E. Brodsky et al., 2005, Mol. Microbiol. 55:954-72; C. Jones et al., 2003, Mol. Microbiol. 49:1213-25; D. M. Monack et al., 2004, J. Exp. Med. 199:231-41; V. G. Monteiro et al., 2005, Parasitol. Res. 97:380-5; J. Rengarajan et al., 2005, Proc. Natl. Acad. Sci. U.S.A. 102:8327-32; and S. J. Way et al., 2002, Virology 301:281-92. A fungus is a further example of a pathogen which may be present in a host immune system. Thus, in one embodiment of a conjugate composition herein, a toxic agent effective to inhibit an organism is a cargo moiety delivered to a cell infected by the organism. Such toxic agents illustratively include an antibacterial agent, an antiviral agent, an antifungal agent and an antiprotozoal agent.

Specific examples of antibacterial agents include tetracyclines such as, but not limited to, doxycycline, tetracycline oxytetracycline, demeclocycline, and minocycline; beta-lactams such as, but not limited to, penicillins and cephalosporins; aminoglycosides such as, but not limited to, gentamycin, neomycin and streptomycin; macrolides such as, but not limited to, azithromycin, clarithromycin, lincomycin and erythromycin; nitroimidazoles such as, but not limited to, metronidazole and tinidazole; quinolones such as, but not limited to, cinoxacin, ciprofloxacin, norfloxacin, ofloxacin, and levofloxacin; rifampin, vancomycin, and clindamycin.

Specific examples of antiviral agents include abacavir, acyclovir, amprenavir, aplaviroc, atazanavir, brecanavir, darunavir, delavirdine, dexelvucitabine, didanosine, disoproxil, efavirenz, emtricitabine, enfuvirtide, etravirine, famciclovir, fosamprenavir, ganciclovir, indinavir, lamivudine, lopinavir, maraviroc, nelfinavir, nevirapine, ritonavir, saquinavir, stavudine, tenofovir fumarate, tipranavir, vicriviroc, zalcitabine, and zidovudine.

Specific examples of antiprotozoal agents include azanidazole, chloroquine, metronidazole, nimorazole, ornidazole, secnidazole, sinefungin, tenonitrozole, temidazole, tinidazole.

Examples of antifungal agents include azoles illustratively including, but not limited to, miconazole, ketonazole, itraconazole, fluconazole, voriconazole, posaconazole, ravuconazole, terconazole, clotrimazole, sertaconazole, econazole, and fenticonazole; and polyenes illustratively including, but not limited to, natamycin, filipin, nystatin and amphotericin B.

A cargo moiety is a nucleic acid in particular embodiments. A cargo nucleic acid may be DNA, RNA, a polynucleotide, an oligonucleotide, an antisense polynucleotide or oligonucleotide, or siRNA for example. The nucleic acid may encode a protein or peptide, such as an mRNA. A cargo nucleic acid may be linear, circular, supercoiled, single stranded, or double, triple or quadruple stranded.

In particular embodiments, a cargo nucleic acid includes an expression construct. Delivery of a conjugate including a sialoadhesin binding moiety and a cargo nucleic acid expression construct to a cell expressing sialoadhesin allows expression of an expression construct encoded protein or peptide in the cell.

In further embodiments, a cargo moiety which is an inhibitor or stimulator of a target cell may be a nucleic acid. A nucleic acid inhibitor may encode an inhibitor or stimulator for example. Alternatively, the nucleic acid itself may act as a stimulator or inhibitor.

A nucleic acid cargo is an inhibitor in certain embodiments, delivered to a sialoadhesin expressing cell in order to inhibit expression of a protein, and/or transcription and/or translation of a nucleic acid. Illustrative examples of nucleic acid inhibitors include siRNA, an antisense polynucleotide, an antisense oligonucleotide, and a ribozyme. Nucleic acid inhibitors may contain naturally occurring nucleic acids and/or may contain modified nucleic acids such as, but not limited to, phosphorothioates.

Preparation of nucleic acid inhibitors such as these are known in the art, as described, e.g., in S. T. Crooke, Antisense Drug Technology: Principles. Strategies, and Applications, CRC Press, 2001; and D. Engelke, RNA Interference (RNAi): The Nuts & Bolts of siRNA Technology, DNA Press, 2004.

A nucleic acid inhibitor is delivered to inhibit a desired target in a sialoadhesin expressing cell in vitro, ex vivo and/or in vivo, particularly a macrophage. For example, a nucleic acid inhibitor of function or synthesis of a microbial protein or nucleic acid infecting the sialoadhesin expressing cell is delivered to inhibit microbial infection.

In a further example, a nucleic acid inhibitor is delivered to inhibit a process or function of the sialoadhesin expressing cell. For example, it may be desired to inhibit or eliminate a cell expressing sialoadhesin. Inflammation and/or macrophage activation are processes or functions of a sialoadhesin expressing cell that may be inhibited. An inhibitory nucleic acid cargo, such as a nucleotide analog, may be delivered to inhibit or eliminate such a cell.

A nucleic acid cargo is a stimulator in certain embodiments, delivered to a sialoadhesin expressing cell in vitro, ex vivo and/or in vivo in order to stimulate a process or function of the sialoadhesin expressing cell. For example, a nucleic acid cargo includes a plasmid encoding a peptide or protein to which an immune response is desired. The plasmid cargo is delivered to a sialoadhesin expressing macrophage in an organism wherein the peptide or protein is produced and stimulates an immune response.

A plasmid encoding a peptide or protein is preferably an expression construct containing a nucleic acid encoding the peptide or protein along with one or more regulatory polynucleotides required or desirable for expression of the peptide or protein. Such regulatory sequences illustratively include a promoter, an enhancer, a splicing signal, a transcription start site, a transcription termination signal, a polyadenylation signal, an internal ribosome entry site (IRES) and combinations thereof. Suitable promoters include constitutively active promoters, inducible promoters and cell-type specific promoters.

A nucleic acid cargo may be conjugated to a sialoadhesin binding moiety directly or indirectly.

For example, a nucleic acid may be conjugated to a sialoadhesin binding moiety forming a bond between the nucleic acid and the sialoadhesin binding moiety. For example, a carbodiimide, such as 1-ethyl-3-[3-dimethylaminopropyl]carbodiimide hydrochloride (EDC), may be used to form a phosphate ester with a 5′ terminal phosphate group present on a nucleic acid and then coupled with an amine group of a sialoadhesin binding moiety to produce a conjugate including a phosphoramidate linkage.

In a further embodiment, a nucleic acid is indirectly conjugated to a sialoadhesin binding moiety, e.g., through a linker or other molecule. A sialoadhesin binding moiety may, e.g., be conjugated to a positively charged protein. The positively charged protein may be brought into contact with a nucleic acid to allow charge-based bonding between the positively charged protein and the negatively charged nucleic acid. Examples of positively charged proteins in this context include protamine and polylysine.

A cargo moiety may include a microorganism and/or an antigenic molecule derived from such an organism. A cargo moiety may be, e.g. a virus, a bacterium, a protozoan, and/or an antigenic molecule derived from such an organism. A microorganism included in such a conjugate may be inactivated.

In certain embodiments, a viral cargo moiety is an intact virus or portion thereof conjugated to a sialoadhesin binding antibody. Such a virus may be any type of virus, including viruses useful in stimulating an antigenic response to the virus.

In particular embodiments, a virus included in a conjugate as a cargo moiety is a swine viral disease virus. Swine viral disease viruses include PRRSV, Porcine circovirus type 2, Parvovirus and Pseudorabies virus. In particular embodiments, a swine viral disease virus is included as a cargo moiety in a conjugate herein for administration to stimulate an immune response to the virus. A particular swine viral disease virus protein or antigenic portion of a swine viral disease virus protein is included in a conjugate herein as a cargo moiety in particular embodiments. For example, a PRRSV membrane protein GP3, GP4, GP5 or Matrix (M) and/or an antigenic portion thereof is a cargo moiety in some embodiments of a conjugate. In further embodiments, a cargo moiety is a Porcine circovirus type 2 Capsid protein (CAP), a Parvovirus Capsid protein VP2 and/or a Pseudorabies virus gB, gC and/or gD protein, and/or an antigenic portion thereof. A combination of viral proteins and/or antigenic portions thereof may be included as a cargo moiety in embodiments of a conjugate hereof.

PRRSVs are exemplified by European type PRRSV Lelystad virus, Accession No. M96262 and American type PRRSV VR-2332, Accession No. U87392.

In further embodiments, a virus included in a conjugate as a cargo moiety is a human viral disease virus. Human viral disease viruses Herpes simplex virus type 1, Herpes simplex virus type 2, Varicella Zoster Virus, Cytomegalovirus, Measles virus, Mumps virus, Rubella virus, Hepatitis A virus, Hepatitis B virus, Hepatitis C virus, Human immunodeficiency virus (HIV), Poliovirus, Human papillomavirus, and Coronaviruses. In particular embodiments, a human viral disease virus is included as a cargo moiety in a conjugate herein for administration to stimulate an immune response to the virus. A particular human viral disease virus protein or antigenic portion of a human viral disease virus protein is included in a conjugate herein as a cargo moiety in particular embodiments. For example, a cargo moiety may be a Herpes simplex virus type 1 gB, gC and/or gD protein; a Herpes simplex virus type 2 gB, gC and/or gD protein; a Varicella Zoster Virus gH:gL complex, gB, and/or gC protein; a Cytomegalovirus gM:gN complex and/or gB protein; a Measles virus Hemagglutinin protein (H) and/or fusion protein (F); a Mumps virus Hemagglutinin-Neuraminidase protein (HN) and/or fusion protein (F); a Rubella virus Envelope protein E1 and/or E2; a Hepatitis A virus Capsid protein VP1 and/or VP2; a Hepatitis B virus Envelope protein S, M, L and/or HbsAb; a Hepatitis C virus Envelope glycoproteins E1 and/or E2; a Human immunodeficiency virus (HIV) gp120; a Poliovirus VP1, VP2 and/or VP3 protein; a Human papillomavirus L1 protein; a Coronavirus spike protein, such as, but not limited to, SARS Coronavirus Spike protein (S); and/or an antigenic portion of any of these. In particular embodiments, Human papillomavirus L1 protein is a Human papillomavirus type 16, 18, 6 and/or 11 L1 protein and/or an antigenic portion thereof. A combination of viral proteins may be included as a cargo moiety in embodiments of a conjugate hereof.

Influenza viruses are a major cause of human and animal disease. Influenza viruses are classed and named according to the specific characteristics of two proteins on the surface of the virus, hemagglutinin (also called hemagglutinin) and neuraminidase. At least sixteen different influenza virus subtypes have been identified according to hemagglutinin protein characteristics. These subtypes are called H1, H2, H3, H4, H5, H6, H7, H8, H9, H10, H11, H12, H13, H14, H15 and H16. Numerous influenza virus strains of each subtype have been identified and many have been characterized by nucleic acid sequencing and/or protein sequencing of the viral glycoprotein hemagglutinin. Nucleotide and protein sequences of the influenza virus protein hemagglutinin are known in the art and are available to the public via the National Center for Biotechnology Information (NCBI) Entrez protein and nucleotide search and retrieval system which have been compiled from a variety of sources, including GenBank, RefSeq, and PDB, and including SwissProt, PIR, PRF, PDB, genpept and translations from annotated coding regions in GenBank and RefSeq under accession numbers included herein. The protein and polynucleotides associated with the accession numbers included herein characterize influenza virus hemagglutinins suitable for inclusion as a cargo moiety in conjugates herein.

In particular, peptides of influenza virus subtype H1 hemagglutinin suitable for inclusion as a cargo moiety in conjugates herein have the following accession numbers and are available to the public via the National Center for Biotechnology Information (NCBI) Entrez protein search and retrieval system:

AAF87274; AAF87282; AAF87284; AAN64900; AAN64902; AAN83988; AAQ10372; AAQ10387; AAQ10394; AAT81327; AAT81329; AAT81330; AAT81336; AAT81338; ABB9551; ABB19618; ABB79979; ABC02277; ABD62781; ABD94965; ABD95075; ABD95152; ABD95229; ABD95284; ABD95306; ABF47869; ABF82896; ABG66974; ABI21200; ABI21222; ABJ09327; ABK40028; ABO21716; ABO21724; ABO32992; ABP49327; ABP49382; ABP49481; BAE53729; BAE96533; BAE96534; BAE96536; BAF03629; CAA40728; AAF87278; AAF87279; AAF87280; AAF87281; AAN64903; AAN64904; AAN64905; AAQ10390; AAQ10391; AAR90881; AAT81331; AAT81332; AAT81333; AAT81334; AAT81335; AAT93388; AAY56898; ABA43189; ABB19562; ABB21772; ABC86237; ABD61518; ABD62843; ABD79255; ABD94987; ABD95240; ABD95251; ABD95273; ABF47891; ABF82662; ABF82684; ABF82841; ABF82863; ABF82918; ABI84478; ABI84948; ABI93028; ABJ16686; ABK40006; ABK40557; ABN50917; ABN51066; ABN51088; ABO21709; ABO21723; CAA40730; CAA86563; CAA91082; AAA16808; AAA16813; AAA16880; AAA19935; AAA43142; AAA43153; AAA43168; AAA43170; AAA43171; AAA43175; AAA43231; AAA43240; AAC57415; AAK40318; AAK51344; AAK51345; AAK51347; AAN46827; AAN64894; AAN64896; AAP69688; AAP69691; AAP79971; AAU09400; ABA08519; ABA42247; ABB03123; ABB04972; ABB80045; ABB84190; ABB86887; ABD77675; ABD78071; ABD94789; ABE12248; ABF47693; ABG72868; ABG72869; ABG79952; ABI54437; ABI54444; ABI54445; ABO21730; ABO21731; ABO33006; ABO52038; ABO52258; ABP49305; ABP49360; BAF31892; CAA86560; CAA86561; CAA86562; CAC18524; AAA43158; AAA43169; AAA43173; AAA43176; AAA43190; AAA43194; AAA43225; AAA43232; AAA43283; AAK40314; AAK40315; AAK51348; AAN64897; AAN64898; AAN64899; AAP79977; AAX56530; AAZ79549; ABA08497; ABA12707; ABA42258; ABB51962; ABB53603; ABB82194; ABB82205; ABB82216; ABB86877; ABB86917; ABB86929; ABB86937; ABB86946; ABC40522; ABD77708; AAA43661; AAA43680; AAF06945; AAF06946; AAF87275; AAF87276; AAF87283; AAN64901; AAQ10369; AAQ10373; AAQ10380; AAQ10385; AAQ10386; AAQ10388; AAQ10395; AAQ10396; AAT81328; AAT81337; AAT81339; AAT81340; AAT85679; ABA12729; ABB19571; ABB19607; ABB19628; ABB20429; ABB79990; ABC40631; ABD61540; ABD61735; ABD62061; ABD79101; ABD85261; ABD95053; ABD95064; ABD95086; ABD95163; ABD95218; ABD95295; ABF71860; ABF82852; ABF82874; ABF82885; ABF82907; ABI92379; ABI96088; ABI96091; ABI96093; ABI96097; ABI96098; ABI96101; ABI96107; ABI96108; ABI96111; ABJ09151; ABJ53493; ABK40510; ABK40579; ABK40601; ABM22246; ABO38384; ABO38406; BAA96109; BAA96114; BAA96115; BAA96122; BAA96124; BAA96125; CAA35094; AAA58799; AAA58801; AAA65544; AAA65546; AAA65551; AAA65553; AAA65554; AAA74285; AAA74289; AAA74291; AAA74293; AAA74299; AAA91616; AAA92279; AAB03291; AAB27052; AAB29091; AAB39351; AAB50958; AAB50966; AAG22555; ABD77917; ABD77928; ABD77950; ABD78038; ABD94778; ABE12634; ABE26991; ABF47583; ABG48049; ABG72867; ABI20826; ABI51313; ABI54438; ABI84617; ABI92302; ABI95250; ABI96094; ABI96095; ABI96096; ABI96104; ABI96105; ABI96106; ABJ53427; ABJ53504; ABK40689; ABM22224; ABO38318; ABO38340; ABO44046; BAA96111; BAA96112; BAA96117; BAA96118; BAA96121; BAA96126; BAA96127; BAA96128; BAA96131; BAF47397; AAA65547; AAA65548; AAA65549; AAA65550; AAA65556; AAA65557; ABG37362; ABG66973; ABG66975; ABI21211; ABI21233; ABJ16609; ABM21960; ABM66864; ABN50928; ABN51077; ABN59423; ABN59434; ABO21725; ABO52104; ABP49316; ABP49338; ABP49349; BAE53730; BAE96535; BAE96537; BAE96541; BAE96542; BAF03627; CAA40729; CAA82950; CAA91083; AAA 16779; AAA 16809; AAA 16812; AAA 16879; AAA 16905; AAA43161; AAA43167; AAA43172; AAA43206; AAA43209; AAA43233; AAA43234; AAA43238; AAC53845; AAC53846; AAC57166; AAK40317; AAK51342; AAK51343; AAK51346; AAF06947; AAF75994; AAF80098; AAF80099; AAF87277; AAQ10367; AAQ10368; AAQ10389; AAQ10392; AAQ10393; AAZ38627; ABA42575; ABB19574; ABB19667; ABD63063; ABD79112; ABD85123; ABD94976; ABD94998; ABD95130; ABD95141; ABD95262; ABF47880; ABF82673; ABF82819; ABF82830; ABF82929; ABG66976; ABG66977; ABG88344; ABK39995; ABK40534; ABK40546; ABK40568; ABN50756; ABN50900; ABO33025; ABO52280; ABP49393; ABP49448; BAE96538; BAE96539; BAE96540; CAA40731; CAA42444; AAL60444; AAL87869; AAL87871; AAM76686; AAM76689; AAM76690; AAP34322; AAP69678; AAP69679; AAP69681; AAZ74374; ABA08464; ABA18037; ABB19518; ABB19529; ABB19540; ABC66233; ABC66236; ABD77719; ABD77807; ABD77818; ABD77972; ABD94811; ABD95328; ABD95339; ABE11657; ABE11723; ABE11812; ABE11922; ABE12032; ABF21277; ABG26243; ABG26791; ABG26813; ABG26824; ABG26945; ABG67477; ABI21530; ABI21552; ABI21574; ABI22109; ABI30367; ABI96117; ABI96118; ABI96123; AAA67338; AAA72339; AAA74296; AAA74297; AAA74298; AAA79714; AAA79727; AAB03292; AAB39352; AAB50960; AAB50961; AAB50962; AAB50963; AAB50964; AAB50965; AAL60449; AAL87868; AAM22277; AAM22278; AAM76691; AAP34323; AAP34324; AAT65329; AAZ83977; ABA06510; ABA42324; ABC66239; ABD78093; ABD95350; ABD95712; ABE11668; ABE11690; ABE11834; ABE11856; ABE11878; ABE11889; ABF21278; ABF47561; ABF82940; ABG26835; ABG88300; ABG88311; ABG88333; ABG88542; ABI85225; AAK51352; AAN64893; AAN64895; AAP69687; AAP69692; AAP79964; AAZ79392; AAZ79538; ABA12715; ABA42280; ABB03134; ABB03145; ABB86899; ABB86907; ABC40533; ABD77939; ABD94800; ABF47605; ABF47704; ABG72870; ABG88256; ABI20848; ABI54442; ABI54443; ABI54446; ABI95217; ABI96089; ABI96090; ABI96092; ABI96099; ABI96100; ABI96102; ABI96109; ABI96110; ABO38065; ABO38362; ABO38373; ABO38395; BAA96110; BAA96113; BAA96116; BAA96123; CAA24272; CAA35097; AAA58800; ABI96114; ABI96115; ABI96120; ABI96121; ABI96122; ABI96127; ABI96130; ABI96137; ABI96140; ABI96141; ABI96145; ABI96146; ABI96147; ABI96150; ABK79959; ABL67253; ABM22202; ABO37988; ABO38010; ABO38021; AAB52910; AAB81460; AAB81463; AAD25308; AAK67319; AAK67320; AAK67325; AAK67326; AAK67327; AAK67328; AAK67335; AAK67336; AAK67337; AAK67344; AAK70451; AAK70452; AAK70453; AAK70458; AAK70459; AAK71687; AAK73325; AAK73326; AAK73331; AAK73332; AAK73333; AAA65545; AAA65552; AAA65555; AAA74286; AAA74290; AAA74292; AAA74300; AAA99877; AAB39851; AAB50957; AAB50959; AAL60443; AAL87870; AAM76687; AAM76688; AAP69676; AAP69677; AAP69680; AAP69682; ABA18145; ABB19507; ABB53729; ABB53740; ABC66234; ABC66235; ABD77730; ABD77796; ABD77961; ABD77983; ABD78082; ABD78104; ABD95317; ABE11701; ABE11712; ABE11734; ABE11823; ABE11900; ABE11942; ABF21274; ABF21276; ABF47572; ABG26242; ABG26244; ABG26245; ABG26780; AAK73334; AAK73341; AAK73342; AAK73343; AAK73344; AAL29701; AAL29707; AAO65612; AAV68006; AAW50829; AAW50830; AAW50831; AAW50832; AAY42117; AAY42118; AAY42121; AAY42122; AAZ17358; AAZ17359; AAZ79604; ABA87057; ABB02792; ABB02814; ABB02913; ABB02936; ABB83026; ABB83127; ABC66240; ABD59849; ABD60944; ABD60955; ABD78016; ABD94756; ABD95042; ABD95119; ABD95174; ABF47638; ABF47660; ABF47715; ABF47759; ABF47770; ABF47792; ABF47814; ABG37395; ABG47807; ABI96124; ABI96126; ABI96134; ABI96143; ABI96144; ABI96151; ABI96153; ABK79948; ABK80036; ABK80047; ABM22169; ABO38032; ABO52225; BAA00308; BAA00718; BAA00720; BAA01280; BAA21641; AAB52905; AAB52907; AAB57740; AAB81456; AAB81457; AAB81459; AAC14275; AAD25304; AAD25305; AAD25307; AAK67322; AAK67324; AAK67329; AAK67332; AAK67338; AAK67339; AAK67341; AAK67343; AAK70449; AAK70450; AAK70456; AAK73322; AAK73324; AAK73328; AAK73330; AAK73338; AAK73340; ABI21541; ABI21563; ABI30378; ABI85231; ABI96116; ABI96119; ABI96125; ABI96132; ABI96135; ABI96142; ABI96152; ABJ09184; ABK80025; ABO38043; BAA00309; BAA00719; BAA02765; BAA21642; AAB52904; AAB52906; AAB52908; AAB81458; AAD05215; AAD17229; AAD25303; AAD25306; AAD25312; AAK67321; AAK67323; AAK67330; AAK67331; AAK67333; AAK67340; AAK67342; AAK70455; AAK70457; AAK70464; AAK73321; AAK73323; AAK73327; AAK73329; AAK73336; AAK73337; AAK73339; AAL29694; AAK73345; AAL02002; AAL29695; AAL29702; AAL29708; AAL29710; AAL29711; AAL47668; AAO65768; AAW50828; AAW50836; AAY42114; AAY42115; AAZ15839; AAZ15840; AAZ15842; AAZ83253; ABA87080; ABA87231; ABB03101; ABB53707; ABB83015; ABC66243; ABC66246; ABD15515; ABD60779; ABD60856; ABD60878; ABD60900; ABD60933; ABD60966; ABD94943; ABD95020; ABD95097; ABD95207; ABF47748; ABF47825; ABF47847; ABG80183; ABG88212; ABI30565; ABI55088; ABI96154; ABI96160; ABI96166; ABG47829; ABI20870; ABI54447; ABI95272; ABI96155; ABI96156; ABI96157; ABI96162; ABI96163; ABI96171; ABI96172; ABI96173; ABJ51891; ABM22279; ABM66886; ABM66908; ABN51143; ABO32948; ABO32970; ABO32981; BAC82844; BAC82847; BAC82848; BAC82853; BAC82854; BAC82860; BAC82869; BAC82870; BAC82880; BAC82889; BAC82890; BAC82898; BAD02346; CAC86333; CAC86334; CAC86621; CAD29905; CAD29906; CAD29915; CAD29916; CAD29921; CAD29922; CAD29923; CAD29931; CAD29932; CAD29941; AAL29696; AAL29697; AAL29703; AAL29709; AAL29712; AAL29713; AAL47667; AAO88265; AAT00437; AAT00438; AAV67984; AAW22156; AAW50827; AAW50834; AAW50835; AAW50837; AAW56635; AAY42116; AAZ15838; AAZ15841; AAZ15843; ABA87045; ABA87091; ABB02825; ABB80103; ABB83138; ABC66244; ABC66245; ABD15258; ABD59847; ABD60867; ABD60889; ABD60911; ABD95108; ABD95185; ABD95196; ABF47726; ABF47737; ABF47836; ABG88201; ABI96159; ABI96161; ABI96165; ABI96167; ABI96168; ABI96169; ABI96170; ABJ51892; ABJ51894; ABJ51895; ABJ53449; ABK40634; ABK57093; ABL67055; ABL67066; ABL67209; ABM22026; ABM22268; ABN50940; ABN50962; ABN50973; ABO44123; BAC82843; BAC82850; BAC82851; BAC82857; BAC82859; BAC82866; BAC82867; BAC82873; BAC82876; BAC82877; BAC82879; BAC82886; BAC82887; BAC82893; BAC82896; BAC82897; BAD02356; CAC86337; CAC86605; CAC86608; CAC86609; CAC86617; CAC86618; CAC86620; CAC86625; CAD29902; CAD29909; CAD29912; CAD29918; ABJ16719; ABJ51890; ABJ51893; ABK57092; ABL67187; ABM22257; ABO44134; ABP49217; BAC82842; BAC82849; BAC82852; BAC82858; BAC82864; BAC82865; BAC82868; BAC82874; BAC82875; BAC82878; BAC82884; BAC82885; BAC82888; BAC82894; BAC82895; CAC86336; CAC86606; CAC86607; CAC86610; CAC86611; CAC86616; CAC86619; CAD29900; CAD29901; CAD29903; CAD29910; CAD29911; CAD29917; CAD29919; CAD29926; CAD29927; CAD29933; CAD29936; CAD29937; CAD29939; CAD29943; CAD29946; CAD29947; CAD29924; CAD29925; CAD29928; CAD29934; CAD29935; CAD29938; CAD29944; CAD29945; CAD29948; CAD35680; CAD35682; CAD57617; CAD57619; CAA86567; CAA91080; CAA91081; AAA16778; AAA16810; AAA16811; AAA16814; AAA16815; AAA19934; AAA43157; AAA43166; AAA43235; AAA43236; AAC57167; AAC57168; AAC57169; AAK40313; AAK40316; AAK51341; AAK51349; AAK51350; AAK51351; AAN64892; AAP69685; AAP69686; AAP69689; AAP69690; AAU25851; ABA08475; ABA08486; ABA08508; ABA12696; ABA42236; ABB96487; ABC41714; ABD78060; ABE27153; ABF47671; ABG72863; ABG72864; ABG72865; ABG72866; ABI20837; ABI20859; ABI54439; ABI54440; ABI54441; ABI84855; ABI92181; ABI92313; ABI95294; ABI96103; ABJ53438; ABJ53515; ABK40590; ABM22213; ABM22235; ABO32678; ABO38329; ABO38351; ABO52797; BAA96119; BAA96120; BAA96129; BAA96130; AAA67181; AAA67182; AAA67183; AAA74287; AAA74288; AAA74294; AAA74295; AAA92280; AAB39353; AAL87865; AAL87866; AAL87867; AAL87872; AAM75158; AAP34325; AAP60036; AAP60037; AAP69673; AAP69674; AAP69675; AAP69683; AAP69684; AAZ83299; AAZ85126; ABA06542; ABC66232; ABC66237; ABC66238; ABD77994; ABE11679; ABE11845; ABE11867; ABF21272; ABG26246; ABG37120; ABG67491; ABG88322; ABG88553; ABI21519; ABI96112; ABI96113; ABI96128; ABI96129; ABI96138; ABI96139; ABI96148; ABI96149; ABJ16675; ABK40039; ABK40050; ABK79970; ABL67264; ABM22180; ABM22191; ABN59401; ABN59412; ABO37999; ABO38054; BAA00721; BAA00722; BAA01027; BAA02766; BAA02767; BAA02768; BAA02769; BAA05874; BAA06719; AAB52909; AAB81461; AAB81462; AAD05216; CAD29942; CAD35678; CAD35679; CAD35686; CAD35687; CAD35688; CAD57616; CAD57620; CAD57623; CAD35681; CAD35683; CAD35684; CAD57618; AAD05217; AAD05218; AAD05219; AAD17218; AAD17219; AAD25301; AAD25302; AAD25309; AAD25310; AAD25311; AAK67334; AAK70454; AAK73320; AAK73335; AAL15459; AAL29693; AAL29698; AAL29699; AAL29700; AAL29704; AAL29705; AAL29706; AAL29714; AAL29715; AAO65769; AAT00436; AAT12706; AAW50833; AAY42119; AAY42120; AAY78939; AAZ15844; ABB02781; ABB02803; ABB02924; ABC42750; ABC66241; ABC66242; ABD59848; ABD78005; ABD78027; ABD95009; ABD95031; ABF47627; ABF47649; ABF47781; ABF47803; ABF47955; ABG37384; ABG47818; ABG47840; ABG80172; ABI21189; ABI95261; ABI95283; ABI96158; ABI96164; ABI96174; ABJ16642; ABJ16653; ABJ16664; ABJ16730; ABM22158; ABM22290; ABM66897; ABM67051; ABN50951; ABO32959; BAC82845; BAC82846; BAC82855; BAC82856; BAC82861; BAC82862; BAC82863; BAC82871; BAC82872; BAC82881; BAC82882; BAC82883; BAC82891; BAC82892; CAC86335; CAC86612; CAC86613; CAC86614; CAC86615; CAC86622; CAC86623; CAC86624; CAD29898; CAD29899; CAD29904; CAD29907; CAD29908; CAD29913; CAD29914; CAD29920; CAD29929; CAD29930; CAD29940; CAD29958; CAD35685; CAD57621 and CAD57622.

Protein sequences of influenza virus subtype H2 hemagglutinin suitable for inclusion as a cargo moiety in conjugates herein have the following accession numbers and are available to the public via the National Center for Biotechnology Information (NCBI) Entrez protein search and retrieval system:

BAC43764; BAF02312; AAO46270; AAO46271; AAO46272; AAO46273; AAO46274; AAO46275; AAO46276; AAO46277; AAO46278; AAO46279; AAO46280; AAO46281; AAO46282; AAO46283; AAO46284; AAO46285; AAO46286; AAO46287; AAO46288; AAO46289; AAO46290; AAO46291; AAO46292; AAO46293; AAO46294; AAO46295; AAO46296; AAO46297; AAO46298; AAO46299; AAO46300; AAO46301; AAO46302; AAO46303; AAO46304; AAO46305; AAS57527; AAS57528; AAS57529; AAS57530; AAT65325; AAT65327; AAT65331; AAT65348; AAT65351; AAV91219; ABB17150; ABB17670; ABB17681; ABB17692; ABB17703; ABB17714; ABB18378; ABB17725; ABB17736; ABB17756; ABB17813; ABB18025; ABB18036; ABB18047; ABB18058; ABB18069; ABB18080; ABB19639; ABB20141; ABB20229; ABB20240; ABB20466; ABB20509; ABI84382; ABI84384; ABI84450; ABI84458; ABI84459; ABI84588; ABI84744; ABI84755; ABI84959; ABI85183; ABL67022; ABM21949; ABO38098; ABO38296; ABO38307; ABO38701; ABO38712; ABO38723; ABO38734; ABO44057; ABO44090; ABO44101; ABO52236; ABO52247; ABO52302; ABO52379; ABP49437; ABP49459; ABP49470; BAA02770; BAA02771; BAA02772; BAA02773; BAA02774; BAA02775; AAY23639; AAY23640; AAY28987; AAY87410; AAY87419; ABF21270; ABF21275; AAA43185; AAA43196; AAA43089; AAA43090; AAA43659; AAA43243; AAA43117; AAA43284; AAA43450; AAA43096; AAA43247; AAA43248; AAA43088; AAA43345; AAA43576; AAA43578; AAA43658; AAA43660; AAA43662; AAA43678; AAA64362; AAA64364; AAA64365; AAA64363; AAA64366; BAF33428; BAF33438; BAF33398; BAF33408; BAF34322; BAF34377; BAF47131; BAF48641; BAF49415; AAD43235; AAD43236; AAD43237; AAD43238; AAD43239; AAD43240; AAD43241; AAD43242; AAD43243; AAD43244; AAD43245; AAD43246; AAD43247; AAD43248; AAD43249; AAK14980; AAF82100; AAF82101; AAF82102; AAF82103; AAF82104; AAF82105; AAF82106; AAF82107; AAF82108; AAF82109; AAF82110; AAF82111; AAF82112; AAN83926; AAN83927; AAN83928; AAN83929; AAN83930; AAN83931; AAO46268 and AAO46269.

Protein sequences of influenza virus subtype H3 hemagglutinin suitable for inclusion as a cargo moiety in conjugates herein have the following accession numbers and are available to the public via the National Center for Biotechnology Information (NCBI) Entrez protein search and retrieval system:

BAA77284; BAA77285; BAA77286; BAA77287; BAA77288; BAA77289; BAA77290; BAA77291; BAA77292; BAA77293; BAA77294; BAA86062; BAA86063; BAA86064; BAA86065; BAA96300; BAA96301; BAA96302; BAA96303; BAE75900; BAE75901; BAE75902; BAE75903; BAE75904; BAE75905; BAE75906; BAE75907; BAE75908; BAE75909; BAE75910; BAE75911; BAE75912; BAE75913; BAE75914; BAE75915; BAE75916; BAE75917; BAE75918; BAE75919; BAE54256; BAE54257; BAE54258; BAE54259; BAE54260; BAE54261; BAE54262; BAE75854; BAE94240; BAE94241; BAE94242; BAE94243; BAE94244; BAE94245; BAE94246; BAE94247; BAE94248; BAE94249; BAE94250; BAE94251; BAE94568; BAE94569; BAE94570; BAE96004; BAE96005; BAE96006; BAE96007; BAF46357; BAF46358; BAF46359; BAF46360; BAF46361; BAF46362; BAF46363; BAF46364; BAF46365; BAF46366; BAF46367; BAF46375; BAF46376; BAF46377; BAF46378; BAF46379; BAF46380; BAF46381; BAF46382; BAF46383; BAF46384; BAF46317; BAF46318; BAF46319; BAF46320; BAF46325; BAF46326; BAF46327; BAF46328; BAF46329; BAF46330; BAF46331; BAF46338; BAF46339; BAF46340; BAF46341; BAF46342; BAF46343; BAF46344; BAF47998; BAF47999; BAF48000; BAF48001; BAF48002; BAF48003; BAF48004; BAF48005; BAF48006; BAF48007; BAF48008; BAF48009; BAF48010; BAF48011; BAF48012; BAF48013; BAF48014; BAF48015; BAF48016; BAF48017; BAF48018; BAF48019; BAF48020; BAF48021; BAF48022; BAF48023; BAF48024; BAF48025; BAF48026; BAF48027; BAF48028; BAF48029; BAF48030; BAF48031; BAF48032; BAF48033; BAF48034; BAF48035; BAF48036; BAF48037; BAF48038; BAF48039; BAF33059; BAF34375; BAF34924; BAF37221; BAF43466; BAF46752; BAF46760; BAF46902; BAF46910; BAF48361; AAB66723; AAB66724; AAB66725; AAB66726; AAB66727; AAB66728; AAB66729; AAB66730; AAB66731; AAB66732; AAB66733; AAB66734; AAB66735; AAB66736; AAB66737; AAB66738; AAB66739; AAB66740; AAB66741; AAB66742; AAB66743; AAB66744; AAB66745; AAB66746; AAB66747; AAB66748; AAB66749; AAB66750; AAB66751; AAB66752; AAB66753; AAB66754; AAB66755; AAB66756; AAB66757; AAB66758; AAB66759; AAB66760; AAB66761; AAB66762; AAB66763; AAB66764; AAB66765; AAB66766; AAB66767; AAB66768; AAB66769; AAB66770; AAB66771; AAB66772; AAB66773; AAB66774; AAB66775; AAB66776; AAB66777; AAB66778; AAB66779; AAB66780; AAB66781; AAB66782; AAB66783; AAB66784; AAB66785; AAB66786; AAB66787; AAB66788; AAB66789; AAB66790; AAB66791; AAB66792; AAB66793; AAB66794; AAB66795; AAB66796; AAB66797; AAB66798; AAB66799; AAB66800; AAB66801; AAB66802; AAB66803; AAB66804; AAB66805; AAB66806; AAB66807; AAB66808; AAB66809; AAB66810; AAB63681; AAB63682; AAB63683; AAB63684; AAB63685; AAB63686; AAB63687; AAB63688; AAB63689; AAB63690; AAB63691; AAB63692; AAB63693; AAB63694; AAB63695; AAB63696; AAB63697; AAB63698; AAB63699; AAB63700; AAB63701; AAB63702; AAB63703; AAB63704; AAB63705; AAB63706; AAB63707; AAB63708; AAB63709; AAB63710; AAB63711; AAB63712; AAB63713; AAB63714; AAB63715; AAB63716; AAB63717; AAB63718; AAB63719; AAB63720; AAB63721; AAB63722; AAB63723; AAB63724; AAB63725; AAB63726; AAB63727; AAB63728; AAB63729; AAB63730; AAB63731; AAB63732; AAB63733; AAB63734; AAB63735; AAB63736; AAB63737; AAB63738; AAB63739; AAB63740; AAB63741; AAB63742; AAB63743; AAB63744; AAB63745; AAB63746; AAB63747; AAB63748; AAB63749; AAB63750; AAB63751; AAB63752; AAB63753; AAB63754; AAB63755; AAB63756; AAB63757; AAB63758; AAB63759; AAB63760; AAB63761; AAB63762; AAB63763; AAB63764; AAB63765; AAB63766; AAB63767; AAB69773; AAB69774; AAB69775; AAB69776; AAB69777; AAB69778; AAB69779; AAB69780; AAB69781; AAB69782; AAB69783; AAB69784; AAB69785; AAB69786; AAB69787; AAB69788; AAB69789; AAB69790; AAB69791; AAB69792; AAB69793; AAB69794; AAB69795; AAB69796; AAB69797; AAB69798; AAB69799; AAB69800; AAB69801; AAB69802; AAB69803; AAB69804; AAB69805; AAB69806; AAB69807; AAB69808; AAB69809; AAB69810; AAB69811; AAB69812; AAB69813; AAB69814; AAB69815; AAB69816; AAB69817; AAB69818; AAB69819; AAB69820; AAB69821; AAB69822; AAB69823; AAB69824; AAB69825; AAB69826; AAB69827; AAB69828; AAB69829; AAB69830; AAB69831; AAB69832; AAB69833; AAB69834; AAB69835; AAB69836; AAB69837; AAB69838; AAB69839; AAB69840; AAB69841; AAB69842; AAB69843; AAB69844; AAB69845; AAB69846; AAB69847; AAB69848; AAB69849; AAB69850; AAB69851; AAC59602; AAC59603; AAC59604; AAC63474; AAC63475; AAC63476; AAC63477; AAC63478; AAC31556; AAC36729; AAC36730; AAC36731; AAC36732; AAC36733; AAC36734; AAC36735; AAC36736; AAC36737; AAC36738; AAC78086; AAC78087; AAC78088; AAC78089; AAC78090; AAC78091; AAC78092; AAC78093; AAC78094; AAC78095; AAC78096; AAC78097; AAC78098; AAC83790; AAC83791; AAC83792; AAC83793; AAC83794; AAC83795; AAC83796; AAC83797; AAC83798; AAC83799; AAC83800; AAF06948; AAF06949; AAF06950; AAD34847; AAD34848; AAD34849; AAD34850; AAD34851; AAD34852; AAD34853; AAD34854; AAD34855; AAD34856; AAD34857; AAD51239; AAD51240; AAD51241; AAD51242; AAF16416; AAF16417; AAF16418; AAF16419; AAF16420; AAF16421; AAF16422; AAF16423; AAF16424; AAF16425; AAF16426; AAF16427; AAF16428; AAF16429; AAF16430; AAF16431; AAF16432; AAF16433; AAF16434; AAF16435; AAF16436; AAF16437; AAF16438; AAF16439; AAF16440; AAF16441; AAF16442; AAF16443; AAF16444; AAF16445; AAF16446; AAF16447; AAF16448; AAF16449; AAF16450; AAF16451; AAF16452; AAF16453; AAF16454; AAF16455; AAF16456; AAF16457; AAF16458; AAF16459; AAF16460; AAF16461; AAF16462; AAF16463; AAF16464; AAF16465; AAF16466; AAF16467; AAF16468; AAF16469; AAF16470; AAF16471; AAF16472; AAF16473; AAF16474; AAF16475; AAF16476; AAF16477; AAF16478; AAF16479; AAF16480; AAF16481; AAF16482; AAF16483; AAF16484; AAF16485; AAF16486; AAF16487; AAF16488; AAF16489; AAF16490; AAF16491; AAF16492; AAF16493; AAF16494; AAF16495; AAF16496; AAF16497; AAF16498; AAF16499; AAF16500; AAF16501; AAF16502; AAF16503; AAF16504; AAF16505; AAF16506; AAF16507; AAF16508; AAF16509; AAF16510; AAF16511; AAF16512; AAF16513; AAF16514; AAF16515; AAF16516; AAF16517; AAF16518; AAF22345; AAF22346; AAF22347; AAF22348; AAF22349; AAF22350; AAF22351; AAF22352; AAF22353; AAF18089; AAF18090; AAF18091; AAF18092; AAF18093; AAF13705; AAF13706; AAF19421; AAL59048; AAL59049; AAL59050; AAL59051; AAO15354; AAO15355; AAO15356; AAO15357; AAF60285; AAG01749; AAG01758; AAG01767; AAG01776; AAG01785; AAK49194; AAK49195; AAK49196; AAK49197; AAK49198; AAK49199; AAK49200; AAK49201; AAK49202; AAK49203; AAK49204; AAG10735; AAG10736; AAG10737; AAG10738; AAG10739; AAG10740; AAG33221; AAG33222; AAG33223; AAG33224; AAG47797; AAG47798; AAG47799; AAG47800; AAG47801; AAG47802; AAG47803; AAG47804; AAG47805; AAG47806; AAG47807; AAG47808; AAG47809; AAG47810; AAG47811; AAG47812; AAG47813; AAG47814; AAG47815; AAG47816; AAG47817; AAG47818; AAG47819; AAG49302; AAG49303; AAG49304; AAG49305; AAG49306; AAG49307; AAG49308; AAG49309; AAG49310; AAG49311; AAG49312; AAG49313; AAG49314; AAG49335; AAG49336; AAG49337; AAG49338; AAG49339; AAK51718; AAL18558; AAL18559; AAL18560; AAL18561; AAL18562; AAL18563; AAL18564; AAL18565; AAL18566; AAL18567; AAL18568; AAL18569; AAL18570; AAL18571; AAL18572; AAL18573; AAL18574; AAL18575; AAL18576; AAL18577; AAL18578; AAL18579; AAL18580; AAL18581; AAL18582; AAL18583; AAL18584; AAL18585; AAL18586; AAL18587; AAL18588; AAL18589; AAL18590; AAL18591; AAL18592; AAL18593; AAL18594; AAL18595; AAL18596; AAL18597; AAL18598; AAK82853; AAK82854; AAK82855; AAK82856; AAK82857; AAK82858; AAK82859; AAK82860; AAK82861; AAK82862; AAK82863; AAK82864; AAK82865; AAK82866; AAK82867; AAK82868; AAK82869; AAK52910; AAK52911; AAK52912; AAK54141; AAK54142; AAK54143; AAK54144; AAK54145; AAK54146; AAK54147; AAK54148; AAK54149; AAK54150; AAK54151; AAK63816; AAK63817; AAK63818; AAK63819; AAK63820; AAK63821; AAK63822; AAK63823; AAK63824; AAK63825; AAK63826; AAK67171; AAK67172; AAK67173; AAK67174; AAK67175; AAK67176; AAK67177; AAK67178; AAK67179; AAK67180; AAK67181; AAK67182; AAK67183; AAK67184; AAK67185; AAK67186; AAK67187; AAK67188; AAK67189; AAK67190; AAK67191; AAK67192; AAK67193; AAK67194; AAK67195; AAK67196; AAK67197; AAK67198; AAK67199; AAK67200; AAK67201; AAL30462; AAL30463; AAL30464; AAL60147; AAL60148; AAL60149; AAL60150; AAL60151; AAL60152; AAL60153; AAL77301; AAL77302; AAL77303; AAL77304; AAL77305; AAL77306; AAL77307; AAL77308; AAL77309; AAL77310; AAL77311; AAL77312; AAL77313; AAL77314; AAL77315; AAL77316; AAL77317; AAL77318; AAL77319; AAL77320; AAL77321; AAL77322; AAL77323; AAL77324; AAL77325; AAL77326; AAL77327; AAL77328; AAL77329; AAL62329; AAM46871; AAM46872; AAM46873; AAM46874; AAM46875; AAM46876; AAM46877; AAM46878; AAM46879; AAM46880; AAM46881; AAM46882; AAM46883; AAM46884; AAM46885; AAM46886; AAM46887; AAM46888; AAM46889; AAM46890; AAM46891; AAM82560; AAM82561; AAM82562; AAM88280; AAM88283; AAN01150; AAN01151; AAN01152; AAN01153; AAN01154; AAN01155; AAN01156; AAN01157; AAN01158; AAN01159; AAN01160; AAN01161; AAN01162; AAN01163; AAN01164; AAN01165; AAN01166; AAN01167; AAQ10355; AAQ10356; AAQ10357; AAQ10358; AAQ10359; AAQ10360; AAQ10361; AAQ10362; AAQ10363; AAQ10364; AAQ10365; AAQ10366; AAQ10370; AAQ10371; AAQ10374; AAQ10375; AAQ10376; AAQ10377; AAQ10378; AAQ10379; AAQ10381; AAQ10382; AAQ10383; AAQ10384; AAQ10397; AAQ10398; AAQ10399; AAQ10400; AAQ10401; AAQ10402; CAA11167; CAA11168; CAA11169; CAA11170; CAA11171; CAA11172; CAC81013; CAC81016; CAC81017; CAC81018; CAC40044; CAC40045; CAC40046; CAC40047; CAC40048; CAC40049; CAC40050; CAC40051; CAC36995; CAC37007; CAC37327; CAC86626; CAD20322; CAD20336; CAD44999; CAG27339; CAG27340; CAG27341; CAG27342; CAG28960; CAG28961; CAG28962; CAG34129; CAH56424; CAJ32551; CAJ32558; CAD22811; CAD22818; AAK53066; AAK62039; AAK62040; AAK62041; AAK62042; AAK62043; AAL06634; AAL06635; AAL06636; AAL06637; AAL06638; AAN17779; AAN63953; AAN63954; AAN63955; AAN63956; AAN63957; AAN63958; AAN83932; AAN83933; AAN83934; AAN83935; AAN83936; AAN83937; AAN83938; AAN83939; AAN83940; AAN83941; AAN83942; AAN83943; AAN83944; AAN83945; AAN83946; AAN83947; AAN83948; AAN83949; AAN83950; AAN83951; AAN83952; AAN83953; AAN83954; AAN83955; AAN83956; AAN83957; AAN83958; AAN83959; AAN83960; AAN83961; AAP21996; AAP21997; AAP23238; AAQ18434; AAQ18435; AAP79943; AAP79947; AAP79953; AAP79961; AAP79966; AAP79973; AAP79975; AAR12332; AAR12333; AAR12334; AAR12335; AAR12336; AAR12337; AAR12338; AAR12339; AAR12340; AAR12341; AAR12342; AAR12343; AAR12344; AAR12345; AAR12346; AAR12347; AAR12348; AAR12349; AAQ86988; AAQ85081; AAQ85082; AAQ85083; AAQ85084; AAQ85085; AAQ85086; AAQ85087; AAQ85088; AAQ85089; AAQ85090; AAQ85091; AAT12703; AAT12704; AAR90879; AAQ90291; AAQ92920; AAQ92921; AAQ92922; AAQ92923; AAQ92924; AAQ92925; AAQ92926; AAQ92927; AAQ92928; AAQ92929; AAQ92930; AAQ92931; AAR25201; AAR33033; AAT07998; AAT08000; AAT08002; AAT08004; AAT12654; AAT12655; AAT12656; AAT12657; AAT12658; AAT12659; AAT12660; AAT12661; AAT12662; AAT12663; AAT12664; AAT12665; AAT12666; AAT12667; AAT12668; AAT12669; AAT12670; AAT12671; AAT12672; AAT12673; AAT12674; AAT12675; AAT12676; AAS93870; AAS93871; AAS93872; AAS93873; AAS93874; AAS93875; AAS93876; AAS93877; AAS93878; AAS93879; AAS93880; AAS93881; AAS93882; AAS93883; AAS93884; AAT09637; AAT09638; AAT09639; AAT81341; AAT81342; AAT81343; AAT81344; AAT81345; AAT81346; AAT81347; AAT81348; AAT81349; AAT81350; AAT81351; AAT81352; AAT81353; AAT81354; AAT81355; AAT81356; AAT81357; AAT81358; AAT81359; AAT81360; AAT81361; AAT81362; AAU25861; AAU25871; AAT79527; AAT79528; AAT79529; AAT65319; AAT65321; AAT65324; AAT65333; AAT65334; AAT65345; AAT65349; AAT51806; AAT51807; AAT51808; AAT51809; AAT51810; AAT51811; AAT51812; AAT51813; AAT51814; AAT51815; AAT51816; AAT51817; AAT51818; AAT51819; AAT51820; AAT51821; AAT51822; AAT51823; AAT51824; AAT51825; AAT51826; AAT51827; AAT51828; AAT51829; AAT51830; AAT51831; AAT51832; AAT51833; AAT51834; AAT51835; AAT51836; AAT51837; AAT51838; AAT51839; AAT51840; AAT51841; AAT51842; AAT51843; AAT51844; AAT51845; AAT51846; AAT51847; AAT51848; AAT51849; AAT51850; AAT51851; AAT51852; AAT51853; AAT51854; AAT51855; AAT51856; AAT51857; AAT51858; AAT51859; AAT64666; AAT64667; AAT64668; AAT64669; AAT64670; AAT64671; AAT64672; AAT64673; AAT64674; AAT64675; AAT64676; AAT64677; AAT64678; AAT64679; AAT64680; AAT64681; AAT64682; AAT64683; AAT64684; AAT64685; AAT64686; AAT64687; AAT64688; AAT64689; AAT64690; AAT64691; AAT64692; AAT64693; AAT64694; AAT64695; AAT64696; AAT64697; AAT64698; AAT64699; AAT64700; AAT64701; AAT64702; AAT64703; AAT64704; AAT64705; AAT64706; AAT64707; AAT64708; AAT64709; AAT64710; AAT64711; AAT64712; AAT64713; AAT64714; AAT64715; AAT64716; AAT64717; AAT64718; AAT64719; AAT64720; AAT64721; AAT64722; AAT64723; AAT64724; AAT64725; AAT64726; AAT64727; AAT64728; AAT64729; AAT64730; AAT64731; AAT64732; AAT64733; AAT64734; AAT64735; AAT64736; AAT64737; AAT64738; AAT64739; AAT64740; AAT64741; AAT64742; AAT64743; AAT64744; AAT64745; AAT64746; AAT64747; AAT64748; AAT64749; AAT64750; AAT64751; AAT64752; AAT64753; AAT64754; AAT64755; AAT64756; AAT64757; AAT64758; AAT64759; AAT64760; AAT64761; AAT64762; AAT64763; AAT64764; AAT64765; AAT64766; AAT64767; AAT64768; AAT64769; AAT64770; AAT64771; AAT64772; AAT64773; AAT64774; AAT64775; AAT64776; AAT64777; AAT64778; AAT64779; AAT64780; AAT64781; AAT64782; AAT64783; AAT64784; AAT64785; AAT64786; AAT64787; AAT64788; AAT64789; AAT64790; AAT64791; AAT64792; AAT64793; AAT64794; AAT64795; AAT64796; AAT64797; AAT64798; AAT64799; AAT64800; AAT64801; AAT64802; AAT64803; AAT64804; AAT64805; AAT64806; AAT64807; AAT64808; AAT64809; AAT64810; AAT64811; AAT64812; AAT64813; AAT64814; AAT64815; AAT64816; AAT64817; AAT64818; AAT64819; AAT64820; AAT64821; AAT64822; AAT64823; AAT64824; AAT64825; AAT64826; AAT64827; AAT64828; AAT64829; AAT64830; AAT64831; AAT64832; AAT64833; AAT64834; AAT64835; AAT64836; AAT64837; AAT64838; AAT64839; AAT64840; AAT64841; AAT64842; AAT64843; AAT64844; AAT64845; AAT64846; AAT64847; AAT64848; AAT64849; AAT64850; AAT64851; AAT64852; AAT64853; AAT64854; AAT64855; AAT64856; AAT64857; AAT64858; AAT64859; AAT64860; AAT64861; AAT64862; AAT64863; AAT64864; AAT64865; AAT64866; AAT64867; AAT64868; AAT64869; AAT64870; AAT64871; AAT64872; AAT64873; AAT64874; AAT64875; AAT64876; AAT64877; AAT64878; AAT64879; AAT64880; AAT64881; AAT64882; AAT64883; AAT64884; AAT64885; AAT64886; AAU07825; AAU07826; AAU07827; AAU07828; AAU07829; AAU07830; AAU07831; AAU09399; AAW24444; AAW24445; AAW24446; AAW24447; AAW24448; AAW24449; AAW24450; AAW24451; AAU11522; AAU25949; AAV80797; AAV80798; AAW65986; AAW65987; AAW65988; AAW65989; AAW65990; AAW34374; AAW34375; AAW34376; AAW34377; AAW34378; AAW50838; AAW50839; AAW50840; AAW50841; AAX23575; AAW78047; AAW78048; AAW78049; AAW78050; AAW78051; AAW78052; AAX77666; AAX77667; AAX77668; AAX77669; AAX77670; AAX77671; AAX77672; AAX77673; AAX77674; AAX14851; AAX47732; AAX47733; AAX47734; AAX47735; AAX47736; AAX47737; AAX47738; AAX47739; AAX47740; AAX47741; AAX47742; AAX47743; AAX47744; AAX47745; AAX47746; AAX47747; AAX47748; AAX47749; AAX47750; AAX47751; AAX47752; AAX47753; AAX47754; AAX47755; AAX47756; AAX47757; AAX49559; AAX49562; AAY85891; AAY85892; AAY85893; AAY85894; AAY85895; AAY85896; AAY85897; AAY85898; AAY85899; AAY85900; AAY85901; AAY85902; AAY85903; AAY85904; AAY85905; AAY85906; AAY85907; AAY85908; AAY85909; AAY85910; AAY85911; AAX63815; AAX63816; AAX63817; AAX63818; AAX63819; AAX63820; AAX63821; AAX63822; AAX63823; AAX63824; AAX63825; AAX63826; AAX63827; AAX63828; AAY42043; AAY42044; AAY42045; AAY42046; AAY42047; AAY42048; AAY42049; AAY42050; AAY42051; AAY42052; AAY42053; AAY42054; AAY42055; AAY42056; AAY42057; AAY42058; AAY42059; AAY42060; AAY42061; AAY42062; AAY42063; AAY42064; AAY42065; AAY42066; AAY42067; AAX84524; AAX84525; AAX84526; AAX84527; AAX84528; AAX84529; AAX84530; AAX84531; AAX84532; AAX84533; AAX84534; AAX84535; AAX84536; AAX84537; AAX84538; AAX84539; AAX84540; AAX84541; AAX84542; AAX84543; AAX84544; AAX84545; AAX84546; AAX84547; AAX84548; CAL40875; AAX11455; AAX11475; AAX11485; AAX11495; AAX56420; AAX11505; AAY28295; AAX11515; AAX11565; AAX11575; AAX11585; AAX11595; AAX11605; AAX11615; AAX11635; AAX12731; AAX11465; AAY28571; AAX12751; AAX11525; AAX11535; AAX11545; AAX11555; AAX11625; AAX12741; AAX12761; AAX12771; AAX12781; AAX12791; AAX12801; AAX12811; AAX34061; AAX35821; AAX35831; AAX38237; AAX35841; AAX35851; AAX47525; AAX56490; AAX47515; AAX35861; AAX47535; AAX35871; AAX56380; AAX56390; AAX56400; AAX56410; AAX56430; AAX56440; AAX56450; AAX56460; AAX56470; AAX56480; AAX56500; AAX56510; AAX56520; AAX56540; AAX56550; AAX56560; AAX56570; AAX56580; AAX56590; AAX56600; AAX57644; AAX57654; AAX57664; AAX57674; AAX57684; AAX57694; AAX57704; AAX57714; AAX57733; AAX57734; AAX57744; AAX57754; AAX57764; AAX57774; AAX57784; AAX57794; AAX57804; AAX57814; AAX57824; AAX57834; AAX57844; AAX57854; AAX57864; AAX57874; AAX57884; AAX57894; AAX57904; AAX57914; AAX57924; AAX57934; AAX57944; AAX76623; AAX76633; AAX76643; AAX76653; AAX76663; AAY59035; AAX76673; AAX76683; AAX76693; AAX76703; AAX76713; AAY28375; AAX76723; AAX76733; AAX76743; AAX76753; AAX76763; AAY18086; AAY18096; AAY18611; AAY18585; AAY18106; AAY18116; AAY18564; AAY18126; AAY18136; AAY18146; AAY18156; AAY18166; AAY18176; AAY18186; AAY18196; AAY27863; AAY28385; AAY27843; AAY28561; AAY28345; AAY28395; AAY27853; AAY28325; AAY27959; AAY27994; AAY28004; AAY28014; AAY28265; AAY28275; AAY28285; AAY28363; AAY28648; AAY28305; AAY28315; AAY28335; AAY28355; AAY28638; AAY28405; AAY28486; AAY28521; AAY28531; AAY28541; AAY28628; AAY28551; AAY28618; AAY28581; AAY28591; AAY28608; AAY44610; AAY44906; AAY44620; AAY44621; AAY44631; AAY44896; AAY44641; AAY44796; AAY44795; AAY44785; AAY44775; AAY44765; AAY44755; AAY44651; AAY44661; AAY46371; AAY46381; AAY47013; AAY47023; AAY47052; AAY46391; AAY47075; AAY47085; AAY46416; AAY46426; AAY46436; AAY64192; AAY64202; AAY64212; AAY64252; AAY64272; AAY64292; AAY64222; AAY64312; AAY64232; AAY64242; AAY64322; AAY64262; AAY64282; AAY64342; AAY64302; AAY64352; AAY64332; AAY64392; AAY64362; AAY64372; AAY64382; AAY64402; AAY98770; AAY98037; AAY98047; AAY98187; AAY98057; AAY98067; AAY98077; AAY98087; AAY98097; AAY98107; AAY98117; AAY98127; AAY98137; AAY98147; AAY98157; AAY98167; AAY98177; AAY98195; AAY98217; AAY98207; AAY98237; AAY98227; AAY98247; AAY98319; AAY98329; AAY98339; AAY98353; AAY98366; AAY98376; AAY98386; AAY98396; AAY98406; AAZ38539; AAZ38561; AAZ38462; AAZ38473; AAZ38484; AAZ38495; AAZ38506; AAZ38517; AAZ38528; AAZ38583; AAZ38605; AAZ38550; AAZ38572; AAZ38594; AAZ38616; AAZ38638; AAZ38650; AAZ43370; AAZ43383; AAZ43394; AAZ43405; AAZ74386; AAZ74352; AAZ74363; AAZ74430; AAZ74397; AAZ74408; AAZ74419; AAZ74441; AAZ74452; AAZ74507; AAZ74463; AAZ74474; AAZ74485; AAZ74496; AAZ74529; AAZ74518; AAZ74540; AAZ74573; AAZ74606; AAZ74551; AAZ74595; AAZ74562; AAZ74584; AAZ74617; AAZ79505; AAZ79516; AAZ79527; AAZ79560; AAZ79571; AAZ79582; AAZ79626; AAZ79944; AAZ79593; AAZ79615; AAZ79627; AAZ79941; AAZ79963; AAZ79974; AAZ79985; AAZ80017; AAZ79996; AAZ80007; AAZ80030; AAZ83288; AAZ83242; AAZ83312; AAZ83266; AAZ83277; AAZ83323; AAZ83371; AAZ83382; AAZ83649; AAZ83688; ABA12740; ABA12751; ABA12762; ABA12780; ABA12773; ABA16214; ABA18048; ABA18134; ABA18156; ABA18026; ABA18123; ABA26799; ABA26700; ABA26711; ABA26722; ABA26733; ABA26744; ABA26755; ABA26766; ABA26777; ABA26788; ABA42269; ABA43167; ABA43178; ABA42291; ABA43336; ABA43200; ABA42302; ABA42313; ABA42335; ABA42346; ABA42989; ABA42357; ABA42368; ABA42379; ABA42390; ABA42401; ABA42412; ABA42443; ABA42454; ABA42465; ABA42476; ABA42487; ABA42498; ABA42978; ABA42939; ABA42928; ABA42509; ABA42520; ABA42531; ABA42542; ABA42553; ABA42564; ABA87242; ABA87253; ABB96509; ABB02836; ABB02847; ABB02858; ABB02869; ABB02880; ABB02891; ABB02902; ABB04283; ABB04294; ABB04305; ABB04316; ABB04327; ABB04338; ABB04349; ABB04360; ABB04371; ABB02947; ABB02958; ABB02969; ABB02980; ABB02991; ABB03002; ABB03013; ABB03024; ABB03035; ABB03046; ABB03057; ABB03068; ABB03079; ABB03090; ABB03112; ABB04906; ABB04917; ABB04928; ABB04939; ABB04950; ABB04961; ABB04983; ABB05183; ABB05194; ABB05205; ABB05216; ABB04994; ABB05005; ABB19704; ABB19712; ABB19723; ABB19744; ABB19758; ABB86785; ABB86796; ABB87034; ABB87377; ABB87388; ABB87399; ABB87410; ABB87421; ABB87429; ABB87440; ABB87451; ABB87462; ABB87789; ABB88149; ABB88150; ABB88152; ABB88162; ABB88173; ABB88183; ABB88256; ABB88309; ABB88342; ABB88369; ABB46547; ABB46392; ABB46403; ABB46414; ABB46425; ABB46436; ABB46447; ABB46458; ABB53614; ABB53625; ABB53652; ABB53663; ABB53674; ABB53685; ABB53696; ABB53718; ABB53751; ABB54514; ABB52376; ABB77853; ABB59996; ABB77864; ABC50167; ABC50178; ABC50189; ABB79716; ABB60007; ABB79731; ABB80034; ABB80023; ABB79788; ABB79799; ABB79810; ABB79957; ABB79968; ABB80001; ABB80012; ABB80137; ABB80148; ABB80159; ABB80081; ABB80092; ABB80185; ABB80196; ABB80207; ABB80229; ABB80492; ABB80503; ABB80514; ABB80748; ABB80218; ABB80529; ABB80641; ABB80661; ABB80240; ABB80251; ABB80672; ABB80682; ABB80693; ABB80704; ABB80737; ABB80715; ABB80724; ABB82183; ABB82227; ABB96319; ABB96330; ABB96341; ABB96352; ABB96363; ABB96374; ABB96395; ABB96498; ABB96520; ABB96531; ABC02234; ABC02288; ABC02299; ABC02332; ABC02321; ABC02255; ABC02266; ABC02310; ABC39805; ABC40642; ABC40544; ABC40555; ABC40608; ABC40619; ABC41692; ABC41703; ABC41725; ABC41952; ABC41736; ABC41953; ABC41964; ABC42014; ABC42114; ABC42125; ABC42136; ABC42147; ABC42929; ABC42940; ABC42158; ABC42169; ABC42180; ABC42192; ABC42951; ABC42307; ABC42318; ABC42346; ABC42461; ABC42962; ABC42494; ABC42505; ABC42516; ABC42527; ABC42973; ABC42984; ABC42995; ABC43006; ABC43017; ABC42574; ABC43028; ABC43039; ABC43050; ABC43061; ABC43072; ABC43083; ABC43094; ABC43105; ABC43116; ABC42585; ABC42596; ABC43127; ABC42607; ABC42618; ABC42629; ABC43138; ABC43149; ABC42640; ABC43160; ABC42651; ABC43171; ABC43182; ABC42662; ABC42673; ABC42684; ABC43475; ABC42695; ABC43486; ABC43497; ABC43508; ABC42706; ABC42717; ABC42728; ABC42739; ABC42761; ABC42772; ABC42783; ABC42794; ABC42805; ABC43519; ABC43530; ABC42816; ABC43541; ABC42827; ABC42838; ABC42849; ABC43552; ABC42860; ABC42871; ABC42882; ABC42893; ABC46554; ABC46565; ABC46576; ABC54668; ABC54679; ABC50200; ABC50211; ABC50222; ABC50233; ABC50244; ABC50255; ABC50266; ABC50277; ABC50288; ABC50299; ABC50310; ABC50321; ABC50332; ABC50343; ABC50354; ABC50365; ABC50376; ABC50387; ABC50398; ABC50409; ABC50420; ABC67319; ABC67817; ABC68233; ABC67850; ABC67883; ABC67894; ABC67967; ABC67978; ABC67454; ABC67989; ABC68000; ABC68049; ABC68060; ABC67471; ABC68071; ABC68093; ABC67543; ABC67554; ABC68082; ABC67565; ABC67576; ABC67587; ABC67598; ABC67609; ABC67620; ABC67631; ABC67642; ABC67653; ABC67664; ABC67675; ABC67686; ABC67697; ABC67708; ABC67719; ABC67733; ABC67806; ABC67828; ABC67839; ABC67861; ABC67872; ABC68222; ABC84389; ABC84400; ABC86148; ABC84411; ABC86124; ABC86040; ABC84422; ABC86029; ABC86018; ABC84433; ABC86007; ABC85996; ABC85985; ABC85974; ABC84444; ABC85963; ABC85952; ABD38134; ABC85941; ABC85930; ABC85919; ABC85908; ABC85897; ABC85886; ABC85875; ABC84498; ABC84509; ABC85864; ABC85853; ABC85842; ABC84520; ABC85831; ABC85765; ABC85820; ABC85809; ABC85798; ABC84531; ABC84542; ABC86137; ABC85787; ABC85776; ABC85754; ABC84560; ABC84571; ABD15526; ABD15537; ABD16538; ABD16527; ABD16516; ABD16560; ABD16505; ABD16494; ABD16483; ABD16472; ABD16358; ABD16347; ABD16336; ABD16325; ABD16314; ABD16303; ABD16549; ABD16762; ABD17334; ABD17323; ABC97374; ABD16751; ABD16740; ABD16729; ABD16718; ABD16593; ABD16582; ABD16571; ABD16292; ABD15790; ABD15779; ABD15768; ABD15757; ABD15746; ABD15735; ABD15724; ABD15713; ABD15702; ABD15691; ABD15680; ABD15669; ABD15658; ABD15647; ABD15504; ABD15493; ABD15482; ABD15471; ABD15460; ABD15449; ABD15625; ABD15614; ABD15603; ABD15592; ABD15581; ABD15570; ABD15559; ABD15548; ABD15636; ABD60790; ABD60801; ABD61293; ABD60812; ABD61304; ABD60823; ABD61529; ABD61757; ABD61777; ABD61315; ABD61326; ABE12078; ABD60834; ABD61337; ABD61348; ABD61359; ABD61370; ABD61381; ABD60845; ABD61392; ABD61403; ABD61260; ABD61271; ABD60922; ABD61282; ABD61551; ABD61249; ABE12532; ABE12623; ABD62833; ABD62794; ABD77598; ABD77609; ABD77620; ABD77631; ABD77642; ABD77653; ABD77664; ABD79123; ABD79134; ABD79145; ABD77686; ABE12645; ABD77697; ABD79156; ABD79167; ABD79178; ABD77741; ABD77752; ABD77763; ABD77774; ABD77785; ABD79032; ABD77829; ABD79189; ABD77840; ABD79200; ABD79211; ABD77851; ABD79222; ABD77862; ABD77873; ABD79233; ABD77884; ABD77895; ABD79244; ABD77906; ABD78049; ABD78115; ABD78126; ABD94734; ABD94745; ABD94767; ABD94822; ABD94833; ABD94844; ABD94855; ABD94866; ABD94877; ABD94888; ABD94899; ABD94910; ABD94921; ABD94932; ABD94954; ABE11911; ABE12123; ABE27164; ABE13076; ABE13323; ABE13471; ABE13555; ABE13595; ABE13606; ABE13617; ABE13628; ABE13639; ABE13652; ABE13824; ABE14019; ABE14030; ABE14041; ABE14052; ABE14063; ABE14124; ABE14464; ABE14840; ABE15578; ABF47550; ABF47594; ABF47616; ABF47858; ABF47902; ABI47947; ABI48006; ABF82651; ABF83447; ABF82695; ABF82706; ABG26758; ABG26769; ABG26802; ABG26846; ABG26857; ABG26868; ABG26879; ABG26890; ABG26901; ABG26912; ABG26923; ABG26934; ABG26956; ABG37131; ABG37142; ABG37153; ABG37164; ABG37175; ABG37186; ABG37197; ABG37208; ABG37219; ABG37230; ABG37241; ABG37252; ABG37263; ABG37274; ABG37285; ABG37296; ABG37307; ABG37318; ABG37329; ABG37340; ABG37351; ABG37373; ABG37406; ABG37417; ABG37428; ABG37439; ABG37450; ABG37461; ABG37472; ABG37483; ABG37494; ABG37505; ABG37516; ABG37527; ABG37538; ABG37549; ABG37560; ABG37571; ABG37582; ABG37593; ABG37604; ABG37615; ABG47851; ABG47862; ABG47873; ABG47884; ABG47895; ABG47906; ABG47917; ABG47928; ABG47939; ABG47950; ABG47961; ABG47972; ABG47983; ABG47994; ABG48005; ABG48016; ABG48027; ABG48038; ABG48060; ABG48071; ABG48082; ABG48093; ABG48104; ABG48115; ABG48126; ABG48137; ABG48148; ABG48159; ABG48170; ABG48181; ABG48192; ABG48203; ABG48214; ABG48225; ABG48236; ABG48247; ABG48258; ABG48269; ABG48280; ABG48291; ABG48302; ABG48313; ABG48324; ABG48335; ABG48346; ABG48357; ABG48368; ABG67135; ABG67146; ABG67667; ABG67157; ABG67168; ABG67179; ABG67190; ABG67201; ABG67212; ABG67223; ABG67234; ABG67245; ABG67502; ABG67513; ABG67524; ABG67535; ABG67546; ABG67557; ABG67568; ABG67579; ABG67590; ABG67601; ABG67612; ABG67623; ABG67634; ABG67645; ABG67656; ABG79941; ABG79963; ABG79974; ABG79985; ABG79996; ABG80007; ABG80018; ABG80029; ABG80040; ABG80051; ABG80062; ABG80073; ABG80084; ABG80095; ABG80106; ABG80117; ABG80128; ABG80139; ABG80150; ABG80161; ABG80194; ABG80205; ABG80216; ABG80227; ABG80238; ABG80249; ABG80260; ABG80271; ABG80282; ABG80293; ABG80304; ABG80315; ABG80326; ABG80337; ABG80348; ABG80359; ABG80370; ABG80381; ABG80392; ABG80403; ABG80414; ABG80425; ABG80436; ABG88289; ABG88355; ABG88366; ABG88377; ABG88388; ABG88399; ABG88410; ABG88421; ABG88432; ABG88443; ABG88454; ABG88465; ABG88476; ABG88487; ABG88498; ABG88509; ABG88520; ABG88531; ABG88564; ABG88575; ABG88586; ABG88597; ABG88608; ABG88619; ABG88630; ABG88641; ABG88652; ABG88663; ABG88674; ABG88685; ABG88696; ABG88707; ABG88718; ABG88729; ABG88740; ABG88751; ABG88762; ABG88773; ABG88784; ABG88795; ABG88806; ABG88817; ABI20793; ABI20815; ABI20881; ABI20892; ABI20903; ABI20914; ABI20925; ABI20936; ABI20947; ABI20958; ABI20969; ABI21736; ABI20980; ABI20991; ABI21002; ABI21013; ABI21024; ABI21035; ABI21046; ABI21057; ABI21068; ABI21079; ABI21090; ABI30444; ABI21101; ABI21112; ABI21123; ABI26646; ABI21134; ABI21145; ABI21156; ABI21167; ABI21178; ABI21244; ABI21255; ABI21266; ABI21277; ABI21288; ABI21299; ABI21310; ABI21321; ABI21332; ABI21343; ABI21354; ABI21365; ABI21376; ABI21387; ABI21398; ABI21409; ABI21420; ABI21431; ABI21442; ABI21453; ABI21464; ABI21475; ABI21486; ABI21497; ABI22159; ABI21508; ABI30389; ABI30400; ABI30411; ABI30422; ABI30433; ABI30455; ABI30466; ABI30477; ABI30488; ABI30499; ABI30510; ABI30521; ABI30532; ABI30543; ABI30554; ABI30576; ABI30587; ABI30598; ABI30609; ABI30620; ABI30733; ABI30744; ABI30755; ABI30766; ABI30777; ABI30788; ABI30799; ABI30810; ABI30821; ABI30832; ABI30843; ABI30854; ABI30865; ABI30876; ABI84400; ABI84412; ABI84471; ABI84486; ABI84577; ABI84806; ABI84938; ABI92258; ABI92269; ABI92280; ABI92291; ABI92324; ABI92335; ABI92346; ABI92357; ABI92368; ABI92390; ABI92401; ABI92412; ABI92423; ABI92434; ABI92445; ABI92456; ABI92467; ABI92478; ABI92489; ABI92500; ABI92511; ABI92522; ABI92533; ABI92544; ABI92555; ABI92566; ABI92577; ABI92588; ABI92599; ABI92610; ABI92621; ABI92632; ABI92643; ABI92654; ABI92665; ABI92676; ABI92687; ABI92698; ABI92709; ABI92720; ABI92731; ABI92742; ABI92753; ABI92764; ABI92775; ABI92786; ABI92797; ABI92808; ABI92819; ABI92830; ABI92841; ABI92852; ABI92863; ABI92874; ABI92885; ABI92896; ABI92907; ABI92918; ABI92929; ABI92940; ABI92951; ABI92962; ABI92973; ABI92984; ABI92995; ABI93006; ABI93017; ABI93039; ABI93050; ABI93061; ABI93072; ABI93083; ABI93094; ABI93105; ABI93116; ABI95474; ABK79937; ABI95228; ABI95239; ABI95305; ABJ09096; ABJ09107; ABJ09118; ABJ09140; ABJ09162; ABJ09173; ABJ09195; ABJ09206; ABJ09217; ABJ09228; ABJ09239; ABJ09250; ABJ09261; ABJ09272; ABJ09283; ABJ09294; ABJ09305; ABJ09316; ABJ09338; ABJ09349; ABJ09360; ABJ09371; ABJ09382; ABJ16587; ABJ16598; ABJ16620; ABJ16631; ABJ16697; ABJ16708; ABJ16741; ABJ16752; ABJ16763; ABJ16774; ABJ16785; ABJ53460; ABJ53471; ABJ53482; ABK39951; ABK39962; ABK39973; ABK39984; ABK40017; ABK40061; ABK40075; ABK40612; ABK40623; ABK40645; ABK40656; ABK40667; ABK40678; ABK40700; ABK40711; ABK40722; ABK40733; ABK40744; ABK79981; ABK79992; ABK80014; ABK80058; ABK80069; ABK80080; ABK80091; ABK80102; ABK80113; ABK80124; ABK80300; ABK80135; ABK80146; ABK80157; ABK80168; ABK80179; ABK80190; ABK80201; ABK80212; ABK80223; ABK80234; ABK80245; ABK80256; ABK80267; ABK80278; ABK80289; ABK80311; ABL67044; ABL67110; ABL67132; ABL67165; ABL67176; ABL67198; ABL67220; ABL67841; ABL67275; ABL67286; ABL67297; ABL67308; ABL67319; ABL67330; ABL67341; ABL67352; ABL67363; ABL67374; ABL67385; ABL67396; ABL67407; ABL67418; ABL75563; ABM21938; ABM22015; ABM22037; ABM22059; ABM22070; ABM22081; ABM22092; ABM22103; ABM22114; ABM22125; ABO32816; ABM22136; ABM22147; ABM22301; ABM22312; ABM22323; ABM22334; ABM22345; ABM22356; ABM22367; ABM66853; ABM66875; ABM66919; ABM66930; ABM66941; ABM66952; ABM66963; ABM66974; ABM66985; ABM66996; ABM67007; ABM67018; ABM67029; ABM67040; ABN50767; ABN50984; ABN50995; ABN51010; ABN51021; ABN51032; ABN51043; ABN51054; ABN51099; ABN51110; ABN51121; ABN51132; ABN51154; ABN59390; ABO32656; ABO32667; ABO32692; ABO32751; ABO32762; ABO32775; ABO32790; ABO32803; ABO32827; ABO32838; ABO32849; ABO32860; ABO32871; ABO32882; ABO32893; ABO32904; ABO32915; ABO32926; ABO32937; ABO33036; ABO33047; ABO33058; ABO33069; ABO38076; ABO38087; ABO38230; ABO38241; ABO38252; ABO38274; ABO38285; ABO38417; ABO44035; ABO44068; ABO44079; ABO51829; ABO51862; ABO51884; ABO76913; ABO52071; ABO52126; ABO52214; ABO52291; ABO52313; ABO52324; ABO52335; ABO52346; ABO52357; ABO52368; ABO52423; ABO52522; ABO52566; ABO52577; ABO52588; ABO52599; ABO52632; ABO52643; ABO52676; ABO64343; ABO76946; ABO76957; ABP49184; ABP49371; ABP49404; ABP49415; ABP49426; ABP49492; ABP49503; ABP49514; BAA00769; BAA00770; BAA00771; BAA00772; BAA01025; BAA01026; BAA02776; BAA02777; BAA02778; BAA02779; BAA04707; BAA04708; BAA04709; BAA04710; BAA04711; BAA04712; BAA04713; BAA04714; BAA04715; BAA04716; BAA04717; BAA04718; BAA04719; BAA21644; BAA21645; BAA21646; BAA21647; BAA21648; BAA33866; BAA33938; BAA33939; BAA33940; BAA33941; BAA33942; BAA33943; BAA33944; BAA33945; BAA33946; BAA33947; BAA07844; BAA07845; BAA07846; BAA07847; BAA07848; BAA07849; BAA07850; BAA08713; BAA08714; BAA21070; BAA08715; BAA08716; BAA08717; BAA08718; BAA21071; BAA08719; BAA13091; AAY23641; AAY23642; AAY25498; AAY46201; AAY46202; AAY58320; AAZ06795; AAZ32943; AAZ32944; AAZ32945; AAZ32946; AAZ32947; AAZ32948; AAZ32949; AAZ32950; AAZ32951; AAZ32952; AAZ32953; AAZ29151; AAZ29152; AAZ29153; AAZ29154; AAZ29155; AAZ29156; AAZ29157; AAZ29158; AAZ29159; AAZ29160; AAZ29161; AAZ29162; AAZ29163; AAZ29164; AAZ29165; AAZ29166; AAZ29167; AAZ29168; AAZ29169; AAZ29170; AAZ29171; AAZ29172; AAZ29173; AAZ29174; AAZ29175; AAZ29176; AAZ29177; AAZ29178; AAZ29179; AAZ29180; AAZ29181; AAZ29182; AAZ29183; AAZ29184; AAZ29185; AAZ29186; AAZ29187; AAZ29188; AAZ29189; AAZ29190; AAZ29191; AAZ29192; ABA39842; ABA39843; ABA39844; ABA39845; ABA39846; ABA39847; ABA39848; ABA39849; ABA39850; AAZ57437; AAZ74350; ABA46957; ABA41538; ABA27432; ABA27440; AAZ91962; AAZ91963; AAZ91964; ABA60253; ABA60254; ABA60255; ABA60256; ABA60257; ABA60258; ABA60259; ABA60260; ABA60261; ABA60262; ABA60263; ABA60264; ABA60265; ABA60266; ABA60267; ABA60268; ABA60269; ABA60270; ABA60271; ABA60272; ABA60273; ABA60274; ABA60275; ABA60276; ABA60277; ABA60278; ABA60279; ABA60280; ABA60281; ABA60282; ABA60283; ABA60284; ABA60285; ABA60286; ABA60287; ABA60288; ABA60289; ABA60290; ABA60291; ABA60292; ABA60293; ABA60294; ABA60295; ABA60296; ABA60297; ABA60298; ABA60299; ABA60300; ABA60301; ABA60302; ABA60303; ABA60304; ABA60305; ABA60306; ABA60307; ABA60308; ABA60309; ABA06580; ABA06581; ABA06582; ABA06583; ABA06584; ABA06585; ABA60900; ABA60901; ABA60902; ABA60903; ABA60904; ABA60905; ABA60906; ABA60907; ABA60908; ABA60909; ABA60910; ABA60911; ABA60912; ABA60913; ABA60914; ABA60915; ABA60916; ABA60917; ABA60918; ABA60919; ABA60920; ABA60921; ABA60922; ABA60923; ABA60924; ABA60925; ABA60926; ABA60927; ABA60928; ABA60929; ABA60930; ABA60931; ABA60932; ABA60933; ABA60934; ABA60935; ABA60936; ABA60937; ABA60938; ABA60939; ABA60940; ABA60941; ABA60942; ABA60943; ABA60944; ABA60945; ABA60946; ABA60947; ABA60948; ABA60949; ABA60950; ABA60951; ABA60952; ABA60953; ABA60954; ABA60955; ABA60956; ABA60957; ABA60958; ABA60959; ABA60960; ABA60961; ABA60962; ABA60963; ABA60964; ABA60965; ABA60966; ABA60967; ABA60968; ABA60969; ABA60970; ABA60971; ABA60972; ABA60973; ABA60974; ABA60975; ABA60976; ABA60977; ABA60978; ABA60979; ABA60980; ABA60981; ABA60982; ABA60983; ABA60984; ABA60985; ABA60986; ABA60987; ABA60988; ABA60989; ABA60990; ABA60991; ABA60992; ABA60993; ABA60994; ABA60995; ABA60996; ABA60997; ABA60998; ABA60999; ABA61000; ABA61001; ABA61002; ABA61003; ABA61004; ABA61005; ABA61006; ABA61007; ABA61008; ABA61009; ABA61010; ABA61011; ABA61012; ABA61013; ABA61014; ABA61015; ABA61016; ABA61017; ABA61018; ABA61019; ABA61020; ABA61021; ABA61022; ABA61023; ABA61024; ABA61025; ABA61026; ABA61027; ABA61028; ABA61029; ABA61030; ABA61031; ABA61032; ABA61033; ABA61034; ABA61035; ABA61036; ABA61037; ABA61038; ABA61039; ABA61040; ABA61041; ABA61042; ABA61043; ABA61044; ABA61045; ABB17173; ABB71825; ABB71826; ABB71827; ABB71828; ABB71829; ABB71830; ABB71831; ABB71832; ABB71833; ABB51981; ABB51982; ABB51983; ABB51961; ABB51963; ABB51964; ABB76697; ABB76698; ABB76699; ABB76700; ABB84191; ABB84192; ABB84193; ABB84194; ABB84195; ABB84196; ABB84197; ABB84198; ABB84199; ABB84200; ABB84201; ABB84202; ABC59709; ABC66247; ABC66248; ABC66249; ABC66250; ABC66251; ABC66252; ABC66253; ABC66254; ABC66255; ABC66256; ABC66257; ABC66258; ABD59850; ABD59851; ABD59852; ABD59853; ABD59854; ABD59855; ABD59856; ABD59857; ABD85122; ABF17954; ABF17955; ABF17956; ABF17957; ABF17958; ABF17959; ABE73114; ABE73115; ABF21268; ABF21269; ABF21271; ABF21273; ABF21281; ABG26247; ABG26248; ABG26249; ABG26250; ABG26251; ABG26252; ABG26253; ABG26254; ABG26255; ABG26256; ABG02860; ABG02861; ABG02862; ABG02863; ABH00993; ABH00994; ABH00995; ABH00996; ABH00997; ABH00998; ABH00999; ABH01000; ABH01001; ABH01002; ABH01003; ABH01004; ABH01005; ABH01006; ABH01007; ABH01008; ABH01009; ABH01010; ABH01011; ABH01012; ABH01013; ABH01014; ABH01015; ABH01016; ABH01017; ABH01018; ABH01019; ABH01020; ABH01021; ABH01022; ABI22056; ABI22057; ABI22058; ABI22059; ABI22060; ABI22061; ABI22062; ABI22063; ABI22064; ABI22065; ABI22066; ABI22067; ABI22068; ABI22069; ABI22070; ABI22071; ABI22072; ABI22073; ABI22074; ABI22075; ABI22076; ABI22077; ABI22078; ABI22079; ABI22080; ABI22081; ABI22082; ABI22083; ABI22084; ABI22085; ABI22086; ABI22087; ABI22088; ABI22089; ABI22090; ABI22091; ABI22092; ABI22093; ABI22094; ABI22095; ABI22096; ABI22097; ABI22098; ABI22099; ABI22100; ABI22101; ABI22102; ABI54388; ABI54389; ABJ51896; ABI49169; ABI49170; ABI49171; ABI49172; ABI49173; ABI49174; ABI49175; ABI49176; ABI49177; ABI49178; ABI49179; ABI49180; ABI49181; ABI49182; ABI49183; ABI49184; ABI51314; ABI51315; ABI51316; ABI55257; ABI55258; ABI55259; ABI55260; ABI55261; ABI55262; ABI55263; ABI55264; ABI55265; ABI55266; ABI55267; ABI55268; ABI55269; ABI55270; ABI55271; ABI55272; ABI55273; ABI55274; ABI55275; ABI55276; ABI55277; ABI55278; ABJ53158; ABM47075; ABK60213; ABK60214; ABK60215; ABL86145; ABM98421; ABM98422; ABO10165; ABO10166; ABO10167; ABO10168; ABO10169; ABO10170; ABO10171; ABO10172; ABO10173; ABO10174; ABO10175; ABO10182; ABO20947; ABO20952; ABO20953; ABO20954; ABO20955; ABO20956; ABO20957; ABO20958; ABO21717; ABO21718; ABO21719; ABO21720; ABO21721; ABO21722; ABO21726; ABO21727; ABO21728; ABO21729; ABO20959; ABO20960; ABO21733; ABO37477; ABO37478; ABO37479; ABO37480; ABO37481; ABO37482; ABO37483; ABO37484; ABO37485; ABO37486; ABO37487; ABO37488; ABO37489; ABO37490; ABO37491; ABO37492; ABO37493; ABO37494; ABO37495; ABO37496; ABO37497; ABO37498; ABO37499; ABO37500; ABO37501; ABO37502; ABO37503; ABO37504; ABO37505; ABO37506; ABO37507; ABO37508; ABO37509; ABO37510; ABO37511; ABO37512; ABO37513; ABO37514; ABO37515; ABO37516; ABO37517; ABO37518; ABO37519; ABO37520; ABO37522; ABO37523; ABO37524; ABO37525; ABO37526; ABO37527; ABO37528; ABO37529; ABO37530; ABO37531; ABO37532; ABO37533; ABO37534; ABO37535; ABO37536; ABO37537; ABO37538; ABO37539; ABO37540; ABO37541; ABO37542; ABO37543; ABO37544; ABO37545; ABO37546; ABO37547; ABO37548; ABO37549; ABO37550; ABO37551; ABO37552; ABO37553; ABO37554; ABO37555; ABO37556; ABO37557; ABO37558; ABO37559; ABO37560; ABO37561; ABO37562; ABO37563; ABO37564; ABO37565; ABO37566; ABO37567; ABO37568; ABO37569; ABO37570; ABO37571; ABO37572; ABO37573; ABO37574; ABO37575; ABO37576; ABO37577; ABO37578; ABO37579; ABO37580; ABO37581; ABO37582; ABO37583; ABO37584; ABO37585; ABO37586; ABO37587; ABO37588; ABO37589; ABO37590; ABO37591; ABO37592; ABO37593; ABO37594; ABO37595; ABO37596; ABO37597; ABO37598; ABO37599; ABO37600; ABO37601; ABO37602; ABO37603; ABO37604; ABO37605; ABO37606; ABO37607; ABO37608; ABO37609; ABO37610; ABO37611; ABO37612; ABO37613; ABO37614; ABO37615; ABO37616; ABO37617; ABO37618; ABO37619; ABO37620; ABO37621; ABO37622; ABO37623; ABO37624; ABO37625; ABO37626; ABO37627; ABO37628; ABO37629; ABO37630; ABO37631; ABO37632; ABO37633; ABO37634; ABO37635; ABO37636; ABO37637; ABO37638; ABO37639; ABO37640; ABO37641; ABO37642; ABO37643; ABO37644; ABO37645; ABO37646; ABO37647; ABO37648; ABO37649; ABO37650; ABO37651; ABO37652; ABO37653; ABO37654; ABO37655; ABO37656; ABO37657; ABO37658; ABO37659; ABO37660; ABO37661; ABO37662; ABO37663; ABO37664; ABO37665; ABO37666; ABO37667; ABO37668; ABO37669; ABO37670; ABO37671; ABO37672; ABO37673; ABO37674; ABO37675; ABO37676; ABO37677; ABO37678; ABO37679; ABO37680; ABO37681; ABO37682; ABO37683; ABO37684; ABO37685; ABO37686; ABO37687; ABO37688; ABO37689; ABO37690; ABO37691; ABO37692; ABO37693; ABO37694; ABO37695; ABO37696; ABO37697; ABO37698; ABO37699; ABO37700; ABO37701; ABO37702; ABO37703; ABO37704; ABO37705; ABO37706; ABO37707; ABO37708; ABO37709; ABO37710; ABO37711; ABO37712; ABO37713; ABO37714; ABO37715; ABO37716; ABO37717; ABO37718; ABO37719; ABO37720; ABO37721; ABO37722; ABO37723; ABO37724; ABO37725; ABO37726; ABO37727; ABO37728; ABO37729; ABO37730; ABO37731; ABO37732; ABO37733; ABO37734; ABO37735; ABO37736; ABO37737; ABO37738; ABO37739; ABO37740; ABO37741; ABO37742; ABO37743; ABO37744; ABO37745; ABO37746; ABO37747; ABO37748; ABO37749; ABO37750; ABO37751; ABO37752; ABO37753; ABO37754; ABO37755; ABO37756; ABO37757; ABO37758; ABO37759; ABO37760; ABO37761; ABO37762; ABO37763; ABO37764; ABO37765; ABO37766; ABO37768; ABO37769; ABO37770; ABO37771; ABO37772; ABO37773; ABO37774; ABO37775; ABO37776; ABO37777; ABO37778; ABO37779; ABO37780; ABO37781; ABO37782; ABO37783; ABP35587; ABP35588; ABP35589; ABP35590; ABP35591; ABP35592; ABP35593; ABP35594; ABP35595; ABP35596; ABP35597; ABP35598; ABP35599; ABP35600; ABP35601; ABP35602; AAA43178; AAA43182; AAA43187; AAA43200; AAA43184; AAA43195; AAA62328; AAA62335; AAA62329; AAA62331; AAA62327; AAA62330; AAA62339; AAA62338; AAA62332; AAA43230; AAA43229; AAA43228; AAA43227; AAA43226; ABG66978; ABE73717; ABG66979; ABG66980; ABG66981; ABG57281; ABG57282; ABG57283; ABG57284; AAA62470; AAA64229; AAA64228; AAB36975; AAB36976; AAB36977; AAB36978; AAB36979; AAB36980; AAB19009; AAB19010; AAB19011; AAB19012; AAB19013; AAB19014; AAB19015; AAB19016; AAB19017; AAB19018; AAB19019; AAB19020; AAB19021; AAB19022; AAB19023; AAB19024; AAB19025; AAB19028; AAB19026; AAB19027; AAA43143; AAA43144; AAA43145; AAA43146; AAA43147; AAA43148; AAA43149; AAA43211; AAA43212; AAA43275; AAA43114; AAA43105; AAA43111; AAA43100; AAA43107; AAA43101; AAA43109; AAA43110; AAA43112; AAA43102; AAA43103; AAO49821; AAA43163; AAA43164; AAA43099; AAA43239; AAA43155; AAA43156; AAA43162; AAA43165; AAA43151; ABF60581; ABF60577; ABF60576; ABF60580; ABF60579; ABF60578; AAB27733; AAB33340; AAA85781; AAA18781; AAA18782; AAC79579; AAA87553; AAB09413; AAB09414; AAB09415; AAB09416; AAB09417; AAB09418; AAB09419; AAB09420; AAB09421; AAA92927; AAB02560; AAD00123; AAD00124; AAC80152; AAD00125; AAD00126; AAC80153; AAD00127; AAD00128; AAC80154; AAF24003; AAC08288; AAC08289; AAC08290; AAC08291; AAC08292; AAC08293; AAC08294; AAC08295; AAC08296; AAC08297; AAB58297; CAA24269; CAA24270; CAA24271; CAA24273; CAA24281; CAA24290; CAA24291; CAA29337; CAA48482; CAA51904; CAA51905; CAA51906; CAA53437; CAA59412; CAA59413; CAA59414; CAA59415; CAA59416; CAA59417; CAA64893; CAA64894; CAA74382; CAA74383; CAA74384; CAA74385; CAA74386; CAA74387; CAA74388; CAA86526; CAA86527; CAA86528; CAA86529; CAA86530; CAA86531; CAA86532; CAA86533; CAA86534; CAA86535; CAA86536; CAA86537; CAA86538; CAA86539; CAA86540; CAA86541; CAA86542; CAA86543; CAA86544; CAA86545; CAA86546; CAA86547; CAA86548; CAA86549; CAA86550; CAA86551 and CAA86552.

Protein sequences of influenza virus subtype H4 hemagglutinin suitable for inclusion as a cargo moiety in conjugates herein have the following accession numbers and are available to the public via the National Center for Biotechnology Information (NCBI) Entrez protein search and retrieval system:

BAF43432; BAF43456; ABB87539; ABB87550; ABB87561; ABB87572; ABB87583; ABB87594; ABB87604; ABB87615; ABB87626; ABB87637; ABB87648; ABB87656; ABB87667; ABB87678; ABB87689; ABB87700; ABB88194; ABB88267; ABB88298; ABI47995; ABI48017; ABG88223; ABG88234; ABI92225; ABI84388; ABI84423; ABI84483; ABI84604; ABI84643; ABI84795; ABI84885; ABI84894; ABI84905; ABI92203; ABI92214; ABI97487; ABL67033; ABL67088; ABO51840; ABO51851; ABO51873; ABO51895; ABO51906; ABO51928; ABO51939; ABO51950; ABO52192; ABO52500; ABO52511; ABO52533; ABO52654; ABO52665; ABO52687; BAA14332; AAY88147; AAY88148; AAY88149; AAY88150; AAY88151; AAY88152; AAY88153; AAY88154; AAY88155; AAY88156; AAY88157; AAY88158; AAY88159; AAY88160; AAY88161; AAY88162; AAY88163; AAY88164; AAY88165; AAY88166; AAY88167; ABB80525; ABC59902; ABI17551; ABJ53168; AAA43179; AAA43216; AAA43217; AAA43218; AAA43219; AAA43220; AAA43221; AAA43222; AAA43223; AAA43224; BAF43458; BAF46756; BAF46758; BAF46904; BAF48476; BAF48478; AAG17427; AAG17429; AAF99711; CAD45001; CAD45000; AAN83962; AAN83963; AAN83964; AAN83965; AAN83966; AAN83967; AAN83968; AAN83969; AAN83970; AAN83971; AAT09640; AAT09641; AAT09642; AAT65318; AAT65320; AAT65322; AAT65335; AAT65336; AAT65338; AAT65346; AAT65347; ABB19802; ABB19847; ABB19867; ABB19878; ABB19886; ABB20362; ABB20372; ABB87473; ABB87484; ABB87495; ABB87506; ABB90165; ABB87517 and ABB87528.

Protein sequences of influenza virus subtype H5 hemagglutinin suitable for inclusion as a cargo moiety in conjugates herein have the following accession numbers and are available to the public via the National Center for Biotechnology Information (NCBI) Entrez protein search and retrieval system:

AB166862; AB188816; AB188824; AB189053; AB189061; AB212054; AB212280; AB212649; AB233319; AB233320; AB233321; AB233322; AB239125; AB241614; AB241615; AB241616; AB241617; AB241618; AB241619; AB241620; AB241621; AB241622; AB241623; AB241624; AB241625; AB241626; AB259712; AB261853; AB263192; AB263752; AB275420; AB275421; AB275422; AB275423; AB275424; AB275425; AB275426; AB275427; AB275428; AB275429; AB275430; AB275431; AB275432; AB275433; AB275434; AB284324; AB295603; AF028709; AF036356; AF046080; AF046088; AF046096; AF046097; AF046098; AF046099; AF046100; AF057291; AF082034; AF082035; AF082036; AF082037; AF082038; AF082039; AF082040; AF082041; AF082042; AF082043; AF084279; AF084280; AF084281; AF084532; AF098537; AF098538; AF098539; AF098540; AF098541; AF098542; AF098543; AF098544; AF098545; AF098546; AF100179; AF100180; AF102671; AF102672; AF102673; AF102674; AF102675; AF102676; AF102677; AF102678; AF102679; AF102680; AF102681; AF102682; AF144305; AF148678; AF164655; AF164656; AF164657; AF164658; AF164659; AF164660; AF164661; AF164662; AF164663; AF164664; AF164665; AF194169; AF194990; AF194991; AF194992; AF216713; AF216721; AF216729; AF216737; AF290443; AF303057; AF364334; AF377870; AF398417; AF398418; AF420254; AF439407; AF439408; AF468837; AF501234; AF501235; AF509016; AF509017; AF509018; AF509019; AF509020; AF509021; AF509022; AF509023; AF509024; AF509025; AF509026; AF509027; AF509028; AF509029; AF509030; AF509031; AF509032; AF509033; AF509034; AF509035; AF509036; AF509037; AF509038; AF509039; AJ305306; AJ621807; AJ621811; AJ632268; AJ632269; AJ715872; AJ867074; AJ971297; AJ971298; AJ972673; AM087222; AM183669; AM183670; AM183671; AM183672; AM183673; AM183674; AM183675; AM183676; AM183677; AM231714; AM236074; AM262541; AM262542; AM262543; AM262546; AM262547; AM262553; AM262572; AM397634; AM400972; AM400973; AM400974; AM400975; AM400976; AM400977; AM400978; AM400979; AM400980; AM400981; AM403460; AM403461; AM403462; AM403463; AM403464; AM403465; AM403466; AM403467; AM403468; AM403469; AM403470; AM403471; AM403472; AM403473; AM403474; AM403475; AM408209; AM408210; AM408211; AM408212; AM408213; AM408214; AM408215; AM408216; AM492165; AY059474; AY059475; AY059476; AY059477; AY059478; AY059479; AY059480; AY059481; AY059482; AY075027; AY075030; AY075033; AY221521; AY221522; AY221523; AY221524; AY221525; AY221526; AY221527; AY221528; AY221529; AY296064; AY296065; AY296066; AY296067; AY296068; AY296069; AY296070; AY296071; AY296072; AY296073; AY296074; AY296075; AY296076; AY296077; AY296078; AY296079; AY296080; AY296081; AY296082; AY296083; AY296084; AY296085; AY296086; AY497063; AY497064; AY497065; AY497066; AY497067; AY497068; AY497069; AY497070; AY497071; AY497072; AY497073; AY497074; AY497075; AY497076; AY497077; AY497078; AY497079; AY497080; AY497081; AY497082; AY497083; AY497084; AY497085; AY497086; AY497087; AY497088; AY497089; AY497090; AY497091; AY497092; AY497093; AY497094; AY497095; AY497096; AY500365; AY518362; AY526745; AY531029; AY534913; AY534914; AY535020; AY535021; AY535022; AY535023; AY536212; AY552000; AY552001; AY553784; AY553785; AY553786; AY553787; AY553788; AY553789; AY553790; AY553791; AY553792; AY553793; AY553794; AY553795; AY553796; AY553797; AY553798; AY553799; AY553800; AY553801; AY553802; AY553803; AY553804; AY553805; AY553806; AY553807; AY553808; AY553809; AY553810; AY553811; AY555150; AY555153; AY573917; AY574187; AY574190; AY575869; AY575870; AY575871; AY575872; AY575873; AY575874; AY575875; AY575876; AY575877; AY575878; AY575879; AY575880; AY576927; AY576930; AY577314; AY585357; AY585358; AY585359; AY585360; AY585361; AY585362; AY585363; AY585364; AY585365; AY585366; AY585367; AY585368; AY585369; AY585370; AY585371; AY585372; AY585373; AY585374; AY585375; AY585376; AY585377; AY590563; AY590568; AY590569; AY590570; AY590571; AY590572; AY590577; AY609312; AY623430; AY626143; AY627885; AY639405; AY646167; AY646175; AY646424; AY649382; AY651320; AY651321; AY651322; AY651323; AY651324; AY651325; AY651326; AY651327; AY651328; AY651329; AY651330; AY651331; AY651332; AY651333; AY651334; AY651335; AY651336; AY651337; AY651338; AY651339; AY651340; AY651341; AY651342; AY651343; AY651344; AY651345; AY651346; AY651347; AY651348; AY651349; AY651350; AY651351; AY651352; AY651353; AY651354; AY651355; AY651356; AY651357; AY651358; AY651359; AY651360; AY651361; AY651362; AY651363; AY651364; AY651365; AY651366; AY651367; AY651368; AY651369; AY651370; AY651371; AY651372; AY651373; AY653200; AY676033; AY676034; AY676035; AY676036; AY679514; AY684706; AY684894; AY720942; AY720945; AY720950; AY724783; AY724785; AY724787; AY724789; AY724791; AY724793; AY724795; AY728892; AY728894; AY737289; AY737296; AY737304; AY741213; AY741215; AY741217; AY741219; AY741221; AY747609; AY747617; AY770079; AY770991; AY779048; AY779050; AY786078; AY818135; AY818136; AY818137; AY830774; AY834279; AY842935; AY849793; AY854190; AY861372; AY866475; AY950230; AY950231; AY950232; AY950233; AY950234; AY950235; AY950236; AY972539; AY972540; AY972541; AY972542; AY995883; AY995884; AY995885; AY995886; AY995887; AY995888; AY995889; AY995890; AY995891; AY995892; AY995893; AY995894; AY995895; AY995896; AY995897; AY995898; CY005575; CY005918; CY005926; CY005927; CY005969; CY006028; CY006036; CY006040; CY011248; CY014168; CY014177; CY014185; CY014193; CY014197; CY014198; CY014199; CY014200; CY014201; CY14202; CY014203; CY014204; CY014205; CY014206; CY014207; CY014208; CY014209; CY014210; CY014211; CY014212; CY014213; CY014272; CY014280; CY014288; CY014296; CY014303; CY014311; CY014368; CY014376; CY014384; CY014393; CY014401; CY014409; CY014417; CY014425; CY014433; CY014441; CY014449; CY014457; CY014465; CY014477; CY014481; CY014489; CY014497; CY014510; CY014518; CY014529; CY014537; CY014543; CY014580; CY014615; CY014640; CY014642; CY014717; CY014722; CY014726; CY014849; CY014872; CY014984; CY015073; CY015081; CY015089; CY015115; CY016276; CY016284; CY016292; CY016300; CY016611; CY016779; CY016787; CY016795; CY016803; CY016811; CY016819; CY016827; CY016835; CY016843; CY016851; CY016859; CY016867; CY016875; CY016883; CY016891; CY016899; CY016907; CY016915; CY016923; CY016931; CY016939; CY16947; CY17027; CY017035; CY017043; CY017051; CY017059; CY017067; CY017179; CY017187; CY017403; CY017638; CY017646; CY017654; CY017662; CY017670; CY017678; CY017688; CY018949; CY019352; CY019360; CY019368; CY019376; CY019384; CY019392; CY019400; CY019408; CY019416; CY019424; CY019432; CY020229; CY020349; CY020621; CY020629; CY020637; CY020645; CY020653; CY020661; CY020669; CY020677; CY020693; CY020701; CY020709; CY021373; CY021381; CY021389; CY021397; CY021517; CY021525; DQ003215; DQ007623; DQ017270; DQ017271; DQ017272; DQ017273; DQ017274; DQ017275; DQ017276; DQ017277; DQ017278; DQ017279; DQ017280; DQ017281; DQ017282; DQ017283; DQ017284; DQ017285; DQ017286; DQ017287; DQ017288; DQ017289; DQ017290; DQ017291; DQ017292; DQ017293; DQ017294; DQ017295; DQ017296; DQ017297; DQ017298; DQ017299; DQ017300; DQ017301; DQ017302; DQ017303; DQ017304; DQ017305; DQ017306; DQ017307; DQ017308; DQ023145; DQ076201; DQ080022; DQ083550; DQ083551; DQ083552; DQ083553; DQ083554; DQ083555; DQ083556; DQ083557; DQ083558; DQ083559; DQ083560; DQ083561; DQ083562; DQ083563; DQ083564; DQ083565; DQ083566; DQ083567; DQ083568; DQ083569; DQ083570; DQ083571; DQ083572; DQ083573; DQ083574; DQ083575; DQ083576; DQ083577; DQ083578; DQ083579; DQ083580; DQ083581; DQ083582; DQ083583; DQ083584; DQ083585; DQ092869; DQ095612; DQ095613; DQ095614; DQ095615; DQ095616; DQ095617; DQ095618; DQ095619; DQ095620; DQ095621; DQ095622; DQ095623; DQ095624; DQ095625; DQ095626; DQ095627; DQ095628; DQ095629; DQ095630; DQ095631; DQ099755; DQ099756; DQ099757; DQ099758; DQ099759; DQ099760; DQ100554; DQ100555; DQ100556; DQ100557; DQ104701; DQ122147; DQ137873; DQ153251; DQ153252; DQ182483; DQ188905; DQ188906; DQ188907; DQ188908; DQ190857; DQ190858; DQ190859; DQ190860; DQ190861; DQ191688; DQ191689; DQ201829; DQ211922; DQ211923; DQ211924; DQ211925; DQ212792; DQ230521; DQ230522; DQ231240; DQ231241; DQ231242; DQ236077; DQ236085; DQ251447; DQ251796; DQ251797; DQ251798; DQ251799; DQ251800; DQ256383; DQ279301; DQ309440; DQ320137; DQ320875; DQ320876; DQ320877; DQ320878; DQ320879; DQ320880; DQ320881; DQ320882; DQ320883; DQ320884; DQ320885; DQ320886; DQ320887; DQ320888; DQ320889; DQ320890; DQ320891; DQ320892; DQ320893; DQ320894; DQ320895; DQ320896; DQ320897; DQ320898; DQ320899; DQ320900; DQ320901; DQ320902; DQ320903; DQ320904; DQ320905; DQ320906; DQ320907; DQ320908; DQ320909; DQ320910; DQ320911; DQ320912; DQ320913; DQ320914; DQ320915; DQ320916; DQ320917; DQ320918; DQ320919; DQ320920; DQ320921; DQ320922; DQ320923; DQ320924; DQ320925; DQ320926; DQ320927; DQ320928; DQ320929; DQ320930; DQ320931; DQ320932; DQ320933; DQ320934; DQ320935; DQ320936; DQ320937; DQ320938; DQ320939; DQ320940; DQ323672; DQ334760; DQ334768; DQ334776; DQ340848; DQ343150; DQ343151; DQ343152; DQ343502; DQ356886; DQ358746; DQ360835; DQ363918; DQ363923; DQ364996; DQ365004; DQ366306; DQ366314; DQ366322; DQ366330; DQ366338; DQ371928; DQ371929; DQ371930; DQ372591; DQ387854; DQ389158; DQ399540; DQ399547; DQ406728; DQ407519; DQ412997; DQ434889; DQ435200; DQ435201; DQ435202; DQ440535; DQ447199; DQ449031; DQ449632; DQ449640; DQ453141; DQ458992; DQ464354; DQ464377; DQ497642; DQ497643; DQ497644; DQ497645; DQ497646; DQ497647; DQ497648; DQ497649; DQ497650; DQ497651; DQ497652; DQ497653; DQ497654; DQ497655; DQ497656; DQ497657; DQ497658; DQ497659; DQ497660; DQ497661; DQ497662; DQ497663; DQ497664; DQ497665; DQ497666; DQ497667; DQ497668; DQ497669; DQ497670; DQ497671; DQ497672; DQ497673; DQ497674; DQ497675; DQ497676; DQ497677; DQ497678; DQ497679; DQ497680; DQ497681; DQ497682; DQ497683; DQ497684; DQ497685; DQ497686; DQ497687; DQ497688; DQ497689; DQ497690; DQ497691; DQ497692; DQ497693; DQ497694; DQ497695; DQ497696; DQ497697; DQ497698; DQ497699; DQ497700; DQ497701; DQ497702; DQ497703; DQ497704; DQ497705; DQ497706; DQ497707; DQ497708; DQ497709; DQ497710; DQ497711; DQ497712; DQ497713; DQ497714; DQ497715; DQ497716; DQ497717; DQ497718; DQ497719; DQ497720; DQ497721; DQ497722; DQ497723; DQ497724; DQ497725; DQ497726; DQ497727; DQ497728; DQ497729; DQ515984; DQ530173; DQ535724; DQ643809; DQ643982; DQ644955; DQ644956; DQ644957; DQ644958; DQ644959; DQ650659; DQ650663; DQ659113; DQ659326; DQ659327; DQ659679; DQ661910; DQ666146; DQ673901; DQ676830; DQ676834; DQ676838; DQ676840; DQ767725; DQ826532; DQ835313; DQ836043; DQ837587; DQ837588; DQ837589; DQ837590; DQ838508; DQ838509; DQ838516; DQ838517; DQ840519; DQ840533; DQ842487; DQ842489; DQ845348; DQ851561; DQ852600; DQ861291; DQ861999; DQ862000; DQ862001; DQ862002; DQ862003; DQ863503; DQ864711; DQ864715; DQ864716; DQ864717; DQ864718; DQ864719; DQ864720; DQ864721; DQ885610; DQ885612; DQ885614; DQ885616; DQ885618; DQ914808; DQ914814; DQ991231; DQ992714; DQ992715; DQ992716; DQ992717; DQ992718; DQ992719; DQ992720; DQ992721; DQ992722; DQ992723; DQ992724; DQ992725; DQ992726; DQ992727; DQ992728; DQ992729; DQ992730; DQ992731; DQ992732; DQ992733; DQ992734; DQ992735; DQ992736; DQ992737; DQ992738; DQ992739; DQ992740; DQ992741; DQ992742; DQ992743; DQ992744; DQ992745; DQ992746; DQ992747; DQ992748; DQ992749; DQ992750; DQ992751; DQ992752; DQ992753; DQ992754; DQ992755; DQ992756; DQ992757; DQ992758; DQ992759; DQ992760; DQ992761; DQ992762; DQ992763; DQ992764; DQ992765; DQ992766; DQ992767; DQ992768; DQ992769; DQ992770; DQ992771; DQ992772; DQ992773; DQ992774; DQ992775; DQ992776; DQ992777; DQ992778; DQ992779; DQ992780; DQ992781; DQ992782; DQ992783; DQ992784; DQ992785; DQ992786; DQ992787; DQ992788; DQ992789; DQ992790; DQ992791; DQ992792; DQ992793; DQ992794; DQ992795; DQ992796; DQ992797; DQ992798; DQ992799; DQ992800; DQ992801; DQ992802; DQ992803; DQ992804; DQ992805; DQ992806; DQ992807; DQ992808; DQ992809; DQ992810; DQ992811; DQ992812; DQ992813; DQ992814; DQ992815; DQ992816; DQ992817; DQ992818; DQ992819; DQ992820; DQ992821; DQ992822; DQ992823; DQ992824; DQ992825; DQ992826; DQ992827; DQ992828; DQ992829; DQ992830; DQ992831; DQ992832; DQ992833; DQ992834; DQ992835; DQ992836; DQ992837; DQ992838; DQ992839; DQ992840; DQ992841; DQ992842; DQ992843; DQ992844; DQ992845; DQ992846; DQ992847; DQ992848; DQ992849; DQ992850; DQ992851; DQ992852; DQ992853; DQ992854; DQ992855; DQ992856; DQ992857; DQ992858; DQ992859; DQ992860; DQ992861; DQ992862; DQ992863; DQ992864; DQ992865; DQ992866; DQ992867; DQ992868; DQ992869; DQ992870; DQ992871; DQ992872; DQ992873; DQ992874; DQ992875; DQ992876; DQ992877; DQ992878; DQ992879; DQ992880; DQ992881; DQ992882; DQ992883; DQ992884; DQ992885; DQ992886; DQ992887; DQ992888; DQ992889; DQ992890; DQ992891; DQ992892; DQ992893; DQ992894; DQ992895; DQ992896; DQ992897; DQ992898; DQ992899; DQ992900; DQ992901; DQ992902; DQ992903; DQ992904; DQ992905; DQ992906; DQ992907; DQ992908; DQ992909; DQ992910; DQ992911; DQ992912; DQ992913; DQ992914; DQ992915; DQ992916; DQ992917; DQ992918; DQ992919; DQ992920; DQ992921; DQ992922; DQ992923; DQ992924; DQ992925; DQ992926; DQ992927; DQ992928; DQ992929; DQ992930; DQ992931; DQ992932; DQ992933; DQ992934; DQ992935; DQ992936; DQ992937; DQ992938; DQ992939; DQ992940; DQ992941; DQ992942; DQ992943; DQ992944; DQ992945; DQ992946; DQ992947; DQ992948; DQ992949; DQ992950; DQ992951; DQ992952; DQ992953; DQ992954; DQ992955; DQ992956; DQ992957; DQ992958; DQ992959; DQ992960; DQ992961; DQ992962; DQ992963; DQ992964; DQ992965; DQ992966; DQ992967; DQ992968; DQ992969; DQ992970; DQ992971; DQ992972; DQ992973; DQ992974; DQ992975; DQ992976; DQ992977; DQ992978; DQ992979; DQ992980; DQ992981; DQ992982; DQ992983; DQ992984; DQ992985; DQ992986; DQ992987; DQ992988; DQ992989; DQ992990; DQ992991; DQ992992; DQ992993; DQ992994; DQ992995; DQ992996; DQ992997; DQ992998; DQ992999; DQ993000; DQ993001; DQ993002; DQ993003; DQ993004; DQ993005; DQ993006; DQ993007; DQ993008; DQ993009; DQ993010; DQ993011; DQ993012; DQ993013; DQ993014; DQ993015; DQ993016; DQ993017; DQ993018; DQ993019; DQ993020; DQ993021; DQ993022; DQ993023; DQ993024; DQ993025; DQ993026; DQ993027; DQ993028; DQ993029; DQ993030; DQ993031; DQ993032; DQ993033; DQ993034; DQ993035; DQ993036; DQ993037; DQ993038; DQ993039; DQ993040; DQ993041; DQ993042; DQ993043; DQ993044; DQ993045; DQ993046; DQ993047; DQ993048; DQ993049; DQ993050; DQ993051; DQ993052; DQ993053; DQ993054; DQ993055; DQ993056; DQ993057; DQ993058; DQ993059; DQ993060; DQ993061; DQ993062; DQ993063; DQ993064; DQ993065; DQ993066; DQ993067; DQ993068; DQ993069; DQ993070; DQ993071; DQ993072; DQ993073; DQ993074; DQ993075; DQ993076; DQ993077; DQ993078; DQ993079; DQ993080; DQ993081; DQ993082; DQ993083; DQ993084; DQ993085; DQ993086; DQ993087; DQ993088; DQ993089; DQ993090; DQ993091; DQ993092; DQ993093; DQ993094; DQ993095; DQ993096; DQ993097; DQ993098; DQ993099; DQ993100; DQ993101; DQ993102; DQ993103; DQ993104; DQ993105; DQ993106; DQ993107; DQ993108; DQ993109; DQ993110; DQ993111; DQ993112; DQ993113; DQ993114; DQ993115; DQ993116; DQ993117; DQ997076; DQ997087; DQ997094; DQ997102; DQ997111; DQ997122; DQ997123; DQ997133; DQ997163; DQ997182; DQ997218; DQ997219; DQ997253; DQ997262; DQ997268; DQ997276; DQ997283; DQ997308; DQ997325; DQ997352; DQ997355; DQ997361; DQ997370; DQ997377; DQ997392; DQ997396; DQ997405; DQ997410; DQ997513; DQ997522; DQ997531; DQ997538; DQ997547; DQ999872; DQ999880; DQ999887; EF041479; EF042614; EF042615; EF042616; EF042617; EF042618; EF042619; EF042620; EF042621; EF042622; EF042623; EF042624; EFO61116; EF090647; EF090648; EF090649; EF090650; EF107522; EF110518; EF110519; EF124794; EF165048; EF165049; EF165050; EF165051; EF165052; EF165053; EF165054; EF165055; EF165056; EF165057; EF165058; EF165059; EF165060; EF165061; EF165062; EF165063; EF165064; EF165065; EF165066; EF200512; EF200513; EF205154; EF205155; EF205156; EF205157; EF205158; EF205159; EF205160; EF382359; EF395844; EF395845; EF419242; EF419243; EF441263; EF441276; EF441277; EF441278; EF441279; EF441280; EF441281; EF446771; EF446779; EF447430; EF451059; EF456780; EF456781; EF456795; EF456798; EF456799; EF456802; EF456803; EF456805; EF467802; EF469650; EF469651; EF469652; EF469653; EF469654; EF469655; EF469656; EF469657; EF469658; EF469659; EF469660; EF473068; EF473069; EF473070; EF473073; EF473074; EF473075; EF473080; EF473081; EF474450; J02160; J04325; L46585; L46586; L46587; M10243; M18001; M18450; M18451; M30122; S68489; U05330; U05331; U05332; U20460; U20472; U20473; U20474; U20475; U28919; U28920; U37165; U37166; U37167; U37168; U37169; U37170; U37171; U37172; U37173; U37174; U37175; U37176; U37177; U37178; U37179; U37180; U37181; U37182; U67783; U69277; U79448; U79449; U79450; U79451; U79452; U79453; U79454; U79455; U79456; X07826 and X07869.

Protein sequences of influenza virus subtype H6 hemagglutinin suitable for inclusion as a cargo moiety in conjugates herein have the following accession numbers and are available to the public via the National Center for Biotechnology Information (NCBI) Entrez protein search and retrieval system:

BAF36386; BAF41914; CAC83641; CAC83642; CAC83644; CAC83645; CAC83646; CAC81274; CAC81276; CAC81279; CAC84237; CAC84238; CAC84239; CAC84240; CAC84241; CAC84242; CAC84243; CAC84244; CAD20327; CAD45192; CAD45193; CAD45194; CAD45195; CAD45196; CAD45197; CAD45198; CAG27343; CAG27344; CAG27345; CAG27346; CAG27347; AAT65326; AAT65328; AAT65330; AAT65332; AAT65340; AAT65341; AAT65342; AAT65343; AAT65344; AAT65350; AAV91218; AAX07773; AAV41833; AAW78053; AAX78820; ABB18391; ABB18402; ABB18476; ABB18951; ABB18962; ABB18973; ABB18978; ABB18994; ABB19011; ABB19020; ABB19026; ABB19032; ABB19042; ABB19055; ABB19072; ABB19083; ABB19094; ABB19101; ABB19107; ABB19118; ABB19129; ABB19140; ABB19151; ABB19162; ABB19173; ABB19184; ABB19195; ABB19206; ABB19217; ABB19228; ABB19239; ABB19360; ABB19371; ABB19382; ABB19393; ABB19404; ABB19585; ABB19596; ABB19947; ABB20283; ABB20294; ABB20387; ABB21783; ABO52005; ABG88267; ABI20804; ABI30356; ABI84387; ABI84457; ABI84466; ABI84473; ABI84516; ABI84663; ABI84827; ABI84838; ABI84866; ABI84916; ABI84927; ABI85172; ABI92192; ABI92236; ABI92247; ABI95151; ABI95162; ABI95173; ABI95184; ABI95195; ABJ16576; ABL67154; ABL75574; ABM21971; ABM21993; ABM22004; ABO51917; ABO51961; ABO51972; ABO51983; ABO51994; ABO52016; ABO52027; ABO52049; ABO52159; ABO52181; ABO52203; ABO52478; ABO52489; ABO76979; ABP49283; BAA14333; AAZ04680; AAZ04681; AAZ04682; AAZ04683; AAZ04684; AAZ04685; AAZ04686; AAZ04687; AAZ04688; AAZ04689; AAZ04690; AAZ04691; AAZ04692; AAZ04693; AAZ04694; AAZ04695; AAZ04696; AAZ04697; AAZ04698; AAZ04699; AAZ04700; AAZ04701; AAZ04702; AAZ04703; AAZ04704; AAZ04706; AAZ04707; AAZ04708; AAZ04709; AAZ04710; AAZ04711; AAZ04712; AAZ04713; AAZ04714; AAZ04715; ABB88830; ABD35522; ABD35523; ABD35524; ABD35525; ABD35526; ABD35527; ABD35528; ABD35529; ABD35530; ABD35531; ABD35532; ABD35533; ABD35534; ABD35535; ABD35536; ABD35537; ABD35538; ABD35539; ABD35540; ABD35541; ABD35542; ABD35543; ABD35544; ABD35545; ABD35546; ABD35547; ABD35548; ABD35549; ABD35550; ABD35551; ABD35552; ABD35553; ABD35554; ABD35555; ABD35556; ABD35557; ABD65973; ABD65981; ABD65988; ABH03489; ABH03497; AAA43198; BAF47393; BAF47395; BAF47399; BAF48480; BAF48639; BAF49413; AAF04721; AAF87507; AAG38550; AAG38551; AAG38552; AAM69944; AAM69945; AAM69946; AAM69947; AAM69948; AAM69949; AAM69950; AAM69951; AAM69962; AAM69973; AAM69983; AAM69993; AAM70005; AAM70007; AAO33479; AAO33480; AAO33481; AAO33482; AAO33483; AAO33484; AAO33485; AAO33486; AAO33487; AAO33488; CAC81746; CAC81747; CAC84981; CAC84982; CAC85087; CAC84852; CAC84860; CAC85080; CAC85081; CAC85082; CAC85083; CAC85084 and CAC85085.

Protein sequences of influenza virus subtype H7 hemagglutinin suitable for inclusion as a cargo moiety in conjugates herein have the following accession numbers and are available to the public via the National Center for Biotechnology Information (NCBI) Entrez protein search and retrieval system:

BAE96029; AAD26924; AAG10680; AAL37237; AAL37238; AAL37239; AAL37240; AAL37241; AAL37242; AAK58912; AAK58913; AAK58914; AAK58915; AAK58916; AAK58917; AAK58918; AAK58919; AAK58920; AAK58921; AAK58922; AAK58923; AAK58924; AAK58925; AAK58926; AAK58927; AAK58928; AAK58929; AAK58930; AAK58931; AAK58932; AAK58933; AAK58934; AAK58935; AAK58936; AAK58937; AAK58938; AAK58939; AAK58940; AAK58941; AAK58942; AAK58943; AAK58944; AAK58945; AAK58946; AAK58947; AAK58948; AAK58949; AAK58950; AAK58951; AAM19228; AAM19229; AAM19230; AAM19231; AAM19232; AAM19233; AAM19234; AAM19235; AAM19236; CAD33826; CAD37074; CAD38049; CAD38050; CAD38051; CAD38052; CAD38053; CAD38054; CAD38282; CAD38283; CAD38284; CAD38285; CAD38286; CAD38287; CAD38288; CAE45011; CAE48276; CAF04466; CAF33017; CAF33020; CAG27348; CAG27349; CAG28943; CAG28944; CAG28945; CAG28956; CAG28957; CAG28958; CAG28959; CAJ32548; CAJ32557; AAO86904; AAO86905; AAO86906; AAO86907; AAO86908; AAO86909; AAO86910; AAO86911; AAO86912; AAO86913; AAO86914; AAO86915; AAO86916; AAO86917; AAO86918; AAO86919; AAO86920; AAO86921; AAO86922; AAO86923; AAO86924; AAO86925; AAO86926; AAO86927; AAO86928; AAO86929; AAO86930; AAO86931; AAO86932; AAO86933; AAO86934; AAO86935; AAO86936; AAO86937; AAO86938; AAO86939; AAO86940; AAO86941; AAO86942; AAO86943; AAO86944; AAO86945; AAO86946; AAO86947; AAO86948; AAO86949; AAO86950; AAO86951; AAO86952; AAQ77402; AAQ77403; AAQ77404; AAQ77405; AAQ77406; AAQ77407; AAR02636; AAR02637; AAR02638; AAR02639; AAR02640; AAR02641; AAR02642; AAR02643; AAQ90292; AAS68158; AAT37403; AAT37404; AAT37405; AAT37406; AAT02538; AAT38819; AAT66415; AAT78582; AAT70170; AAT69348; AAV74187; AAU00821; AAU25838; AAU25943; AAU85295; AAU33999; AAU44367; AAU50675; AAV98693; AAV98694; AAV98695; AAY20940; AAY46207; AAY46208; AAY46209; AAY46210; AAY46211; AAY46212; AAY46213; AAY46214; AAY46215; AAY46216; AAY46217; AAY46218; AAY46219; AAY46220; AAY46221; ABB87303; ABB87751; ABB87762; ABB87773; ABB87784; ABB87800; ABB87822; ABB87833; ABB87854; ABB88289; ABB88359; ABI84433; ABI84462; ABI84599; ABI84602; ABI84683; ABI84694; ABI84849; ABI84981; ABI85000; ABI85011; ABI85029; ABI85038; ABI85084; ABI95206; ABM21982; ABO44145; ABO44156; ABO44167; ABO44178; ABO44189; ABO45248; ABO52060; ABO52698; ABO52709; ABO52764; ABO52775; ABO52786; ABO76990; ABO77001; ABO77012; ABO77056; ABO77067; ABO77078; ABO77089; ABP49206; ABP49228; AAY21164; AAY87433; AAY87443; ABF69256; ABG57088; ABG57089; ABG57090; ABG57091; ABG57092; ABG57093; ABH04379; ABH04385; ABH05673; ABI26074; ABI26075; ABJ90226; ABJ90237; ABJ90248; ABJ90259; ABJ90270; ABJ90280; ABO21714; ABO21715; AAA43192; AAR96248; AAA56803; AAA92244; AAA92245; AAA92246; AAA43152; AAA43154; AAA43150; AAA43237; AAA43087; AAA43174; AAC54376; AAC54377; AAC54379; AAC54380; AAC54381; AAC54382; AAC54383; AAC54384; AAC54385; AAC54386; AAC54387; AAC54388; AAC54389; CAA43815; CAA44429; CAA44430; CAA44431; CAA44432; CAA44433; CAA44434; CAA44435; CAA44436; CAA44437; CAA78263; CAA87393; BAE96040; BAE96041; BAE96042; BAE96043; BAE96044; BAE96045; BAF02913; BAF02930; BAF02931; BAF02932; BAF02933; BAF02934; BAF03206; BAF03525; BAF03526; BAF49200; BAF49202; BAF49411; AAC40998; AAC40999; AAD19847; AAD19848; AAD26922; AAD26923; AAD26925; AAD26926; AAD26927; AAD26928; AAD26929; AAD26930; AAD26931; AAD26932; AAD26933; AAD26934; AAD26935; AAD26936; AAD26937; AAD26938; AAD26939; AAD26940; AAD26941; AAD37422; AAG10650; AAG10651; AAG10652; AAG10653; AAG10654; AAG10655; AAG10656; AAG10657; AAG10658; AAG10659; AAG10660; AAG10661; AAG10662; AAG10663; AAG10664; AAG10665; AAG10666; AAG10667; AAG10668; AAG10669; AAG10670; AAG10671; AAG10672; AAG10673; AAG10674; AAG10675; AAG10676; AAG10677; AAG10678 and AAG10679.

Protein sequences of influenza virus subtype H8 hemagglutinin suitable for inclusion as a cargo moiety in conjugates herein have the following accession numbers and are available to the public via the National Center for Biotechnology Information (NCBI) Entrez protein search and retrieval system:

BAF43468; AAG38554; AAG38555; AAG38556; ABB87722; ABB87729; ABB87740; ABI84428; ABI84519; ABI85240; ABL67099; BAA14334; ABK32094 and AAA43177.

Protein sequences of influenza virus subtype H9 hemagglutinin suitable for inclusion as a cargo moiety in conjugates herein have the following accession numbers and are available to the public via the National Center for Biotechnology Information (NCBI) Entrez protein search and retrieval system:

AAA43208; AAD48995; AAD48996; AAD48997; AAD48998; AAD48999; AAD49000; AAF00701; AAF00702; AAF00703; AAF00704; AAF00705; AAF00706; AAF00707; AAF00708; AAF00709; AAF00710; AAF00711; AAF00712; AAF15580; AAF15581; AAF15582; AAF15583; AAF69255; AAF69256; AAF69257; AAF69258; AAF69259; AAF69260; AAF69261; AAF69262; AAG48164; AAG48165; AAG48166; AAG48167; AAG48168; AAG48169; AAG48170; AAG48171; AAG53035; AAG53036; AAG53037; AAG53038; AAG53039; AAG53040; AAG53041; AAG53042; AAG53043; AAG53044; AAG53045; AAG53046; AAG53047; AAG53048; AAG53049; AAG53050; AAG53051; AAG53052; AAG53053; AAG53054; AAG53055; AAG53056; AAG53057; AAG53058; AAG53059; AAG53060; AAG53061; AAG53062; AAG53063; AAG53064; AAG53065; AAG53066; AAG53067; AAG53068; AAG53069; AAK62979; AAK64189; AAL14080; AAL14081; AAL30486; AAL30487; AAL32475; AAL32476; AAL32477; AAL32478; AAL32479; AAL65235; AAL65236; AAL65237; AAL65238; AAL65239; AAL65240; AAL65241; AAL65242; AAL65243; AAL65244; AAL65245; AAL65246; AAL65247; AAL65248; AAL65249; AAL65250; AAL65251; AAL65252; AAL65253; AAL65254; AAL65255; AAL65256; AAL65257; AAL65258; AAM03341; AAM03342; AAN05676; AAN05677; AAN05678; AAN05679; AAN05680; AAN05681; AAN05682; AAN05683; AAN05684; AAN05685; AAN83972; AAN83973; AAN83974; AAN83975; AAN83976; AAN83977; AAN83978; AAN83979; AAN83980; AAN83981; AAN83982; AAN83983; AAN83984; AAN83985; AAN83986; AAN83987; AAO46077; AAO46078; AAO46079; AAO46080; AAO46081; AAO46082; AAO46083; AAO46084; AAO46085; AAO46086; AAO47744; AAO47745; AAO47746; AAO47747; AAO47748; AAO47749; AAO47750; AAO47751; AAO47752; AAP23303; AAP41031; AAP41032; AAP41033; AAP41034; AAP41035; AAP47821; AAP49029; AAP49030; AAP49031; AAP49032; AAP49033; AAP49034; AAP49035; AAP49036; AAP49037; AAP49038; AAP49039; AAP49040; AAP49041; AAP49042; AAP49043; AAP49044; AAP49045; AAP49046; AAP49047; AAP97867; AAQ04843; AAQ04844; AAQ04845; AAQ04846; AAQ04847; AAQ04848; AAQ04849; AAQ04850; AAQ04851; AAQ04852; AAQ04853; AAQ04854; AAQ04855; AAQ04856; AAQ04857; AAQ04858; AAQ04859; AAQ04860; AAQ04861; AAQ04862; AAQ04863; AAQ63104; AAQ63105; AAQ63106; AAQ63107; AAQ63108; AAQ63109; AAQ63110; AAQ63111; AAQ63112; AAQ63113; AAQ63114; AAQ63115; AAQ63116; AAQ63117; AAQ63118; AAQ63119; AAQ67246; AAQ97375; AAQ97376; AAQ97377; AAQ97378; AAQ97379; AAR08917; AAR08918; AAR98872; AAS48376; AAS48377; AAS48378; AAS48379; AAS48380; AAS48381; AAS48382; AAS48383; AAS48384; AAS48385; AAS48386; AAS48387; AAS48388; AAS48389; AAS48390; AAS48391; AAS48392; AAT12413; AAT37508; AAT45076; AAT65317; AAT65323; AAT65337; AAT65339; AAT70836; AAU00107; AAU00108; AAU00109; AAU11147; AAU11148; AAU11149; AAU11150; AAU11151; AAU11152; AAU11153; AAU11154; AAU11155; AAU11156; AAU11157; AAU11158; AAU11159; AAU11160; AAU11161; AAU11162; AAU11163; AAU11164; AAU11165; AAV30213; AAV52598; AAV52599; AAV52600; AAV52601; AAV52602; AAV52603; AAV52604; AAV52605; AAV67992; AAV68000; AAV68014; AAV68022; AAV68030; AAV68031; AAV68032; AAV68037; AAW29075; AAW29076; AAW29077; AAW29078; AAW29079; AAW29080; AAW50825; AAW50826; AAW78038; AAW78039; AAW78040; AAW78041; AAW78042; AAW78043; AAW78044; AAW78045; AAW78046; AAX32895; AAX32896; AAX51299; AAY27556; AAY52492; AAY52493; AAY52494; AAY52495; AAY52496; AAY52497; AAY52498; AAY52499; AAY52500; AAY52501; AAY52502; AAY52503; AAY52504; AAY52505; AAY52506; AAY52507; AAY52508; AAY52509; AAY52510; AAY52511; AAY52512; AAY52513; AAY52514; AAY52515; AAY52516; AAY52517; AAY52518; AAY52519; AAZ14102; AAZ14103; AAZ14104; AAZ14105; AAZ14106; AAZ14107; AAZ14108; AAZ14109; AAZ14110; AAZ14111; AAZ14112; AAZ14113; AAZ14114; AAZ14115; AAZ14116; AAZ14117; AAZ14118; AAZ14119; AAZ14120; AAZ14121; AAZ14122; AAZ14123; AAZ14124; AAZ14125; AAZ14126; AAZ14127; AAZ14128; AAZ14129; AAZ14977; AAZ14978; AAZ14979; AAZ14980; AAZ14981; AAZ14982; AAZ14983; AAZ14984; AAZ14985; AAZ14986; AAZ14987; AAZ14988; AAZ14989; AAZ14990; AAZ14991; AAZ14992; AAZ14993; AAZ14994; AAZ14995; AAZ14996; AAZ14997; AAZ14998; AAZ14999; AAZ15000; AAZ15001; AAZ15002; AAZ15003; AAZ15004; AAZ15005; AAZ15006; AAZ15007; AAZ15008; AAZ15009; AAZ15010; AAZ15011; AAZ15012; AAZ15013; AAZ15014; ABB03902; ABB17027; ABB17191; ABB19481; ABB19693; ABB20314; ABB20324; ABB20444; ABB51137; ABB58945; ABB58946; ABB58947; ABB58948; ABB58949; ABB58950; ABB58951; ABB58952; ABB58953; ABB58954; ABB58955; ABB87163; ABB87314; ABB87366; ABB87864; ABB87875; ABB87886; ABB87896; ABB87907; ABB87918; ABB87929; ABB87939; ABB87950; ABB88247; ABB88390; ABB90182; ABB90203; ABB90214; ABC48798; ABC48808; ABC48818; ABC48828; ABC48838; ABD61024; ABE02148; ABE27712; ABE27713; ABE27714; ABE27715; ABE27716; ABE27717; ABE27718; ABE28413; ABF56623; ABF56632; ABF56641; ABG27038; ABG27042; ABG27051; ABG27056; ABH12262; ABI17549; ABI17550; ABI84463; ABI84523; ABI94767; ABI94782; ABI96694; ABI96715; ABI96777; ABI97307; ABJ15706; ABK00113; ABK00119; ABK00143; ABK41621; ABK59023; ABM21875; ABM21876; ABM21877; ABM21878; ABM21879; ABM21880; ABM21881; ABM46227; ABM46228; ABM46229; ABM46230; ABM46231; ABM46232; ABM46233; ABM46234; ABM46235; ABM46236; ABM46237; ABM46238; ABM46239; ABM46240; ABM46241; ABM46242; ABM46243; ABM46244; ABM46245; ABM46246; ABM46247; ABM46248; ABM46249; ABM46250; ABM46251; ABM46252; ABM46253; ABM46254; ABM46255; ABM46256; ABM46257; ABM46258; ABM46259; ABM46260; ABM46261; ABM46262; ABM46263; ABM46264; ABM46265; ABM46266; ABM46267; ABM46268; ABM46269; ABM46270; ABM46271; ABM46272; ABM46273; ABM46274; ABM46275; ABM46276; ABM46277; ABM46278; ABM46279; ABM46280; ABM46281; ABM46282; ABM46283; ABM46284; ABM46285; ABM46286; ABM46287; ABM46288; ABM46289; ABM46290; ABM46291; ABM46292; ABM46293; ABM46294; ABM46295; ABM46296; ABM46297; ABM46298; ABM46299; BAA14335; BAB39511; BAB39512; BAB85614; BAB85615; BAB85616; BAB85617; BAB85618; BAD01514; BAD01515; BAD01516; BAD01517; BAD01518; BAE96033; BAF34373; BAF46427; BAF46437; BAF46447; BAF46457; BAF46467; BAF46477; BAF46487; BAF46497; BAF46507; BAF46517; BAF46527; BAF48357; CAB95856; CAB95857; CAC19694; CAD60401; CAD60402; CAD60403; CAH04111; CAH04112; CAH04113; CAH04114; CAH04115; CAH04116; CAH04117; CAH04118; CAH04119; CAH04120; CAJ32552; CAJ32553; CAL15444 and CAL15445.

Protein sequences of influenza virus subtype H10 hemagglutinin suitable for inclusion as a cargo moiety in conjugates herein have the following accession numbers and are available to the public via the National Center for Biotechnology Information (NCBI) Entrez protein search and retrieval system:

BAF03631; BAF31846; ABB87989; ABB88000; ABB88011; ABB88022; ABB88033; ABB88044; ABI84469; ABI84499; ABI84534; ABI84626; ABL67143; ABO52082; ABO52093; ABO52115; ABD23975; AAA43186; AAA79774; AAA79775; BAF43464; BAF46762; BAF46908; BAF47127; BAF48645; AAG33016; CAJ32549; CAJ32550; ABB87206; ABB87217; ABB87325; ABB87844; ABB87956; ABB87967 and ABB87978.

Protein sequences of influenza virus subtype H11 hemagglutinin suitable for inclusion as a cargo moiety in conjugates herein have the following accession numbers and are available to the public via the National Center for Biotechnology Information (NCBI) Entrez protein search and retrieval system:

BAF34926; BAF43435; ABD91535; ABD66294; ABD66295; ABD66296; ABD66297; ABD66298; ABF22671; ABM54148; AAA43188; AAA43191; AAA43183; AAA43203; AAA43181; BAF47125; BAF47129; BAF48643; BAF49417; AAG38553; AAV91221; ABB87228; ABB87239; ABB88055; ABB88066; ABB88077; ABB88088; ABI84440; ABI84442; ABI84545; ABI84556; ABI84600; ABI84723; ABJ53570; ABL67121; ABL67231; ABL75585; ABO52137; ABO52148; ABO52170; ABO52390; ABO52401; ABO52412; ABO52434; ABO52445; ABO52456; ABO52544; ABO52555; ABO76924; ABO76935; ABO76968; ABP49195; ABP49239; ABP49250; ABP49261; ABP49272; ABP49294; BAA14336; AAY85533; ABC59903; ABD91532; ABD91533 and ABD91534.

Protein sequences of influenza virus subtype H12 hemagglutinin suitable for inclusion as a cargo moiety in conjugates herein have the following accession numbers and are available to the public via the National Center for Biotechnology Information (NCBI) Entrez protein search and retrieval system:

BAF43416; BAF43433; AAG38557; AAG38558; AAG38559; CAL15446; ABB87195; ABB87249; ABB88099; ABB88110; ABB88121; ABG88278; ABI84446; ABI84489; ABJ09129; ABL67077; ABL67242; ABO52610; ABO52621; BAA14337; ABI17552 and AAA43180.

Protein sequences of influenza virus subtype H13 hemagglutinin suitable for inclusion as a cargo moiety in conjugates herein have the following accession numbers and are available to the public via the National Center for Biotechnology Information (NCBI) Entrez protein search and retrieval system:

BAF37821; BAF38383; BAF46906; CAJ32554; CAJ32555; AAV91212; AAV91213; ABB86511; ABB87334; ABB87345; ABB87811; ABI84452; ABI84566; ABI84601; BAA14338; ABG57285; AAA43213; AAA43214 and AAA43215.

Protein sequences of influenza virus subtype H14 hemagglutinin suitable for inclusion as a cargo moiety in conjugates herein have the following accession numbers and are available to the public via the National Center for Biotechnology Information (NCBI) Entrez protein search and retrieval system:

BAF43460 and ABI84453.

Protein sequences of influenza virus subtype H15 hemagglutinin suitable for inclusion as a cargo moiety in conjugates herein have the following accession numbers and are available to the public via the National Center for Biotechnology Information (NCBI) Entrez protein search and retrieval system:

ABB88138; ABB88320; ABB88331; AAA96134; ABB90704; BAF48363; ABB88132 and AAA92247.

Protein sequences of influenza virus subtype H16 hemagglutinin suitable for inclusion as a cargo moiety in conjugates herein have the following accession numbers and are available to the public via the National Center for Biotechnology Information (NCBI) Entrez protein search and retrieval system:

ABB87356; ABI84447; ABI85221; AAV91214; AAV91215; AAV91216 and AAV91217.

Polynucleotides encoding influenza virus subtype H1 hemagglutinins suitable for inclusion as a cargo moiety in conjugates herein have the following accession numbers and are available to the public via the National Center for Biotechnology Information (NCBI) Entrez nucleotide search and retrieval system:

AF222026; AF222034; AF222036; AF503481; AF503483; AY180460; AF534030; AF534045; AF534052; AY604795; AY604797; AY604798; AY604804; AY604806; CY004490; CY004539; CY006355; CY006779; CY009324; CY010092; CY010172; CY010228; CY010284; CY010324; CY010340; CY010996; CY011208; M59325; CY013565; CY013581; CY016563; CY017139; EF462556; EF462564; CY020173; CY021709; CY021749; CY021821; AB243744; AB255389; AB255390; AB255392; AB271115; X57491; AF222030; AF222031; AF222032; AF222033; AF503484; AF503485; AF503486; AF534048; AF534049; AY377936; AY604799; AY604800; AY604801; AY604802; AY604803; AY684125; DQ058215; CY003016; CY004498; CY005866; CY008148; CY009204; CY009276; CY009628; CY010108; CY010292; CY010300; CY010316; CY011012; CY011072; CY011088; CY011168; CY011184; CY011224; CY014627; CY014968; CY016052; CY016699; CY017123; CY017219; CY019763; CY019867; CY019883; EF467821; EF462563; X57493; Z46437; Z54288; L20111; L20113; L25072; U11858; L33780; L19022; L19016; L19014; M38312; L19006; L19024; K01331; U85986; AY029292; AF320059; AF320060; AF320062; AY129156; AF503475; AF503477; AY299509; AY297156; AY303741; AY701753; CY002672; CY002984; CY003688; CY003761; CY006419; DQ265706; DQ280203; CY009444; CY009868; CY009964; CY010556; CY010868; DQ666649; DQ666650; CY012440; DQ978382; DQ978389; DQ978390; EF462570; EF462571; CY020181; CY020861; CY021029; CY021693; CY021733; AB274304; Z46434; Z46435; Z46436; AJ289702; L19028; L19019; L19018; L19008; L19027; J02144; L19017; L19026; L24362; AY029288; AY029289; AF320063; AF503478; AF503479; AF503480; AY303747; CY000449; CY002360; CY002648; CY002688; CY003024; DQ249260; CY006107; CY003833; CY006667; CY006675; DQ280195; DQ280227; DQ280236; DQ280243; DQ280250; CY006867; CY009532; M34335; M33748; AF131993; AF131994; AF222027; AF222028; AF222035; AF503482; AF534027; AF534031; AF534038; AF534043; AF534044; AF534046; AF534053; AF534054; AY604796; AY604805; AY604807; AY604808; AY682833; CY002704; CY004504; CY004531; CY004546; CY005735; CY006363; CY003769; CY009220; CY009332; DQ397950; CY009452; DQ431990; CY010156; CY010164; CY010180; CY010236; CY010276; CY010332; M81707; CY011176; CY011192; CY011200; CY011216; CY015580; CY016308; CY016311; CY016313; CY016317; CY016318; CY016321; CY016327; CY16328; CY016331; CY016435; CY17003; CY017195; CY017235; CY17251; CY019101; CY020469; CY020485; ABO43478; ABO43483; ABO43484; ABO43491; ABO43493; ABO43494; X17221; J04572; J04574; L33480; L33482; L33487; L33489; L33490; L33755; L33743; L33745; L33747; L33753; U11703; U08903; U53162; S62154; S67220; U80948; U45451; U46943; AF305218; CY009756; CY009764; CY009780; CY009844; CY009956; CY010572; CY010740; CY010780; CY011952; DQ666648; CY013271; DQ981739; DQ978383; CY014733; CY015524; CY016228; CY016314; CY016315; CY016316; CY016324; CY016325; CY016326; CY016955; CY017011; CY017315; CY019085; CY020421; CY020437; CY020509; ABO43480; ABO43481; ABO43486; ABO43487; ABO43490; ABO43495; ABO43496; ABO43497; ABO43500; AB294217; L33483; L33484; L33485; L33486; L33492; L33493; CY011584; M59324; M59326; CY013573; CY013589; CY016643; CY018885; CY019205; CY019771; CY019875; CY019963; CY019971; EF462565; CY020917; CY021701; CY021717; CY021725; AB243745; AB255391; AB255393; AB255397; AB255398; AB271113; X57492; Z30276; Z54289; L20109; L20112; L20110; L25071; M73975; L19015; L19549; M38353; K01330; J02176; L19025; L19013; L19023; U03719; U03720; U72666; AY029291; AF320057; AF320058; AF320061; AF131995; AF250124; AF268312; AF268313; AF222029; AF534025; AF534026; AF534047; AF534050; AF534051; CY0001952; CY003288; CY004507; CY004592; CY009212; CY009596; DQ447187; CY010100; CY010116; CY010212; CY010220; CY010308; CY011004; CY011080; CY011152; CY011160; CY011232; M59327; M59328; CY012888; CY017115; CY017203; CY017211; CY017227; CY019739; CY019755; CY020189; CY021053; CY021757; CY021797; AB255394; AB255395; AB255396; X57494; X59778; AF398875; AF455679; AF455681; AF494246; AF494249; AF494250; AY289927; AY299499; AY299500; AY299502; CY002152; CY002616; CY002800; CY004466; CY004474; CY004482; DQ335992; DQ335995; CY009540; CY009612; CY009620; CY009796; CY009980; CY010356; CY010364; CY010380; CY010428; CY010444; CY010524; CY010540; DQ508897; DQ534416; CY011280; CY011296; CY011304; CY011392; CY012296; CY013821; CY013837; CY013853; CY013597; CY013871; CY016337; CY016338; CY016343; U02085; L09063; L33750; L33751; L33752; U37727; U38242; U53163; U80949; U46020; U46021; U46782; U46783; U46941; U46942; AF398878; AF455678; AY095226; AY095227; AF494251; AY289928; AY289929; AY633212; CY002536; CY002632; CY003000; DQ335998; CY009884; CY010372; CY009916; CY010388; CY010404; CY010460; CY010476; CY010492; CY010500; DQ508905; CY010764; CY011240; CY011312; CY012856; CY012864; CY012880; CY013032; CY015163; AF320067; AF503474; AF503476; AY299508; AY297157; AY303734; DQ139320; CY002352; CY002696; CY002992; CY003696; CY003704; DQ280212; DQ280219; CY006875; CY009772; CY009972; CY010804; CY010876; DQ666651; CY012824; CY013287; DQ978387; DQ978388; DQ978391; CY016196; CY016309; CY016310; CY016312; CY016319; CY016320; CY016322; CY016329; CY016330; CY020293; CY020453; CY020461; CY020477; ABO43479; ABO43482; ABO43485; ABO43492; V01088; X17224; J04573; CY016334; CY016335; CY016340; CY016341; CY016342; CY016347; CY016350; CY016357; CY016360; CY016361; CY016365; CY016366; CY016367; CY016370; CY017371; CY017869; CY019069; CY020237; CY020253; CY020261; U47310; AF026157; AF026160; AF091313; AF362778; AF362779; AF362784; AF362785; AF362786; AF362787; AF362794; AF362795; AF362796; AF362803; AF386775; AF386776; AF386777; AF386782; AF386783; AY038014; AY038338; AY038339; AY038344; AY038345; AY038346; L33481; L33488; L33491; L33756; L33744; L33746; L33754; L19005; K00992; U44482; U45452; AF398874; AF455680; AF494247; AF494248; AY299497; AY299498; AY299501; AY299503; CY002808; CY004458; CY006187; CY006195; DQ335993; DQ335994; CY009548; CY009604; CY009788; CY009804; CY009876; CY009892; CY010348; CY010412; CY010420; CY010436; CY010452; CY010508; CY010532; DQ508873; DQ508889; CY010772; DQ534415; DQ534417; DQ534418; CY011272; AY038347; AY038354; AY038355; AY038356; AY038357; AY060038; AY060044; AY233393; AY790289; AY851464; AY851465; AY851466; AY851467; AY971006; AY971007; AY971010; AY971011; DQ100426; DQ100427; CY002392; CY003296; CY003376; CY003392; CY003464; CY003480; CY006395; CY006403; DQ335999; DQ415318; CY009292; CY009316; CY009828; CY009940; CY010148; CY010204; CY010244; CY010828; CY010844; CY010884; CY010916; CY010924; CY010940; CY010956; CY011608; CY011776; CY016344; CY016346; CY016354; CY016363; CY016364; CY016371; CY016373; CY017363; CY017427; CY017435; CY019045; CY020269; CY021005; D00406; D00837; D00839; D10477; D29656; U47305; U47307; U96766; AF026153; AF026154; AF026156; AF055426; AF091309; AF091310; AF091312; AF362781; AF362783; AF362788; AF362791; AF362797; AF362798; AF362800; AF362802; AF386773; AF386774; AF386780; AY038335; AY038337; AY038341; AY038343; AY038351; AY038353; CY013829; CY013845; CY013879; CY0115167; CY016336; CY016339; CY016345; CY016352; CY016355; CY016362; CY016372; CY016459; CY017419; CY020277; D00407; D00838; D13570; D29657; U47304; U47306; U47308; AF026155; AF085413; AF117241; AF091308; AF091311; AF091317; AF362780; AF362782; AF362789; AF362790; AF362792; AF362799; AF362801; AF386779; AF386781; AF387491; AY038334; AY038336; AY038340; AY038342; AY038349; AY038350; AY038352; AY060031; AY038358; AF408859; AY060032; AY060039; AY060045; AY060047; AY060048; AY063229; AY184805; AY851463; AY851471; AY971003; AY971004; DQ118159; DQ118160; DQ118162; CY002528; CY003304; CY003328; CY003672; CY006171; CY006387; DQ336002; DQ336005; CY008524; CY008996; CY009172; CY009188; CY009228; CY009284; CY009340; CY010076; CY010132; CY010188; CY010268; CY010908; CY010964; CY010980; CY012608; CY011040; CY014007; DQ986134; CY016374; CY016380; CY016386; CY011792; CY013303; DQ978392; CY016244; CY016375; CY016376; CY016377; CY016382; CY016383; CY016391; CY016392; CY016393; DQ973300; CY019125; CY019221; CY019237; CY019923; CY020141; CY020157; CY020165; AB117167; AB117170; AB117171; AB117176; AB117177; AB117183; AB117192; AB117193; AB117203; AB117212; AB117213; AB117221; AB126622; AJ412708; AJ412709; AJ344013; AJ457868; AJ457869; AJ457878; AJ457879; AJ457884; AJ457885; AJ457886; AJ457894; AJ457895; AJ457904; AY060033; AY060034; AY060040; AY060046; AY060049; AY060050; AY063228; AF342821; AY590823; AY590824; AY790267; AY701755; AY851462; AY851469; AY851470; AY851472; AY861443; AY971005; DQ118158; DQ118161; DQ118163; CY003312; CY003320; CY003400; CY006427; CY006411; DQ336003; DQ336004; CY008988; DQ415316; CY009180; CY009196; CY009236; CY010196; CY010252; CY010260; CY010892; CY1900; CY010972; CY012800; CY016379; CY016381; CY016385; CY016387; CY016388; CY016389; CY016390; DQ973301; DQ973303; DQ973304; CY016971; CY017275; EF101749; CY017717; CY017725; CY017829; CY018933; CY019117; CY019779; CY019795; CY019803; CY020565; AB117166; AB117173; AB117174; AB117180; AB117182; AB117189; AB17190; AB117196; AB117199; AB117200; AB117202; AB117209; AB117210; AB17216; AB117219; AB117220; AB126630; AJ412712; AJ344002; AJ344019; AJ344020; AJ344009; AJ344010; AJ344012; AJ344022; AJ457865; AJ457872; AJ457875; AJ457881; CY016723; DQ973299; DQ973302; EF101741; CY017813; CY019109; CY020573; CY021629; AB117165; AB117172; AB117175; AB117181; AB117187; AB17188; AB17191; AB17197; AB17198; AB17201; AB17207; AB17208; AB117211; AB117217; AB117218; AJ412711; AJ344017; AJ344018; AJ344021; AJ344003; AJ344008; AJ344011; AJ457863; AJ457864; AJ457866; AJ457873; AJ457874; AJ457880; AJ457882; AJ457889; AJ457890; AJ457896; AJ457899; AJ457900; AJ457902; AJ457906; AJ457909; AJ457910; AJ457887; AJ457888; AJ457891; AJ457897; AJ457898; AJ457901; AJ457907; AJ457908; AJ457911; AJ489854; AJ489856; AJ517814; AJ517816; Z46441; Z54286; Z54287; L20106; L20117; L20116; L20108; L20107; U11857; L19021; L19011; L19020; L19012; U72667; U72668; U72669; AY029287; AY029290; AF320056; AF320064; AF320065; AF320066; AF503473; AY299506; AY299507; AY297154; AY297155; AY619961; CY002624; CY002640; CY002664; CY002680; CY003008; CY006747; CY006915; CY009860; CY010580; CY010852; DQ666644; DQ666645; DQ666646; DQ666647; CY013279; CY013295; DQ978384; DQ978385; DQ978386; CY014901; CY015443; CY015532; CY016260; CY016323; CY016963; CY017019; CY017243; CY019077; CY019093; CY019997; CY020429; CY020445; CY021037; ABO43488; ABO43489; ABO43498; ABO43499; U04857; U04858; U04859; L33757; L33758; L33748; L33749; U08904; U80950; AF455675; AF455676; AF455677; AF455682; AF389118; AY289930; AY282756; AY282757; AY299494; AY299495; AY299496; AY299504; AY299505; CY002568; CY002400; CY002656; DQ335991; DQ335996; DQ335997; CY009812; CY010396; CY010468; CY010484; DQ508857; DQ534419; CY011408; CY012304; CY012872; CY013040; CY013813; CY016332; CY016333; CY016348; CY016349; CY016358; CY016359; CY016368; CY016369; CY016691; CY017147; CY017155; CY017379; CY017877; CY019053; CY019061; CY019947; CY019955; CY020245; CY020285; D00840; D00841; D10163; D13571; D13572; D13573; D13574; D28518; D31949; U47309; AF026158; AF026159; AF085414; AJ457905; AJ489852; AJ489853; AJ489860; AJ489861; AJ489862; AJ517813; AJ517817; AJ517820; AJ489855; AJ489857; AJ489858; AJ517815; AF085415; AF085416; AF085417; AF116575; AF116576; AF091306; AF091307; AF091314; AF091315; AF091316; AF362793; AF386778; AY038333; AY038348; AY052778; AY060030; AY060035; AY060036; AY060037; AY060041; AY060042; AY060043; AY060051; AY060052; AY184806; AY590822; AY377929; AY851468; AY971008; AY971009; CY001680; DQ18164 and CY003368.

Polynucleotides encoding influenza virus subtype H2 hemagglutinins suitable for inclusion as a cargo moiety in conjugates herein have the following accession numbers and are available to the public via the National Center for Biotechnology Information (NCBI) Entrez nucleotide search and retrieval system:

AB056699; AB266382; AY209954; AY209955; AY209956; AY209957; AY209958; AY209959; AY209960; AY209961; AY209962; AY209963; AY209964; AY209965; AY209966; AY209967; AY209968; AY209969; AY209970; AY209971; AY209972; AY209973; AY209974; AY209975; AY209976; AY209977; AY209978; AY209979; AY209980; AY209981; AY209982; AY209983; AY209984; AY209985; AY209986; AY209987; AY209988; AY209989; AY422014; AY422015; AY422016; AY422017; AY633180; AY633196; AY633228; AY633364; AY633388; AY684893; CY003847; CY003855; CY003863; CY003871; CY003879; CY003887; CY003907; CY003914; CY003922; CY003936; CY003944; CY003952; CY003960; CY003968; CY003976; CY003984; CY003992; CY004554; CY005413; CY005538; CY005546; CY005765; CY005808; CY014556; CY014558; CY014601; CY014608; CY014609; CY014710; CY014821; CY014829; CY014976; CY015135; CY017693; CY018877; CY020317; CY020373; CY020381; CY020389; CY020397; CY020405; CY020413; CY020517; CY020541; CY020549; CY021013; CY021021; CY021069; CY021125; CY021789; CY021805; CY021813; D13575; D13576; D13577; D13578; D13579; D13580; DQ006282; DQ006283; DQ009917; DQ017486; DQ017493; DQ508841; DQ508881; J02127; J02154; L11125; L11126; L11127; L11128; L11129; L11130; L11131; L11132; L11133; L11134; L11135; L11136; L11137; L11138; L11139; L11140; L11141; L11142; L20406; L20407; L20408; L20409; L20410; AB275406; AB275414; AB275620; AB275628; AB275861; AB276115; AB292785; AB296074; AB298281; AF116197; AF116198; AF116199; AF116200; AF116201; AF16202; AF116203; AF16204; AF16205; AF16206; AF16207; AF16208; AF16209; AF116210; AF16211; AF231356; AF270716; AF270717; AF270718; AF270719; AF270720; AF270721; AF270722; AF270723; AF270724; AF270725; AF270726; AF270727; AF270728; AY180398; AY180399; AY180400; AY180401; AY180402; AY180403; AY209952 and AY209953.

Polynucleotides encoding influenza virus subtype H3 hemagglutinins suitable for inclusion as a cargo moiety in conjugates herein have the following accession numbers and are available to the public via the National Center for Biotechnology Information (NCBI) Entrez nucleotide search and retrieval system:

AB013806; AB013807; AB013808; AB013809; AB013810; AB013811; AB013812; AB013813; AB014060; AB014061; AB014062; AB019354; AB019355; AB019356; AB019357; AB043705; AB043706; AB043707; AB043708; AB221016; AB221017; AB221018; AB221019; AB221020; AB221021; AB221022; AB221023; AB221024; AB221025; AB221026; AB221027; AB221028; AB221029; AB221030; AB221031; AB221032; AB221033; AB221034; AB221035; AB243867; AB243868; AB243869; AB243870; AB243871; AB243872; AB243873; AB246366; AB259101; AB259102; AB259103; AB259104; AB259105; AB259106; AB259107; AB259108; AB259109; AB259110; AB259111; AB259112; AB259739; AB259740; AB259741; AB262301; AB262302; AB262303; AB262304; AB270992; AB270993; AB270994; AB270995; AB270996; AB270997; AB270998; AB270999; AB271000; AB271001; AB271002; AB271489; AB271490; AB271491; AB271492; AB271493; AB271494; AB271495; AB271496; AB271497; AB271498; AB271503; AB271504; AB271505; AB271506; AB271511; AB271512; AB271513; AB271514; AB271515; AB271516; AB271517; AB271524; AB271525; AB271526; AB271527; AB271528; AB271529; AB271530; AB271809; AB271810; AB271811; AB271812; AB271813; AB271814; AB271815; AB271816; AB271817; AB271818; AB271819; AB271820; AB271821; AB271822; AB271823; AB271824; AB271825; AB271826; AB271827; AB271828; AB271829; AB271830; AB271831; AB271832; AB271833; AB271834; AB271835; AB271836; AB271837; AB271838; AB271839; AB271840; AB271841; AB271842; AB271843; AB271844; AB271845; AB271846; AB271847; AB271848; AB271849; AB271850; AB275283; AB276113; AB277754; AB284320; AB289341; AB292402; AB292410; AB292660; AB292668; AB295605; AF008656; AF008657; AF008658; AF008659; AF008660; AF008661; AF008662; AF008663; AF008664; AF008665; AF008666; AF008667; AF008668; AF008669; AF008670; AF008671; AF008672; AF008673; AF008674; AF008675; AF008676; AF008677; AF008678; AF008679; AF008680; AF008681; AF008682; AF008683; AF008684; AF008685; AF008686; AF008687; AF008688; AF008689; AF008690; AF008691; AF008692; AF008693; AF008694; AF008695; AF008696; AF008697; AF008698; AF008699; AF008700; AF008701; AF008702; AF008703; AF008704; AF008705; AF008706; AF008707; AF008708; AF008709; AF008710; AF008711; AF008712; AF008713; AF008714; AF008715; AF008716; AF008717; AF008718; AF008719; AF008720; AF008721; AF008722; AF008723; AF008724; AF008725; AF008726; AF008727; AF008728; AF008729; AF008730; AF008731; AF008732; AF008733; AF008734; AF008735; AF008736; AF008737; AF008738; AF008739; AF008740; AF008741; AF008742; AF008743; AF008744; AF008745; AF008746; AF008747; AF008748; AF008749; AF008750; AF008751; AF008752; AF008753; AF008754; AF008755; AF008756; AF008757; AF008758; AF008759; AF008760; AF008761; AF008762; AF008763; AF008764; AF008765; AF008766; AF008767; AF008768; AF008769; AF008770; AF008771; AF008772; AF008773; AF008774; AF008775; AF008776; AF008777; AF008778; AF008779; AF008780; AF008781; AF008782; AF008783; AF008784; AF008785; AF008786; AF008787; AF008788; AF008789; AF008790; AF008791; AF008792; AF008793; AF008794; AF008795; AF008796; AF008797; AF008798; AF008799; AF008800; AF008801; AF008802; AF008803; AF008804; AF008805; AF008806; AF008807; AF008808; AF008809; AF008810; AF008811; AF008812; AF008813; AF008814; AF008815; AF008816; AF008817; AF008818; AF008819; AF008820; AF008821; AF008822; AF008823; AF008824; AF008825; AF008826; AF008827; AF008828; AF008829; AF008830; AF008831; AF008832; AF008833; AF008834; AF008835; AF008836; AF008837; AF008838; AF008839; AF008840; AF008841; AF008842; AF008843; AF008844; AF008845; AF008846; AF008847; AF008848; AF008849; AF008850; AF008851; AF008852; AF008853; AF008854; AF008855; AF008856; AF008857; AF008858; AF008859; AF008860; AF008861; AF008862; AF008863; AF008864; AF008865; AF008866; AF008867; AF008868; AF008869; AF008870; AF008871; AF008872; AF008873; AF008874; AF008875; AF008876; AF008877; AF008878; AF008879; AF008880; AF008881; AF008882; AF008883; AF008884; AF008885; AF008886; AF008887; AF008888; AF008889; AF008890; AF008891; AF008892; AF008893; AF008894; AF008895; AF008896; AF008897; AF008898; AF008899; AF008900; AF008901; AF008902; AF008903; AF008904; AF008905; AF008906; AF008907; AF008908; AF008909; AF017270; AF017271; AF017272; AF038266; AF038267; AF038268; AF038269; AF038270; AF079570; AF087700; AF087701; AF087702; AF087703; AF087704; AF087705; AF087706; AF087707; AF087708; AF087709; AF092052; AF092053; AF092054; AF092055; AF092056; AF092057; AF092058; AF092059; AF092060; AF092061; AF092062; AF092063; AF092064; AF096306; AF096307; AF096308; AF096309; AF096310; AF096311; AF096312; AF096313; AF096314; AF096315; AF096316; AF131996; AF131997; AF131998; AF139930; AF139931; AF139932; AF139933; AF139934; AF139935; AF139936; AF139937; AF139938; AF139939; AF139940; AF153232; AF153233; AF153234; AF153235; AF180564; AF180565; AF180566; AF180567; AF180568; AF180569; AF180570; AF180571; AF180572; AF180573; AF180574; AF180575; AF180576; AF180577; AF180578; AF180579; AF180580; AF180581; AF180582; AF180583; AF180584; AF180585; AF180586; AF180587; AF180588; AF180589; AF180590; AF180591; AF180592; AF180593; AF180594; AF180595; AF180596; AF180597; AF180598; AF180599; AF180600; AF180601; AF180602; AF180603; AF180604; AF180605; AF180606; AF180607; AF180608; AF180609; AF180610; AF180611; AF180612; AF180613; AF180614; AF180615; AF180616; AF180617; AF180618; AF180619; AF180620; AF180621; AF180622; AF180623; AF180624; AF180625; AF180626; AF180627; AF180628; AF180629; AF180630; AF180631; AF180632; AF180633; AF180634; AF180635; AF180636; AF180637; AF180638; AF180639; AF180640; AF180641; AF180642; AF180643; AF180644; AF180645; AF180646; AF180647; AF180648; AF180649; AF180650; AF180651; AF180652; AF180653; AF180654; AF180655; AF180656; AF180657; AF180658; AF180659; AF180660; AF180661; AF180662; AF180663; AF180664; AF180665; AF180666; AF197241; AF197242; AF197243; AF197244; AF197245; AF197246; AF197247; AF197248; AF197249; AF201842; AF201843; AF201844; AF201845; AF201846; AF201874; AF201875; AF204238; AF213900; AF213901; AF213902; AF213903; AF225542; AF225543; AF225544; AF225545; AF233691; AF251395; AF251403; AF251411; AF251419; AF251427; AF255019; AF255020; AF255021; AF255022; AF255023; AF255024; AF255025; AF255026; AF255027; AF255028; AF255029; AF268123; AF268124; AF268125; AF268126; AF268127; AF268128; AF297094; AF297095; AF297096; AF297097; AF311676; AF311677; AF311678; AF311679; AF311680; AF311681; AF311682; AF311683; AF311684; AF311685; AF311686; AF311687; AF311688; AF311689; AF311690; AF311691; AF311692; AF311693; AF311694; AF311695; AF311696; AF311697; AF311698; AF315559; AF315560; AF315561; AF315562; AF315563; AF315564; AF315565; AF315566; AF315567; AF315568; AF315569; AF315570; AF315571; AF316817; AF316818; AF316819; AF316820; AF316821; AF348176; AF357929; AF357930; AF357931; AF357932; AF357933; AF357934; AF357935; AF357936; AF357937; AF357938; AF357939; AF357940; AF357941; AF357942; AF357943; AF357944; AF357945; AF357946; AF357947; AF357948; AF357949; AF357950; AF357951; AF357952; AF357953; AF357954; AF357955; AF357956; AF357957; AF357958; AF357959; AF357960; AF357961; AF357962; AF357963; AF357964; AF357965; AF357966; AF357967; AF357968; AF357969; AF362804; AF362805; AF362806; AF362807; AF362808; AF362809; AF362810; AF362811; AF362812; AF362813; AF362814; AF362815; AF362816; AF362817; AF362818; AF362819; AF362820; AF363502; AF363503; AF363504; AF368436; AF368437; AF368438; AF368439; AF368440; AF368441; AF368442; AF368443; AF368444; AF368445; AF368446; AF382318; AF382319; AF382320; AF382321; AF382322; AF382323; AF382324; AF382325; AF382326; AF382327; AF382328; AF386604; AF386605; AF386606; AF386607; AF386608; AF386609; AF386610; AF386611; AF386612; AF386613; AF386614; AF386615; AF386616; AF386617; AF386618; AF386619; AF386620; AF386621; AF386622; AF386623; AF386624; AF386625; AF386626; AF386627; AF386628; AF386629; AF386630; AF386631; AF386632; AF386633; AF386634; AF400752; AF400753; AF400754; AF405206; AF405207; AF405208; AF405209; AF405210; AF405211; AF405212; AF442455; AF442456; AF442457; AF442458; AF442459; AF442460; AF442461; AF442462; AF442463; AF442464; AF442465; AF442466; AF442467; AF442468; AF442469; AF442470; AF442471; AF442472; AF442473; AF442474; AF442475; AF442476; AF442477; AF442478; AF442479; AF442480; AF442481; AF442482; AF442483; AF450246; AF501515; AF501516; AF501517; AF501518; AF501519; AF501520; AF501521; AF501522; AF501523; AF501524; AF501525; AF501526; AF501527; AF501528; AF501529; AF501530; AF501531; AF501532; AF501533; AF501534; AF501535; AF525217; AF525218; AF525219; AF525686; AF525799; AF533712; AF533713; AF533714; AF533715; AF533716; AF533717; AF533718; AF533719; AF533720; AF533721; AF533722; AF533723; AF533724; AF533725; AF533726; AF533727; AF533728; AF533729; AF534013; AF534014; AF534015; AF534016; AF534017; AF534018; AF534019; AF534020; AF534021; AF534022; AF534023; AF534024; AF534028; AF534029; AF534032; AF534033; AF534034; AF534035; AF534036; AF534037; AF534039; AF534040; AF534041; AF534042; AF534055; AF534056; AF534057; AF534058; AF534059; AF534060; AJ223192; AJ223193; AJ223194; AJ223195; AJ223196; AJ223197; AJ252129; AJ252130; AJ252131; AJ252132; AJ293926; AJ293927; AJ293928; AJ293929; AJ293930; AJ293931; AJ293932; AJ293933; AJ311454; AJ311466; AJ311511; AJ344023; AJ427297; AJ427304; AJ506781; AJ697863; AJ697864; AJ697865; AJ697866; AJ704814; AJ704815; AJ704816; AJ746251; AJ841293; AM087217; AM087224; AX350190; AX350204; AY032978; AY035588; AY035589; AY035590; AY035591; AY035592; AY048077; AY048078; AY048079; AY048080; AY048081; AY137206; AY138513; AY138515; AY138516; AY138517; AY138518; AY138519; AY180404; AY180405; AY180406; AY180407; AY180408; AY180409; AY180410; AY180411; AY180412; AY180413; AY180414; AY180415; AY180416; AY180417; AY180418; AY180419; AY180420; AY180421; AY180422; AY180423; AY180424; AY180425; AY180426; AY180427; AY180428; AY180429; AY180430; AY180431; AY180432; AY180433; AY262744; AY262745; AY271794; AY273167; AY273168; AY303713; AY303717; AY303723; AY303731; AY303736; AY303743; AY303745; AY363509; AY363510; AY363511; AY363512; AY363513; AY363514; AY363515; AY363516; AY363517; AY363518; AY363519; AY363520; AY363521; AY363522; AY363523; AY363524; AY363525; AY363526; AY377129; AY377537; AY377538; AY377539; AY377540; AY377541; AY377542; AY377543; AY377544; AY377545; AY377546; AY377547; AY377924; AY377927; AY377933; AY383755; AY389349; AY389350; AY389351; AY389352; AY389353; AY389354; AY389355; AY389356; AY389357; AY389358; AY389359; AY389360; AY462237; AY479982; AY531031; AY531033; AY531035; AY531037; AY531039; AY531040; AY531041; AY531042; AY531043; AY531044; AY531045; AY531046; AY531047; AY531048; AY531049; AY531050; AY531051; AY531052; AY531053; AY531054; AY531055; AY531056; AY531057; AY531058; AY531059; AY531060; AY531061; AY589647; AY589648; AY589649; AY589650; AY589651; AY589652; AY589653; AY589654; AY589655; AY589656; AY589657; AY589658; AY589659; AY589660; AY589661; AY596799; AY596800; AY596801; AY604809; AY604810; AY604811; AY604812; AY604813; AY604814; AY604815; AY604816; AY604817; AY604818; AY604819; AY604820; AY604821; AY604822; AY604823; AY604824; AY604825; AY604826; AY604827; AY604828; AY604829; AY604830; AY619969; AY619977; AY625729; AY625730; AY625731; AY633132; AY633148; AY633172; AY633244; AY633252; AY633340; AY633372; AY633996; AY633997; AY633998; AY633999; AY634000; AY634001; AY634002; AY634003; AY634004; AY634005; AY634006; AY634007; AY634008; AY634009; AY634010; AY634011; AY634012; AY634013; AY634014; AY634015; AY634016; AY634017; AY634018; AY634019; AY634020; AY634021; AY634022; AY634023; AY634024; AY634025; AY634026; AY634027; AY634028; AY634029; AY634030; AY634031; AY634032; AY634033; AY634034; AY634035; AY634036; AY634037; AY634038; AY634039; AY634040; AY634041; AY634042; AY634043; AY634044; AY634045; AY634046; AY634047; AY634048; AY634049; AY660991; AY660992; AY660993; AY660994; AY660995; AY660996; AY660997; AY660998; AY660999; AY661000; AY661001; AY661002; AY661003; AY661004; AY661005; AY661006; AY661007; AY661008; AY661009; AY661010; AY661011; AY661012; AY661013; AY661014; AY661015; AY661016; AY661017; AY661018; AY661019; AY661020; AY661021; AY661022; AY661023; AY661024; AY661025; AY661026; AY661027; AY661028; AY661029; AY661030; AY661031; AY661032; AY661033; AY661034; AY661035; AY661036; AY661037; AY661038; AY661039; AY661040; AY661041; AY661042; AY661043; AY661044; AY661045; AY661046; AY661047; AY661048; AY661049; AY661050; AY661051; AY661052; AY661053; AY661054; AY661055; AY661056; AY661057; AY661058; AY661059; AY661060; AY661061; AY661062; AY661063; AY661064; AY661065; AY661066; AY661067; AY661068; AY661069; AY661070; AY661071; AY661072; AY661073; AY661074; AY661075; AY661076; AY661077; AY661078; AY661079; AY661080; AY661081; AY661082; AY661083; AY661084; AY661085; AY661086; AY661087; AY661088; AY661089; AY661090; AY661091; AY661092; AY661093; AY661094; AY661095; AY661096; AY661097; AY661098; AY661099; AY661100; AY661101; AY661102; AY661103; AY661104; AY661105; AY661106; AY661107; AY661108; AY661109; AY661110; AY661111; AY661112; AY661113; AY661114; AY661115; AY661116; AY661117; AY661118; AY661119; AY661120; AY661121; AY661122; AY661123; AY661124; AY661125; AY661126; AY661127; AY661128; AY661129; AY661130; AY661131; AY661132; AY661133; AY661134; AY661135; AY661136; AY661137; AY661138; AY661139; AY661140; AY661141; AY661142; AY661143; AY661144; AY661145; AY661146; AY661147; AY661148; AY661149; AY661150; AY661151; AY661152; AY661153; AY661154; AY661155; AY661156; AY661157; AY661158; AY661159; AY661160; AY661161; AY661162; AY661163; AY661164; AY661165; AY661166; AY661167; AY661168; AY661169; AY661170; AY661171; AY661172; AY661173; AY661174; AY661175; AY661176; AY661177; AY661178; AY661179; AY661180; AY661181; AY661182; AY661183; AY661184; AY661185; AY661186; AY661187; AY661188; AY661189; AY661190; AY661191; AY661192; AY661193; AY661194; AY661195; AY661196; AY661197; AY661198; AY661199; AY661200; AY661201; AY661202; AY661203; AY661204; AY661205; AY661206; AY661207; AY661208; AY661209; AY661210; AY661211; AY695084; AY695085; AY695086; AY695087; AY695088; AY695089; AY695090; AY701752; AY702440; AY702441; AY702442; AY702443; AY702444; AY702445; AY702446; AY702447; AY714347; AY738729; AY779253; AY779254; AY851473; AY851474; AY851475; AY851476; AY851477; AY852273; AY852274; AY852275; AY852276; AY852277; AY854046; AY854047; AY854048; AY854049; AY855341; AY862607; AY862608; AY862609; AY862610; AY862611; AY862612; AY884276; AY884277; AY884278; AY884279; AY884280; AY884281; AY884282; AY884283; AY884284; AY919314; AY945263; AY945264; AY945265; AY945266; AY945267; AY945268; AY945269; AY945270; AY945271; AY945272; AY945273; AY945274; AY945275; AY945276; AY945277; AY945278; AY945279; AY945280; AY945281; AY945282; AY945283; AY945284; AY945285; AY945286; AY945287; AY945288; AY947474; AY947476; AY961997; AY961998; AY961999; AY962000; AY962001; AY962002; AY962003; AY962004; AY962005; AY962006; AY962007; AY962008; AY962009; AY962010; AY962011; AY962012; AY962013; AY962014; AY962015; AY962016; AY962017; AY963782; AY963783; AY963784; AY963785; AY963786; AY963788; AY963789; AY963790; AY963791; AY963792; AY963793; AY963794; AY963795; AY963796; AY968017; AY968018; AY968019; AY968020; AY968021; AY968022; AY968023; AY968024; AY968025; AY968026; AY968027; AY968028; AY968029; AY968030; AY968031; AY968032; AY968033; AY968034; AY968035; AY968036; AY968037; AY968038; AY968039; AY968040; AY968041; AY972827; AY972828; AY972829; AY972830; AY972831; AY972832; AY972833; AY972834; AY972835; AY972836; AY972837; AY972838; AY972839; AY972840; AY972841; AY972842; AY972843; AY972844; AY972845; AY972846; AY972847; AY972848; AY972849; AY972850; AY972851; CS406467; CY000001; CY000009; CY000017; CY000025; CY000033; CY000041; CY000049; CY000057; CY000065; CY000073; CY000081; CY000089; CY000097; CY000105; CY000113; CY000121; CY000129; CY000137; CY000145; CY000153; CY000161; CY000169; CY000177; CY000185; CY000193; CY000201; CY000209; CY000217; CY000225; CY000233; CY000241; CY000249; CY000257; CY000265; CY000273; CY000281; CY000289; CY000297; CY000305; CY000313; CY000321; CY000329; CY000337; CY000345; CY000353; CY000361; CY000369; CY000377; CY000385; CY000393; CY00041; CY000409; CY000417; CY000425; CY000433; CY000441; CY000457; CY000465; CY000473; CY000481; CY000489; CY000497; CY000505; CY000513; CY000521; CY000529; CY000537; CY000545; CY000553; CY000561; CY000569; CY000584; CY000585; CY000593; CY0006001; CY000609; CY000617; CY000625; CY000633; CY000641; CY000649; CY000657; CY000665; CY000673; CY000681; CY000689; CY000697; CY000705; CY000713; CY000721; CY000729; CY000737; CY000745; CY000753; CY000761; CY000769; CY000777; CY000785; CY000793; CY00081; CY000809; CY000817; CY000825; CY000833; CY000841; CY000849; CY000857; CY000865; CY000873; CY000881; CY000889; CY000901; CY000909; CY000917; CY000925; CY000933; CY000941; CY000949; CY000957; CY000965; CY000973; CY000981; CY000989; CY000997; CY001005; CY001013; CY001021; CY001029; CY001037; CY001045; CY001053; CY001061; CY001064; CY001072; CY001080; CY001088; CY001096; CY001104; CY001112; CY001120; CY001128; CY001136; CY001144; CY001152; CY001160; CY001168; CY001176; CY001184; CY001197; CY001205; CY001213; CY001221; CY001229; CY001237; CY001245; CY001253; CY001261; CY001269; CY001277; CY001285; CY001293; CY001301; CY001309; CY001317; CY001325; CY001333; CY001341; CY001349; CY001357; CY001365; CY001373; CY001381; CY001397; CY001405; CY001413; CY001421; CY001429; CY001437; CY001445; CY001453; CY001461; CY001469; CY001477; CY001485; CY001493; CY001504; CY0001512; CY0001520; CY0001528; CY0001536; CY0001544; CY0001552; CY0001560; CY0001568; CY001576; CY001584; CY001592; CY001600; CY001608; CY001616; CY001624; CY001632; CY001640; CY001648; CY001656; CY001664; CY001672; CY001688; CY001696; CY001704; CY001712; CY001720; CY0001728; CY001736; CY001744; CY0001752; CY0001760; CY001768; CY001776; CY0001784; CY001792; CY001800; CY0001808; CY0001816; CY0001824; CY0001832; CY0001840; CY0001848; CY0001856; CY0001864; CY001872; CY001880; CY001888; CY001896; CY001904; CY001912; CY001920; CY001928; CY001936; CY001944; CY001960; CY001968; CY001976; CY001984; CY001992; CY002000; CY002008; CY002016; CY002024; CY002032; CY002040; CY002048; CY002056; CY002064; CY002072; CY002080; CY002088; CY002096; CY002104; CY002112; CY002120; CY002128; CY002136; CY002144; CY002160; CY002168; CY002176; CY002184; CY002192; CY002200; CY002208; CY002216; CY002224; CY002232; CY002240; CY002248; CY002256; CY002264; CY002272; CY002280; CY002288; CY002296; CY002304; CY002312; CY002328; CY002336; CY002344; CY002368; CY002376; CY002384; CY002408; CY002416; CY002424; CY002432; CY002440; CY002448; CY002456; CY002464; CY002472; CY002480; CY002488; CY002496; CY002504; CY002512; CY002520; CY002544; CY002552; CY002560; CY002576; CY002584; CY002592; CY002600; CY002608; CY002712; CY002720; CY002728; CY002736; CY002744; CY002752; CY002760; CY002768; CY002776; CY002784; CY002816; CY002904; CY002905; CY002906; CY002914; CY002922; CY002930; CY002938; CY002946; CY002954; CY002962; CY003032; CY003040; CY003048; CY003056; CY003064; CY003072; CY003080; CY003088; CY003096; CY003104; CY003112; CY003120; CY003123; CY003136; CY003144; CY003152; CY003160; CY003168; CY003176; CY003184; CY003192; CY003200; CY003208; CY003216; CY003224; CY003232; CY003240; CY003248; CY003256; CY003264; CY003272; CY003280; CY003336; CY003344; CY003352; CY003408; CY003416; CY003424; CY003432; CY003440; CY003448; CY003456; CY003488; CY003496; CY003504; CY003512; CY003520; CY003528; CY003536; CY003544; CY003552; CY003560; CY003568; CY003576; CY003584; CY003592; CY003600; CY003608; CY003616; CY003624; CY003632; CY003640; CY003648; CY003656; CY003664; CY003680; CY003712; CY003720; CY003728; CY003736; CY003744; CY003752; CY003777; CY003785; CY003793; CY003801; CY003809; CY003817; CY003825; CY004657; CY004662; CY004670; CY004692; CY004702; CY005915; CY005916; CY005917; CY005935; CY005936; CY005937; CY005938; CY005939; CY005940; CY005941; CY005942; CY005943; CY005977; CY006011; CY006012; CY006013; CY006014; CY006015; CY006016; CY006026; CY006031; CY006035; CY006038; CY006043; CY006044; CY006052; CY006060; CY006068; CY006076; CY006084; CY006092; CY006115; CY006123; CY006131; CY006139; CY006147; CY006155; CY006163; CY006179; CY006203; CY006211; CY006219; CY006227; CY006235; CY006243; CY006251; CY006259; CY006267; CY006275; CY006283; CY006291; CY006299; CY006307; CY006315; CY006323; CY006331; CY006339; CY006347; CY006371; CY006379; CY006435; CY006443; CY006451; CY006459; CY006467; CY006475; CY006483; CY006491; CY006499; CY006507; CY006515; CY006523; CY006531; CY006539; CY006547; CY006555; CY006563; CY006571; CY006579; CY006587; CY006595; CY006603; CY006611; CY006619; CY006627; CY006635; CY006659; CY006683; CY006691; CY006699; CY006707; CY006715; CY006723; CY006731; CY006739; CY006755; CY006763; CY006771; CY006787; CY006795; CY006803; CY006811; CY006819; CY006827; CY006835; CY006843; CY006851; CY006859; CY006883; CY006891; CY006899; CY006907; CY006923; CY006931; CY006939; CY006947; CY006955; CY006963; CY006971; CY006979; CY006987; CY006995; CY007003; CY007011; CY007019; CY007027; CY007035; CY007043; CY007051; CY007059; CY007067; CY007075; CY007083; CY007091; CY007099; CY007107; CY007115; CY007123; CY007131; CY007139; CY007147; CY007155; CY007163; CY007171; CY007179; CY007187; CY007195; CY007203; CY007211; CY007219; CY007227; CY007235; CY007243; CY007251; CY007259; CY007267; CY007275; CY007283; CY007291; CY007299; CY007307; CY007315; CY007323; CY007331; CY007339; CY007347; CY007355; CY007363; CY007371; CY007379; CY007387; CY007395; CY007403; CY007411; CY007419; CY007427; CY007435; CY007443; CY007451; CY007459; CY007475; CY007483; CY007491; CY007499; CY007507; CY007515; CY007523; CY007531; CY007539; CY007547; CY007555; CY007563; CY007571; CY007579; CY007587; CY007595; CY007603; CY007611; CY007619; CY007627; CY007635; CY007643; CY007651; CY007659; CY007667; CY007675; CY007683; CY007691; CY007699; CY007707; CY007715; CY007723; CY007731; CY007739; CY007747; CY007755; CY007763; CY007771; CY007779; CY007787; CY007795; CY007803; CY007811; CY007819; CY007827; CY007835; CY007843; CY007851; CY007859; CY007867; CY007875; CY007883; CY007891; CY007899; CY007907; CY007915; CY007923; CY007931; CY007939; CY007947; CY007955; CY007963; CY007971; CY007979; CY007987; CY007995; CY008003; CY008011; CY008019; CY008027; CY008035; CY008043; CY008051; CY008059; CY008067; CY008075; CY008083; CY008091; CY008099; CY008107; CY008115; CY008123; CY008131; CY008139; CY008156; CY008164; CY008172; CY008180; CY008196; CY008204; CY008212; CY008220; CY008228; CY008236; CY008244; CY008252; CY008260; CY008268; CY008276; CY008284; CY008292; CY008300; CY008308; CY008316; CY008324; CY008332; CY008340; CY008348; CY008356; CY008364; CY008372; CY008380; CY008388; CY008396; CY008404; CY008412; CY008420; CY008428; CY008436; CY008444; CY008452; CY008460; CY008468; CY008476; CY008484; CY008492; CY008500; CY008508; CY008516; CY008532; CY008540; CY008548; CY008556; CY008564; CY008572; CY008580; CY008588; CY008596; CY008604; CY008612; CY008620; CY008628; CY008636; CY008644; CY008652; CY008660; CY008668; CY008676; CY008684; CY008692; CY008700; CY008708; CY008716; CY008724; CY008732; CY008740; CY008748; CY008756; CY008764; CY008772; CY008780; CY008788; CY008796; CY008804; CY008812; CY008820; CY008828; CY008836; CY008844; CY008852; CY008860; CY008868; CY008876; CY008884; CY008892; CY008900; CY008908; CY008916; CY008924; CY008932; CY008940; CY008948; CY008956; CY008964; CY008972; CY008980; CY009004; CY009012; CY009020; CY009028; CY009036; CY009044; CY009052; CY009060; CY009068; CY009076; CY009084; CY009092; CY009100; CY009108; CY009116; CY009124; CY009132; CY009140; CY009148; CY009156; CY009164; CY009244; CY009252; CY009260; CY009268; CY009300; CY009308; CY009348; CY009356; CY009372; CY009380; CY009388; CY009396; CY009404; CY009412; CY009420; CY009428; CY009436; CY009460; CY009468; CY009476; CY009484; CY009492; CY009500; CY009508; CY009516; CY009524; CY009556; CY009564; CY009572; CY009580; CY009588; CY009636; CY009644; CY009652; CY009660; CY009668; CY009676; CY009684; CY009692; CY009700; CY009708; CY009716; CY009724; CY009732; CY009740; CY009748; CY009852; CY009900; CY009908; CY009924; CY009932; CY009948; CY009988; CY009996; CY010004; CY010012; CY010020; CY010028; CY010036; CY010044; CY010052; CY010060; CY010068; CY010084; CY0110516; CY010548; CY010564; CY010588; CY010596; CY010604; CY010612; CY010620; CY010628; CY010636; CY010644; CY010652; CY010660; CY010668; CY010676; CY010684; CY010692; CY010700; CY010708; CY010716; CY010724; CY010732; CY010748; CY010756; CY010796; CY010812; CY010988; CY011020; CY011028; CY011048; CY011064; CY011120; CY011128; CY011136; CY011256; CY011264; CY011288; CY011320; CY011328; CY011336; CY011344; CY011352; CY011360; CY011368; CY011376; CY011384; CY011400; CY011416; CY011424; CY011432; CY011440; CY011448; CY01456; CY0111464; CY011472; CY011480; CY011488; CY011496; CY011504; CY011512; CY011520; CY011528; CY0111536; CY011544; CY011552; CY011560; CY011568; CY011576; CY011592; CY011616; CY011624; CY0111632; CY011640; CY011648; CY011656; CY011664; CY011672; CY011680; CY011688; CY011696; CY011704; CY011712; CY011720; CY011728; CY011736; CY011744; CY011752; CY011760; CY01768; CY0111808; CY011816; CY011824; CY011832; CY011840; CY011848; CY011856; CY011864; CY01872; CY0111880; CY011888; CY011896; CY011904; CY011912; CY011920; CY011928; CY011936; CY011944; CY011960; CY011968; CY011976; CY011984; CY011992; CY012000; CY012008; CY012016; CY012024; CY012032; CY012040; CY012048; CY012056; CY012064; CY012072; CY012080; CY012088; CY012096; CY012104; CY012112; CY012120; CY012128; CY012136; CY012144; CY012152; CY012160; CY012168; CY12176; CY012184; CY012200; CY012208; CY12216; CY12224; CY12232; CY12240; CY12248; CY12256; CY012264; CY012272; CY012280; CY012288; CY012312; CY012320; CY012328; CY012336; CY012344; CY012352; CY012360; CY012368; CY012376; CY012384; CY012392; CY012400; CY012408; CY012416; CY012424; CY012432; CY012448; CY012456; CY012464; CY012472; CY012480; CY012488; CY012496; CY012504; CY012512; CY012520; CY012528; CY012536; CY012544; CY012552; CY012560; CY12568; CY012576; CY012584; CY012592; CY012616; CY012624; CY012632; CY012640; CY012648; CY012656; CY012664; CY012672; CY012680; CY012688; CY012696; CY012704; CY012712; CY012720; CY012728; CY012736; CY012744; CY012752; CY012760; CY012768; CY012776; CY012784; CY012792; CY012848; CY012896; CY012904; CY012912; CY012920; CY012928; CY012936; CY012944; CY012952; CY012960; CY012968; CY012976; CY012984; CY012992; CY013000; CY013008; CY013016; CY013024; CY013048; CY013056; CY013064; CY013072; CY013080; CY013088; CY013096; CY013104; CY013112; CY013120; CY013128; CY013136; CY013144; CY13152; CY13160; CY13168; CY13176; CY13184; CY13192; CY013200; CY013208; CY013216; CY013224; CY013232; CY013240; CY013263; CY013311; CY013319; CY013327; CY013335; CY013343; CY013351; CY013359; CY013367; CY013375; CY013383; CY13389; CY013397; CY013405; CY013413; CY013421; CY013429; CY013437; CY013445; CY13453; CY013461; CY013469; CY013477; CY13485; CY13493; CY013501; CY13509; CY13517; CY13525; CY13533; CY013541; CY013549; CY013605; CY013613; CY013621; CY013629; CY013637; CY013645; CY13653; CY013661; CY013669; CY013677; CY013685; CY013693; CY013701; CY013709; CY013717; CY13725; CY013733; CY013741; CY013749; CY013757; CY013765; CY013773; CY013781; CY013789; CY013797; CY013805; CY013887; CY013895; CY013903; CY013911; CY013919; CY013927; CY013935; CY13943; CY013951; CY013959; CY013967; CY013975; CY013983; CY013991; CY013999; CY14015; CY14023; CY014031; CY014039; CY014047; CY014055; CY014063; CY014071; CY014079; CY014087; CY014095; CY014103; CY014111; CY14119; CY14127; CY14135; CY14143; CY014151; CY14159; CY14548; CY014571; CY014621; CY14633; CY14702; CY14865; CY014961; CY15492; CY15500; CY15508; CY015516; CY015540; CY015548; CY015556; CY015564; CY015572; CY015588; CY015596; CY015604; CY015612; CY015620; CY015628; CY015636; CY015644; CY015652; CY015660; CY015668; CY015676; CY015684; CY015692; CY015700; CY015708; CY015716; CY015724; CY015732; CY015740; CY015748; CY015756; CY015764; CY015772; CY015780; CY015788; CY015796; CY015804; CY015812; CY015820; CY015828; CY015836; CY015844; CY015852; CY015860; CY015868; CY015876; CY015884; CY015892; CY015900; CY015908; CY015916; CY015924; CY015932; CY015940; CY015948; CY015956; CY015964; CY015972; CY015980; CY015988; CY015996; CY016004; CY016012; CY016020; CY016028; CY016036; CY016044; CY016060; CY016068; CY016076; CY016084; CY016092; CY016100; CY016108; CY016116; CY016140; CY016180; CY016204; CY016212; CY016268; CY016395; CY016403; CY016411; CY016427; CY016443; CY016451; CY016467; CY016475; CY016483; CY016491; CY016499; CY016507; CY016515; CY016523; CY016531; CY016539; CY016547; CY016555; CY016571; CY016579; CY016587; CY016595; CY016603; CY016627; CY016635; CY016651; CY016659; CY016707; CY016715; CY016739; CY016747; CY016755; CY016763; CY016771; CY016979; CY016987; CY016995; CY017083; CY017091; CY017099; CY017107; CY017131; CY017163; CY017171; CY017259; CY017267; CY017283; CY017291; CY017299; CY017307; CY017323; CY017331; CY017339; CY017347; CY017355; CY017387; CY017395; CY017411; CY017443; CY017451; CY017459; CY017467; CY017475; CY017483; CY017491; CY017499; CY17507; CY017515; CY017523; CY017531; CY017539; CY017547; CY017555; CY017563; CY017571; CY17579; CY017587; CY017595; CY017603; CY017611; CY017619; CY017627; CY017709; CY017757; CY017773; CY017797; CY017805; CY017821; CY017837; CY017861; CY017885; CY017893; CY017901; CY17909; CY017917; CY017925; CY017933; CY017941; CY017949; CY017957; CY017965; CY017973; CY017981; CY017989; CY017999; CY18869; CY18925; CY018941; CY18957; CY185; CY18973; CY018981; CY018989; CY018997; CY019005; CY019013; CY019021; CY019029; CY019141; CY019149; CY019157; CY019165; CY019173; CY019181; CY019189; CY019197; CY019213; CY019245; CY019253; CY019261; CY019269; CY019277; CY019285; CY019293; CY019301; CY019309; CY019317; CY019325; CY019333; CY019747; CY019811; CY019819; CY019827; CY019835; CY019843; CY019851; CY19859; CY019891; CY019899; CY019907; CY019915; CY019931; CY019939; CY019981; CY019989; CY020005; CY020013; CY020021; CY020029; CY020037; CY020045; CY020053; CY020061; CY020069; CY020077; CY020085; CY020093; CY02101; CY020109; CY02117; CY020125; CY020133; CY020197; CY020205; CY020213; CY020221; CY020301; CY020309; CY020325; CY020333; CY020341; CY020357; CY020365; CY020493; CY020501; CY020525; CY020533; CY020717; CY020741; CY020757; CY020877; CY020893; CY020933; CY020997; CY021061; CY021077; CY021085; CY021093; CY021101; CY021109; CY021117; CY021157; CY021229; CY021261; CY021269; CY021277; CY021285; CY021309; CY021317; CY021341; CY021429; CY021453; CY021461; CY021597; CY021741; CY021765; CY021773; CY021781; CY021829; CY021837; CY021845; D00929; D00930; D00931; D00932; D10161; D10162; D13581; D13582; D13583; D13584; D21171; D21172; D21173; D21174; D21175; D21176; D21177; D21178; D21179; D21180; D21181; D21182; D21183; D30662; D30663; D30664; D30665; D30668; D30669; D30677; D30678; D30679; D30680; D30681; D30682; D30683; D30684; D30685; D30686; D43786; D43787; D43788; D43789; D43790; D43791; D43792; D49959; D49960; D49961; D49962; D49963; D49964; D49965; D49966; D49967; D86469; DQ006284; DQ006285; DQ007622; DQ021910; DQ021911; DQ059385; DQ066936; DQ086157; DQ086158; DQ086159; DQ086160; DQ086161; DQ089634; DQ089635; DQ089636; DQ089637; DQ089638; DQ089639; DQ114496; DQ114497; DQ114498; DQ114499; DQ114500; DQ114501; DQ114502; DQ114503; DQ114504; DQ114505; DQ114506; DQ114507; DQ114508; DQ114509; DQ114510; DQ114511; DQ114512; DQ114513; DQ114514; DQ114515; DQ114516; DQ114517; DQ114518; DQ114519; DQ114520; DQ114521; DQ114522; DQ114523; DQ114524; DQ114525; DQ114526; DQ114527; DQ114528; DQ114529; DQ114530; DQ114531; DQ114532; DQ114533; DQ114534; DQ114535; DQ114536; DQ114537; DQ124157; DQ124189; DQ124190; DQ124191; DQ124192; DQ124193; DQ124194; DQ124195; DQ124196; DQ132433; DQ141307; DQ145537; DQ146419; DQ150425; DQ150433; DQ159065; DQ159066; DQ159067; DQ167251; DQ167252; DQ167253; DQ167254; DQ167255; DQ167256; DQ167257; DQ167258; DQ167259; DQ167260; DQ167261; DQ167262; DQ167263; DQ167264; DQ167265; DQ167266; DQ167267; DQ167268; DQ167269; DQ167270; DQ167271; DQ167272; DQ167273; DQ167274; DQ167275; DQ167276; DQ167277; DQ167278; DQ167279; DQ167280; DQ167281; DQ167282; DQ167283; DQ167284; DQ167285; DQ167286; DQ167287; DQ167288; DQ167289; DQ167290; DQ167291; DQ167292; DQ167293; DQ167294; DQ167295; DQ167296; DQ167297; DQ167298; DQ167299; DQ167300; DQ167301; DQ167302; DQ167303; DQ167304; DQ167305; DQ167306; DQ167307; DQ174263; DQ174264; DQ174265; DQ174266; DQ174267; DQ174268; DQ179382; DQ179383; DQ179384; DQ179385; DQ179386; DQ179387; DQ179388; DQ179389; DQ179390; DQ179391; DQ179392; DQ179393; DQ179394; DQ179395; DQ179396; DQ179397; DQ179398; DQ179399; DQ179400; DQ179401; DQ179402; DQ179403; DQ179404; DQ179405; DQ179406; DQ179407; DQ179408; DQ179409; DQ179410; DQ179411; DQ179412; DQ179413; DQ179414; DQ179415; DQ179416; DQ179417; DQ179418; DQ179419; DQ179420; DQ179421; DQ179422; DQ179423; DQ179424; DQ179425; DQ179426; DQ179427; DQ179428; DQ179429; DQ179430; DQ179431; DQ179432; DQ179433; DQ179434; DQ179435; DQ179436; DQ179437; DQ179438; DQ179439; DQ179440; DQ179441; DQ179442; DQ179443; DQ179444; DQ179445; DQ179446; DQ179447; DQ179448; DQ179449; DQ179450; DQ179451; DQ179452; DQ179453; DQ179454; DQ179455; DQ179456; DQ179457; DQ179458; DQ179459; DQ179460; DQ179461; DQ179462; DQ179463; DQ179464; DQ179465; DQ179466; DQ179467; DQ179468; DQ179469; DQ179470; DQ179471; DQ179472; DQ179473; DQ179474; DQ179475; DQ179476; DQ179477; DQ179478; DQ179479; DQ179480; DQ179481; DQ179482; DQ179483; DQ179484; DQ179485; DQ179486; DQ179487; DQ179488; DQ179489; DQ179490; DQ179491; DQ179492; DQ179493; DQ179494; DQ179495; DQ179496; DQ179497; DQ179498; DQ179499; DQ179500; DQ179501; DQ179502; DQ179503; DQ179504; DQ179505; DQ179506; DQ179507; DQ179508; DQ179509; DQ179510; DQ179511; DQ179512; DQ179513; DQ179514; DQ179515; DQ179516; DQ179517; DQ179518; DQ179519; DQ179520; DQ179521; DQ179522; DQ179523; DQ179524; DQ179525; DQ179526; DQ179527; DQ222913; DQ227423; DQ227424; DQ227425; DQ227426; DQ227427; DQ227428; DQ227429; DQ227430; DQ227431; DQ241761; DQ241762; DQ241763; DQ249259; DQ249261; DQ249262; DQ256372; DQ256373; DQ256374; DQ256375; DQ265707; DQ265708; DQ265709; DQ265710; DQ265711; DQ265712; DQ265713; DQ265714; DQ265715; DQ265716; DQ265717; DQ265718; DQ335771; DQ336006; DQ336007; DQ336008; DQ336009; DQ336010; DQ336011; DQ336012; DQ336013; DQ336014; DQ336015; DQ336016; DQ336017; DQ415319; DQ415320; DQ415321; DQ415322; DQ415323; DQ415324; DQ415325; DQ415326; DQ447186; DQ469962; DQ469970; DQ469978; DQ469986; DQ469994; DQ470002; DQ487340; DQ487341; DQ508825; DQ508833; DQ508849; DQ508865; DQ508929; DQ534420; DQ534421; DQ534422; DQ534423; DQ534424; DQ534425; DQ534426; DQ534427; DQ534428; DQ534429; DQ632594; DQ632595; DQ632596; DQ632597; DQ865945; DQ865946; DQ865947; DQ865948; DQ865949; DQ865950; DQ865951; DQ865952; DQ865953; DQ865954; DQ865955; DQ865956; DQ865957; DQ865958; DQ865959; DQ865960; DQ865961; DQ865962; DQ865963; DQ865964; DQ865965; DQ865966; DQ865967; DQ865968; DQ865969; DQ865970; DQ865971; DQ865972; DQ865973; DQ865974; DQ883582; DQ883583; DQ883584; DQ883585; DQ883586; DQ883587; DQ883588; DQ883589; DQ883590; DQ883591; DQ883592; DQ883593; DQ883594; DQ883595; DQ883596; DQ883597; DQ883598; DQ883599; DQ883600; DQ883601; DQ883602; DQ883603; DQ883604; DQ883605; DQ883606; DQ883607; DQ883608; DQ883609; DQ883610; DQ883611; DQ883612; DQ883613; DQ883614; DQ883615; DQ883616; DQ883617; DQ883618; DQ883619; DQ883620; DQ883621; DQ883622; DQ883623; DQ883624; DQ883625; DQ883626; DQ883627; DQ883628; DQ923506; DQ923507; DQ973305; DQ975252; DQ975253; DQ975254; DQ975255; DQ975256; DQ975257; DQ975258; DQ975259; DQ975260; DQ975261; DQ975262; DQ975263; DQ975264; DQ975265; DQ975266; DQ975267; DQ981740; DQ981741; DQ981742; DQ983746; DQ983747; DQ983748; DQ983749; DQ983750; DQ983751; DQ983752; DQ983753; DQ983754; DQ983755; DQ983756; DQ983757; DQ983758; DQ983759; DQ983760; DQ983761; DQ983762; DQ983763; DQ983764; DQ983765; DQ983766; DQ983767; EF041487; EF117330; EF118172; EF118173; EF118174; EF151958; EF199897; EF199898; EF456782; EF456783; EF456784; EF456785; EF456786; EF456787; EF456788; EF456789; EF456790; EF456791; EF456792; EF456797; EF462544; EF462549; EF462550; EF462551; EF462552; EF462553; EF462554; EF462555; EF462557; EF462558; EF462559; EF462560; EF462561; EF462562; EF462566; EF462567; EF462568; EF462569; EF467799; EF467800; EF467827; EF473329; EF473330; EF473331; EF473332; EF473333; EF473334; EF473335; EF473336; EF473337; EF473338; EF473339; EF473340; EF473341; EF473342; EF473343; EF473344; EF473345; EF473346; EF473347; EF473348; EF473349; EF473350; EF473351; EF473352; EF473353; EF473354; EF473355; EF473356; EF473357; EF473358; EF473359; EF473360; EF473362; EF473363; EF473364; EF473365; EF473366; EF473367; EF473368; EF473369; EF473370; EF473371; EF473372; EF473373; EF473375; EF473376; EF473377; EF473378; EF473379; EF473380; EF473381; EF473382; EF473383; EF473384; EF473385; EF473386; EF473387; EF473388; EF473389; EF473390; EF473391; EF473392; EF473393; EF473394; EF473395; EF473396; EF473398; EF473399; EF473400; EF473401; EF473402; EF473403; EF473404; EF473405; EF473406; EF473408; EF473409; EF473410; EF473411; EF473412; EF473413; EF473414; EF473415; EF473416; EF473417; EF473418; EF473419; EF473420; EF473421; EF473422; EF473423; EF473424; EF473425; EF473426; EF473427; EF473428; EF473429; EF473430; EF473431; EF473432; EF473433; EF473434; EF473435; EF473436; EF473437; EF473438; EF473439; EF473440; EF473441; EF473442; EF473443; EF473444; EF473445; EF473446; EF473447; EF473449; EF473450; EF473451; EF473452; EF473453; EF473454; EF473455; EF473456; EF473457; EF473458; EF473459; EF473460; EF473461; EF473462; EF473463; EF473464; EF473465; EF473466; EF473467; EF473468; EF473469; EF473470; EF473471; EF473472; EF473473; EF473474; EF473475; EF473476; EF473477; EF473478; EF473479; EF473480; EF473481; EF473482; EF473483; EF473484; EF473485; EF473486; EF473487; EF473488; EF473489; EF473490; EF473491; EF473492; EF473493; EF473494; EF473495; EF473496; EF473497; EF473498; EF473499; EF473500; EF473504; EF473505; EF473506; EF473507; EF473508; EF473509; EF473510; EF473511; EF473512; EF473513; EF473514; EF473515; EF473516; EF473517; EF473518; EF473519; EF473520; EF473521; EF473522; EF473523; EF473524; EF473525; EF473526; EF473527; EF473528; EF473529; EF473530; EF473531; EF473532; EF473533; EF473534; EF473535; EF473536; EF473537; EF473538; EF473539; EF473540; EF473541; EF473542; EF473543; EF473544; EF473545; EF473546; EF473547; EF473548; EF473549; EF473550; EF473551; EF473552; EF473553; EF473555; EF473556; EF473557; EF473558; EF473559; EF473560; EF473561; EF473562; EF473563; EF473564; EF473565; EF473566; EF473567; EF473568; EF473569; EF473570; EF473571; EF473572; EF473573; EF473574; EF473575; EF473576; EF473577; EF473578; EF473579; EF473581; EF473582; EF473583; EF473584; EF473585; EF473586; EF473588; EF473589; EF473590; EF473591; EF473592; EF473593; EF473594; EF473595; EF473596; EF473597; EF473598; EF473599; EF473600; EF473601; EF473602; EF473603; EF473604; EF473605; EF473607; EF473608; EF473609; EF473611; EF473612; EF473613; EF473614; EF473615; EF473616; EF473617; EF473618; EF473619; EF473620; EF473621; EF473622; EF473623; EF473624; EF473625; EF473626; EF473627; EF473628; EF473629; EF473630; EF473632; EF473633; EF473634; EF473635; EF473636; EF473638; EF473639; EF473640; EF473641; EF473642; EF473643; EF473644; EF473645; EF473646; EF473647; EF473648; EF541428; EF541429; EF541430; EF541431; EF541432; EF541433; EF541434; EF541435; EF541436; EF541437; EF541438; EF541439; EF541440; EF541441; EF541442; EF541443; J02090; J02092; J02132; J02538; K03335; K03338; L18994; L18996; L18997; L18998; L19000; L9001; L19002; L19003; L19004; L19412; L19413; L19414; L19415; L19416; L20101; L20102; L20103; L20104; L20105; L20114; L20115; L20118; L20119; L27597; L31949; L32024; L39913; L39914; L39915; L39916; L39917; L39918; L75975; L75976; L75977; L75978; L75979; L75980; L75981; L75982; L75983; L75984; L75985; L75986; L75987; L75988; L75989; L75990; L75991; L76035; L76036; L76037; M16737; M16738; M16739; M16740; M16741; M16742; M16743; M19056; M19057; M21648; M24718; M24719; M24720; M24721; M24722; M24723; M24724; M24725; M24726; M24727; M24728; M25044; M25434; M29257; M54895; M55059; M57630; M57631; M57632; M57644; M65018; M73771; M73772; M73773; M73774; M73775; M73776; S64310; S77429; U07146; U08858; U08859; U08905; U26830; U48439; U48440; U48441; U48442; U48443; U48444; U48445; U48446; U48447; U49722; U58195; U65552; U65553; U65554; U65555; U65556; U65557; U65558; U65559; U65560; U77830; U77831; U77832; U77833; U77834; U77835; U77836; U77837; U77838; U77839; U77840; U97740; V01085; V01086; V01087; V01089; V01098; V01103; X05907; X68437; X73489; X73490; X73491; X75800; X85085; X85086; X85087; X85088; X85089; X85090; X95637; X95638; Y14053; Y14055; Y14056; Y14057; Y14058; Y14059; Y14060; Z46391; Z46392; Z46393; Z46394; Z46395; Z46396; Z46397; Z46398; Z46399; Z46400; Z46401; Z46402; Z46403; Z46404; Z46405; Z46406; Z46407; Z46408; Z46409; Z46410; Z46411; Z46412; Z46413; Z46414; Z46415; Z46416 and Z46417.

Polynucleotides encoding influenza virus subtype H4 hemagglutinins suitable for inclusion as a cargo moiety in conjugates herein have the following accession numbers and are available to the public via the National Center for Biotechnology Information (NCBI) Entrez nucleotide search and retrieval system:

AB288842; AB289331; CY005952; CY005953; CY005954; CY005955; CY005956; CY005957; CY005958; CY005959; CY005961; CY005962; CY005963; CY005964; CY005965; CY005966; CY005967; CY005968; CY006017; CY006027; CY006030; CY011036; CY011056; CY012808; CY012816; CY013248; CY014562; CY014579; CY014630; CY014723; CY014751; CY014857; CY014922; CY014929; CY014937; CY015459; CY015467; CY016148; CY017701; CY017741; CY020725; CY020733; CY020749; CY020765; CY020773; CY020789; CY020797; CY020805; CY020981; CY021213; CY021221; CY021237; CY021325; CY021333; CY021349; D90302; DQ021848; DQ021849; DQ021850; DQ021851; DQ021852; DQ021853; DQ021854; DQ021855; DQ021856; DQ021857; DQ021858; DQ021859; DQ021860; DQ021861; DQ021862; DQ021863; DQ021864; DQ021865; DQ021866; DQ021867; DQ021868; DQ236166; DQ327834; DQ787806; EF041495; J02102; M25283; M25284; M25285; M25286; M25287; M25288; M25289; M25290; M25291; AB289333; AB292406; AB292408; AB292662; AB295609; AB295611; AF285883; AF285885; AF290436; AJ506780; AJ506782; AY180434; AY180435; AY180436; AY180437; AY180438; AY180439; AY180440; AY180441; AY180442; AY180443; AY596802; AY596803; AY596804; AY633124; AY633141; AY633156; AY633260; AY633268; AY633284; AY633348; AY633356; CY004847; CY004911; CY004925; CY004933; CY004939; CY005672; CY005679; CY005944; CY005945; CY005946; CY005947; CY005948; CY005950 and CY005951.

Polynucleotides encoding influenza virus subtype H5 hemagglutinins suitable for inclusion as a cargo moiety in conjugates herein have the following accession numbers and are available to the public via the National Center for Biotechnology Information (NCBI) Entrez nucleotide search and retrieval system:

BAD89305; BAD89315; BAD89325; BAD89335; BAD89345; BAE07201; BAE07155; BAE47131; BAE48315; BAE48316; BAE48317; BAE48318; BAE46949; BAE48684; BAE48685; BAE48686; BAE48687; BAE48688; BAE48689; BAE48690; BAE48691; BAE48692; BAE48693; BAE48694; BAE48695; BAE48696; BAE94699; BAF37962; BAE96567; BAE96961; BAF49662; BAF49663; BAF49664; BAF49665; BAF49666; BAF49667; BAF49668; BAF49669; BAF49670; BAF49671; BAF49672; BAF49673; BAF49674; BAF49675; BAF49676; BAF37387; BAF48359; AAC40508; AAC34263; AAC32078; AAC32088; AAC32098; AAC32099; AAC32100; AAC32101; AAC32102; AAC14418; AAD13566; AAD13567; AAD13568; AAD13569; AAD13570; AAD13571; AAD13572; AAD13573; AAD13574; AAD13575; AAF74329; AAF74330; AAF74331; AAD52043; AAF02300; AAF02301; AAF02302; AAF02303; AAF02304; AAF02305; AAF02306; AAF02307; AAF02308; AAF02309; AAF04719; AAF04720; AAD21153; AAD21154; AAD21155; AAD21156; AAD21157; AAD21158; AAD21159; AAD21160; AAD21161; AAD21162; AAD21163; AAD21164; AAD51927; AAD37782; AAF89536; AAF89537; AAF89538; AAF89539; AAF89540; AAF89541; AAF89542; AAF89543; AAF89544; AAF89545; AAF89546; AAG28424; AAG60347; AAG60348; AAG60349; AAG01195; AAG01205; AAG01215; AAG01225; AAF99718; AAG38534; AAK38298; AAK57506; AAL59142; AAL59143; AAL16033; AAL84323; AAL84324; AAM49555; AAM22457; AAM22458; AAO52859; AAO52860; AAO52861; AAO52862; AAO52863; AAO52864; AAO52865; AAO52866; AAO52867; AAO52868; AAO52869; AAO52870; AAO52871; AAO52872; AAO52873; AAO52874; AAO52875; AAO52876; AAO52877; AAO52878; AAO52879; AAO52880; AAO52881; AAO52882; CAC28131; CAF21870; CAF21874; CAG14996; CAG14997; CAG29661; CAI29278; CAI96162; CAI96163; CAI99404; CAJ32556; CAJ75440; CAJ75441; CAJ75442; CAJ75443; CAJ75444; CAJ75445; CAJ75446; CAJ75447; CAJ75448; CAJ77761; CAJ84721; CAK18565; CAK18566; CAK18567; CAK18570; CAK18571; CAK18577; CAK18596; CAL37103; CAL51387; CAL51388; CAL51389; CAL51390; CAL51391; CAL51392; CAL51393; CAL51394; CAL51395; CAL51396; CAL48277; CAL48276; CAL48275; CAL48274; CAL48273; CAL48272; CAL48271; CAL48270; CAL48269; CAL48268; CAL48267; CAL48279; CAL48278; CAL48266; CAL48265; CAL48280; CAL59784; CAL59783; CAL59782; CAL59781; CAL59780; CAL59779; CAL59786; CAL59785; CAM33521; AAL31380; AAL31381; AAL31382; AAL31383; AAL31384; AAL31385; AAL31386; AAL31387; AAL31388; AAL75839; AAL75843; AAL75847; AAO46797; AAO46798; AAO46799; AAO46800; AAO46801; AAO46802; AAO46803; AAO46804; AAO46805; AAP71989; AAP71990; AAP71991; AAP71992; AAP71993; AAP71994; AAP71995; AAP71996; AAP71997; AAP71998; AAP71999; AAP72000; AAP72001; AAP72002; AAP72003; AAP72004; AAP72005; AAP72006; AAP72007; AAP72008; AAP72009; AAP72010; AAP72011; AAR88808; AAR88809; AAR88810; AAR88811; AAR88812; AAR88813; AAR88814; AAR88815; AAR88816; AAR88817; AAR88818; AAR88819; AAR88820; AAR88821; AAR88822; AAR88823; AAR88824; AAR88825; AAR88826; AAR88827; AAR88828; AAR88829; AAR88830; AAR88831; AAR88832; AAR88833; AAR88834; AAR88835; AAR88836; AAR88837; AAR88838; AAR88839; AAR88840; AAR88841; AAS07023; AAR99628; AAR98819; AAT07996; AAS50166; AAS50167; AAS57873; AAS57874; AAS57875; AAS57876; AAS45134; AAS84275; AAS84276; AAS84247; AAS84248; AAS84249; AAS84250; AAS84251; AAS84252; AAS84253; AAS84254; AAS84255; AAS84256; AAS84257; AAS84258; AAS84259; AAS84260; AAS84261; AAS84262; AAS84263; AAS84264; AAS84265; AAS84266; AAS84267; AAS84268; AAS84269; AAS84270; AAS84271; AAS84272; AAS84273; AAS84274; AAS65615; AAS65618; AAS87596; AAS87577; AAS87580; AAT39065; AAT39066; AAT39067; AAT39068; AAT39073; AAT39074; AAT39075; AAT39076; AAT39077; AAT39078; AAT39079; AAT39080; AAS79356; AAS79359; AAS89004; AAT12022; AAT12023; AAT12024; AAT12025; AAT12026; AAT12027; AAT12028; AAT12029; AAT12030; AAT12031; AAT12032; AAT12033; AAT12034; AAT12035; AAT12036; AAT12037; AAT12038; AAT12039; AAT12040; AAT12041; AAT12042; AAS89267; AAS89268; AAS89269; AAS89270; AAS89271; AAS89272; AAS89273; AAT37563; AAT90337; AAV34704; AAV32636; AAT65209; AAT70210; AAT70218; AAT72505; AAV65826; AAT73260; AAT73261; AAT73262; AAT73263; AAT73264; AAT73265; AAT73266; AAT73267; AAT73268; AAT73269; AAT73270; AAT73271; AAT73272; AAT73273; AAT73274; AAT73275; AAT73276; AAT73277; AAT73278; AAT73279; AAT73280; AAT73281; AAT73282; AAT73283; AAT73284; AAT73285; AAT73286; AAT73287; AAT73288; AAT73289; AAT73290; AAT73291; AAT73292; AAT73293; AAT73294; AAT73295; AAT73296; AAT73297; AAT73298; AAT73299; AAT73300; AAT73301; AAT73302; AAT73303; AAT73304; AAT73305; AAT73306; AAT73307; AAT73308; AAT73309; AAT73310; AAT73311; AAT73312; AAT73313; AAT76166; AAV97601; AAV97602; AAV97603; AAV97604; AAT84153; AAT90832; AAV91220; AAV73972; AAV73975; AAV73980; AAW59548; AAW59550; AAW59552; AAW59554; AAW59556; AAW59558; AAW59559; AAU08349; AAU08351; AAW59390; AAW59398; AAW59408; AAW19638; AAW19640; AAW19642; AAW19644; AAW19646; AAV30828; AAV30836; AAV48546; AAV41002; AAV48778; AAV48780; AAV74400; AAW80717; AAW80718; AAW80719; AAV91149; AAV97886; AAW30657; AAX47288; AAW72226; AAX59694; AAW66002; AAX53504; AAX53505; AAX53506; AAX53507; AAX53508; AAX53509; AAX53510; AAX83395; AAX83396; AAX83397; AAX83398; AAY57183; AAY57184; AAY57185; AAY57186; AAY57187; AAY57188; AAY57189; AAY57190; AAY57191; AAY57192; AAY57193; AAY57194; AAY57195; AAY57196; AAY57197; AAY57198; ABB20262; ABB87042; ABB87281; ABB87292; ABB87711; ABB88278; ABB88348; ABB88379; ABG88245; ABI36041; ABI36012; ABI36023; ABI36034; ABI36040; ABI36042; ABI36043; ABI36044; ABI36045; ABI36046; ABI36047; ABI36048; ABI36049; ABI36050; ABI36051; ABI36052; ABI36053; ABI36054; ABI36055; ABI36056; ABI36057; ABI36144; ABI36155; ABI36166; ABI36177; ABI36187; ABI36198; ABI36275; ABI36286; ABI36295; ABI36307; ABI36318; ABI36329; ABI36340; ABI36351; ABI36362; ABI36373; ABI36384; ABI36395; ABI36406; ABI36423; ABI36428; ABI36439; ABI36450; ABI36469; ABI36480; ABI49396; ABI49407; ABI49415; ABI84424; ABI84465; ABI84495; ABI84497; ABI84598; ABI84603; ABI84608; ABI84784; ABI84816; ABI84970; ABI85095; ABI85106; ABI85117; ABI85155; ABI95316; ABI95327; ABI95338; ABI95349; ABJ16565; ABJ16796; ABJ16807; ABJ16818; ABJ16928; ABJ16917; ABJ16829; ABJ16939; ABJ51728; ABJ51717; ABJ51706; ABJ51695; ABJ51739; ABJ51684; ABJ51673; ABJ16950; ABJ16840; ABJ16851; ABJ16862; ABJ16873; ABJ16884; ABJ16895; ABJ16906; ABJ53526; ABJ53537; ABJ53548; ABJ53594; ABJ53583; ABJ53559; ABK40087; ABK40492; ABK80003; ABL07008; ABL07019; ABL07030; ABL31744; ABL31755; ABL31766; ABL31780; ABM22048; ABM90434; ABM90445; ABM90456; ABM90467; ABM90478; ABM90489; ABM90500; ABM90511; ABM90522; ABM90533; ABM90544; ABO37977; ABO38263; ABO44200; ABO44211; ABO44222; ABO44233; ABO44244; ABO44255; ABO44266; ABO44277; ABO44288; ABO44299; ABO44310; ABO52720; ABO52731; ABO52742; ABO52753; ABO77034; ABO77045; AAY21163; AAY25499; AAY46328; AAY46329; AAY46330; AAY46331; AAY46332; AAY46333; AAY46334; AAY46335; AAY46336; AAY46337; AAY46338; AAY46339; AAY46340; AAY46341; AAY46342; AAY46343; AAY46344; AAY46345; AAY46346; AAY46347; AAY46348; AAY46349; AAY46350; AAY46351; AAY46352; AAY46353; AAY46354; AAY46355; AAY46356; AAY46357; AAY46358; AAY46359; AAY46360; AAY46361; AAY46362; AAY46363; AAY46364; AAY46365; AAY46366; AAY56367; AAY68363; AAY78953; AAZ29946; AAZ29947; AAZ29948; AAZ29949; AAZ29950; AAZ29951; AAZ29952; AAZ29953; AAZ29954; AAZ29955; AAZ29956; AAZ29957; AAZ29958; AAZ29959; AAZ29960; AAZ29961; AAZ29962; AAZ29963; AAZ29964; AAZ29965; AAZ29966; AAZ29967; AAZ29968; AAZ29969; AAZ29970; AAZ29971; AAZ29972; AAZ29973; AAZ29974; AAZ29975; AAZ29976; AAZ29977; AAZ29978; AAZ29979; AAZ29980; AAZ29981; AAZ76389; ABE68921; ABE68922; AAZ16275; ABE68923; ABE68924; ABE68925; ABE68926; AAZ16276; AAZ16277; ABE68927; AAZ16278; AAZ16279; ABE68928; ABE68929; AAZ16280; ABE68930; ABE68931; ABE68932; AAZ16281; AAZ16282; AAZ72734; AAZ72735; AAZ72736; AAZ72737; AAZ72738; AAZ72739; AAZ17522; AAZ17523; AAZ17524; AAZ23154; AAZ80486; AAZ78315; ABA29447; AAZ82496; AAZ82497; ABA70758; ABB00917; ABB00918; ABB00919; ABB00920; ABA39516; ABA39517; ABA39518; ABA39519; ABA39520; ABA87102; ABA87103; ABA54915; ABA55714; ABA55715; ABA55716; ABA55717; ABB00582; ABB43058; ABB43059; ABB22773; ABB22774; ABB22775; ABB43119; ABB43127; ABB83598; ABB58817; ABB58818; ABB58819; ABB58820; ABB58821; ABB80546; ABB86287; ABC47656; ABC59833; ABC66517; ABC66518; ABC66519; ABC66520; ABC66521; ABC66522; ABC66523; ABC66524; ABC66525; ABC66526; ABC66527; ABC66528; ABC66529; ABC66530; ABC66531; ABC66532; ABC66533; ABC66534; ABC66535; ABC66536; ABC66537; ABC66538; ABC66539; ABC66540; ABC66541; ABC66542; ABC66543; ABC66544; ABC66545; ABC66546; ABC66547; ABC66548; ABC66549; ABC66550; ABC66551; ABC66552; ABC66553; ABC66554; ABC66555; ABC66556; ABC66557; ABC66558; ABC66559; ABC66560; ABC66561; ABC66562; ABC66563; ABC66564; ABC66565; ABC66566; ABC66567; ABC66568; ABC66569; ABC66570; ABC66571; ABC66572; ABC66573; ABC66574; ABC66575; ABC66576; ABC66577; ABC66578; ABC66579; ABC66580; ABC66581; ABC66582; ABC48787; ABC69216; ABC69224; ABC69232; ABC70167; ABC69148; ABC69149; ABC69150; ABC70712; ABC72082; ABC87315; ABC72655; ABD32123; ABD32128; ABC88573; ABC88583; ABD14806; ABD14807; ABD14808; ABD14809; ABD14810; ABD28180; ABD28181; ABD28182; ABD16284; ABD46889; ABD49489; ABD60336; ABD60345; ABD46740; ABD73284; ABD52284; ABD65415; ABD66291; ABD66292; ABD66293; ABD73804; ABD85144; ABD83818; ABD92945; ABD92953; ABD85374; ABD95991; ABE26829; ABE01046; ABE97547; ABE97548; ABE97549; ABE97550; ABE97551; ABE97552; ABE97553; ABE97554; ABE97555; ABE97556; ABE97557; ABE97558; ABE97559; ABE97560; ABE97561; ABE97562; ABE97563; ABE97564; ABE97565; ABE97566; ABE97567; ABE97568; ABE97569; ABE97570; ABE97571; ABE97572; ABE97573; ABE97574; ABE97575; ABE97576; ABE97577; ABE97578; ABE97579; ABE97580; ABE97581; ABE97582; ABE97583; ABE97584; ABE97585; ABE97586; ABE97587; ABE97588; ABE97589; ABE97590; ABE97591; ABE97592; ABE97593; ABE97594; ABE97595; ABE97596; ABE97597; ABE97598; ABE97599; ABE97600; ABE97601; ABE97602; ABE97603; ABE97604; ABE97605; ABE97606; ABE97607; ABE97608; ABE97609; ABE97610; ABE97611; ABE97612; ABE97613; ABE97614; ABE97615; ABE97616; ABE97617; ABE97618; ABE97619; ABE97620; ABE97621; ABE97622; ABE97623; ABE97624; ABE97625; ABE97626; ABE97627; ABE97628; ABE97629; ABE97630; ABE97631; ABE97632; ABE97633; ABE97634; ABF56528; ABF58847; ABF56648; ABG23657; ABF61761; ABG20463; ABG20464; ABG20465; ABG20466; ABG20467; ABG38185; ABG38189; ABF72802; ABF93440; ABF93441; ABG49439; ABF84066; ABG45944; ABG75543; ABG20468; ABG20472; ABG20476; ABG20478; ABG35546; ABG65732; ABI16504; ABG65733; ABG67711; ABG67712; ABG67713; ABG67714; ABG57086; ABG57087; ABG57094; ABG57095; ABG78549; ABG78567; ABI34140; ABI34142; ABG67978; ABG75831; ABG75616; ABI23979; ABG81037; ABG81038; ABG81039; ABG81040; ABG81041; ABI18096; ABH85395; ABH09484; ABH09485; ABH09486; ABH09487; ABH09488; ABH09489; ABH09490; ABJ98523; ABJ98525; ABJ98527; ABJ98529; ABJ98531; ABI34124; ABK34764; ABJ88847; ABJ96647; ABJ96648; ABJ96649; ABJ96650; ABJ96651; ABJ96652; ABJ96653; ABJ96654; ABJ96655; ABJ96656; ABJ96657; ABJ96658; ABJ96659; ABJ96660; ABJ96661; ABJ96662; ABJ96663; ABJ96664; ABJ96665; ABJ96666; ABJ96667; ABJ96668; ABJ96669; ABJ96670; ABJ96671; ABJ96672; ABJ96673; ABJ96674; ABJ96675; ABJ96676; ABJ96677; ABJ96678; ABJ96679; ABJ96680; ABJ96681; ABJ96682; ABJ96683; ABJ96684; ABJ96685; ABJ96686; ABJ96687; ABJ96688; ABJ96689; ABJ96690; ABJ96691; ABJ96692; ABJ96693; ABJ96694; ABJ96695; ABJ96696; ABJ96697; ABJ96698; ABJ96699; ABJ96700; ABJ96701; ABJ96702; ABJ96703; ABJ96704; ABJ96705; ABJ96706; ABJ96707; ABJ96708; ABJ96709; ABJ96710; ABJ96711; ABJ96712; ABJ96713; ABJ96714; ABJ96715; ABJ96716; ABJ96717; ABJ96718; ABJ96719; ABJ96720; ABJ96721; ABJ96722; ABJ96723; ABJ96724; ABJ96725; ABJ96726; ABJ96727; ABJ96728; ABJ96729; ABJ96730; ABJ96731; ABJ96732; ABJ96733; ABJ96734; ABJ96735; ABJ96736; ABJ96737; ABJ96738; ABJ96739; ABJ96740; ABJ96741; ABJ96742; ABJ96743; ABJ96744; ABJ96745; ABJ96746; ABJ96747; ABJ96748; ABJ96749; ABJ96750; ABJ96751; ABJ96752; ABJ96753; ABJ96754; ABJ96755; ABJ96756; ABJ96757; ABJ96758; ABJ96759; ABJ96760; ABJ96761; ABJ96762; ABJ96763; ABJ96764; ABJ96765; ABJ96766; ABJ96767; ABJ96768; ABJ96769; ABJ96770; ABJ96771; ABJ96772; ABJ96773; ABJ96774; ABJ96775; ABJ96776; ABJ96777; ABJ96778; ABJ96779; ABJ96780; ABJ96781; ABJ96782; ABJ96783; ABJ96784; ABJ96785; ABJ96786; ABJ96787; ABJ96788; ABJ96789; ABJ96790; ABJ96791; ABJ96792; ABJ96793; ABJ96794; ABJ96795; ABJ96796; ABJ96797; ABJ96798; ABJ96799; ABJ96800; ABJ96801; ABJ96802; ABJ96803; ABJ96804; ABJ96805; ABJ96806; ABJ96807; ABJ96808; ABJ96809; ABJ96810; ABJ96811; ABJ96812; ABJ96813; ABJ96814; ABJ96815; ABJ96816; ABJ96817; ABJ96818; ABJ96819; ABJ96820; ABJ96821; ABJ96822; ABJ96823; ABJ96824; ABJ96825; ABJ96826; ABJ96827; ABJ96828; ABJ96829; ABJ96830; ABJ96831; ABJ96832; ABJ96833; ABJ96834; ABJ96835; ABJ96836; ABJ96837; ABJ96838; ABJ96839; ABJ96840; ABJ96841; ABJ96842; ABJ96843; ABJ96844; ABJ96845; ABJ96846; ABJ96847; ABJ96848; ABJ96849; ABJ96850; ABJ96851; ABJ96852; ABJ96853; ABJ96854; ABJ96855; ABJ96856; ABJ96857; ABJ96858; ABJ96859; ABJ96860; ABJ96861; ABJ96862; ABJ96863; ABJ96864; ABJ96865; ABJ96866; ABJ96867; ABJ96868; ABJ96869; ABJ96870; ABJ96871; ABJ96872; ABJ96873; ABJ96874; ABJ96875; ABJ96876; ABJ96877; ABJ96878; ABJ96879; ABJ96880; ABJ96881; ABJ96882; ABJ96883; ABJ96884; ABJ96885; ABJ96886; ABJ96887; ABJ96888; ABJ96889; ABJ96890; ABJ96891; ABJ96892; ABJ96893; ABJ96894; ABJ96895; ABJ96896; ABJ96897; ABJ96898; ABJ96899; ABJ96900; ABJ96901; ABJ96902; ABJ96903; ABJ96904; ABJ96905; ABJ96906; ABJ96907; ABJ96908; ABJ96909; ABJ96910; ABJ96911; ABJ96912; ABJ96913; ABJ96914; ABJ96915; ABJ96916; ABJ96917; ABJ96918; ABJ96919; ABJ96920; ABJ96921; ABJ96922; ABJ96923; ABJ96924; ABJ96925; ABJ96926; ABJ96927; ABJ96928; ABJ96929; ABJ96930; ABJ96931; ABJ96932; ABJ96933; ABJ96934; ABJ96935; ABJ96936; ABJ96937; ABJ96938; ABJ96939; ABJ96940; ABJ96941; ABJ96942; ABJ96943; ABJ96944; ABJ96945; ABJ96946; ABJ96947; ABJ96948; ABJ96949; ABJ96950; ABJ96951; ABJ96952; ABJ96953; ABJ96954; ABJ96955; ABJ96956; ABJ96957; ABJ96958; ABJ96959; ABJ96960; ABJ96961; ABJ96962; ABJ96963; ABJ96964; ABJ96965; ABJ96966; ABJ96967; ABJ96968; ABJ96969; ABJ96970; ABJ96971; ABJ96972; ABJ96973; ABJ96974; ABJ96975; ABJ96976; ABJ96977; ABJ96978; ABJ96979; ABJ96980; ABJ96981; ABJ96982; ABJ96983; ABJ96984; ABJ96985; ABJ96986; ABJ96987; ABJ96988; ABJ96989; ABJ96990; ABJ96991; ABJ96992; ABJ96993; ABJ96994; ABJ96995; ABJ96996; ABJ96997; ABJ96998; ABJ96999; ABJ97000; ABJ97001; ABJ97002; ABJ97003; ABJ97004; ABJ97005; ABJ97006; ABJ97007; ABJ97008; ABJ97009; ABJ97010; ABJ9711; ABJ97012; ABJ97013; ABJ97014; ABJ97015; ABJ97016; ABJ97017; ABJ97018; ABJ97019; ABJ97020; ABJ97021; ABJ97022; ABJ97023; ABJ97024; ABJ97025; ABJ97026; ABJ97027; ABJ97028; ABJ97029; ABJ97030; ABJ97031; ABJ97032; ABJ97033; ABJ97034; ABJ97035; ABJ97036; ABJ97037; ABJ97038; ABJ97039; ABJ97040; ABJ97041; ABJ97042; ABJ97043; ABJ97044; ABJ97045; ABJ97046; ABJ97047; ABJ97048; ABJ97049; ABJ97050; ABK000133; ABI94741; ABI94747; ABI94754; ABI94764; ABI96729; ABI96730; ABI96741; ABJ09476; ABI96767; ABJ09545; ABI96701; ABJ16473; ABJ15720; ABI98911; ABJ09528; ABI98919; ABI97335; ABJ52562; ABJ80592; ABK00083; ABK00087; ABK00096; ABI98929; ABK000132; ABI97303; ABJ09511; ABJ09498; ABJ09466; ABJ09518; ABJ09488; ABK000104; ABI98938; ABK13783; ABK13784; ABK13782; ABJ53148; ABK32775; ABK32776; ABK32777; ABK32778; ABK32779; ABK32780; ABK32781; ABK32782; ABK34511; ABK34512; ABK34513; ABJ90343; ABK79301; ABK79302; ABK79303; ABK79304; ABL10088; ABL74499; ABL74500; ABL75919; ABL63754; ABL63755; ABL63756; ABL63757; ABL63758; ABL63759; ABL63760; ABL63761; ABL63762; ABL63763; ABL63764; ABL63765; ABL63766; ABL63767; ABL63768; ABL63769; ABL63770; ABL63771; ABL63772; ABM54179; ABM54180; ABO76638; ABO76639; ABO76640; ABO76641; ABO76642; ABO76643; ABO76644; ABM92273; ABN54791; ABN54792; ABO14789; ABO14790; ABO30505; ABN70706; ABN70707; ABN70708; ABN70709; ABN70710; ABN70711; ABO13912; ABO13920; ABO38179; ABO20946; ABO10162; ABO10163; ABO10181; ABO10183; ABO10184; ABO10185; ABO10186; ABO10187; ABO20962; ABO64687; ABO64688; ABO64689; ABO64690; ABO64691; ABO64692; ABO64693; ABO64694; ABO64695; ABO64696; ABO64697; ABO30353; ABO30354; ABO30355; ABO30359; ABO30360; ABO30361; ABO30346; ABO30347; ABO31434; AAA43199; AAA43094; AAL34297; AAL34298; AAL34299; AAA43159; AAA43160; AAA43082; AAA43083; AAA43205; AAB29507; AAB82064; AAA74909; AAA74910; AAC54378; AAC54390; AAC54391; AAC54392; AAC54393; AAB49654; AAB49655; AAB19072; AAB19073; AAB19074; AAB19075; AAB19076; AAB19077; AAB19078; AAB19079; AAB19080; AAB19081; AAB19082; AAB19083; AAB19084; AAB19085; AAB19086; AAB19087; AAB19088; AAB19089; AAC58999; AAB39639; AAC58990; AAC58991; AAC58992; AAC58993; AAC58994; AAC58995; AAC58996; AAC58997; AAC58998; CAA30680 and CAA30719.

Polynucleotides encoding influenza virus subtype H6 hemagglutinins suitable for inclusion as a cargo moiety in conjugates herein have the following accession numbers and are available to the public via the National Center for Biotechnology Information (NCBI) Entrez nucleotide search and retrieval system:

AB278600; AB286875; AJ410532; AJ410533; AJ410534; AJ410535; AJ410536; AJ410537; AJ410538; AJ410539; AJ410540; AJ410541; AJ410542; AJ410543; AJ410544; AJ410545; AJ410546; AJ410547; AJ427308; AJ507203; AJ507204; AJ507205; AJ507206; AJ507207; AJ507208; AJ507209; AJ697867; AJ697868; AJ697869; AJ697870; AJ697871; AY633188; AY633204; AY633220; AY633236; AY633300; AY633308; AY633316; AY633324; AY633332; AY633380; AY684892; AY703832; AY773907; AY862613; AY968676; CY004034; CY004035; CY004036; CY004037; CY004038; CY004039; CY004043; CY004054; CY004066; CY004072; CY004076; CY004080; CY004086; CY004094; CY004114; CY004129; CY004137; CY004142; CY004146; CY004154; CY004162; CY004170; CY004178; CY004186; CY004194; CY004202; CY004210; CY004218; CY004226; CY004234; CY004242; CY004250; CY004258; CY004266; CY004274; CY004282; CY004515; CY004523; CY005106; CY005597; CY005605; CY005691; CY005881; CY01112; CY012832; CY013255; CY013863; CY014561; CY014607; CY014616; CY014623; CY014656; CY014764; CY014880; CY014888; CY014909; CY014945; CY014953; CY015127; CY015451; CY015476; CY015484; CY016124; CY016132; CY016156; CY016164; CY016172; CY016619; CY017789; CY018007; CY018893; CY018909; CY018917; CY020781; CY020813; CY020821; CY020829; CY020837; CY020845; CY020853; CY020869; CY020957; CY020973; CY020989; CY021197; CY021205; CY021477; CY021677; D90303; DQ021649; DQ021650; DQ021651; DQ021652; DQ021653; DQ021654; DQ021655; DQ021656; DQ021657; DQ021658; DQ021659; DQ021660; DQ021661; DQ021662; DQ021663; DQ021664; DQ021665; DQ021666; DQ021667; DQ021668; DQ021669; DQ021670; DQ021671; DQ021672; DQ021673; DQ021675; DQ021676; DQ021677; DQ021678; DQ021679; DQ021680; DQ021681; DQ021682; DQ021683; DQ021684; DQ285546; DQ376618; DQ376619; DQ376620; DQ376621; DQ376622; DQ376623; DQ376624; DQ376625; DQ376626; DQ376627; DQ376628; DQ376629; DQ376630; DQ376631; DQ376632; DQ376633; DQ376634; DQ376635; DQ376636; DQ376637; DQ376638; DQ376639; DQ376640; DQ376641; DQ376642; DQ376643; DQ376644; DQ376645; DQ376646; DQ376647; DQ376648; DQ376649; DQ376650; DQ376651; DQ376652; DQ376653; DQ408509; DQ408517; DQ408524; DQ822190; DQ822198; J02158; AB294213; AB294215; AB294219; AB295615; AB296072; AB298279; AF100181; AF250479; AF310983; AF310984; AF310985; AF457663; AF457664; AF457665; AF457666; AF457667; AF457668; AF457669; AF457670; AF457679; AF457688; AF457696; AF457704; AF457713; AF457715; AF474029; AF474030; AF474031; AF474032; AF474033; AF474034; AF474035; AF474036; AF474037; AF474038; AJ410519; AJ410520; AJ410521; AJ410522; AJ410523; AJ410524; AJ410525; AJ410526; AJ410527; AJ410528; AJ410529; AJ410530 and AJ410531.

Polynucleotides encoding influenza virus subtype H7 hemagglutinins suitable for inclusion as a cargo moiety in conjugates herein have the following accession numbers and are available to the public via the National Center for Biotechnology Information (NCBI) Entrez nucleotide search and retrieval system:

AB262459; AF072385; AF202256; AF322021; AF322022; AF322023; AF322024; AF322025; AF322026; AF364133; AF364134; AF364135; AF364136; AF364137; AF364138; AF364139; AF364140; AF364141; AF364142; AF364143; AF364144; AF364145; AF364146; AF364147; AF364148; AF364149; AF364150; AF364151; AF364152; AF364153; AF364154; AF364155; AF364156; AF364157; AF364158; AF364159; AF364160; AF364161; AF364162; AF364163; AF364164; AF364165; AF364166; AF364167; AF364168; AF364169; AF364170; AF364171; AF364172; AF497551; AF497552; AF497553; AF497554; AF497555; AF497556; AF497557; AF497558; AF497559; AJ489520; AJ491720; AJ493212; AJ493213; AJ493214; AJ493215; AJ493216; AJ493217; AJ493466; AJ493467; AJ493468; AJ493469; AJ493470; AJ493471; AJ493472; AJ580353; AJ584647; AJ620350; AJ627491; AJ627493; AJ697872; AJ697873; AJ704797; AJ704798; AJ704799; AJ704810; AJ704811; AJ704812; AJ704813; AM087214; AM087223; AY240877; AY240878; AY240879; AY240880; AY240881; AY240882; AY240883; AY240884; AY240885; AY240886; AY240887; AY240888; AY240889; AY240890; AY240891; AY240892; AY240893; AY240894; AY240895; AY240896; AY240897; AY240898; AY240899; AY240900; AY240901; AY240902; AY240903; AY240904; AY240905; AY240906; AY240907; AY240908; AY240909; AY240910; AY240911; AY240912; AY240913; AY240914; AY240915; AY240916; AY240917; AY240918; AY240919; AY240920; AY240921; AY240922; AY240923; AY240924; AY240925; AY303630; AY303631; AY303632; AY303633; AY303634; AY303635; AY338455; AY338456; AY338457; AY338458; AY338459; AY338460; AY338461; AY338462; AY383756; AY559235; AY586408; AY586409; AY586410; AY586411; AY596307; AY611524; AY644402; AY646078; AY648287; AY650270; AY672090; AY724257; AY724684; AY725855; AY730057; AY731820; AY734541; AY736323; AY831668; AY831669; AY831670; AY943924; AY999977; AY999978; AY999979; AY999980; AY999981; AY999982; AY999983; AY999984; AY999985; AY999986; AY999987; AY999988; AY999989; AY999990; AY999991; CY005928; CY005973; CY005974; CY005975; CY005976; CY005978; CY005980; CY005981; CY005983; CY006029; CY006037; CY014587; CY014612; CY014718; CY014721; CY014778; CY014786; CY014896; CY014992; CY015006; CY015014; CY015027; CY015033; CY015065; CY016188; CY018901; CY020581; CY020589; CY020597; CY020605; CY020613; CY020685; CY020885; CY021357; CY021365; CY021405; CY021413; CY021421; CY021485; CY021493; CY021501; CY021533; CY021541; CY021549; CY021557; CY021621; CY021637; DQ003216; DQ017504; DQ017513; DQ525411; DQ838510; DQ838511; DQ838512; DQ838513; DQ838514; DQ838515; DQ870888; DQ870894; DQ873807; DQ907527; DQ907528; DQ991304; DQ991312; DQ991320; DQ991328; DQ991336; DQ991343; EF467825; EF467826; J02164; K00429; L37794; L43913; L43914; L43915; M17735; M17736; M24457; M24458; M31689; M58657; U20458; U20459; U20461; U20462; U20463; U20464; U20465; U20466; U20467; U20468; U20469; U20470; U20471; X61627; X62552; X62553; X62554; X62555; X62556; X62557; X62558; X62559; X62560; Z12617; Z47199; AB262468; AB262469; AB262470; AB262471; AB262472; AB262473; AB268557; AB269692; AB269693; AB269694; AB269695; AB269696; AB269872; AB270592; AB270593; AB297923; AB297925; AB298277; AF028020; AF028021; AF071775; AF071776; AF072383; AF072384; AF072386; AF072387; AF072388; AF072389; AF072390; AF072391; AF072392; AF072393; AF072394; AF072395; AF072396; AF072397; AF072398; AF072399; AF072400; AF072401; AF072402; AF149295; AF202226; AF202227; AF202228; AF202229; AF202230; AF202231; AF202232; AF202233; AF202234; AF202235; AF202236; AF202237; AF202238; AF202239; AF202240; AF202241; AF202242; AF202243; AF202244; AF202245; AF202246; AF202247; AF202248; AF202249; AF202250; AF202251; AF202252; AF202253; AF202254 and AF202255.

Polynucleotides encoding influenza virus subtype H8 hemagglutinins suitable for inclusion as a cargo moiety in conjugates herein have the following accession numbers and are available to the public via the National Center for Biotechnology Information (NCBI) Entrez nucleotide search and retrieval system:

AB289343; AF310987; AF310988; AF310989; CY005970; CY005971; CY005972; CY014583; CY014659; CY015173; CY017749; D90304; EF061122 and J02089.

Polynucleotides encoding influenza virus subtype H9 hemagglutinins suitable for inclusion as a cargo moiety in conjugates herein have the following accession numbers and are available to the public via the National Center for Biotechnology Information (NCBI) Entrez nucleotide search and retrieval system:

J02166; AF156385; AF156386; AF156387; AF156388; AF156389; AF156390; AF156373; AF156374; AF156375; AF156376; AF156377; AF156378; AF156379; AF156380; AF156381; AF156382; AF156383; AF156384; AF186266; AF186267; AF186268; AF186269; AF203008; AF203009; AF203010; AF203011; AF203012; AF203013; AF203014; AF203015; AF222606; AF222607; AF222608; AF222609; AF222610; AF222611; AF222612; AF222613; AF218086; AF218087; AF218088; AF218089; AF218090; AF218091; AF218092; AF218093; AF218094; AF218095; AF218096; AF218097; AF218098; AF218099; AF218100; AF218101; AF218102; AF218103; AF218104; AF218105; AF218106; AF218107; AF218108; AF218109; AF218110; AF218111; AF218112; AF218113; AF218114; AF218115; AF218116; AF218117; AF218118; AF218119; AF218120; AF384557; AY036880; AF222810; AF222811; AF400776; AF400777; AY043014; AY043015; AY043017; AY043018; AY043019; AF461509; AF461510; AF461511; AF461512; AF461513; AF461514; AF461515; AF461516; AF461517; AF461518; AF461519; AF461520; AF461521; AF461522; AF461523; AF461524; AF461525; AF461526; AF461527; AF461528; AF461529; AF461530; AF461531; AF461532; AY083840; AY083841; AF536689; AF536690; AF536691; AF536692; AF536693; AF536694; AF536695; AF536696; AF536697; AF536698; AY180444; AY180445; AY180446; AY180447; AY180448; AY180449; AY180450; AY180451; AY180452; AY180453; AY180454; AY180455; AY180456; AY180457; AY180458; AY180459; AY206671; AY206672; AY206673; AY206674; AY206675; AY206676; AY206677; AY206678; AY206679; AY206680; AY198313; AY198314; AY198315; AY198316; AY198317; AY198318; AY198319; AY198320; AY198321; AY281745; AY264870; AY264871; AY264872; AY264875; AY264876; AY294658; AF523372; AF523373; AF523374; AF523375; AF523376; AF523377; AF523378; AF523379; AF523380; AF523381; AF523382; AF523383; AF523384; AF523385; AF523386; AF523387; AF523388; AF523389; AF523390; AY336597; AF508554; AF508555; AF508556; AF508557; AF508558; AF508559; AF508560; AF508561; AF508562; AF508563; AF508564; AF508565; AF508566; AF508567; AF508568; AF508569; AF508570; AF508571; AF508572; AF508573; AF508574; AY345925; AY345926; AY345927; AY345928; AY345929; AY345930; AY345931; AY345932; AY345933; AY345934; AY345935; AY345936; AY345937; AY345938; AY345939; AY345940; AY364228; AY330332; AY330333; AY330334; AY330335; AY330336; AY435039; AY435040; AY513715; AY548499; AY548500; AY548501; AY548502; AY548503; AY548504; AY548505; AY548506; AY548507; AY548508; AY548509; AY548510; AY548511; AY548512; AY548513; AY548514; AY548515; AY603067; AY549889; AY623810; AY633116; AY633164; AY633276; AY633292; AY652980; AY594194; AY594195; AY594196; AY664660; AY664661; AY664662; AY664663; AY664664; AY664665; AY664666; AY664667; AY664668; AY664669; AY664670; AY664671; AY664672; AY664673; AY664674; AY664675; AY664676; AY664677; AY664678; AY743216; AY768552; AY768553; AY768554; AY768555; AY768556; AY768557; AY768558; AY768559; AY790275; AY790283; AY790297; AY790305; AY790313; AY790314; AY790315; AY790320; AY738451; AY738452; AY738453; AY738454; AY738455; AY738456; AY851460; AY851461; AY862598; AY862599; AY862600; AY862601; AY862602; AY862603; AY862604; AY862605; AY862606; AY937403; AY937404; AY949989; DQ003335; DQ064354; DQ064355; DQ064356; DQ064357; DQ064358; DQ064359; DQ064360; DQ064361; DQ064362; DQ064363; DQ064364; DQ064365; DQ064366; DQ064367; DQ064368; DQ064369; DQ064370; DQ064371; DQ064372; DQ064373; DQ064374; DQ064375; DQ064376; DQ064377; DQ064378; DQ064379; DQ064380; DQ067444; DQ108905; DQ108906; DQ108907; DQ108908; DQ108909; DQ108910; DQ108911; DQ108912; DQ108913; DQ108914; DQ108915; DQ108916; DQ108917; DQ108918; DQ108919; DQ108920; DQ108921; DQ108922; DQ108923; DQ108924; DQ108925; DQ108926; DQ108927; DQ108928; DQ108929; DQ108930; DQ108931; DQ108932; DQ104448; DQ104449; DQ104450; DQ104451; DQ104452; DQ104453; DQ104454; DQ104455; DQ104456; DQ104457; DQ104458; DQ104459; DQ104460; DQ104461; DQ104462; DQ104463; DQ104464; DQ104465; DQ104466; DQ104467; DQ104468; DQ104469; DQ104470; DQ104471; DQ104472; DQ104473; DQ104474; DQ104475; DQ104476; DQ104477; DQ104478; DQ104479; DQ104480; DQ104481; DQ104482; DQ104483; DQ104484; DQ104485; DQ225271; DQ227352; DQ223544; CY004420; CY004642; CY005632; CY005639; CY005746; DQ234277; DQ226106; DQ226107; DQ226108; DQ226109; DQ226110; DQ226111; DQ226112; DQ226113; DQ226114; DQ226115; DQ226116; CY005919; CY005929; CY005934; CY005984; CY005985; CY005986; CY005987; CY005988; CY005989; CY005990; CY005991; CY005992; CY006025; CY006042; CY006018; CY006021; CY006023; DQ299829; DQ299837; DQ299845; DQ299853; DQ299861; DQ390215; DQ464352; DQ473608; DQ473609; DQ473610; DQ473611; DQ473612; DQ473613; DQ473614; DQ465400; DQ485208; DQ485216; DQ485224; DQ681203; DQ681207; DQ681216; DQ681221; DQ885991; DQ787797; DQ787802; CY014613; CY014663; DQ997505; DQ997481; DQ997474; DQ997437; DQ997460; DQ997187; DQ997465; DQ997490; DQ997451; DQ997419; DQ997448; EF070733; EF063510; EF063511; EF063512; EF063513; EF063514; EF063515; EF063516; EF154907; EF154908; EF154909; EF154910; EF154911; EF154912; EF154913; EF154914; EF154915; EF154916; EF154917; EF154918; EF154919; EF154920; EF154921; EF154922; EF154923; EF154924; EF154925; EF154926; EF154927; EF154928; EF154929; EF154930; EF154931; EF154932; EF154933; EF154934; EF154935; EF154936; EF154937; EF154938; EF154939; EF154940; EF154941; EF154942; EF154943; EF154944; EF154945; EF154946; EF154947; EF154948; EF154949; EF154950; EF154951; EF154952; EF154953; EF154954; EF154955; EF154956; EF154957; EF154958; EF154959; EF154960; EF154961; EF154962; EF154963; EF154964; EF154965; EF154966; EF154967; EF154968; EF154969; EF154970; EF154971; EF154972; EF154973; EF154974; EF154975; EF154976; EF154977; EF154978; EF154979; D90305; ABO49159; ABO49160; ABO80224; ABO80225; ABO80226; ABO80227; ABO80228; AB125927; AB125928; AB125929; AB125930; AB125931; AB262463; AB276111; AB256666; AB256674; AB256682; AB256690; AB256698; AB256706; AB256714; AB256722; AB256730; AB256738; AB256746; AB295601; AJ404626; AJ404627; AJ291392; AJ536330; AJ536331; AJ536332; AJ781818; AJ781819; AJ781820; AJ781821; AJ781822; AJ781823; AJ781824; AJ781825; AJ781826; AJ781827; AM087218; AM087219; AM286688 and AM286689.

Polynucleotides encoding influenza virus subtype H10 hemagglutinins suitable for inclusion as a cargo moiety in conjugates herein have the following accession numbers and are available to the public via the National Center for Biotechnology Information (NCBI) Entrez nucleotide search and retrieval system: AB271117; AB274041; CY005996; CY005997; CY005998; CY005999; CY006000; CY006001; CY014619; CY014644; CY014671; CY014739; CY017781; CY020901; CY020909; CY020925; DQ374399; J02110; M21646; M21647; AB289339; AB292412; AB292666; AB292781; AB296078; AF311750; AM087215; AM087216; CY005921; CY005922; CY005930; CY005982; CY005993; CY005994 and CY005995.

Polynucleotides encoding influenza virus subtype H11 hemagglutinins suitable for inclusion as a cargo moiety in conjugates herein have the following accession numbers and are available to the public via the National Center for Biotechnology Information (NCBI) Entrez nucleotide search and retrieval system: AB277756; AB288845; DQ424861; DQ435281; DQ435282; DQ435283; DQ435284; DQ435285; DQ482667; EF200063; J02100; J02106; J02107; J02108; J02161; AB292779; AB292783; AB296076; AB298283; AF310986; AY684895; CY005923; CY005924; CY006002; CY006003; CY006004; CY006005; CY014593; CY014595; CY014679; CY014687; CY014719; CY014806; CY017075; CY017765; CY017845; CY018015; CY020941; CY020949; CY020965; CY021133; CY021141; CY021149; CY021165; CY021173; CY021181; CY021245; CY021253; CY021437; CY021445; CY021469; CY021613; CY021645; CY021653; CY021661; CY021669; CY021685; D90306; DQ080993; DQ327835; DQ424858; DQ424859 and DQ424860.

Polynucleotides encoding influenza virus subtype H12 hemagglutinins suitable for inclusion as a cargo moiety in conjugates herein have the following accession numbers and are available to the public via the National Center for Biotechnology Information (NCBI) Entrez nucleotide search and retrieval system: AB288334; AB288843; AF310990; AF310991; AF310992; AM286685; CY005920; CY005925; CY006006; CY006007; CY006008; CY012840; CY014598; CY014636; CY016419; CY017733; CY017853; CY021293; CY021301; D90307; DQ787811 and J02104.

Polynucleotides encoding influenza virus subtype H13 hemagglutinins suitable for inclusion as a cargo moiety in conjugates herein have the following accession numbers and are available to the public via the National Center for Biotechnology Information (NCBI) Entrez nucleotide search and retrieval system: AB284988; AB285094; AB292664; AM087220; AM087221; AY684886; AY684887; CY005914; CY005931; CY005932; CY005979; CY014603; CY014694; CY014720; D90308; K00383; M26089; M26090 and M26091.

Polynucleotides encoding influenza virus subtype H14 hemagglutinins suitable for inclusion as a cargo moiety in conjugates herein have the following accession numbers and are available to the public via the National Center for Biotechnology Information (NCBI) Entrez nucleotide search and retrieval system: AB289335 and CY014604.

Polynucleotides encoding influenza virus subtype H15 hemagglutinins suitable for inclusion as a cargo moiety in conjugates herein have the following accession numbers and are available to the public via the National Center for Biotechnology Information (NCBI) Entrez nucleotide search and retrieval system: CY006010; CY006033; CY006034; L43917; CY006032; AB295613; CY006009 and L43916.

Polynucleotides encoding influenza virus subtype H16 hemagglutinins suitable for inclusion as a cargo moiety in conjugates herein have the following accession numbers and are available to the public via the National Center for Biotechnology Information (NCBI) Entrez nucleotide search and retrieval system: CY005933; CY014599; CY015160; AY684888; AY684889; AY684890 and AY684891.

Thus, a polynucleotide encoding an influenza virus hemagglutinin may be included as a cargo moiety.

In particular embodiments, influenza virus hemagglutinin protein or an antigenic portion thereof is included in a conjugate composition for administration to a subject to enhance an immune response to influenza virus.

In other embodiments, the virus conjugated to a sialoadhesin binding antibody may be used as a gene transfer vector in order to express a desired nucleic acid in a target cell. Such viruses are known in the art and include herpes viruses, adenoviruses and adeno-associated viruses, for example.

In further embodiments, a viral cargo moiety is a virus or portion thereof expressing no non-viral proteins. A cargo moiety virus is a porcine arterivirus in one embodiment.

### Conjugation

A cargo moiety is conjugated to a sialoadhesin binding moiety by any of various methods. The conjugation method chosen will depend on the chemical identity of the cargo and the sialoadhesin binding moiety.

A conjugate according to embodiments hereof encompasses a sialoadhesin binding moiety and a cargo linked together by chemical bonding, covalent or non-covalent, as well as by recombinant techniques including production of a fusion protein, such as a conjugate produced using a nucleic acid expression construct encoding a sialoadhesin binding moiety and a cargo.

In particular embodiments, a cargo moiety and a sialoadhesin binding moiety are chemically linked via free functional groups on these moieties. Such functional groups illustratively include amino, carboxyl, hydroxyl, and sulfhydryl groups.

A linkage between a cargo moiety and a sialoadhesin binding moiety is illustratively an ester, an ether, a carbamate, a carbonate, a disulfide, a peptide, and an amide. The term “linkage” refers to a bond or group formed by chemical reaction between the two moieties such that the moieties are covalently coupled, directly or indirectly.

In certain embodiments, a linkage between a sialoadhesin binding moiety and a cargo moiety is labile in an intracellular environment, such that the sialoadhesin binding moiety and cargo moiety may be separated following cell uptake. For instance, a linkage may be susceptible to hydrolysis, enzymatic cleavage, or other form of cleavage, such that the cargo moiety provides a desired effect following such separation from the sialoadhesin binding moiety. An ester linkage is one example of a linkage susceptible to hydrolysis in a cell. A disulfide linkage is a further example of a linkage susceptible to cleavage following cell uptake. In other embodiments, a cargo moiety provides a desired effect while conjugated to the sialoadhesin binding moiety.

In certain embodiments, more than one cargo moiety may be included in a conjugate composition. Further, more than one sialoadhesin binding moiety may be included in a conjugate composition.

Where one or both of the sialoadhesin binding moiety and the cargo moiety include a peptide and/or protein, functional group of a cargo moiety and a sialoadhesin binding moiety used to conjugate these moieties can be at N- or C-terminus or at between the termini of one or both peptides or proteins.

A protective group may be added to a sialoadhesin binding moiety and/or cargo moiety in a process to form a conjugate herein. Such groups, their generation and use are described in Protective Groups in Organic Synthesis by T. W. Greene and P. G. M. Wuts, John Wiley & Sons, 1999.

Conjugation chemistries used in conjugation of a cargo moiety and a sialoadhesin binding moiety illustratively include coupling agents such as, but not limited to, glutaraldehyde, carbodiimide, succinimide esters, benzidine, periodate, isothionate and combinations of these.

A conjugate herein may be produced using recombinant techniques. For example, in particular embodiments, a conjugate is an expression product of a nucleic acid construct including an expression construct encoding a fusion protein, the fusion protein including a sialoadhesin binding moiety or portion thereof and a cargo moiety linked directly to the sialoadhesin binding moiety or portion thereof or through an intermediate linker.

In particular embodiments, an expression construct encoding a fusion protein encodes an anti-sialoadhesin antibody or a fragment of an anti-sialoadhesin antibody. Thus, in particular embodiments, an expression construct encodes a fusion protein including a nucleic acid which encodes a cargo moiety and an anti-sialoadhesin antibody or portion thereof. For example, an expression construct encoding a fusion protein herein encodes a cargo attached to a portion of an anti-sialoadhesin antibody including a variable region of an anti-sialoadhesin antibody such as, but not limited to, a heavy chain variable region and/or a light chain variable region, a single chain VL-VH region, and/or an H chain C region in particular embodiments.

In particular embodiments, an expression construct encoding a fusion protein encodes a cargo moiety and mAb 41D3 or a portion of mAb 41D3. In further embodiments, an expression construct encoding a fusion protein encodes an influenza virus hemagglutinin and mAb 41D3 or a portion of mAb 41D3.

In particular embodiments, an expression construct encoding a fusion protein encodes a cargo moiety and mAb 7D2 or a portion of mAb 7D2. In further embodiments, an expression construct encoding a fusion protein encodes an influenza virus hemagglutinin and mAb 7D2 or a portion of mAb 7D2.

In particular embodiments, an expression construct encoding a fusion protein encodes a cargo moiety and mAb MCA2316 or a portion of mAb MCA2316. In further embodiments, an expression construct encoding a fusion protein encodes an influenza virus hemagglutinin and mAb MCA2316 or a portion of mAb MCA2316.

Cloning and expression of nucleic acids encoding antibody regions and fusion proteins including an antibody region are known in the art as exemplified in J. D. Pound (Ed.) Immunochemical Protocols. Methods in Molecular Biology, Humana Press; 2nd ed., 1998, chapter 43; R. Kontermann and S. Dübel (Eds.), Antibody Engineering, Springer Lab Manuals, Springer, 2001; and B. K. C. Lo (Ed.), Antibody Engineering: Methods and Protocols. Methods in Molecular Biology, Humana Press, 2003.

A cargo moiety and a sialoadhesin binding moiety may be linked directly to form a conjugate. Alternatively, a linker may be bound to both a cargo moiety and to a sialoadhesin binding moiety, such that these moieties are indirectly linked through the linker. A linker may be a homo bifunctional linker or a hetero-bifunctional linker, depending on the identity of the moieties to be conjugated. Further, a linker may be multifunctional so as to link more than one cargo moiety and/or more than one sialoadhesin binding moiety.

In general, a linker has about 1-20 backbone carbon atoms. However, a linker may be larger or smaller.

Optionally, a linker is encoded by a nucleic acid in an expression construct.

A linker may be a natural or synthetic polymer in some embodiments. For example, suitable polymers include agarose, carboxymethylcellulose, cellulose, dextran, and polyaminopolystyrene. A preferred polymer is polyacrylamide, PEO (polyethylene) or PEG (polyethylene glycol) spacer.

In certain embodiments, a sialoadhesin binding moiety including a sialic acid and/or sialylated structure may be conjugated to a cargo moiety directly or indirectly. For example, a sialic acid residue may be conjugated to a lipid-containing cargo moiety to form a glycolipid conjugate composition and/or to a protein or peptide cargo moiety by N-linkage or O-linkage to form a glycopeptide or glycoprotein conjugate herein. A sialic acid residue may also be conjugated to a linker.

### Pharmaceutical Compositions and Administration

A conjugate can be administered to a subject alone or as part of a pharmaceutical composition. Conjugate compositions are suitable for administration to patients by a variety of routes illustratively including, but not limited to, intravenous, oral, parenteral, intramuscular, subcutaneous and mucosal.

The pharmaceutical compositions hereof include a conjugate and a pharmaceutically acceptable carrier. The term “pharmaceutically acceptable” refers to a material which can be administered to a subject along with a conjugate composition without causing significant undesirable biological effects and without interacting in a deleterious manner with any other component of the pharmaceutical composition. Pharmaceutical compositions suitable for administration illustratively include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers; diluents; solvents; or vehicles include water, ethanol, polyols such as, but not limited to, propylene glycol, polyethylene glycol, glycerol, and the like, suitable mixtures thereof; vegetable oils such as, but not limited to, olive oil; and injectable organic esters such as, but not limited to, ethyloleate. Proper fluidity can be maintained, e.g., by the use of a coating such as, but not limited to, lecithin, by the maintenance of the required particle size in the case of dispersions, and by the use of surfactants.

Compositions suitable for injection optionally include physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as, but not limited to, ethyl oleate. Proper fluidity can be maintained, e.g., by the use of a coating such as, but not limited to, lecithin, by the maintenance of the required particle size in the case of dispersions and by the use of surfactants.

Pharmaceutical compositions herein may also contain adjuvants such as, but not limited to, preserving, wetting, emulsifying, and dispensing agents. Prevention of the action of microorganisms can be ensured by various antibacterial and antifungal agents, e.g., parabens, chlorobutanol, phenol, sorbic acid, and the like. It may also be desirable to include isotonic agents, e.g., sugars, sodium chloride, and the like. Prolonged absorption of an injectable pharmaceutical form can be brought about by the use of agents delaying absorption, e.g., aluminum monostearate and gelatin.

Further exemplary adjuvants include immunostimulating adjuvants such as, but not limited to, Freund's complete adjuvant; Freund's incomplete adjuvant; aluminum hydroxide such as commercially available as Alhydrogel, Accurate Chemical & Scientific Co, Westbury, N.Y.; and Gerbu adjuvant, available from C—C Biotech, Poway, Calif.

Solid dosage forms for oral administration include capsules, tablets, pills, powders, and granules. In such solid dosage forms, a conjugate is admixed with at least one inert customary excipient (or carrier) such as, but not limited to, sodium citrate or dicalcium phosphate or (a) fillers or extenders, as for example, starches, lactose, sucrose, glucose, mannitol, and silicic acid, (b) binders, as for example, carboxymethylcellulose, alginates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as, but not limited to, tablets, dragees, capsules, pills, and granules can be prepared with coatings and shells, such as, but not limited to, enteric coatings and others well known in the art. They may contain opacifying agents, and can also be of such composition that they release the active compound or compounds in a certain part of the intestinal tract in a delayed manner. Microencapsulated formulations of a conjugate are also contemplated.

Liquid dosage forms for oral administration include pharmaceutically acceptable emulsions, solutions, suspensions, syrups, and elixirs. In addition to a conjugate herein, the liquid dosage forms may contain inert diluents commonly used in the art, such as, but not limited to, water or other solvents, solubilizing agents and emulsifiers, as for example, ethyl alcohol, isopropyl alcohol, ethyl carbonate, ethyl acetate, benzyl alcohol, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, a pharmaceutical composition herein can also include adjuvants, such as, but not limited to, wetting agents, emulsifying and suspending agents, sweetening, flavoring, and perfuming agents.

Suspensions, in addition to a conjugate, may contain suspending agents, e.g., ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Further specific details of pharmaceutical formulation can be found in Pharmaceutical Dosage Forms Tablets, eds. H. A. Lieberman et al., New York: Marcel Dekker, Inc., 1989; L. V. Allen, Jr. et al., Ansel's Pharmaceutical Dosage Forms and Drug Delivery Systems, 8th Ed., Philadelphia, Pa., Lippincott, Williams & Wilkins, 2004; and Remington, The Science and Practice of Pharmacy, 21^{st} ed., Lippincott, Williams & Wilkins, Philadelphia, Pa., 2006.

A conjugate may be delivered in conjunction with a non-conjugated therapeutic and/or diagnostic agent in one embodiment. A therapeutic and/or diagnostic agent suitable in this regard illustratively includes an analgesic, an antibiotic, an antibody, an antigen, an anti-inflammatory, an anti-tumoral agent, an antiviral, a gamma or beta radiation emitting species, an enzyme, and a hormone. In addition, two or more conjugate compositions may be administered to a subject.

The dosage of an inventive pharmaceutical composition will vary based on factors such as, but not limited to, the route of administration; the age, health, and weight of the subject to whom the composition is to be administered; the nature and extent of the subject's symptoms, if any, and the effect desired. Usually a daily dosage of a conjugate is in the range of about 0.001 to 100 milligrams per kilogram of a subject's body weight. A daily dose may be administered as two or more divided doses to obtain the desired effect. An inventive pharmaceutical composition may also be formulated for sustained release to obtain desired results.

For example, a parenteral composition suitable for administration by injection includes 1% by weight of a conjugate in buffered saline.

### Methods

A method of delivering a cargo moiety to a cell is provided which includes contacting a cell expressing sialoadhesin with a conjugate herein. The sialoadhesin binding moiety present in the conjugate binds to the sialoadhesin expressed by the cell and the conjugate is internalized in the cell. The cell may be in vivo, ex vivo or in vitro.

Sialoadhesin is expressed primarily by macrophages. Thus, in one embodiment of an inventive method, a drug delivery system targeting macrophages is provided. Thus, in such an embodiment, a cell contacted with a conjugated sialoadhesin binding moiety and cargo moiety is a macrophage.

A cell contacted with a conjugate composition in a method herein expresses sialoadhesin naturally or may be induced to do so. In such a method, cells other than macrophages may be targeted.

For example, a cell may be transfected with an expression construct encoding sialoadhesin such that sialoadhesin is expressed in the cell. An expression construct includes a nucleic acid encoding full-length sialoadhesin, or a portion thereof, operably linked to a regulatory element. Full-length nucleic acids encoding sialoadhesin have been isolated from various species and exemplary polynucleotides and encoded sialoadhesin proteins are described herein. A regulatory element operably linked to the nucleic acid encoding sialoadhesin illustratively includes a promoter, an enhancer, an origin of replication, a polyadenylation signal, and a transcription termination sequence. Expression constructs and methods for their generation are known in the art, as described, e.g., in Sambrook et al., Molecular Cloning: A Laboratory Manual, Cold Spring Harbor Laboratory Press, 2001; and F. Ausubel et al. (Eds.), Short Protocols in Molecular Biology, Wiley, 2002.

In particular embodiments, an expression construct encoding a sialoadhesin protein encodes the sialoadhesin protein of SEQ ID NO:5, SEQ ID NO:7, SEQ ID NO:9, or a biologically active homologue thereof. In particular embodiments, an expression construct encoding a sialoadhesin protein includes a nucleotide sequence of SEQ ID NO:6, SEQ ID NO:8, or SEQ ID NO:10.

Biological activity of a putative sialoadhesin homologue may be determined by one of skill in the art, e.g., by using a functional assays described herein or other functional assays known in the art.

An expression construct encoding sialoadhesin is generated according to methods known in the art. For example, a pcDNA3.1/Sn plasmid containing the porcine sialoadhesin cDNA cloned into the pcDNA3.1 vector (Invitrogen) described in N. Vanderheijden et al., 2003, J. Virol. 77:8207-15 is a sialoadhesin expression construct.

A cell transfected with an expression construct to induce or enhance sialoadhesin expression in the cell may be transiently transfected in particular embodiments. Alternatively, a stable cell line expressing sialoadhesin is produced.

Any of various cells may be used to produce a cell line stably expressing sialoadhesin, e.g., including, but not limited to THP-1 cells, PK-15 cells, 3D4/31 cells, and HEK293T cells.

Methods of producing a stable cell line expressing a desired protein are known in the art, e.g., in standard molecular biology references such as S. Ozturk and W.-S. Hu (Eds.), Cell Culture Technology for Pharmaceutical and Cell-Based Therapies. Biotechnology and Bioprocessing Series, CRC Press, 2005.

Briefly described, cells are transfected with an expression construct encoding sialoadhesin. For example, cells are transfected with an expression construct including SEQ ID NO:6, SEQ ID NO:8, or SEQ ID NO:10 or another sequence encoding SEQ ID NO:5, SEQ ID NO:7, or SEQ ID NO:9 or a homologue thereof. A transfected expression construct further encodes resistance to a selection agent, including, but not limited to, resistance to neomycin (G418). Expression constructs conferring resistance to a selection agent are known in the art and are commercially available or may be constructed using standard molecular biology techniques.

Cells are transfected according to standard transfection methods illustratively including, but not limited to, calcium phosphate techniques and lipofectin techniques such as described in Sambrook et al., Molecular Cloning: A Laboratory Manual, Cold Spring Harbor Laboratory Press, 2001; and F. Ausubel et al. (Eds.), Short Protocols in Molecular Biology, Wiley, 2002.

Following transfection, cells are incubated, on cell culture plates or in cell culture wells for instance, in medium containing a selection agent, such as 0.5 g/L neomycin. Cells not transfected or not expressing the resistance marker die following incubation with the selection agent, generally after several days. Dead cells are removed from the vicinity of living transfected cells in order to select for particular clones. Transfected cells are typically disposed individually, in wells or in cloning cylinders for example, in order to select one or more stably transfected cell lines. Once individual colonies have grown, they can be assayed for sialoadhesin expression, such as by ELISA. Stably transfected cells are further assayed for binding of a sialoadhesin binding moiety and/or conjugate and uptake of the binding moiety and/or conjugate into the cell.

Stably transfected cells may be used in methods hereof. For example, a stable cell line expressing sialoadhesin is used in a method hereof for transfection of a cell by delivery of a conjugate including a sialoadhesin binding moiety and a nucleic acid.

In further embodiments of methods herein, a cell is treated with an agent effective to induce or enhance expression of sialoadhesin in the cell. In particular embodiments, a cell is treated with a cytokine effective to induce or enhance expression of sialoadhesin in the cell. For example, a cell treated with a cytokine effective to induce or enhance expression of sialoadhesin is a monocyte, a monocyte cell line, a macrophage and a macrophage cell line.

In particular embodiments, a human cell and/or a human-derived cell line is treated with a cytokine effective to induce or enhance expression of sialoadhesin. An example of a human-derived cell line is human monocyte cell line THP-1. A suitable cytokine effective to induce or enhance expression of sialoadhesin is interferon-alpha (INF-alpha).

Human monocytes are treated with INF-alpha to induce or enhance expression of sialoadhesin in a particular embodiment. The monocytes may be isolated, for instance from blood, and treated in vitro with INF-alpha. Sialoadhesin expression may be assessed by assays illustratively including, but not limited to, immunoassay.

In further embodiments, an effective amount of INF-alpha is administered to a subject such that sialoadhesin expression is induced or enhanced in cells in vivo. An effective amount is illustratively between 10 to 500 units IFN-alpha per ml of blood of the subject.

Methods are provided for transfection of a cell using a conjugate herein including a cargo nucleic acid, particularly a cargo expression construct. Cells expressing sialoadhesin are contacted with a conjugate including a sialoadhesin binding moiety and a cargo expression construct in a particular embodiment in order to express an encoded protein or peptide in the cells. Transfection using a sialoadhesin binding moiety and a cargo expression construct is used in sialoadhesin expressing cells in vitro or in vivo. Transfection using a conjugate provided herein is useful to increase the level of a desired protein or peptide in a cell, for instance, to produce recombinantly expressed protein, for instance, to study function of the protein.

A method includes contacting a cell expressing sialoadhesin with a conjugate composition may be used to stimulate an immune response in a subject, for instance, to vaccinate the subject.

Vaccination is one of the earliest used and most powerful tools for stimulating an organism to defend against infection. Broadly described, vaccination is a method of administering an antigen to an organism in order to stimulate the organism's immune system to provide a cellular and/or molecular defensive response.

While vaccination by non-cell targeted administration of an antigen to an organism can be effective, in some cases large amounts of antigen must be administered in order to achieve a desired response. Further, a non-cell targeted administration may require a longer time and/or more booster administrations of the antigen to achieve an effective immune response. Thus, compositions and methods for stimulating an immune response in a subject are needed. Such a method is provided herein and includes administering to a subject an effective amount of a conjugate composition herein which includes a sialoadhesin binding moiety conjugated to an antigen. An immune response may be stimulated in order to inhibit infection by a pathogen, or to stimulate an antitumoral response for instance.

An immune response may be measured, e.g., by assay of the subject's serum for antibodies to an antigen administered as part of a conjugate. Applicable immunoassays include, e.g., ELISA performed on a sample before and at one or more times following administration of the conjugate.

In certain embodiments, administration of a composition effective to target an antigen to an antigen presenting cell, particularly a macrophage, is included in a method provided herein.

In a specific example, vaccination of swine against PRRSV is an embodiment hereof. PRRSV is an infectious disease of swine which can cause severe respiratory disorders, as well as abortion. The viral agent has been identified, as described in G. Weensvoort et al., 1991, Veterinary Review 13:121-130. However, there is currently no effective treatment for this disease which can frequently only be controlled by destruction of the herd, resulting in considerable cost to swine producers.

A vaccine and method for vaccination of a pig against PRRSV is provided. A conjugate composition including a sialoadhesin binding moiety which binds to porcine sialoadhesin is conjugated to a PRRSV, a PRRS protein, or an antigenic portion of a PRRSV or protein. The conjugate composition is administered to a pig in an amount effective to stimulate an immune response. The route of administration may be any convenient route, illustratively including, but not limited to, intravenous, intramuscular, intraperitoneal, subcutaneous, oral, mucosal, and any combination thereof.

In a further example, vaccination of a subject against an influenza virus is an embodiment hereof. Influenza virus is an infectious disease of numerous species which can cause severe respiratory symptoms and death.

A vaccine and method for vaccination of a subject against influenza virus is provided. A conjugate composition including a sialoadhesin binding moiety which binds to sialoadhesin is conjugated to an influenza virus, an influenza virus protein or an antigenic portion of an influenza virus or protein. In particular embodiments, compositions and methods for vaccination of a subject against a type A influenza virus are provided. The conjugate composition is administered to a subject in an amount effective to stimulate an immune response against influenza virus. The route of administration may be any convenient route, illustratively including, but not limited to, intravenous, intramuscular, intraperitoneal, subcutaneous, oral, mucosal, and any combination thereof.

In specific embodiments, compositions and methods for vaccination of a porcine subject against an influenza virus are provided. Inventive methods and compositions for vaccination against influenza virus are not limited to porcine subjects and may be used in other subjects susceptible to influenza virus infection, illustratively including, but not limited to, humans and birds.

A conjugate composition for vaccination of a subject against an influenza virus includes a sialoadhesin binding moiety and an influenza virus hemagglutinin protein or antigenic portion thereof in particular embodiments. In a specific example, a conjugate composition for vaccination of a subject against an influenza virus includes the protein encoded by SEQ ID NO:3 or a homologue thereof. In a further specific example, a conjugate composition for vaccination of a subject against an influenza virus includes the protein identified as SEQ ID NO:4 or a homologue thereof.

Traditionally, achieving desired antibody titers can be difficult with some antigens, such as inactivated or subunit vaccines, requiring multiple administrations of the antigen. Targeted delivery of an antigen to sialoadhesin expressing macrophages using an inventive composition including antigen coupled to a sialoadhesin-specific mAb allows increased titers of antigen-specific antibodies. Targeted delivery elicits an immune response which is more efficient in comparison to administration of an unconjugated antigen, since antibodies appear earlier after administration and higher titers are reached.

Thus, in certain embodiments, a method for stimulating the immune system of a subject includes a single administration of a conjugate composition having an antigen cargo moiety hereof. Additional administrations of such a conjugate may be performed in alternative embodiment hereof.

The term “subject” refers to a vertebrate to which a conjugate is to be administered. A subject is preferably a mammal, and more preferably a human in particular embodiments. In further embodiments, a preferred subject is porcine. However, the term subject is not limited to either human or porcine subjects and methods and compositions hereof may be used in conjunction with any of various animals illustratively including cows, horses, chickens and other poultry, goats, rodents, cats, dogs and birds.

An effective amount is an amount sufficient to achieve an intended beneficial or desired result. In general, an effective amount is in the range of about 0.001 to 100 milligrams per kilogram of a subject's body weight.

In a further embodiment hereof, a cell expressing sialoadhesin is targeted in order to eliminate or inhibit the cell. For example, elimination or inhibition of sialoadhesin expressing macrophages is desirable in certain disease states, such as, but not limited to, rheumatoid arthritis.

Another embodiment hereof relates to delivery of a therapeutic agent to inhibit pathogenic infection. Thus, one embodiment of an inventive method includes targeted delivery to macrophages of a conjugate composition herein including an antimicrobial drug cargo moiety. Such targeted delivery allows the use of antimicrobial drugs that have undesirable side effects when a non-targeted delivery system is used, such as systemic administration of free antimicrobial drug.

The following illustrative examples further describe the invention.

### EXAMPLES

### Example 1

An assay for assessment of binding of a sialoadhesin binding moiety to sialoadhesin is described in this example along with an assay for assessing uptake of the bound sialoadhesin binding moiety into a cell.

In this example, primary porcine alveolar macrophages, cells which express sialoadhesin, are used to assess binding and/or uptake of a sialoadhesin binding moiety.

Porcine alveolar macrophages are isolated from 4- to 6-week old conventional Belgian Landrace pigs from a PRRSV negative herd as described in G. C. Wensvoort et al., 1991, Vet Q 13:121-30. Briefly, the main bronchus of each lung half was clamped and a needle was inserted distally. Cold PBS (3×20 ml) was injected, followed by massage of the lung tissue and aspiration. About 75% of the BAL fluid could be aspirated and was kept on ice. BAL cells were separated from fluids by centrifugation and cells were used in the experiments. Staining with mAb 41D3 showed that this procedure routinely resulted in a purity of more than 95% of sialoadhesin expressing macrophages.

The cells are cultivated in Earle's MEM, supplemented with 10% fetal bovine serum (FBS), 2 mM L-glutamine (BDH Chemicals Ltd.), 1% non-essential amino acids (Gibco BRL), 1 mM sodium pyruvate and antibiotics in a humidified 5% CO2 atmosphere at 37° C. Macrophages are preferably cultivated for 24 hours before use.

Control cells, such as non-sialoadhesin-expressing cells, may be used to assess specificity of binding and uptake. Such cells include, e.g., HEK293T cells, a human embryonic kidney cell line transfected with SV40 large T-Ag (SV40TtsA1609) described in RB. DuBridge et al., 1987, Mol. Cell. Biol. 7:379-8. HEK293T cells are maintained in DMEM supplemented with 10% FBS, 2 mM L-glutamine and a mixture of antibiotics.

Antibodies used in this example include mAb 41D3 directed against sialoadhesin. Control antibodies include isotype matched (IgG1) mAb 13D12, directed against PRV glycoprotein gD described further in H. J. Nauwynck and M. B. Pensaert, 1995, Arch. Virol. 140:1137-46; and mAb 74-22-15, reactive with SWC3, a membrane/surface protein used as a marker of porcine monocytes, macrophages and neutrophils described in M. D. Pescovitz et al., 1984, J. Immunol. 133:368-75.

Antibodies are purified using protein G sepharose column chromatography (Amersham Biosciences), dialyzed to PBS and stored at 4° C. or −70° C. prior to use.

In an assay to assess characteristics of a sialoadhesin binding moiety, cells are incubated with a sialoadhesin binding moiety under various conditions and at various concentrations. In this example, primary macrophages are incubated with purified antibodies at a concentration of 25 μg/ml for 1 hour at 4° C. to allow only attachment, but no internalization. Cells are then washed to remove unbound antibody and shifted to 37° C. to start endocytosis. After different times, cells are fixed with 3% paraformaldehyde (PF), permeabilized with 0.1% TRITON® X-100, and stained with FITC-labeled goat-anti-mouse IgG to visualize antibodies bound to and internalized in the cells. As a control, cells are fixed after the 4° C. incubation (time 0). The number of vesicles internalized in the macrophages and control cells incubated under various conditions may be counted using an appropriate technique, e.g., confocal microscopy.

Confocal analysis is performed using a scanning spectral confocal system, such as a Leica TCS SP2 laser linked to a Leica DM IRBE inverted microscope, from Leica Microsystems GmbH. Image acquisition is performed using a Leica TCS SP2 confocal software package and overlay images are produced with Adobe Photoshop CS.

In a particular example, macrophages are incubated for 60 minutes at 4° C. with the sialoadhesin-specific mAb 41D3 to allow antibody binding, but no internalization. Cells are then washed to remove unbound antibody, and shifted to 37° C. to allow internalization. Cells are fixed and stained at different times for analysis of binding and uptake into cells.

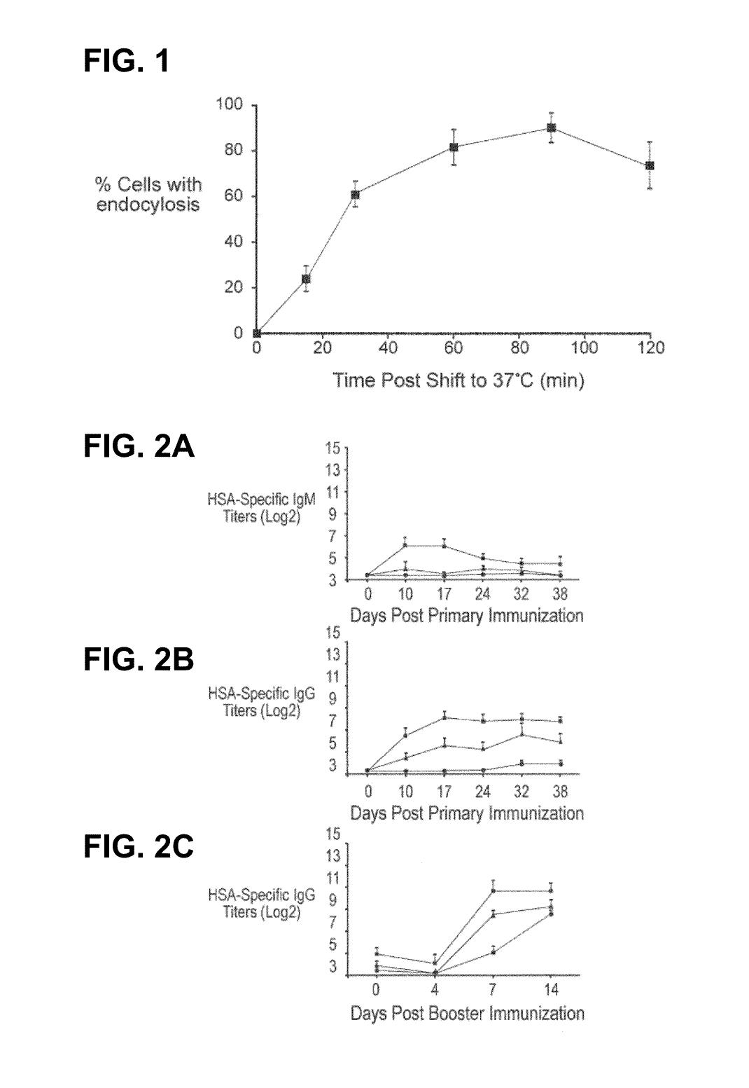

FIG. 1 shows that incubation of primary porcine macrophages with mAb 41D3 induces sialoadhesin and antibody internalization. FIG. 1 is a graph illustrating specific binding and internalization of a sialoadhesin binding moiety at different times after incubation of macrophages at 37° C. with mAb 41D3. Kinetics of uptake are demonstrated by the percentage of cells with internalized sialoadhesin at different times after incubation of macrophages at 37° C. with mAb 41D3. Data in FIG. 1 represent the means±standard deviations of three independent experiments. At time 0, a clear membrane staining is observed, and none of the macrophages contain sialoadhesin positive vesicles in the cytoplasm, as indicated by the point at the origin of the graph.

With increasing time at 37° C., the number of cells which internalized sialoadhesin and antibody increases to a maximum of 90% at 90 minutes after the 37° C. shift (FIG. 1), and then declines to 61% at 120 minutes and 50% at 180 minutes. At early time points, endocytic vesicles are mainly present in the vicinity of the plasma membrane, while with increasing time, endocytosed sialoadhesin is mainly localized to the perinuclear region. As a control, primary porcine macrophages are incubated with a non-sialoadhesin binding antibody, isotype matched mAb 13D12, or mAb 74-22-15. Cells incubated with mAb 13D12 show no staining (data not shown), while mAb 74-22-15 incubated cells show exclusive plasma membrane staining at all time points examined. Further, when mAb 41D3 is added to macrophages directly at 37° C., this results in similar internalization kinetics.

### Example 2

Various cells may be used in an assay to assess uptake and/or internalization of a sialoadhesin binding moiety. A cell line expressing sialoadhesin may be used for assay of binding and/or uptake of a sialoadhesin binding moiety.

A porcine cell line, PK-15, may be used in an assay to assess uptake and/or internalization of a sialoadhesin binding moiety. PK-15 cells are maintained as described by N. Vanderheijden et al., 2003, J. Virol. 77:8207-15. About 25% of PK-15 cells usually express sialoadhesin. PK-15 cells are optionally transfected with a sialoadhesin expression construct to enhance expression of sialoadhesin in the cells for use in an assay to assess uptake and/or internalization of a sialoadhesin binding moiety.

The porcine alveolar macrophage cell line 3D4/31 (37) is maintained in RPMI/MEM (50/50) supplemented with 10% FBS, 2 mM L-glutamine, 1% non-essential amino acids (Gibco) and a mixture of antibiotics. About 5% of 3D4/31 cells usually express sialoadhesin. 3D4/31 are optionally transfected with a sialoadhesin expression construct to enhance expression of sialoadhesin in the cells for use in an assay to assess uptake and/or internalization of a sialoadhesin binding moiety.

HEK293T cells are transfected using calcium phosphate (Cellphect transfection kit, Amersham Biosciences), and PK-15 and 3D4 cells are transfected using Lipofectamine Plus (Invitrogen), following the manufacturers' instructions. Cells are used for experiments 24 hours after transfection.

### Example 3

A primary cell or cell line characterized by little or no expression of sialoadhesin may be treated to express sialoadhesin and/or to enhance sialoadhesin expression. In particular embodiments, a cell is transfected with a sialoadhesin expression construct in order to provide a cell used in an assay for assessment of binding and/or uptake of a sialoadhesin binding moiety. An expression construct including a nucleotide sequence encoding pig, mouse or human sialoadhesin detailed herein may be used. A pcDNA3.1/Sn plasmid containing the porcine sialoadhesin cDNA cloned into the pcDNA3.1 vector (Invitrogen) is described in N. Vanderheijden et al., 2003, J. Virol. 77:8207-15.

### Example 4

In further embodiments, a cell is treated with a stimulator of sialoadhesin expression in order to provide a cell used in an assay for assessment of binding and/or uptake of a sialoadhesin binding moiety. Stimulators of sialoadhesin expression include interferon-alpha. In this example, human peripheral blood mononuclear cells (PBMC) are isolated from heparinized blood from a healthy donor via centrifugation on Ficoll-paque according to the manufacturer's instructions (Amersham Biosciences). Monocytes are semi-purified by plastic adhesion and several washing steps to remove non-adherent lymphocytes. Flow cytometric analysis with a mouse-anti-human CD14 antibody shows that this procedure routinely results in a purity of the monocytes of >90%. Cells are cultivated for 3 days in RPMI medium with 10% FBS (RPMI-FBS) or RPMI-FBS with interferon-gamma, 500 U/ml and Tumor Necrosis Factor-alpha (TNF-alpha), 10 ng/ml, as described in A. Hartnell et al., Blood, 2001. 97(1): p. 288-96, or in RPMI-FBS supplemented with interferon-alpha (100 U/ml).

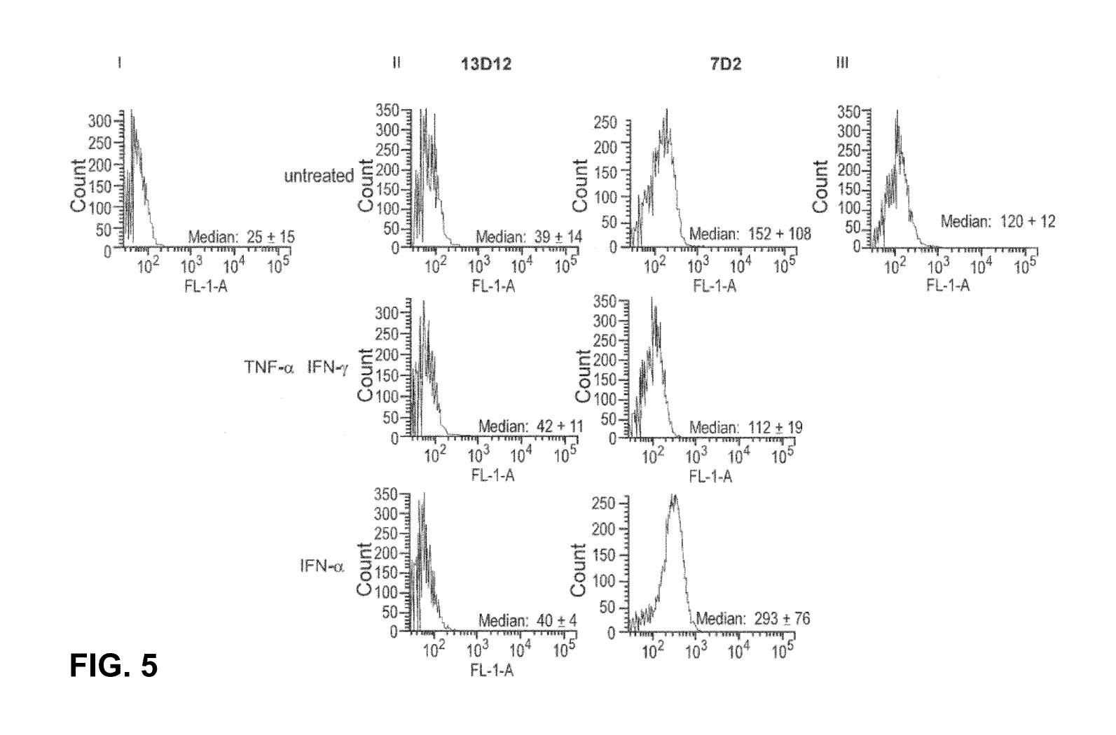

Cells are lifted from a plastic substrate to which they have adhered by incubation with ice-cold PBS for 30 minutes at 4° C. Cells are first incubated at 4° C. with a mouse anti-human-sialoadhesin specific antibody, 7D2, or a isotype-matched irrelevant control antibody, 13D12. Next, cells are fixed with paraformaldehyde (3% in PBS) or incubated at 37° C. for 1 hour for the internalization of the bound antibodies followed by paraformaldehyde fixation. Cells are washed three times and subsequently incubated with FITC-labeled goat-anti-mouse Ab (Molecular Probes). Some of the cells are double stained with APC-labeled mouse-anti-human CD14 (BD Pharmingen). Finally, the cells are washed two times, resuspended in PBS and analyzed with a Becton-Dickinson (San Jose, Calif.) FACScalibur. Ten thousand cells are analyzed for each sample, and four parameters are stored for further analysis: forward light scatter, sideward light scatter, green and red fluorescence, and results of this analysis are shown in FIG. 5.

Flow cytometric analysis of sialoadhesin expression yields data representative of three experiments. The control sample (shown at I in FIG. 5) is treated as the others during staining but without antibodies. Untreated and cytokine treated cells are stained with a control antibody 13D12 and with a human sialoadhesin-specific antibody 7D2, shown at II in FIG. 5. After the binding of 7D2, one sample was incubated for 1 hour at 37° C. to enable the receptor, sialoadhesin, to internalize the antibodies, shown at III in FIG. 5. IFN-alpha treatment clearly induces Sn expression and the induced Sn is able to internalize monoclonal antibody 7D2. Internalization of monoclonal antibody 7D2 is demonstrated by the reduction in the median fluorescence intensity upon surface staining of interferon-alpha treated cells incubated at 37° C. with FITC labeled goat-anti-mouse IgG (Molecular Probes, Invitrogen), as shown in FIG. 5.

In two of the three experiments using human monocytes, low levels of sialoadhesin is present on the untreated cells, in the third experiment it is absent. Treatment of the cells with TNF-alpha and IFN-gamma induces Sn expression; however, treatment with IFN-alpha leads to a significantly higher expression of sialoadhesin. Similar results are obtained using monocytes isolated from peripheral blood from pigs and treated with IFN-alpha to induce Sn expression.

Sialoadhesin induced by IFN-alpha treatment is biologically active as shown by sialic acid binding capacity of IFN-alpha treated monocytes. Red blood cells contain sialic acids on their surface which allows them to bind to monocytes if these have functional expression of sialoadhesin. Monocytes are grown in 96-well plates for three days as described above. Next, they are incubated for 30 minutes at 37° C. with normal medium or medium supplemented with neuraminidase (Roche) 30 U/ml to remove sialic acids present on the surface. After removal of the neuraminidase, monocytes are incubated for 1 hour at room temperature with a 0.1% solution of human erythrocytes. Excess erythrocytes are washed away and binding of red blood cells to sialoadhesin is visualized via light microscopy. When sialic acids present on the surface of the monocytes are not removed, red blood cells are unable to bind to the monocytes under any conditions tested. However when sialic acids are removed from monocytes, red blood cells are able to bind in some conditions. Little binding is observed in cells grown in normal medium. Cells treated with TNF-alpha and IFN-gamma do not bind RBC. However, in the IFN-alpha treated cells clear formation of rosettes, that is, red blood cells bound to monocytes are observed. These data confirm the results obtained in the flow-cytometric analysis showing that biologically active sialoadhesin is induced in monocytes by IFN-alpha treatment.

The ability of cytokine-induced sialoadhesin expression on human monocytes to internalize a sialoadhesin binding moiety is also shown in this example. Human monocytes are isolated as described above and treated with IFN-alpha for three days to induce human sialoadhesin. Cells are then incubated with human sialoadhesin-specific mAb 7D2 for 60 minutes at 37° C. to allow binding and internalization. As a control, the cells are incubated with mAb 7D2 at 4° C. At 4° C., cells are no longer capable of mediating internalization, thus this control should only binding of the sialoadhesin binding moiety mAb 7D2. After 60 minutes, the cells are fixed with 3% paraformaldehyde in PBS and permeabilized by incubation with 0.1% Triton X-100 in PBS for 2 minutes. MAb 7D2 is visualized by incubation with FITC-labeled goat-anti-mouse (Invitrogen). Cortical actin is also visualized, using TexasRed labeled Phalloidin, to allow discrimination of surface bound and internalized sialoadhesin. Surface expression of sialoadhesin and binding of sialoadhesin binding moiety mAb 7D2 is observed at time 0. Following incubation for 60 minutes at 37° C., internalized sialoadhesin and sialoadhesin binding moiety mAb 7D2 is observed in the IFN-alpha treated human monocytes.

Thus, an in vitro system for evaluation of human sialoadhesin binding moieties and conjugates of human sialoadhesin binding moieties is provided which is analogous to the in vitro and in vivo pig system for evaluation of sialoadhesin binding moieties and conjugates of sialoadhesin binding moieties.

### Example 5

The effect of interferon-alpha on sialoadhesin expression in human THP-1 cells, a monocytic continuous cell line, is tested to determine if sialoadhesin is internalized in these cells upon stimulation with an antibody as a sialoadhesin binding moiety. THP-1 cells are deposited with the American Type Culture Collection (ATCC) and are identified by ATCC Number TIB-202. THP-1 cells are cultivated for 3 days in RPMI medium with 10% FBS (RPMI-FBS) or RPMI-FBS with interferon-gamma (500 U/ml) and TNF-alpha (10 ng/ml) or in RPMI-FBS supplemented with interferon-alpha (100 U/ml).

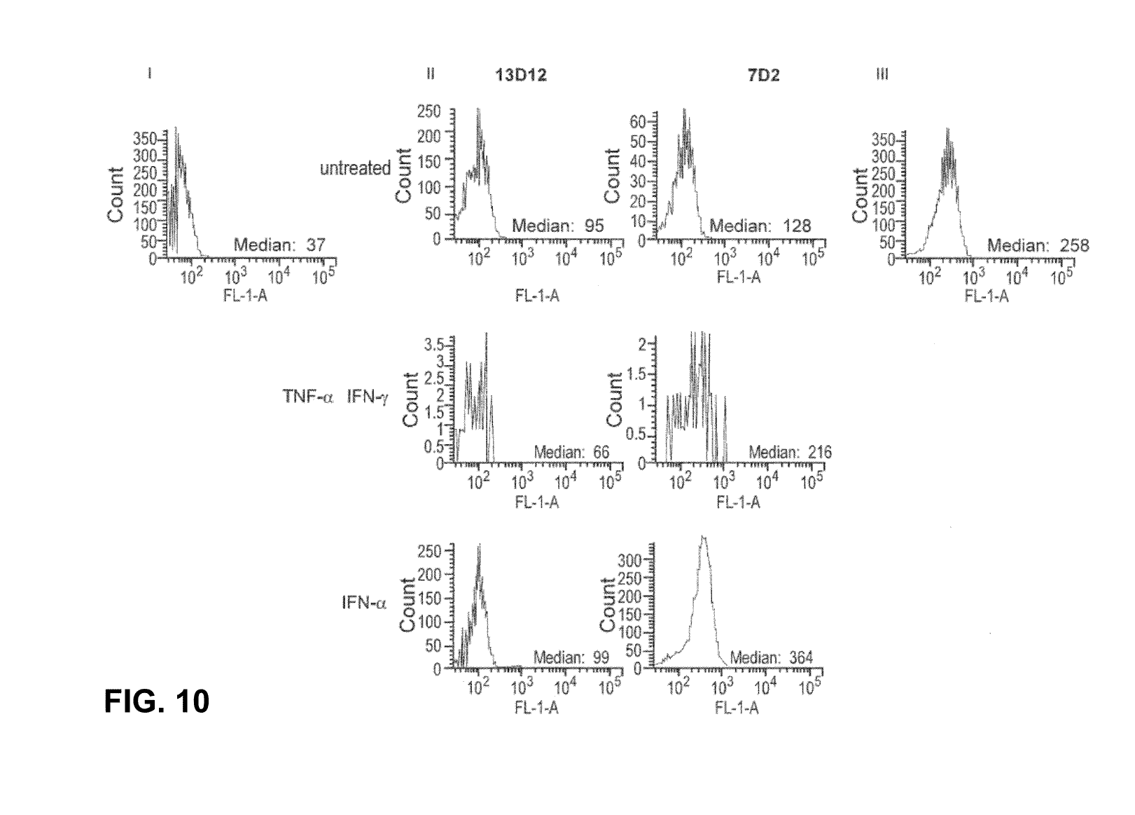

THP-1 cells are incubated at 4° C. with a human-sialoadhesin specific antibody, 7D2, or an isotype-matched irrelevant control antibody, 13D12. Next, cells are fixed with paraformaldehyde (3% in PBS) or incubated at 37° C. for 1 hour to allow antibody induced internalization of sialoadhesin and the bound antibody followed by paraformaldehyde fixation and permeabilization of the cells with 0.1% Triton X-100. Cells are washed 3 times and subsequently incubated with FITC-labeled goat-anti-mouse Ab (Molecular Probes). Some of the cells are double stained with APC-labeled mouse-anti-human CD14 (BD Pharmingen). The cells are washed two times, resuspended in PBS and analyzed with a Becton-Dickinson (San Jose, Calif.) FACScalibur. Ten thousand cells are analyzed for each sample, and four parameters were stored for further analysis: forward light scatter, sideward light scatter, green and red fluorescence (FIG. 10). These data show that IFN-alpha treatment induces human sialoadhesin on THP-1 cells, and that upon stimulation with mAb 7D2 at 37° C., a decrease in cell surface sialoadhesin fluorescence is observed, indicative of internalization of the antibody bound to sialoadhesin.

FIG. 10 shows flow cytometric analysis of sialoadhesin expression and antibody induced sialoadhesin internalization. Histograms are representative for three experiments. The control sample (I) is treated as the others during staining but without antibodies. Untreated and cytokine treated cells are stained with a control antibody 13D12 and with a human sialoadhesin-specific antibody 7D2 (II). After the binding of 7D2, one sample was incubated for 1 hour at 37° C. to enable the receptor to internalize the antibodies (III). IFN-alpha treatment clearly induces sialoadhesin expression and the induced sialoadhesin is able to internalize monoclonal antibody 7D2 as shown by the decreased median which lowers from 364 to 258 upon incubation at 37° C.

Confocal microscopy is used in this example to visualize internalization of sialoadhesin and bound sialoadhesin binding moiety mAb 7D2. THP-1 cells are incubated with human sialoadhesin-specific mAb 7D2 for 60 minutes at 37° C. to allow internalization. As a control, a time 0 was analyzed by incubating the cells with mAb 7D2 at 4° C. At 4° C., cells are no longer capable of mediating internalization, thus this control should only show binding to sialoadhesin at the cell surface without internalization. After 60 minutes, the cells are fixed with 3% paraformaldehyde in PBS and permeabilized by incubation with 0.1% Triton X-100 in PBS for 2 minutes. Internalized antibodies are visualized by incubation and staining with FITC-labeled goat-anti-mouse (Invitrogen). Surface labeling of these cells is observed at time 0, while at time 60, sialoadhesin and bound antibody is observed internalized in the THP-1 cells.

Thus, an in vitro system is provided including the human monocytic THP-1 cell line, allowing further analysis of antibody-induced human sialoadhesin internalization without the need of isolating primary blood monocytes or macrophages.

### Example 6

Chemical cross-linking of a sialoadhesin binding moiety and a cargo moiety is described. In this example, human serum albumin (HSA) is a cargo moiety which is an antigen to be conjugated to mAb 41D3, a sialoadhesin binding moiety, to form a conjugate composition. In addition, as a control, human serum albumin (HSA) is conjugated to a non-sialoadhesin binding antibody, mAb 13D12.

For chemical cross-linking of HSA and the mAb in this example, a two step cross-linking protocol is used. The amine reactive cross-linker LC-SMCC (Pierce) is coupled to the purified mAb 41D3 by incubating 600 micrograms of LC-SMCC with 20 milligrams of mAb in 8 milliliters phosphate buffered saline (PBS) for 30 minutes at room temperature. The amine-reactive cross-linker SPDP (Pierce) is coupled to the purified HSA by incubating 2 milligrams SPDP with 40 milligrams HSA in 8 milliliters PBS, for 30 minutes at 37° C. The SPDP-HSA is then activated by addition of 125 micrograms DTT, which results in the formation of a thiol activated protein. Both the mAb-LC-SMCC and the thiol activated HSA are then dialyzed to PBS at 4° C. using a membrane with a 10-14 kDa cutoff to remove residual unreacted LC-SMMC, SPDP and DTT. The mAb-LC-SMCC and the thiol activated HSA are then mixed together and incubated at 37° C. for 30 minutes to allow the thiol group on HSA to react with the maleimide end of the LC-SMCC on the mAb, resulting in the formation of a covalent thio-ether bond. After the coupling reaction, the mixture is dialyzed again towards PBS using a membrane with a 100 kDa cut off, to remove any unreacted HSA from the mixture.

A similar reaction is performed to generate a control conjugate including human serum albumin (HSA) conjugated to a non-sialoadhesin binding antibody, mAb 13D12.

Samples taken in between different steps of the cross-linking protocol may be analyzed to confirm formation of a conjugate. For example, such samples may be separated by SDS-PAGE on a 7% gel and proteins stained with a reagent such as Coomassie blue in order to visualize the reactants and reaction products.

### Example 7

Internalization of a conjugate composition including a sialoadhesin binding moiety and a cargo moiety is demonstrated in primary macrophages. In this example, the HSA-mAb 41D3 conjugate and HSA-mAb 13D12 conjugate are incubated for 1 hour at 37° C. with sialoadhesin expressing primary porcine macrophages. Cells in separate culture dishes are incubated for 1 hour at 37° C. with mAb 41D3, mAb 13D12 or with HSA alone. Cells are then washed, fixed by incubating with 3% paraformaldehyde for 10 minutes and permeabilized by incubating with 0.1% Triton X-100 for 2 minutes.

HSA is detected in these preparations by incubating the cells with a HSA-specific biotinylated polyclonal pig serum, followed by incubation with FITC-labeled streptavidin FITC (Molecular Probes). The monoclonal antibodies are detected with TxRed-labeled goat-anti-mouse Ig (Molecular Probes). The cells are then analyzed using an appropriate technique, such as confocal microscopy.

Confocal analysis is performed using a scanning spectral confocal system, such as a Leica TCS SP2 laser linked to a Leica DM IRBE inverted microscope, from Leica Microsystems GmbH. Image acquisition is performed using a Leica TCS SP2 confocal software package and overlay images are produced with Adobe Photoshop CS.

Analysis demonstrates mAb 41D3 internalization both when it is coupled to HSA or not, indicating that the coupling reaction had no effect on the ability of mAb 41D3 to bind to sialoadhesin and to induce internalization. Internalization of free HSA is either absent, or at very low levels when it is added to macrophages not coupled to mAb 41D3, but a clear internalization of HSA is observed when it is coupled to mAb 41D3. Further, internalized HSA co-localizes with mAb 41D3 in confocal images of cells treated with the HSA-mAb 41D3 conjugate. Coupling HSA to mAb 41D3 results thus in co-internalization of HSA with mAb 41D3 via the sialoadhesin receptor.

Thus, contact of a conjugate including HSA coupled to the sialoadhesin-specific mAb 41D3 with primary, sialoadhesin expressing macrophages, results in sialoadhesin-dependent uptake of HSA into macrophages, while addition of non-coupled HSA to macrophages did not result in efficient HSA uptake.

### Example 8

Immunization is performed using conjugate compositions herein in this example. Six week old conventional pigs are purchased from a porcine arterivirus negative farm and housed in isolation units with HEPA filtered air following the recommendations of the ethical committee of the Faculty of Veterinary Medicine, Ghent University. Six pigs are immunized with one milligram of a conjugate having HSA coupled to the sialoadhesin-specific mAb 41D3. Three pigs are immunized with one milligram of a control conjugate having HSA coupled to the control mAb 13D12. Each immunization includes administration of the conjugate in 3 milliliters PBS, of which 1.5 milliliters is administered intravenously and 1.5 milliliters is administered intramuscularly. As a control, six pigs are immunized with one milligram unconjugated HSA.

Blood samples are collected before immunization and at days 10, 17, 24, 32 and 38 after immunization. Three months later, blood is sampled again and the pigs are boostered with one milligram HSA by intramuscular injection.

Serum obtained from immunized pigs is analyzed for the presence of HSA-specific IgM and IgG antibodies by ELISA. The HSA-specific IgM, and IgG antibody titers are determined with an indirect ELISA as described in Y. E. Van der Stede et al., 2001, Vaccine 19:1870-8; and F. Verdonck et al., 2005, J. Control Release 104:243-58. Briefly, the wells of a 96-well Polysorb Immuno microtiter plate (NUNC) are coated with HSA at a concentration of 30 micrograms/milliliter in PBS for 2 hours at 37° C. The plates are then washed and the remaining binding sites are blocked overnight at 4° C. with PBS supplemented with 0.2% TWEEN®80. Two-fold serial dilutions of the serum samples (starting from 1/10) in ELISA dilution buffer (PBS+0.05% TWEEN®20) are added to the plate, followed by the swine-specific IgM, or IgG MAb, such as described in D. Van Zaane and M. M. Hulst, 1987, Vet Immunol. Immunopathol. 16:23-36, and peroxidase-conjugated rabbit-anti-mouse polyclonal antibodies (Dako) supplemented with 2% pig serum. ABTS and H₂O₂ are used as chromogen and substrate and the optical density is spectrophotometrically measured at 405 nm (OD405). The cut-off values are calculated as the mean OD₄₀₅-value of all sera (dilution 1/10) at day 0, increased with three times the standard deviation. The antibody titer is the inverse of the highest dilution that still had an OD₄₀₅ higher than the calculated cut-off value.

FIG. 2 shows means of HSA specific IgM (FIG. 2A) and IgG (FIG. 2B) serum titers (±SEM) after primary immunization. FIG. 2C shows means of HSA specific IgG serum titers after booster immunization. Square symbols indicate pigs immunized with HSA coupled to Sn-specific mAb 41D3; triangle symbols indicate pigs immunized with HSA coupled to irrelevant control mAb; and circle symbols indicate pigs immunized with free HSA.

After primary immunization, low to undetectable titers of IgM antibodies are detected in the pigs immunized with HSA alone, or with HSA coupled to the control mAb 13D12. In contrast, IgM antibodies are present starting from 10 days post immunization (dpi) in the pigs immunized with HSA coupled to the sialoadhesin-specific mAb 41D3. These antibodies remained at a nearly constant level until 17 dpi, and started to decline from 24 dpi as illustrated in FIG. 2A.

Similarly, HSA-specific IgG antibodies are undetectable in pigs immunized with HSA alone until 24 dpi, and low titers are detected from 32 dpi. In pigs immunized with HSA coupled to the control mAb 13D12, low titers of HSA-specific IgG antibodies could be detected from 10 dpi which reached maximum titers at 32 dpi. Cross-linking of HSA and a non-sialoadhesin binding antibody stimulates some HSA-specific IgG antibody response. In contrast, pigs immunized with HSA coupled to the sialoadhesin-specific mAb developed high titers of IgG antibodies already starting at 10 dpi. Maximum antibody titers are detected at 17 dpi and these remained constant until 38 dpi as illustrated in FIG. 2B.

To investigate if immunization with HSA coupled to the sialoadhesin-specific mAb 41D3 had an effect on the induction of HSA-specific memory cells, all animals are boosted 3 months after primary immunization with HSA alone. At the time of the booster immunization and at 4 dpi, all animals had low to undetectable HSA-specific IgG titers. Starting from 7 dpi, an IgG antibody response is detected in all animals, but the highest titers are detected in the animals which received HSA coupled to the sialoadhesin-specific mAb 41D3 as the primary immunization as is shown in FIG. 2C.

Pigs immunized with the HSA-mAb41D3 constructs showed the highest IgG and IgM antibody titers throughout the study, which indicates that coupling HSA to the sialoadhesin-specific mAb greatly enhances both the speed of induction and the titers of HSA-specific IgG and IgM antibodies.

Thus, targeted delivery of an immunogen to macrophages is possible by coupling the immunogen to the sialoadhesin-specific mAb, and this affects the humoral immune response, enhancing both the speed of induction and the titers of antigen-specific antibodies.

### Example 9

Sialoadhesin binding moiety/viral protein conjugate, administration and immune response. In this example, influenza virus hemagglutinin (HA) is conjugated to sialoadhesin binding moiety monoclonal antibody (mAb) 41D3. Influenza virus hemagglutinin conjugated to sialoadhesin binding moiety monoclonal antibody 41D3 is either the native protein purified from virus or a recombinant form produced in eukaryotic cells. Hemagglutinin conjugated to sialoadhesin binding moiety monoclonal antibody 41D3 is chemically cross-linked with mAb 41D3 in this example and injected in pigs to demonstrate and evaluate the capacity of the conjugates to induce HA-specific antibodies.

### Purification of Native Hemagglutinin

In order to obtain native hemagglutinin, a split H1N1 component is prepared essentially as described by Van Reeth et al., Vet. Rec., 2003. 153(1): p. 9-13. Ten-day-old embryonated SPF chicken eggs are inoculated with the H1N1 swine influenza strain A/swine/Belgium/1/98. Allantoic fluid is collected 72 hours post inoculation and red blood cells and cell debris are removed via centrifugation. The clarified allantoic fluid is then centrifuged to pellet the virus, 70,000 g at 4° C. for 90 minutes. Virus pellets are resuspended overnight at 4° C. in TSE buffer, 10 mM Tris-HCl pH7.4, 100 mM NaCl and 1 mM EDTA. Presence of influenza virus is confirmed with a hemagglutination (HA) test such as described by K. S. Van Reeth et al., Vet. Rec., 2003. 153(1): p. 9-13 followed by concentration and purification via ultracentrifugation on a linear 20 to 60% (w/v) sucrose gradient, 130,000 g at 4° C. for 14 hours. Gradient fractions containing virus are identified with an HA test, pooled, dialyzed in a slide-a-lyzer dialysis cassette, 10,000 MWCO, against phosphate buffered saline (PBS) to remove sucrose and concentrated by dialysis in a 20% polyethylene glycol (PEG-20,000) solution. Finally, the hemagglutinin is released from the purified and concentrated virus by centrifugation on a linear denaturing 20 to 60% (w/v) sucrose gradient consisting of 0.1% TWEEN® 80 and 1.2% sodium deoxycholate in TSE buffer, 130,000 g at 4° C. for 14 hours. Fractions containing hemagglutinin are identified with an HA test, pooled, dialyzed in a slide-a-lyzer cassette (10,000 MWCO) against PBS and concentrated by dialysis in a 20% PEG solution. Residual infectious virus is inactivated by UV treatment of the solution (5 J/cm²). Complete inactivation is confirmed by inoculation on MDCK cells and two blind passages in 10-day-old embryonated SPF chicken eggs.

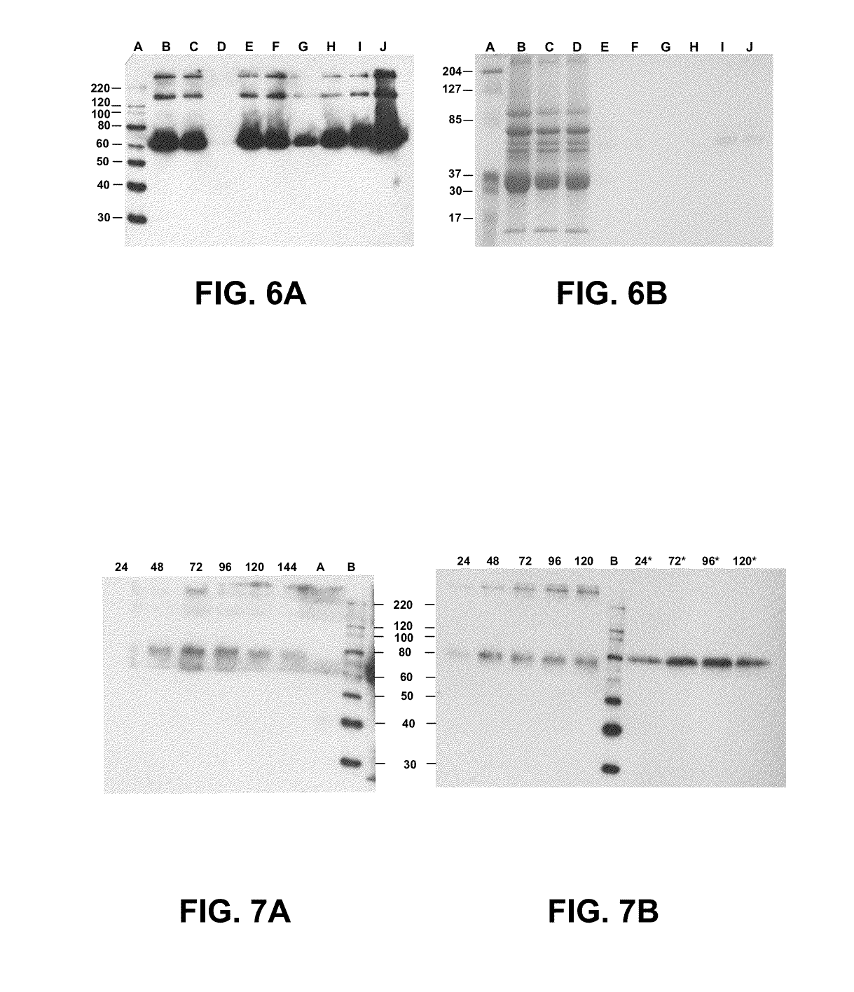

The purification process is analyzed via SDS-PAGE followed by Western blotting and Coomassie blue staining. HA is clearly present in the original allantoic fluid, but also in the purified solution after the denaturing sucrose gradient. HA can be detected as a monomer and as two different multimers, most likely a dimer and a trimer during all steps of the purification process. FIG. 6A shows SDS-PAGE analysis of the presence and purity of native influenzavirus hemagglutinin in different fractions obtained during purification includes detection of HA via western blotting using a monoclonal antibody directed against HA of the H1N1 virus. FIG. 6B shows detection of all proteins in the samples is accomplished via Coomassie blue staining. In both FIGS. 6A and 6B: Lane A: marker, lane B: allantoic fluid after removal of RBC, lane C: allantoic fluid after removal of cell debris, lane D: supernatant after pelleting the virus, lane E: the virus pellet (1/100 dilution), lane F: virus after the first sucrose gradient and after removal of sucrose (1/100 dilution), lanes G-J: virus after denaturing sucrose gradient: lane G: fraction with HAU 64 and 128 (1/100), lane H: fraction with HAU≧256 (1/100), lane I: fraction with HAU 64 and 128 (undiluted), lane J: fraction with HAU≧256 (undiluted). HA can be detected as a monomer and as two different multimers, most likely a dimer and a trimer.

### Production of Recombinant Hemagglutinin

In further embodiments, a recombinant influenza virus hemagglutinin protein is produced. The recombinant influenza virus hemagglutinin protein used in this example includes the extracellular domain of hemagglutinin fused to the V5-His tag in the pcDNA3.1 D/V5-His vector (Invitrogen). Viral RNA is isolated from H1N1 swine influenza strain A/swine/Belgium/1/98 via the RNEASY® mini kit (Qiagen) and subsequently converted into cDNA via random primers (Invitrogen) and SuperScript II reverse transcriptase (Invitrogen) followed by an RNase H (Gibco) treatment. The obtained single stranded cDNA serves as template for PCR amplification of the HA sequence using following primers: forward primer SEQ ID NO:1 and reverse primer SEQ ID NO:2 (Invitrogen). The PCR fragment is then cloned in the pcDNA3.1D/V5-His vector. The sequence is verified via restriction digest and sequencing. The isolated and verified nucleotide sequence encoding the extracellular domain of influenza virus hemagglutinin is shown and referred to as SEQ ID NO:3 herein.

### Extracellular Domain of Influenza Virus Hemagglutinin—SEQ ID NO:3

Production and purification of the recombinant, soluble HA is demonstrated in a human embryonic kidney cell line, HEK293T. HEK293T cells are transfected using calcium phosphate to produce the soluble hemagglutinin. Sixteen hours post-transfection, medium is replaced by fresh medium with or without fetal bovine serum (FBS). Samples are taken every 24 hours post transfection and analyzed via SDS-PAGE and western blotting to determine at what time post transfection the supernatant contains the highest concentrations of soluble HA (FIGS. 7A and 7B). The recombinant, soluble HA is produced in HEK293T cells, no matter whether FBS is present in the serum or not. In the absence of FBS, the maximum amount of HA in the serum is reached at 72 hours post transfection. In the presence of FBS, the amount of HA stays the same until 120 hours post transfection. The recombinant HA is produced as a monomer and, to a lesser extent as a trimer, which is confirmed by the disulfide-reducing agent beta-mercaptoethanol.

FIGS. 7A and 7B show SDS-PAGE analysis of the production of recombinant HA with a V5-His tag. The recombinant HA is produced in the absence, FIG. 7A or in the presence FIG. 7B of fetal bovine serum. Samples are taken every 24 hours post transfection as indicated above the lanes in FIGS. 7A and 7B. HA is detected via a monoclonal antibody recognizing the V5 tag. Under non-reducing conditions, HA is mainly present in the supernatant as a monomer, although it also forms trimers. In the presence of the disulfide-reducing agent beta-mercaptoethanol, indicated with an asterisk* in FIGS. 7A and 7B, HA is only present as a monomer, confirming that the high molecular weight protein was indeed an HA trimer. The molecular weight of the proteins is determined via a marker in lane A (prestained) and B.

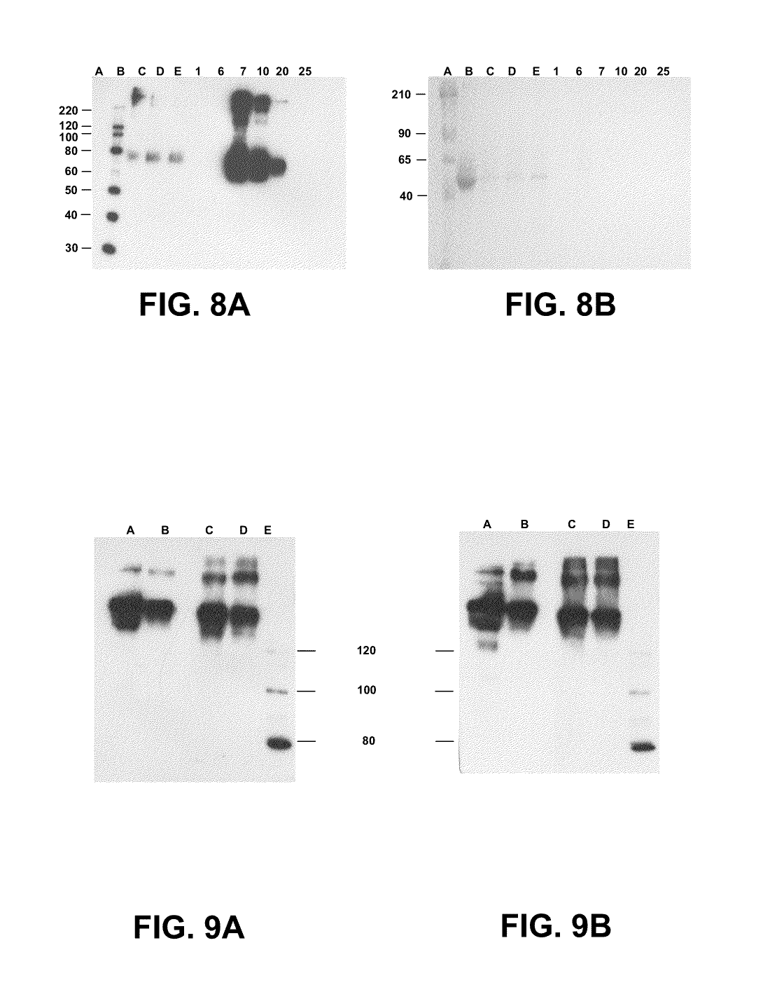

After collection of the supernatant, the recombinant HA is purified via Ni-NTA beads according to the manufacturer's instructions (Qiagen). Because of interference of the FBS with this purification step, HA is further produced without FBS and the supernatant is collected at 72 hours post transfection. Different fractions are taken during the purification process and HA is visualized via SDS-PAGE followed by Western blot or Coomassie blue staining as shown in FIGS. 8A and 8B, respectively. The recombinant HA is present in the original supernatant, but not in the flow through. HA is clearly concentrated, both the monomer and the trimer. FIGS. 8A and 8B show SDS-PAGE analysis of the purification process of recombinant HA-V5-His via Ni-NTA beads. SDS-PAGE is followed by Western blotting and detection of HA via a monoclonal antibody directed against the V5-tag to identify the fractions containing HA, FIG. 8A, or by Coomassie blue staining to visualize the purity of the HA, FIG. 8B. Lane A: marker, lane B: original supernatant with FBS, lane C and D: original supernatant from two different productions without FBS, lane E: flow through of purification, following lanes: elution fractions of 0.8 ml, fractions are indicated with their respective number above the lanes. HA is present in all original supernatants but not in the flow through. HA is clearly concentrated, both the monomer and the trimer.

Conjugation of Antibodies with HA

Hybridomas producing monoclonal antibody 41D3, described in X. Duan et al., Adv. Exp. Med. Biol., 1998. 440: p. 81-8, or monoclonal antibody 13D12, described in H. J. Nauwynck and M. B. Pensaert, Arch. Virol., 1995. 140(6): p. 1137-46, directed against porcine sialoadhesin or an isotype matched (IgG1) irrelevant control antibody, respectively, are cultivated and supernatant is collected every 72 hours. Antibodies are purified via protein G sepharose columns as described by the manufacturer (GE Healthcare).

Influenza virus hemagglutinin Type A/swine/Belgium/1/98 having protein sequence identified as GenPept Accession number AY590824, and herein as SEQ ID NO:4, is used in this example as a cargo moiety conjugated to mAb 41D3.

The purified antibodies are coupled to influenza hemagglutinin (HA) SEQ ID NO:4 via a disulfide-bridge. To accomplish this, the 41D3 monoclonal antibody, the isotype matched control monoclonal antibody and HA are activated with the cross-linker SPDP (N-succinimidyl-3-(2-pyridyldithio)-propionate) according to the manufacturers' instructions (Pierce Biotechnology). For HA, the SPDP is activated via dithiothreitol (DTT). The activated proteins are purified from the unreacted cross-linkers via PD-10 desalting columns (Amersham Biosciences). The activated proteins are mixed in a 1:1 antibody:HA ratio. The uncoupled HA is removed from the coupled products, 41D3-HA and control monoclonal antibody-HA, by dialysis with a float-a-lyzer (Spectra/Por) with a MWCO 100,000.

Coupling of the antibodies and HA to form conjugates is verified via SDS-PAGE followed by western blotting and by analysis of uptake of the coupling products by primary alveolar macrophages. For both antibodies there is a clear shift towards a bigger protein, which confirms that each antibody is coupled with HA. FIGS. 9A and 9B show visualization of coupling of antibodies 13D12, FIG. 9A, or 41D3, FIG. 9B, with isolated native HA. Samples taken during the coupling process are analyzed via SDS-PAGE followed by western blotting and detection via a mixture of 3 monoclonal antibodies recognizing HA of H1N1. Lane A: original antibody, lane B: SPDP treated antibody after PD-10 desalting column, lane C: HA coupled with antibody, lane D: HA coupled with antibody after dialysis and lane E: marker.

### Vaccinations

Twelve (12) six-week-old pigs are obtained from an influenza virus-seronegative farm and randomly assigned to three groups of four pigs. The animals are housed in isolation units with high efficiency particulate air (HEPA) filters. Water and feed are provided ad libitum. The first group of four pigs is immunized with 1 mg HA-13D12 conjugate per pig, the second group with 1 mg HA-41D3 conjugate per pig, and the control group with the same volume of PBS without any protein. For each pig, the conjugate is diluted in 3 ml PBS. Half of the conjugate is injected intravenously and the other half intramuscularly in the neck.

Blood samples are collected from all pigs at the time of immunization and on day 4, 7, 11, 14 and 18 after immunization. The sera are examined in hemagglutination inhibition (HI) tests, virus neutralization (VN) test and in immunoperoxidase monolayer assays (IPMA) as described in K. Van Reeth, S. Van Gucht, and M. Pensaert, Vet. Rec., 2003. 153(1): p. 9-13.

### Hemagglutination Assay (HA)

Samples containing influenzavirus hemagglutinin are serially diluted and mixed with 0.5% chicken erythrocytes for one hour at room temperature. The highest dilution of that still shows hemagglutination is considered to be the hemagglutinating titer.

### Hemagglutination Inhibition (HI)

The sera are examined in a hemagglutination inhibition (HI) test against H1N1 strain A/swine/Belgium/1/98. The inactivated sera are first treated with receptor-destroying enzyme (RDE) from Vibrio cholera, followed by inactivation of the enzyme via sodium citrate treatment. Afterwards, the sera are absorbed on chicken erythrocytes to remove non-specific inhibitors of influenza hemagglutination. The HI test is carried out according to standard procedures including positive and negative controls. Because of the pretreatments, the starting dilution of the sera was 1:10 followed by two-fold serum dilutions. Furthermore, each well was mixed with four hemagglutination units of the H1N1 strain and 0.5% chicken erythrocytes. After 1 hour incubation, the results are interpreted. In the presence of HA recognizing antibodies, no hemagglutination can be observed and the RBC will all be together in one spot on the bottom of the plate. The HI titer is the reverse of the titer needed for complete inhibition of hemagglutination. As a reference, positive and negative control sera are included in the HI tests.

### Virus Neutralization (VN)

Sera are also examined in a virus neutralization (VN) test for the presence of H1N1 neutralizing antibodies. Two-fold serum dilutions are incubated with 100 tissue culture infectious doses (TCID₅₀) of A/swine/Belgium/1/98 virus. Madin-Darby canine kidney (MDCK) cells are then added at a concentration of 600,000 cells per ml. After 24 hours incubation, virus-positive cells are detected by immuno-peroxidase staining. Starting dilution of the sera was 1:2.

### Immuno-Peroxidase Monolayer Assay (IPMA)

Finally, sera are analyzed via an immuno-peroxidase monolayer assay (IPMA) for the presence of influenza recognizing antibodies. Therefore, MDCK cells are grown for 24 hours in the presence of 1000 TCID₅₀ of A/swine/Belgium/1/98 virus. After fixation, cells were incubated with two-fold serum dilutions followed by immuno-peroxidase staining for antibody detection. Starting dilution of the sera is 1:2. Results are shown in FIG. 12. FIG. 12 shows mean immuno-peroxidase monolayer assay (IPMA) titers of pigs immunized with 13D12-HA or 41D3-HA. For each group, four pigs are immunized with native hemagglutinin (HA) coupled with the isotype matched (IgG1) control antibody 13D12, or HA coupled with 41D3, a monoclonal antibody directed against porcine sialoadhesin. Serum is collected at day 0, 4, 7, 11, 14 and 18 after immunization and analyzed via an IPMA. Pigs immunized with HA coupled to the sialoadhesin specific mAb 41D3 show a faster induction and higher titers of IPMA antibodies.

### Example 10

A conjugate composition including a sialoadhesin binding moiety and a cytotoxic agent is generated in this example. The cytotoxic agent saporin is a 30 kDa plant enzyme, belonging to the family of ribosome inactivating proteins (RIP). Saporin may be isolated from seeds of the plant Saponaria officinalis according to methods known in the art or obtained commercially. Saporin is used in this example as a representative of cytotoxic agents which may be included in a conjugate herein.

Saporin alone has no cell binding moiety and can thus not enter the cell. However, following conjugation with a sialoadhesin binding moiety to produce a conjugate, saporin is co-internalized in the cell along with the sialoadhesin binding moiety. Saporin is also representative of cytotoxic agents which are capable of linkage to a sialoadhesin binding moiety by a disulfide bond between the sialoadhesin binding moiety and saporin. Further cytotoxic agents are described in G. R. Thrush et al., Annu. Rev. Immunol., 1996. 14: p. 49-71. The disulfide bond between the sialoadhesin binding moiety and saporin allows dissociation of toxin.

Conjugation of a Cytotoxic Agent with a Sialoadhesin Binding Moiety

The purified antibody 41D3 is conjugated to saporin (Sigma) via a disulfide-bridge. Therefore, the antibody and saporin are activated with the cross-linker SPDP (N-succinimidyl-3-(2-pyridyldithio)-propionate) according to the manufacturers' instructions (Pierce Biotechnology). For saporin, the SPDP is activated with dithiothreitol and the proteins are purified from the unreacted cross-linkers with PD-10 desalting columns (Amersham Biosciences). The activated proteins are mixed in a 1:1 antibody:saporin ratio. The uncoupled saporin was removed from the coupling products by dialysis with a float-a-lyzer (Spectra/Por) with a MWCO 100,000.

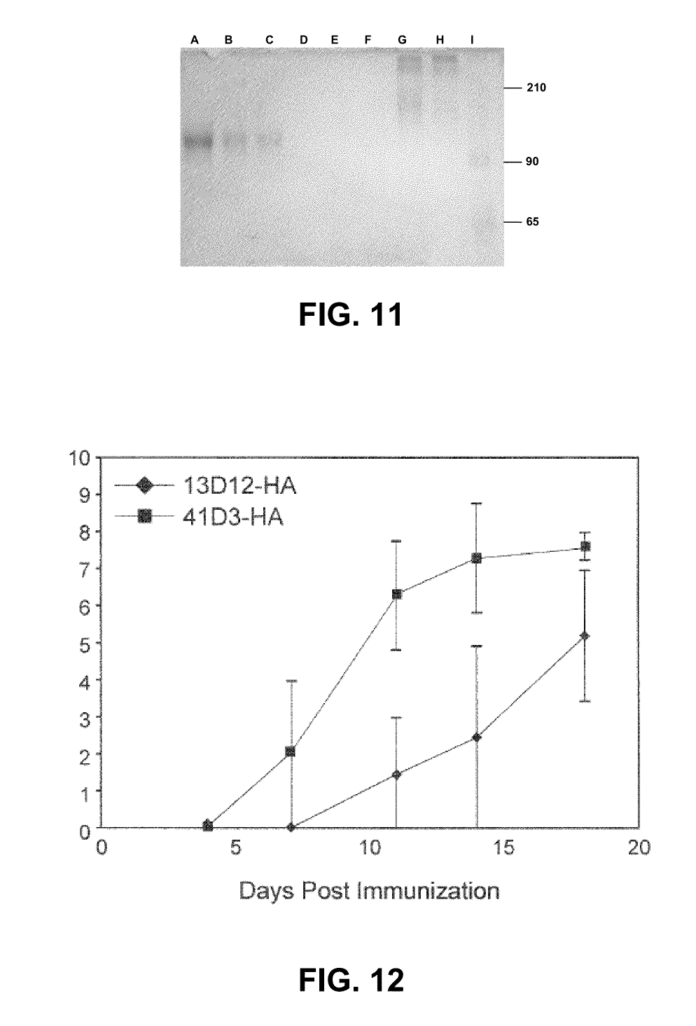

Coupling of saporin and antibody 41D3 is verified via SDS-PAGE followed by Coomassie blue staining and by analysis of uptake of the coupling products by primary alveolar macrophages. Coomassie blue staining of an SDS-PAGE shows a clear, upwards shift after conjugation, indicating an increased size, which confirms that part of the antibodies are coupled with saporin. The uncoupled proteins are clearly removed after dialysis. FIG. 11 shows SDS-PAGE and Coomassie blue staining of different samples taken during the antibody-saporin conjugation protocol. Lane I: marker, lane A: original antibody, lane B: SPDP treated antibody, lane C: SPDP treated antibody after PD-10 desalting column, lane D: original saporin, lane E: SPDP and DTT treated saporin, lane F: SPDP and DTT treated saporin after PD-10 desalting column, lane G: saporin coupled with antibody, lane H: saporin coupled with antibody after dialysis, and finally lane J: saporin coupled with antibody in the presence of the disulfide-reducing agent beta-mercapto-ethanol in the loading dye. For both antibodies there is a clear shift towards a bigger protein, which confirms that part of the antibodies are coupled with saporin. The uncoupled proteins are clearly removed after dialysis.

Saporin is conjugated to the mAb 41D3, resulting in a conjugate composition herein. A control conjugate including a non-sialoadhesin recognizing antibody, mAb 13D12 and saporin is also generated.

The mAb 41D3/saporin conjugate and the control mAb 13D12/saporin conjugate are each separately incubated with sialoadhesin expressing primary porcine macrophages. After an appropriate time, the cells are immunostained and analyzed by confocal microscopy to determine whether the conjugate binds and is internalized into the cells. Incubation of cells with the mAb 41D3/saporin conjugate results in internalization of the conjugate, indicating that the 41D3 mAb is still functional to bind and stimulate internalization of the conjugate. In contrast, the mAb 41D3/saporin conjugate is observed not to be internalized.

### Example 11

Cytotoxic effects of the mAb 41D3/saporin conjugate and the mAb 13D12/saporin conjugate on primary porcine macrophages are tested. Macrophages are incubated with various concentrations of either the mAb 41D3/saporin conjugate or the mAb 13D12/saporin conjugate for various periods of time. An MTT assay is used to colorimetrically assay cell populations and differentiate living and dead cells.

The following table shows OD values as a function of time and the percentage of living cells as a function of conjugate concentration as a result of these treatments:

### Macrophages

OD values:

| | 0 | 10 | 30 | 100 | APOP | Levend |
|---|---|---|---|---|---|---|
| 41D3-Sap | 0.772 | 0.537 | 0.541 | | 0.401 | 2.644 |
| 13D12-Sap | 0.851 | 0.708 | 0.703 | 0.780 | 0.445 | 2.824 |
| Average | 0.389 | | | | 0.423 | |

% Living cells

| | 0 | 10⁻⁷ | 0.3*10⁻⁶ | 10⁻⁶ |
|---|---|---|---|---|
| 41D3-Sap | 89.8 | 29.3 | 30.4 | |
| 13D12-Sap | 110.2 | 73.4 | 72.1 | 91.9 |

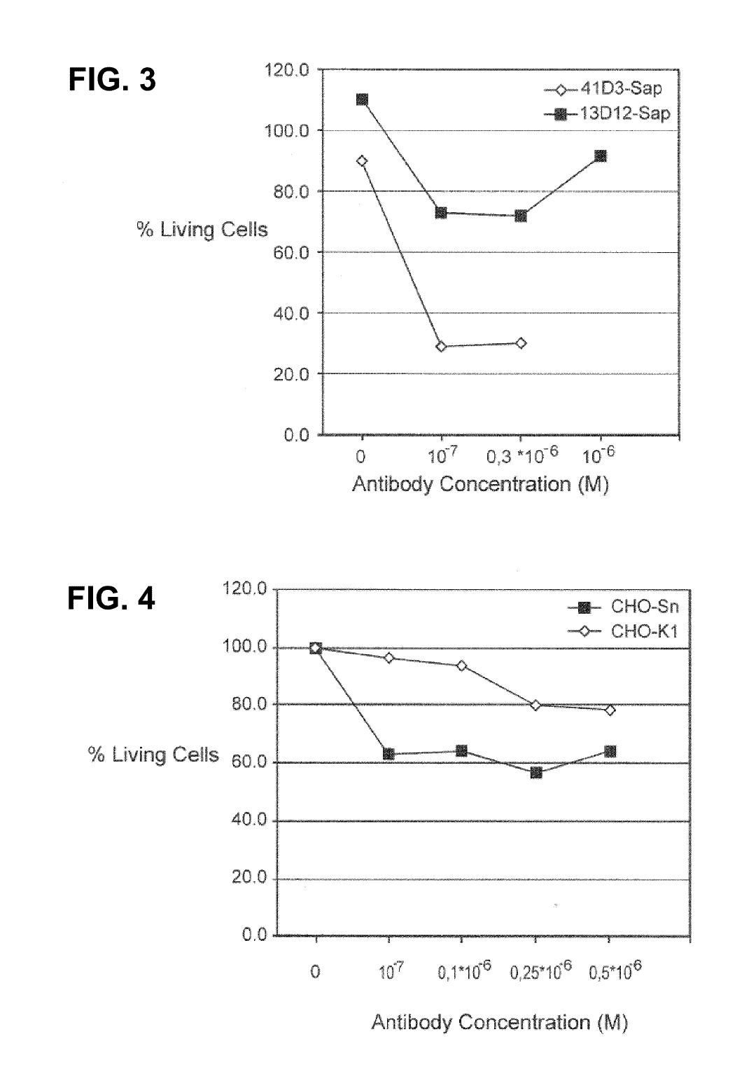

FIG. 3 depicts the percentage of living cells as a function of conjugate concentration in graphical form.

### Example 12

A mAb 41D3/saporin conjugate is incubated with various cells to assess the effect of the cytotoxic agent saporin. CHO cells that express recombinant sialoadhesin and CHO cells do not express sialoadhesin are each incubated with various amounts of the mAb 41D3/saporin conjugate and effects on cell viability are measured at various times following addition of the conjugate. An MTT assay is used to colorimetrically assay cell populations and differentiate living and dead cells.

The following table shows OD values as a function of time and the percentage of living cells as a function of conjugate concentration as a result of these treatments:

### CHO (41D3-Sap)

OD values:

| | 0 | 6.25 | 12.5 | 25 | 50 |
|---|---|---|---|---|---|
| CHO-K1 | 3.475 | 3.379 | 3.299 | 2.873 | 2.822 |
| CHO-Sn | 2.054 | 1.45 | 1.474 | 1.354 | 1.47 |

% Living cells

| | 0 | 10⁻⁷ | 0.1*10⁻⁶ | 0.25*10⁻⁶ | 0.5*10⁻⁶ |
|---|---|---|---|---|---|
| CHO-K1 | 100.0 | 96.9 | 94.2 | 80.3 | 78.6 |
| CHO-Sn | 100.0 | 63.0 | 64.4 | 57.1 | 64.2 |

FIG. 4 depicts the percentage of living cells as a function of conjugate concentration in graphical form. CHO-Sn indicates CHO cells expressing sialoadhesin. CHO-K1 indicates CHO cells which do not express sialoadhesin.

In Vivo Treatment of Pigs with Saporin-41D3 Immunotoxin

Pigs are injected intramuscularly with 0.1 or 1 mg saporin-41D3 conjugate in 1 ml of PBS/kg body weight, either as a single dose, or divided in two doses injected with an interval of 6 hours. Four pigs are used for each saporin-41D3 conjugate condition, four control pigs injected with PBS alone; twenty pigs in total. The pigs are euthanized 24 hours after the first injection and the local, draining lymph nodes are collected analyzed.

### Flow Cytometry Analysis of Lymph Node Immune Cells

Changes in the immune cell population of the lymph nodes are analyzed by flow cytometry. Total immune cells are prepared from lymph nodes by mechanical dissociation or collagenase D digestion. For mechanical dissociation, lymph node samples are dissociated with needles and filtered on a 40 micron pore size nylon filter. Cells are collected into a 50-ml conical tube and washed twice in RPMI. For collagenase D digestion, lymph nodes are incubated for 1 hour at 37° C. in RPMI with 1 mg collagenase D (Sigma)/ml. Cell suspensions are then filtered through a 40 micron pore size nylon filter and collected in RPMI with 30% FBS. After centrifugation for 15 minutes at 400×g, cells are washed three times with RPMI with 5% FBS. For erythrocyte lysis, cells are incubated for 5 minutes in 5 ml lysis solution (NH4Cl [0.15 M], KHC03 [1 mM], Na²⁺EDTA [0.1 mM]) and washed three times in RPMI with 5% FBS. Monocyte/macrophage cells are identified with FITC-labeled SWC3 specific mAb 74-22-15. Sialoadhesin expressing macrophages are stained with biotinylated mAb 41D3, followed by FITC labeled streptavidin. Total T-cells are quantified by staining with a FITC-labeled CD3-specific mAb, while subpopulations of T-cells are quantified by staining with either FITC-labeled CD4 or CD8. B cells are identified with a FITC-labeled mouse monoclonal anti-pig IgM antibody (Clone M160).

### Analysis of Lymph Node Micro-Anatomy

Samples of lymph nodes are fixed in a phosphate-buffered 3.5% formaldehyde solution for 24 hours. After fixation, the samples are embedded in paraffin using an automated system (Shandon Citadel Tissue Processor, Cheshire, UK). Sections of 8 microns in thickness are made, deparaffinized in xylene, rehydrated in descending grades of alcohol, stained, dehydrated in ascending grades of alcohol and xylene, and mounted on slides with DPX. Hematoxylin-eosin staining is done to analyze the morphology and micro-anatomy of the lymph nodes of treated and untreated pigs.

### Immunohistochemical Analysis

Samples from the draining lymph nodes are embedded in methylcellulose medium and frozen at −70° C. Cryostat sections (5 to 8 microns in thickness) are made and fixed in acetone for 20 minutes at −20° C. Sections are stained with one or more of the following:
(1) FITC-labeled goat-anti-mouse IgG antibodies to detect the injected immunotoxin;
(2) biotinylated mAb 41D3 followed by FITC-labeled streptavidin to allow quantification of sialoadhesin expressing cells and evaluate the effect of the immunotoxin on the numbers of sialoadhesin expressing cells;
(3) biotinylated antibody 74-22-15, an anti-SWC3 antibody which stains all monocytes and macrophages, not only the sialoadhesin expressing cells, followed by FITC-labeled streptavidin to assess the effect of the toxin on all cells of the monocyte macrophage lineage; and
(4) biotinylated mAb 41D3 followed by FITC-labeled streptavidin together with a staining with a goat polyclonal antibody specific for activated caspase-3 (apoptosis marker) followed by TexasRed-labeled rabbit-anti-goat antibodies to quantify the total level of apoptosis and the number of apoptotic sialoadhesin expressing macrophages in the draining local lymph nodes of saporin-41D3 conjugate immunotoxin treated pigs.

In vivo administration of a sialoadhesin binding moiety/cytotoxic agent conjugate, e.g., a saporin-41D3 conjugate allows for assessment of the in vivo functionality of a sialoadhesin specific immunotoxin and assessment of the capacity of the conjugate to selectively kill sialoadhesin expressing macrophages in lymph nodes. Both in vitro and in vivo assays provide information on the dose and manner of administration that is optimal for depletion of sialoadhesin expressing macrophages in lymph nodes. Depletion of sialoadhesin expressing macrophages may have utility in treatment of specific diseases that involve macrophages, e.g., rheumatoid arthritis, inflammatory skin diseases, persistent infections and others.

### Example 13

### Patients

Synovial membrane biopsies were obtained by needle arthroscopy of eight patients with CCP positive undifferentiated arthritis (UA CCP), 21 patients with early rheumatoid arthritis (ERA) and 13 patients with methotrexate resistant RA (Mtx res RA). Synovial membrane biopsies of 15 non-diseased (control) patients were used as controls. This study was approved by the local ethical committees and informed consents were signed by the patients.

### Anti-Sn Antibody Production and Coupling to Methotrexate

Monoclonal Abs were generated in rat by immunization with a soluble extracellular part (AA 20-1339) of mouse Sn (Synaptic Systems, Germany). Lymph nodes were isolated and fused with myeloma cells to generate a hybridoma cell line. Clones were tested for their ability to bind the soluble mSn and mSn expressed on CHO cells and subsequently subcloned twice to guarantee their monoclonal nature. The hybridoma for the isotype control was purchased at DSHB, Iowa and treated similar to the rat anti-mSn Ab.

Hybridomas were grown to confluency in DMEM 10% IgG depleted FCS, 1% P/S and medium was harvested up to three times.

The Ab was purified from the medium on a protein G column, protein containing fractions were pooled and either dialyzed to PBS or directly coupled with MTX.

Activated MTX (in dimethylformamide) was coupled to the rat anti-mSn Ab in a 20 to 1 ratio. Coupling of MTX to the rat anti-mSn Ab is done by an active ester intermediate. MTX (45 mg/ml), N-hydrosuccinimide (NHS) (24 mg/ml) and dicyclohexylcarbodiimide (DCC) (42 mg/ml) are dissolved in dimethylformamide (DMF). 1 ml of MTX is mixed with 0.5 ml of NHS and 0.5 ml of DCC. The solution is rotated in the dark at room temperature for 1 hour and subsequently at 4° C. for 18 hours. During this step and activated ester is formed, which will form a stable covalent amide bond with the Lys residues in the rat anti-mSn Ab. After activation of MTX the precipitate is removed by centrifugation and the liquid is stored at −20° C. in a dry environment. Activated MTX is mixed with Ab in a 20-fold molar excess and rotated at 4° C. for 4 hours. Uncoupled MTX is removed by gel permeation. Protein concentrations were measured at 280 nm, the MTX content at 370 nm.

### CIA Induction, Treatments and Evaluation in Mice

Eight-week-old DBA/1 Rj (H-2q background) mice were obtained from Janvier, France. Mice were immunized intradermally at the base of the tail with 200 μg of chicken type II collagen (CII) (Morwell Diagnostics GmbH, Zurich, Switzerland) (in 0.1 M acetic acid) emulsified in Incomplete Freund's Adjuvant+mycobacterium Tuberculosis H37RA (150 μg/mouse) (Difco, Lawrence, Kans., USA). Twenty-one days later, mice were re-challenged with an injection of CII in Incomplete Freund's Adjuvant.

Mice were treated intraperitoneally twice a week from day 14 after induction on until sacrifice. Twelve mice were treated per condition. Treatments consisted out of PBS as a negative control, a high dose of MTX (Sigma) (35 mg/kg=700 μg/mouse), isotype antibody conjugated with MTX (iso-MTX; 200 μg/mouse, equivalent with 4 μg/mouse MTX or 0.2 mg/kg) and the anti-Sn antibody conjugated with MTX (ab-MTX; 200 μg/mouse, equivalent with 4 μg/mouse MTX or 0.2 mg/kg) in PBS.

From Day 21, mice were monitored for clinical symptoms of arthritis until the day of sacrifice (Day 42). Clinical severity was graded as follows: 0=normal; 0.5=erythema and edema in only one digit; 1=erythema and mild edema of the footpad, or ankle or two to five digits; 2=erythema and moderate edema of two joints (footpad, ankle, two to five digits); 3=erythema and severe edema of the entire paw; 4=reduced swelling and deformation leading to incapacitated limb. The individual mouse arthritic score was obtained by summing the scores recorded for each limb. Clinical evaluations were performed by two investigators unaware of mouse identity and the mean of both scores was calculated.

### ELISA

The serum concentrations of Ab-MTX after injection were measured in an ELISA setup. The rat Ab was captured from solution by an anti-rat Ab (goat anti-rat-AF488 A11006, Invitrogen) and detected with an HRP labeled anti-rat Ab (goat anti-rat-HRP 112-035-167, Jackson ImmunoResearch). Concentrations were presented as concentrations with the concentration 5 minute after injected set as 100%.

### Isolation of Alveolar Mouse Macrophages and Detection of Internalized Sn

Alveolar macrophages were isolated from BALB/c mice by lavage with 1% versene in PBS. CHO or macrophages were coated on glass coverslips using poly-L-lysine for 1 hour. Next the cells are incubated with rat anti-mSn, rat anti-mSn-MTX and isotype controls at 1 μg/ml both at 4° C. and 37° C. After 90 minutes the cells were fixed with 4% paraformaldehyde and permeabilized with 0.5% saponin. Surface bound or internalized rat anti-mSn was detected with an Alexa Fluor 488 labeled goat anti-rat Ab.

### Immunohistochemistry and Immunofluorescence on Human Synovial Tissue

IHC and IF were performed on frozen sections of synovial biopts after fixation in aceton. The primary mouse anti-human Sn antibody (clone 7D2; Santa Cruz) was detected using the universal LSAB kit (Dako) containing an anti-mouse biotinylated antibody and streptavidin-HRP. Chromogen deposition was obtained by the use of AEC substrate (Dako). The level of expression was independently scored by two observers. A semi-quantitative four-point scale was used with zero representing the lowest and three representing the highest level of expression. The scoring was calibrated for synovial lining and sublining tissue separately by examining a representative number of samples. Each section was scored twice per observer and absolute intraclass correlation coefficients were all above 0.85. Multiple comparisons were performed for Sn expression in the lining and sublining synovial layer between the four groups.

For colocalization of Sn, CD209 and CD68, aceton fixed frozen biopts of three patients per group were blocked with 2% goat and 2% donkey serum in 5% BSA in PBS. First mouse anti-CD209 (BD biosciences) antibody was applied and detected with Cy3-labeled anti-mouse (Jackson Immuno Research). Free antigen-binding moieties were blocked by the addition of 10% mouse serum. Mouse IgG1 anti-Sn and biotin-labeled mouse IgG2b anti-CD68 (Immunosource) were then added and respectively detected by Alexa Fluor488-labeled anti-mouse IgG1 and Alexa Fluor647-labeled streptavidin (both Invitrogen). Slides were mounted with Prolong anti-fade mounting medium with DAPI (Invitrogen). Negative control stainings were performed by using isotype controls or omission of one primary antibody. Colocalization was determined by confocal microscopy (Leica TCS LSI).

### Immunohistochemistry and Immunofluorescence on Mouse Tissue

IHC was performed on aceton fixed frozen sections of non-decalcified mouse knee, obtained by the use of the Cryojane tape transfer system (Leica). The primary rat anti-mouse Sn antibody (clone 3D6.112; AbdSerotec) was detected using containing an anti-rat biotinylated antibody (eBioscience), streptavidin-HRP from the universal LSAB⁺ kit (Dako) and visualized by the use of AEC substrate (Dako).

For the detection of the injected anti-Sn antibody conjugated to MTX, aceton fixed frozen biopts of spleen or knee were analyzed. Mouse knees were decalcified by overnight shaking in 0.5 M EDTA pH 7.5 at 4° C. After 1 hour incubation in 10% sucrose, knees were subsequently incubated overnight in 15% sucrose and 15% sucrose/50% tissue teck (Jung) before sectioning. Alexa Fluor546 anti-rat (Invitrogen) was applied for detection of the primary injected antibody. Slides were mounted with prolong anti-fade mounting medium with DAPI (Invitrogen) and visualized using wide field microscopy (Leica TCS SP5 II).

### Flow Cytometry

Paired peripheral blood—synovial fluid samples were obtained from RA and SpA patients with an active knee synovitis. Patients were diagnosed using the ACR 2010 criteria for RA or the ASAS 2010 criteria for axial and peripheral SpA. Additionally, healthy control peripheral blood samples were collected. The study was approved by the local Ethical Committee of the Ghent University hospital. PBMC were isolated from heparinized whole blood by density gradient centrifugation using Histopaque-1077 (Sigma-Aldrich). Likewise, SFMC were extracted from synovial fluid collected in anticoagulant EDTA-coated tubes.

Multi-color flow cytometry was performed using the following antibodies: CD14 V500 (BD; clone M5E2), CD16 PE-Cy7 (eBioscience; clone CB16), HLA-DR APC-eFluor 780 (eBioscience; clone LN3), CD56 V450 (BD; clone B159); Sn PE (Santa Cruz; clone 7D2); mIgG1 isotype (eBioscience; clone P3.6.2.8.1). 7-AAD (BD) was used to distinguish alive from dead cells. Samples were acquired on a BD FACS Canto II and analyzed using FlowJo software (TreeStar). A geometrical mean was calculated by substracting PE signal from the isotype condition from the PE signal derived from the Sn PE antibody.

### Statistical Analysis

Absolute intraclass correlation coefficients were calculated for quality analysis of the semi-quantitative scoring of Sn IHC staining. For analysis of longitudinal clinical scores, mixed model analysis with random intercept was used. Differences in clinical (day of onset and day 42 score) and histological data between the treatment groups and semi-quantitative scores of patient groups were assessed by Kruskal Wallis tests followed by Mann-Whitney-U test with correction using the Holm procedure. Fisher's Exact test was applied to analyze arthritis frequencies. All analyses were performed using SPSS 19.0 statistical software (Chicago, Ill., USA).

### Increase of Slaloadhesin Expressing Cells Early in Disease and Throughout Rheumatoid Arthritis Disease Progression in Synovial Tissue

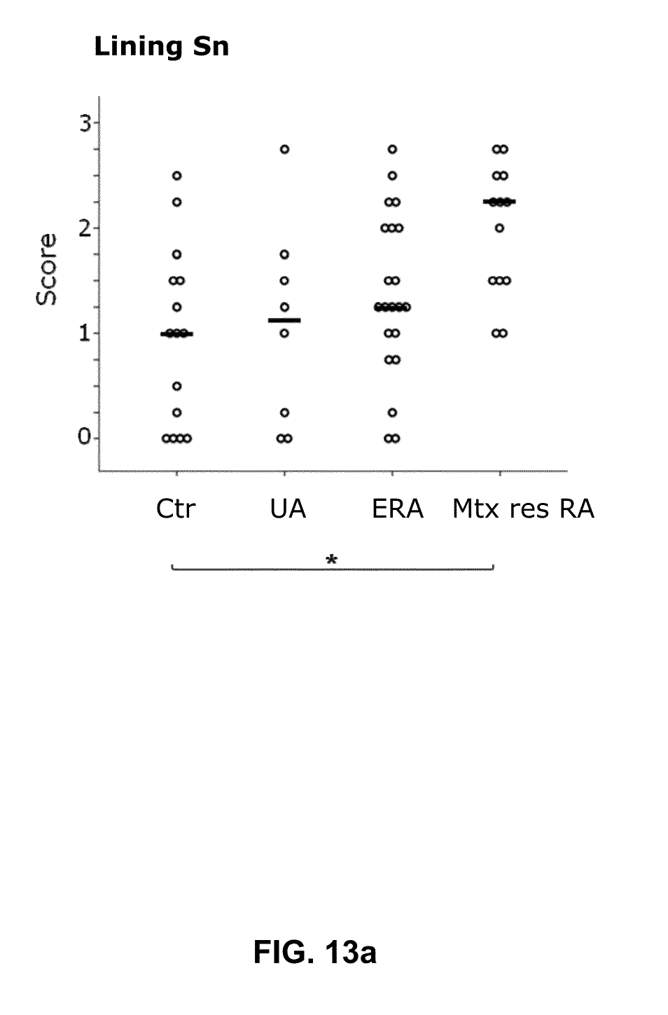

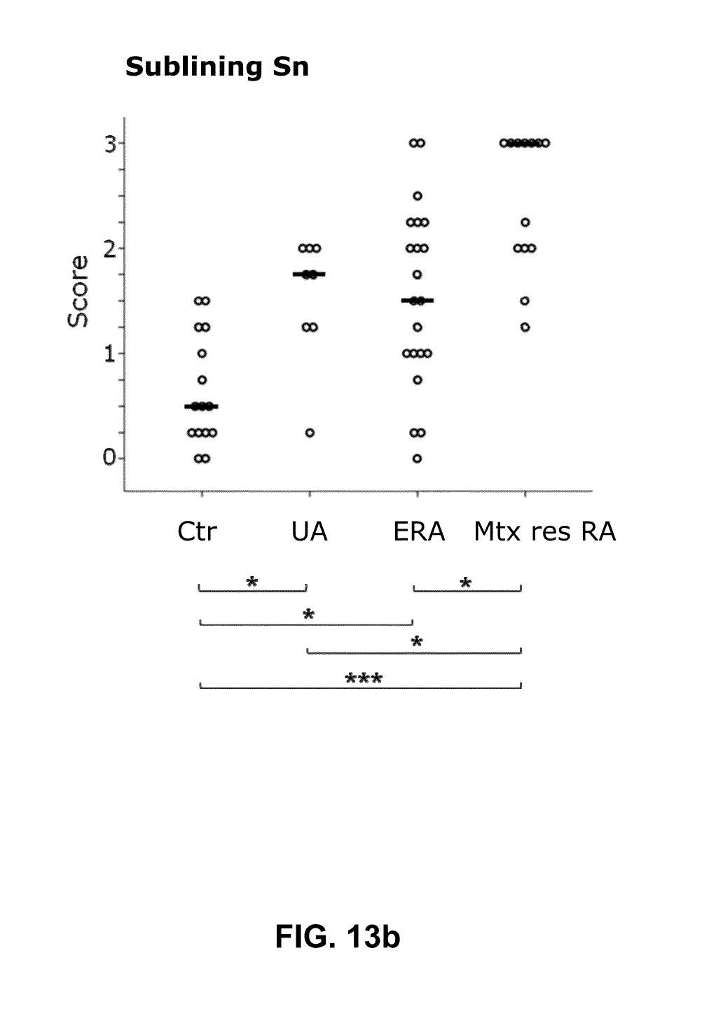

Synovial membrane biopsies from non-diseased controls, patients with anti-CCP positive undifferentiated arthritis (UA CCP⁺), early rheumatoid arthritis (ERA) and methotrexate resistant RA (Mtx res RA) were analyzed for their Sn expression by IHC. Lining and sublining synovial layers were scored separately. The trend for an increased Sn expression in the lining synovial layer (FIG. 13a) according to disease severity, is more pronounced and significantly different between groups in the sublining layer (FIG. 13b). Sn expression in the sublining is already significantly increased between the control group and the group with UA patients. Moreover, Sn levels in the sublining are furthermore significantly elevated between patients with ERA and patients with methotrexate resistant RA. To conclude, Sn expression is clearly up-regulated in early stage, still undifferentiated, arthritis as opposed to a few Sn expressing cells in healthy synovial tissue. Furthermore, aggressive late-stage methotrexate resistant RA displays clearly more Sn expressing cells than early RA.

Sn expressing cells were also shown to be present in synovial tissue of patients with osteoarthritis (OA), spondyloarthritis (SpA), systemic lupus erythematosus (SLE) and psoriatic arthritis (PsA).

### Sn is Mainly Expressed on CD209⁺ CD68⁺ Macrophages in Synovial Tissue

Triple immunofluorescence, analyzed by confocal microscopy clearly shows colocalization of Sn expression, mainly on the same cells as CD209 (DC-SIGN) and CD68 expression. In addition, some Sn expressing cells were single positive for CD209 or CD68. These findings were consistent in all groups (control, UA, ERA, MTX resistant RA, SpA), of which synovial biopts of three patients each group were stained. To conclude, we have shown that sialoadhesin is mainly expressed on CD209⁺ CD68⁺ “Inflammatory MMP producing macrophages” as Van Lent et al. [19] have described in 2003 them to be the macrophages responsible for the matrix tissue degrading MMP1 production.

### High Sn Expression on Monocytes, Peripherally in Blood and Locally in Synovial Fluid

Sialoadhesin was found to be highly expressed in blood of controls and patients on the main monocytic populations, being CD14^{+high} CD16⁻ “classical monocytes” and CD14^{+high} CD16⁺ “intermediate monocytes” as compared to the sialoadhesin negative population of NK cells. Moreover, monocytes in general, but the intermediate monocytes more in particular accumulate massively in the synovial fluid in the inflamed knee joints of patients with RA or SpA. Remarkably Sn expression is dramatically increased on the CD14^{+low} CD16⁻ “non-classical monocytes” locally in synovial fluid as compared to peripheral blood of the same patient.

### Sn is Highly Abundant in the Inflamed Mouse Knee During Collagen Induced Arthritis (CIA)

Sialoadhesin is expressed in knees of healthy mice in the bone marrow, in the thin synovial cell layer, between muscle cells and surrounding the knee. However, its expression was found to be increased in inflamed knee joints of mice during the experimental mouse model for RA, Collagen Induced Arthritis. Especially the hyperproliferated synovium contained many Sn⁺ cells, next to the bone marrow, between muscle cells and surrounding the knee also seen in healthy mice.

Monoclonal Anti-Sn Antibody Conjugated with Methotrexate Detected in Mouse Spleen and Mouse Knee After Injection.

200 micrograms rat monoclonal anti-Sn antibody conjugated to methotrexate (ab-MTX) was intravenously or intraperitoneally injected in mice and detected at different time points in mouse spleen and knee by the use of a fluorescently labeled anti-rat antibody. The injected antibody was detected in spleen at any time point analyzed being from 30 minutes until 7 days after the injection. The methotrexate coupled antibody was also able to reach the knee in a healthy mouse both via IV or IP.

Monoclonal Anti-Sn Antibody Conjugated with Methotrexate Kinetics in Mouse Blood

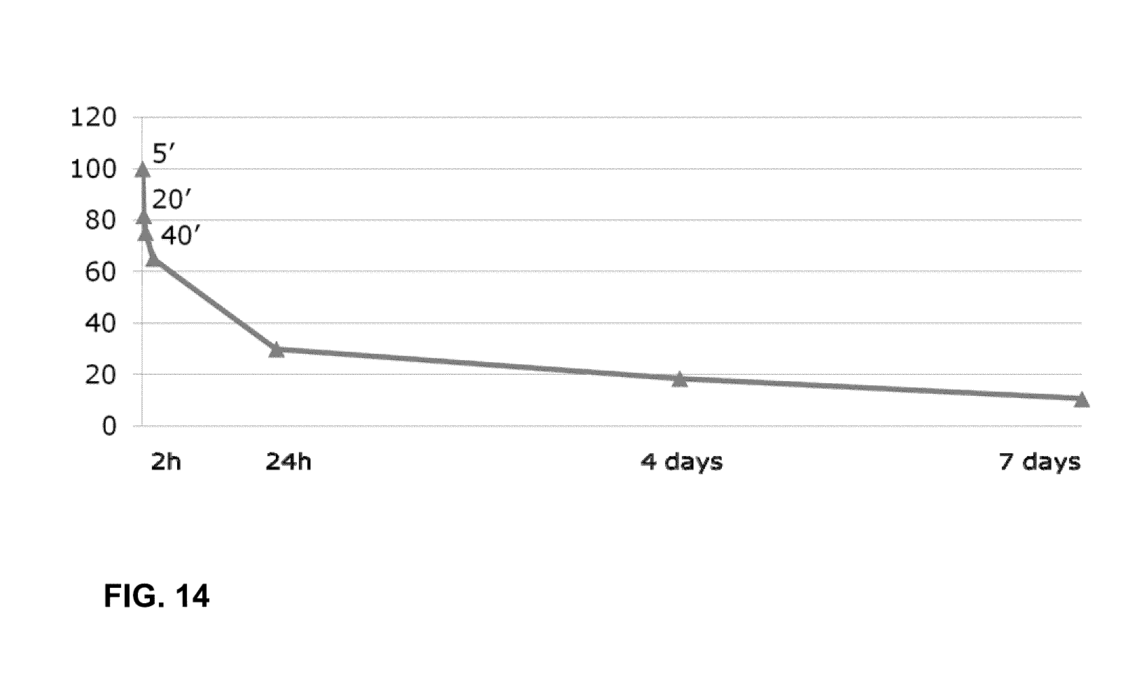

Ab-MTX, ab or iso-MTX were detected by ELISA at time points 5, 20, 40, 120 minutes, 24 u, 4 days and 7 days after injection of 200 micrograms of the corresponding antibody or conjugate in healthy mice. All three products tested, showed the same kinetic profile in the blood (FIG. 14). With ab-MTX being detected at an average of 65% after 2 hours, at 30% after 24 hours, at 19% after 4 days and at 11% after 7 days compared with 100% set at 5 minutes after injection. Based upon these results the mice were injected twice per week in the CIA experiment, but this might be less as the conjugate was still detected after 1 week.

Monoclonal Anti-Sn Antibody Conjugated with Methotrexate is Internalized in Primary Mouse Macrophages

Primary alveolar mouse macrophages and CHO cells expressing mSn internalize both the Ab and the Ab-MTX when incubated at 37° C. At 4° C., only surface staining was observed.

Monoclonal Anti-Sn Antibody Conjugated with Low Dose of Methotrexate (MTX) Prevents Symptoms of Arthritis During Collagen Induced Arthritis in Mice

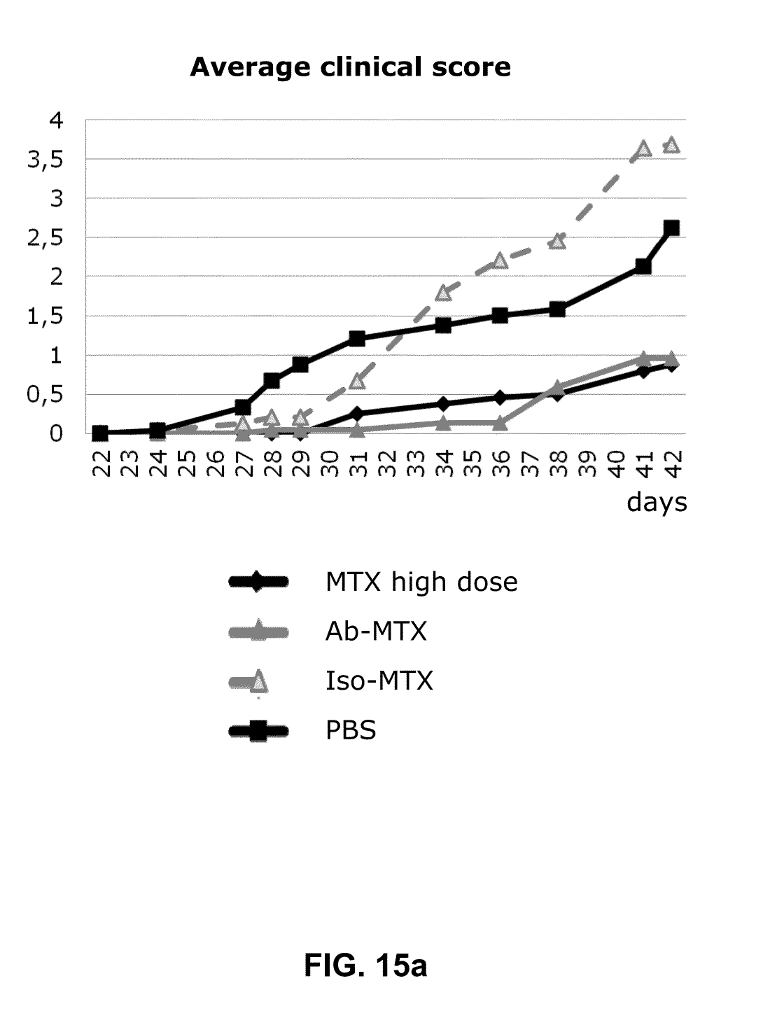

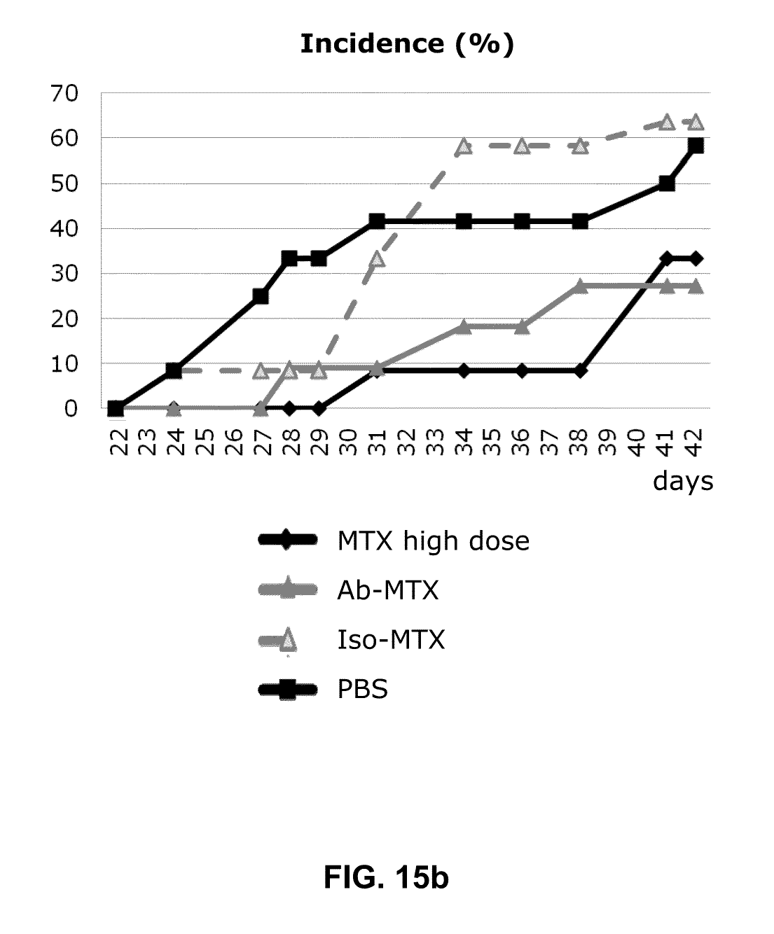

DBA/1 mice were immunized with collagen to initiate collagen induced arthritis. 14 days after immunization mice were treated twice per week with the following conditions: PBS, high dose of MTX (35 mg/kg), negative control isotype conjugated with MTX (iso-MTX) and the anti-Sn antibody conjugated with MTX (ab-MTX; equivalent of 0.2 mg/kg MTX). All mice were clinically scored at least three times a week until 42 days after immunization. The high dose of MTX delayed and prevented symptoms of arthritis as expected in most mice as 33% became arthritic (FIG. 15b) with an average clinical score (FIG. 15a) of 0.875, compared to the PBS treated group with an incidence of 58% and an average clinical score of 2,625. Importantly, the ab-MTX treatment prevented the clinical signs of arthritis as good, as only 3/11 (27%) mice became ill with an average score of 0.955, as compared to the iso-MTX group which had an incidence of 7/11 (64%) with an average clinical score of 3,682. Longitudinal clinical scores (FIG. 15a), determined by mixed model analysis, were significantly (p<0.0001) different between PBS and MTX; PBS and ab-MTX; iso-MTX and ab-MTX; iso-MTX and MTX indicating a significant effect of the treatment over time. However, no significant difference was found between MTX and ab-MTX indicating the same anti-arthritic effects of the low dose coupled to anti-Sn and the 175 times higher dose of soluble MTX.

### Sialoadhesin Protein and Nucleotide Sequences

SEQ ID NO:5 is a protein sequence for pig sialoadhesin identified by GenBank Accession number AF509585.1. SEQ ID NO:5:

SEQ ID NO:6 is a nucleotide sequence encoding pig sialoadhesin identified by GenBank Accession number AF509585.1. SEQ ID NO:6:

SEQ ID NO:7 is a protein sequence for mouse sialoadhesin identified by GenBank Accession number NM_{—}011426. SEQ ID NO:7

SEQ ID NO:8 is a nucleotide sequence encoding mouse sialoadhesin identified by GenBank Accession number NM_{—}011426. SEQ ID NO:8:

SEQ ID NO:9 is a protein sequence for human sialoadhesin identified by GenBank Accession number NM_{—}023068. SEQ ID NO:9

SEQ ID NO:10 is a nucleotide sequence encoding mouse sialoadhesin identified by GenBank Accession number NM_{—}023068. SEQ ID NO: 10:

The patents and publications mentioned herein are incorporated herein by reference. Protein and polynucleotides identified by a database accession number are incorporated herein by reference in their entirety.

What is claimed is:

1. A method of delivering methotrexate, or a prodrug, derivative, homolog or analog thereof to a cell, the method comprising:
contacting a cell expressing sialoadhesin with a conjugate, the conjugate comprising a sialoadhesin binding moiety and methotrexate, or a prodrug, derivative, homolog or analog thereof,
wherein the sialoadhesin binding moiety binds to the sialoadhesin expressed by the cell, thereby delivering methotrexate, or a prodrug, derivative, homolog or analog thereof, to the cell.

2. The method according to claim 1, wherein the cell is selected from the group consisting of a monocyte, a monocyte cell line, a macrophage, and a macrophage cell line.

3. The method according to claim 1, wherein the sialoadhesin binding moiety is an antibody, or a fragment thereof, a sialoadhesin ligand or a small molecule.

4. The method according to claim 1, wherein the sialoadhesin binding moiety is a monoclonal antibody or a fragment thereof, or a domain antibody.

5. The method according to claim 1, to treat a subject diagnosed as suffering from an autoimmune disease.

6. The method according to claim 1, for treating chronic inflammatory arthritides selected from the group consisting of: rheumatoid arthritis (RA), spondyloarthritis (SpA), psoriatic arthritis (PsA), forms of undifferentiated arthritis, and related conditions.

7. The method according to claim 6, comprising administering a therapeutically effective amount of a sialoadhesin binding moiety conjugated to methotrexate to the subject, wherein methotrexate is delivered to a sialoadhesin expressing cell in the subject, thereby treating the pathological condition.

8. The method according to claim 6, applied to a methotrexate-resistant subject.

9. The method according to claim 1, wherein the cell is in vitro.

10. The method according to claim 1, wherein the cell is in vivo.

11. The method according to claim 1, wherein the cell is a mammalian cell.

12. A method to target methotrexate to a sialoadhesin-expressing cell by administering to a subject a composition comprising a sialoadhesin binding moiety and methotrexate, or a prodrug, derivative, homolog or analog thereof.

13. The method according to claim 12, wherein the sialoadhesin-expressing cell is selected from the group consisting of a monocyte, a monocyte cell line, a macrophage, and a macrophage cell line.

14. The method according to claim 12, wherein the sialoadhesin binding moiety is an antibody, or a fragment thereof, a sialoadhesin ligand or a small molecule.

15. A method for treating chronic inflammatory arthritides selected from the group consisting of: rheumatoid arthritis (RA), spondyloarthritis (SpA), psoriatic arthritis (PsA), forms of undifferentiated arthritis, and related conditions, the method comprising:
administering a therapeutically effective amount of a sialoadhesin binding moiety conjugated to methotrexate, or a prodrug, derivative, homolog or analog thereof, to the subject, and wherein methotrexate, or a prodrug, derivative, homolog or analog thereof, is delivered to a sialoadhesin expressing cell in the subject, thereby treating the chronic inflammatory arthritides.

16. The method according to claim 15, wherein the sialoadhesin-expressing cell is selected from the group consisting of a monocyte, a monocyte cell line, a macrophage and a macrophage cell line.

17. The method according to claim 15, wherein the sialoadhesin binding moiety is an antibody, or a fragment thereof, a sialoadhesin ligand, or a small molecule.

18. A composition comprising a sialoadhesin binding moiety conjugated to methotrexate, or a prodrug, derivative, homolog or analog thereof, wherein the sialoadhesin binding moiety is an antibody, or a fragment thereof, a sialoadhesin ligand or a small molecule.
